(19) United States
(12) Patent Application Publication
(10) Pub. No.: US 2022/0348564 A1
(43) Pub. Date: Nov. 3, 2022

(54) PHENYL-[1,3]DIOXOLO[4,5-C]PYRIDINYL-PHENYL-, PHENYL-[1,3]DIOXOLO[4,5-C]PYRIDINYL-HETEROARYL-, OR PHENYL-(1,3)DIOXO[4,5-C]PYRIDINYL-PIPERIDINYL-METHYL-OXETANYLMETHYL-1H-BENZO[D]IMIDAZOLE-CARBOXYLIC ACID DERIVATIVES AND METHODS OF USING SAME

(71) Applicant: Eccogene (Shanghai) Co., Ltd., Shanghai (CN)

(72) Inventors: Zaifang REN, Shanghai (CN); Xuefeng SUN, Shanghai (CN); Jingye ZHOU, Shanghai (CN)

(21) Appl. No.: 17/702,835

(22) Filed: Mar. 24, 2022

(30) Foreign Application Priority Data
Mar. 24, 2021 (CN) PCT/CN2021/082781
Jul. 23, 2021 (CN) PCT/CN2021/108059
Aug. 27, 2021 (CN) PCT/CN2021/114985

(51) Int. Cl. C07D 405/14; C07D 491/056; C07D 417/14; C07D 493/08; C07D 471/08

(52) U.S. Cl. CPC C07D 405/14; C07D 491/056; C07D 417/14; C07D 493/08; C07D 471/08

(57) ABSTRACT

The application relates to a compound of Formula (Y) or Formula (A):

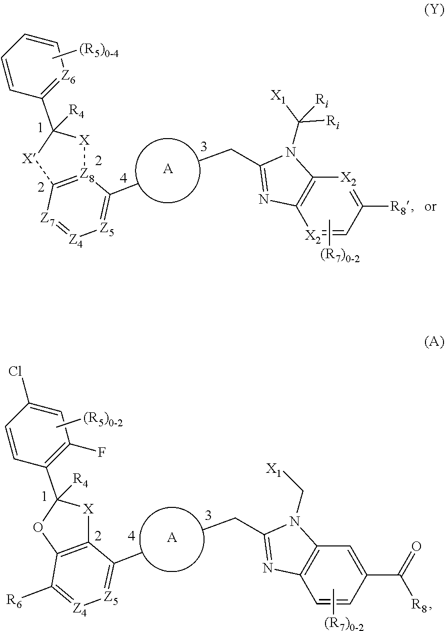

or a pharmaceutically acceptable salt, hydrate, solvate, prodrug, stereoisomer, or tautomer thereof, which modulates the activity of GLP-1 receptor, a pharmaceutical composition comprising a compound of Formula (I), and a method of treating or preventing a disease in which GLP-1 receptor plays a role.

### CROSS REFERENCE TO RELATED APPLICATIONS

This application claims priority to and the benefit of International Application No. PCT/CN2021/082781, filed Mar. 24, 2021, International Application No. PCT/CN2021/108059, filed Jul. 23, 2021, and International Application No. PCT/CN2021/114985, filed Aug. 27, 2021, the contents of each of which are incorporated herein by reference in their entireties.

### BACKGROUND

Glucagon-like peptide-1 (GLP-1) is an incretin of 30 or 31 amino acids, secreted from L cells in the small intestine. GLP-1 exerts a wide range of effects through the GLP-1 receptor, such as promotion of glucose dependent insulin secretion, inhibition of glucagon secretion, delay of gastric emptying, and suppression of feeding. Accordingly, GLP-1 analogs display potent effects in HbA1c reduction and weight loss, and have been developed as effective therapeutic agents for treatment of diabetes and obesity. GLP-1 analogs also demonstrate efficacy on improving cardiovascular outcomes and retaining renal functions in diabetic patients, thus providing therapeutic opportunities for a variety of metabolic disorders and related comorbidities. Recently, Liraglutide and Semaglutide treatment is shown to decrease liver fat and boost NASH resolution in clinical trials, suggesting potential utility for NASH. However, most of these GLP-1 analogs require an invasive subcutaneous administration. Semaglutide in specific formulation can be administrated via oral route, but still suffers from inconvenient dosing regimen and poor bioavailability. Improving metabolic stability and bioavailability of GLP-1 analogs is challenging, likely due to their peptidic nature.

Currently, there is no approved small molecule GLP-1 receptor agonist for the treatment of diabetes or other metabolic disorders where GLP-1 receptor plays a role. Thus, there is a need for small molecule GLP-1 receptor agonists as therapeutic options for the treatment of these disorders. The present application addresses the need.

### SUMMARY

The present application provides novel GLP-1 receptor ligands which are useful in the treatment of a disease or disorder in which GLP-1 receptor plays a role, such as those described herein, including but not limited to diabetes, obesity, overweight condition, hyperlipidemia, hypercholesteremia, hypertriglyceridemia, atherosclerosis, hypertension, stroke, coronary heart disease, congestive heart failure, cardiac arrhythmias, diabetic kidney disease, dementia, Parkinson's disease, Alzheimer's disease, and liver diseases such as nonalcoholic fatty liver disease (NAFLD) and nonalcoholic steatohepatitis (NASH).

A first aspect of the application relates to a compound of Formula (Y), (Y′), (A), or (I):

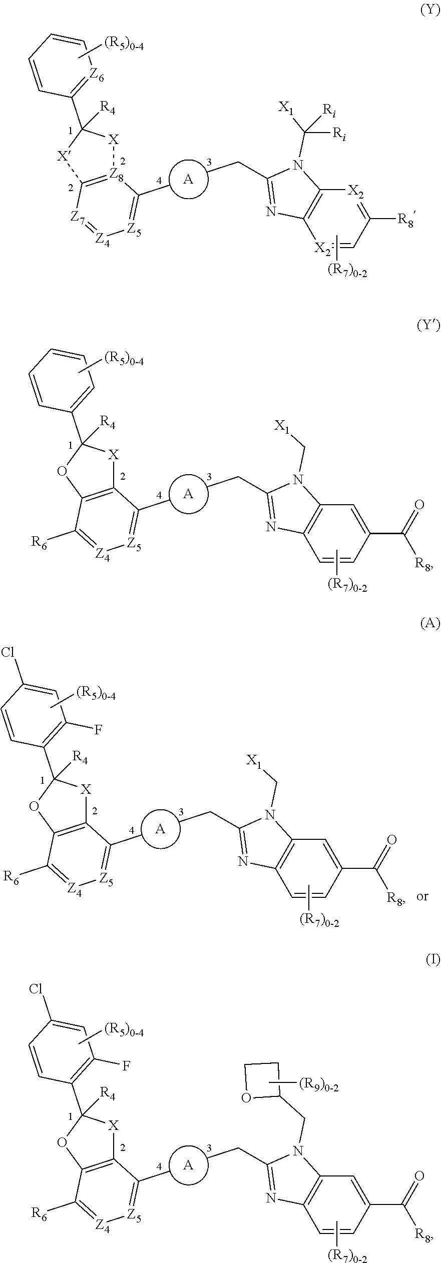

or a pharmaceutically acceptable salt, solvate, prodrug, stereoisomer, or tautomer thereof, wherein X₁, X₂, R₁, A, X, X′, Z₄, Z₅, Z₆, Z₇, Z₈, and R₄-R₉ are as described in detail below.

Another aspect of the application relates to a pharmaceutical composition comprising a compound of Formula (Y), (Y′), (A), or (I) or a compound described herein, or a pharmaceutically acceptable salt, solvate, prodrug, stereoisomer, or tautomer thereof, and a pharmaceutically acceptable diluent, excipient, or carrier.

Another aspect of the application relates to a method of treating or preventing a GLP-1 receptor-mediated disease or disorder (e.g., a disease or disorder in which GLP-1 receptor plays a role or which is associated with modulation of GLP-1 receptor), as described herein (e.g., diabetes, obesity, overweight condition, hyperlipidemia, hypercholesteremia, hypertriglyceridemia, atherosclerosis, hypertension, stroke, coronary heart disease, congestive heart failure, cardiac arrhythmias, diabetic kidney disease, dementia, Parkinson's disease, Alzheimer's disease, and liver diseases such as NAFLD and NASH). The method comprises administering to a subject in need of such a treatment a therapeutically effective amount of a compound of Formula (Y), (Y′), (A), or (I) or a compound described herein, or a pharmaceutically acceptable salt, solvate, prodrug, stereoisomer, or tautomer thereof, or a therapeutically effective amount of a pharmaceutical composition comprising a compound of Formula (Y), (Y′), (A), or (I) or a compound described herein, or a pharmaceutically acceptable salt, solvate, prodrug, stereoisomer, or tautomer thereof, and a pharmaceutically acceptable diluent, excipient, or carrier.

Another aspect of the application relates to a method of modulating (e.g., activating or stimulating) GLP-1 receptor. The method comprises administering to a subject in need of such modulation a therapeutically effective amount of a compound of Formula (Y), (Y′), (A), or (I) or a compound described herein, or a pharmaceutically acceptable salt, solvate, prodrug, stereoisomer, or tautomer thereof, or a therapeutically effective amount of a pharmaceutical composition comprising a compound of Formula (Y), (Y′), (A), or (I) or a compound described herein, or a pharmaceutically acceptable salt, solvate, prodrug, stereoisomer, or tautomer thereof, and a pharmaceutically acceptable diluent, excipient, or carrier.

Another aspect of the application relates to a compound of Formula (Y), (Y′), (A), or (I) or a compound described herein, or a pharmaceutically acceptable salt, solvate, prodrug, stereoisomer, or tautomer thereof, or a pharmaceutical composition comprising a compound of Formula (Y), (Y′), (A), or (I) or a compound described herein, or a pharmaceutically acceptable salt, solvate, prodrug, stereoisomer, or tautomer thereof, and a pharmaceutically acceptable diluent, excipient, or carrier, for use in a method of treating or preventing a GLP-1 receptor-mediated disease or disorder or of modulating (e.g., activating or stimulating) GLP-1 receptor.

Another aspect of the application relates to use of a compound of Formula (Y), (Y′), (A), or (I) or a compound described herein, or a pharmaceutically acceptable salt, solvate, prodrug, stereoisomer, or tautomer thereof, or a pharmaceutical composition comprising a compound of Formula (Y), (Y′), (A), or (I) or a compound described herein, or a pharmaceutically acceptable salt, solvate, prodrug, stereoisomer, or tautomer thereof, and a pharmaceutically acceptable diluent, excipient, or carrier, in the manufacture of a medicament for treating or preventing a GLP-1 receptor-mediated disease or disorder or for modulating (e.g., activating or stimulating) GLP-1 receptor.

The present application provides modulators (e.g., agonists) of GLP-1 receptor that are therapeutic agents in the treatment of diseases such as diabetes, obesity, metabolic diseases, cardiovascular diseases, liver diseases, NASH, kidney diseases, neurodegenerative diseases, and other diseases or disorders associated with the modulation of GLP-1 receptor.

The present application further provides compounds and compositions with an improved therapeutic profile (e.g., efficacy, pharmacodynamics, safety) relative to known GLP-1 receptor agonists and alternative routes of administration, toward the treatment of various types of diseases including diabetes, obesity, metabolic diseases, cardiovascular diseases, liver diseases, NASH, kidney diseases, neurodegenerative diseases, and other diseases associated with the modulation of GLP-1 receptor.

### DETAILED DESCRIPTION

### Compounds of the Application

The present application relates to compounds and compositions thereof that are capable of modulating the activity of GLP-1 receptor. The application features methods of treating, preventing, or ameliorating a disease or disorder in which GLP-1 receptor plays a role by administering to a subject in need thereof a therapeutically effective amount of a compound of the present application, or a pharmaceutically acceptable salt, solvate, prodrug, stereoisomer, or tautomer thereof. The compounds of the present application can be used in the treatment of a variety of GLP-1-mediated diseases and disorders by stimulating GLP-1 receptor. Activation or stimulation of GLP-1 receptor provides treatment, prevention, or amelioration of diseases including, but not limited to, diabetes, obesity, metabolic diseases, cardiovascular diseases, liver diseases, nonalcoholic steatohepatitis (NASH), and other diseases associated with the modulation of GLP-1 receptor.

In one aspect, a compound of Formula (Y) is described:

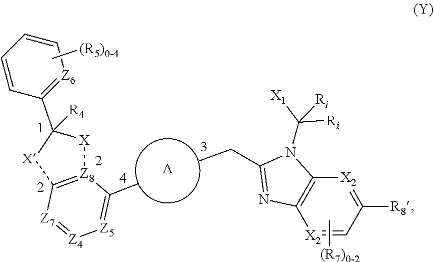

or a pharmaceutically acceptable salt, solvate, prodrug, stereoisomer, or tautomer thereof, wherein:
X₁ is H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S, wherein the carbocyclyl, heterocyclyl, aryl, or heteroaryl is optionally substituted with one or more substituents independently selected from C₁-C₄ alkyl, halo-C₁-C₄ alkyl, C₁-C₄ alkoxy, halo-C₁-C₄ alkoxy, halogen, OH, CN, and NH₂;
each Rᵢ is independently H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S; or two Rᵢ, together with the carbon atom to which they are attached, form a C₃-C₆ carbocyclyl or 4- to 6-membered heterocyclyl comprising 1 or 2 heteroatoms selected from N, O, and S; or one Rᵢ and X₁, together with the carbon atom to which they are attached, form a C₃-C₆ carbocyclyl or 4- to 6-membered heterocyclyl comprising one or two 5- or 6-membered rings and 1 to 4 heteroatoms selected from N, O, and S;
each X₂ is independently CR₇ or N;
Ring A is

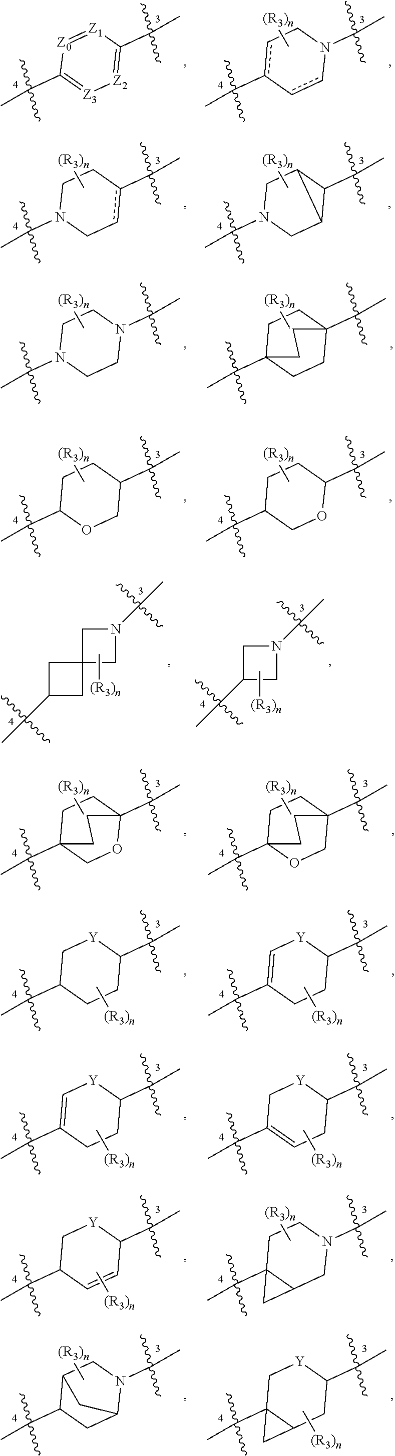

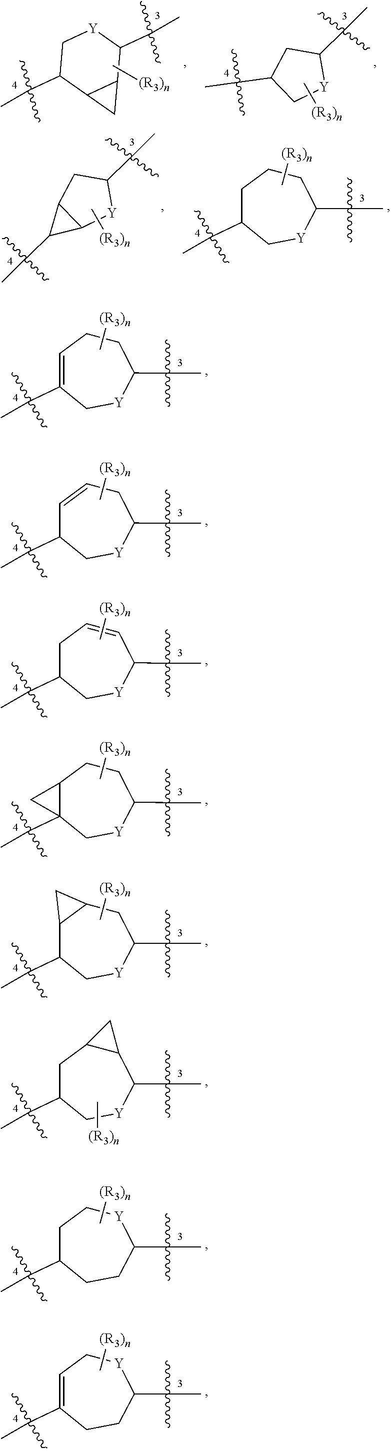

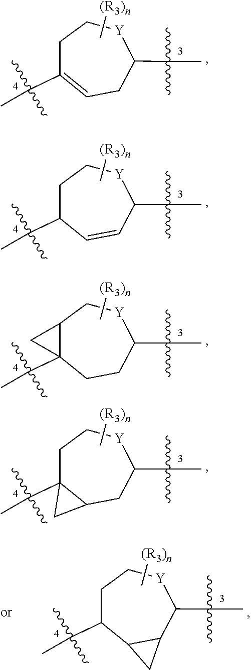

wherein
Z₀, Z₁, Z₂, and Z₃ are each independently CR₃₁, S, or N;
each R₃₁ is independently H or R₃;
Y is O, NR_{b}, NR_{b}C(O), NR_{b}S(O)₂, S, SO₂, or C(R_{c})₂,
R_{b} is H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S;
each R_{c} is independently H, C₁-C₄ alkyl, halo-C₁-C₄ alkyl, C₁-C₄ alkoxy, halo-C₁-C₄ alkoxy, halogen, OH, CN, NH₂, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S; or
two R_{c} are taken together to form an oxo; or
two R_{c}, together with the carbon atom to which they are attached, form a C₃-C₆ carbocylyl or 4- to 6-membered heterocylyl comprising 1 or 2 heteroatoms selected from N, O, and S;
each R₃ is independently C₁-C₄ alkyl, halo-C₁-C₄ alkyl, C₁-C₄ alkoxy, halo-C₁-C₄ alkoxy, halogen, OH, CN, or NH₂; or
two geminal R₃ are taken together to form an oxo; and
n is an integer selected from 0 to 8;

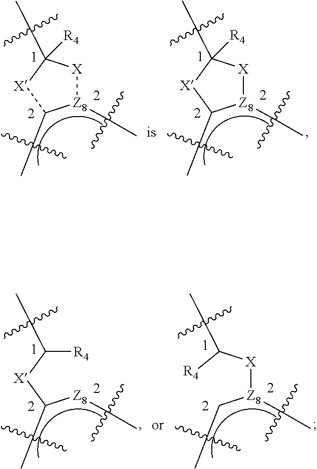

X and X′ are each independently O, NR₁₀, CR₁R₂, C(R₁₁)₂O, or C(R₁₁)₂NR₁₀;
R₁ and R₂ are each independently H, C₁-C₄ alkyl, halo-C₁-C₄ alkyl, C₁-C₄ alkoxy, halo-C₁-C₄ alkoxy, halogen, or OH, or R₁ and R₂, together with the carbon atom to which they are attached, form a C₃-C₆ carbocyclic ring or 4- to 6-membered heterocyclic ring comprising 1 or 2 heteroatoms selected from N, O, and S;
R₁₀ is H, C₁-C₄ alkyl, halo-C₁-C₄ alkyl, or C(O)Rₐ;
Rₐ is H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S;
each R₁₁ is independently H, C₁-C₄ alkyl, halo-C₁-C₄ alkyl, C₁-C₄ alkoxy, halo-C₁-C₄ alkoxy, halogen, or OH; or two R₁₁, together with the carbon atom to which they are attached, form a C₃-C₆ carbocylyl or 4- to 6-membered heterocylyl comprising 1 or 2 heteroatoms selected from N, O, and S; or two R₁₁ are taken together to form an oxo;
Z₄, Z₅, Z₆, and Z₇ are independently CR₆ or N;
Z₈ is C or N, wherein when Z₈ is N, then X is absent;
each R₆ is independently H, C₁-C₄ alkyl, halo-C₁-C₄ alkyl, C₁-C₄ alkoxy, halo-C₁-C₄ alkoxy, halogen, OH, CN, or NH₂;
R₄ is H, C₁-C₄ alkyl, halo-C₁-C₄ alkyl, or C₃-C₆ carbocyclyl;
each R₅ is independently C₁-C₄ alkyl, halo-C₁-C₄ alkyl, C₃-C₆ carbocyclyl, C₁-C₄ alkoxy, halo-C₁-C₄ alkoxy, halogen, OH, CN, or NH₂;
each R₇ is independently C₁-C₄ alkyl, halo-C₁-C₄ alkyl, C₁-C₄ alkoxy, halo-C₁-C₄ alkoxy, halogen, OH, CN, or NH₂; and
R₈′ is C(O)R₈, NHC(O)R₁₁, or 5- to 10-membered heteroaryl comprising one or two 5- or 6-membered rings and 1 to 4 heteroatoms selected from N, O, and S;
R₈ is ORᵢᵢ, N(Rᵢᵢ)₂, or NRᵢᵢSO₂Rᵢᵢᵢ, wherein
each Rᵢᵢ is independently H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S;
Rᵢᵢᵢ is C₁-C₄ alkyl, halo-C₁-C₄ alkyl, C₁-C₄ alkoxy, C₁-C₄ alkylamino, C₁-C₄ dialkylamino, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S; and
   provided that:
1) when Ring A is

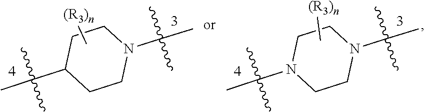

X and X′ are each O, and Z₄ and Z₅ are each CH, then R₄ is not H or methyl;
2) when

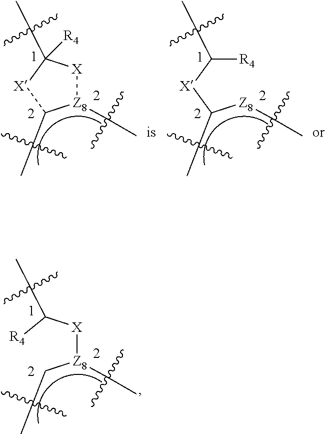

then Ring A is not

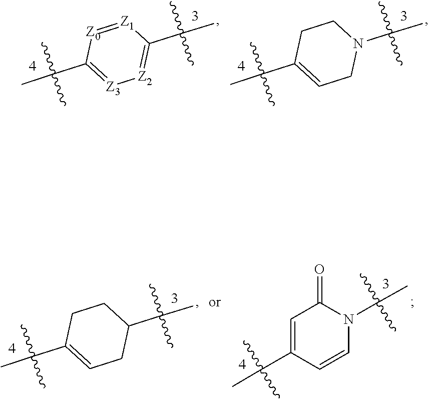

and
3) when

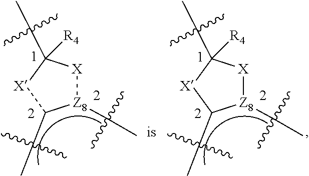

### Ring A is

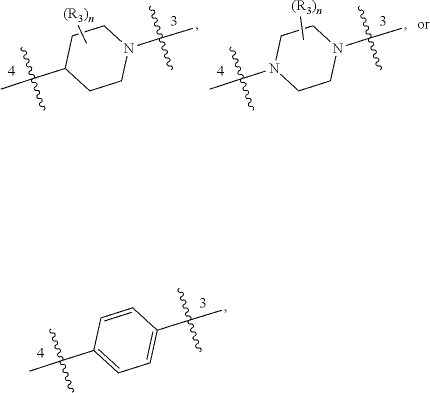

each of Z₄, Z₅, Z₆, Z₇, and Z₈ is CR₆, and X is C(R₁₁)₂O, then X′ is not O, NR₁₀, or CR₁R₂;
4) when

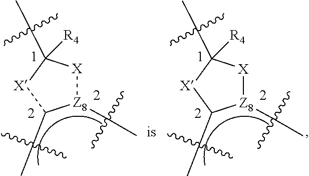

### is Ring A is

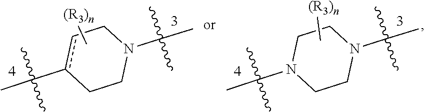

each of Z₄, Z₅, Z₇, and Z₈ is CR₆, Z₆ is CR₆ or N, and X is O, then X′ is not C(R₁₁)₂O;
5) when

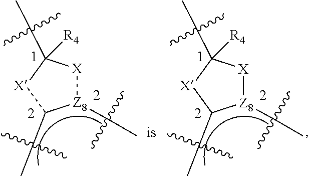

### Ring A is

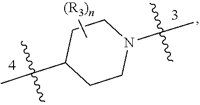

each of Z₄, Z₅, Z₆, Z₇, and Z₈ is CR₆, and X is C(R₁₁)₂O, then X′ is not O;
6) when

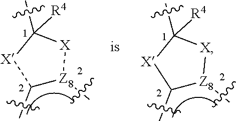

### Ring A is

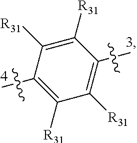

at least one R₃₁ is F, and X is O, NR₁₀, or CR₁R₂, then X′ is not O, NR₁₀, or CR₁R₂; and
7) the compound is not
2-((4-(2-(hydroxymethyl)-2-phenylbenzo[d][1,3]dioxol-4-yl)piperidin-1-yl)methyl)-1-(2-methoxyethyl)-1H-benzo[d]imidazole-6-carboxylic acid;
2-((4-(2-(4-cyano-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid;
2-((4-(2-(4-chloro-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid,
2-((5-(2-(5-chloropyridin-2-yl)-2-methylbenzo[d][1,3]dioxol-4-yl)-3-fluoropyridin-2-yl)methyl)-1-(((S)-oxetan-2-yl)methyl)-1H-benzo[d]imidazole-6-carboxylic acid,
2-((4-(2-(5-chloropyridin-2-yl)-2-methylbenzo[d][1,3]dioxol-4-yl)-2-oxopyridin-1(2H)-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)pyridin-3-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)-2-methoxypyridin-3-yl)methyl)-1-(2-methoxyethyl)-1H-benzo[d]imidazole-6-carboxylic acid, or
2-((6-(2-(4-chloro-2-fluorophenyl)-2-methylbenzo[d][1,3]dioxol-4-yl)-2-oxo-1,2-dihydropyridin-3-yl)methyl)-1-(2-methoxyethyl)-1H-benzo[d]imidazole-6-carboxylic acid.

In one aspect of the application, a compound of Formula (Y) is described:

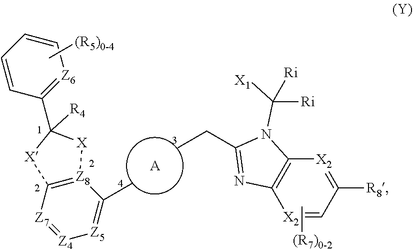

or a pharmaceutically acceptable salt, solvate, prodrug, stereoisomer, or tautomer thereof, wherein:
X₁ is H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S, wherein the carbocyclyl, heterocyclyl, aryl, or heteroaryl is optionally substituted with one or more substituents independently selected from C₁-C₄ alkyl, halo-C₁-C₄ alkyl, C₁-C₄ alkoxy, halo-C₁-C₄ alkoxy, halogen, OH, CN, and NH₂;
each Rᵢ is independently H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S; or two Rᵢ, together with the carbon atom to which they are attached, form a C₃-C₆ carbocyclyl or 4- to 6-membered heterocyclyl comprising 1 or 2 heteroatoms selected from N, O, and S; or one Rᵢ and X₁, together with the carbon atom to which they are attached, form a C₃-C₆ carbocyclyl or 4- to 6-membered heterocyclyl comprising one or two 5- or 6-membered rings and 1 to 4 heteroatoms selected from N, O, and S;
each X₂ is independently CR₇ or N;
Ring A is

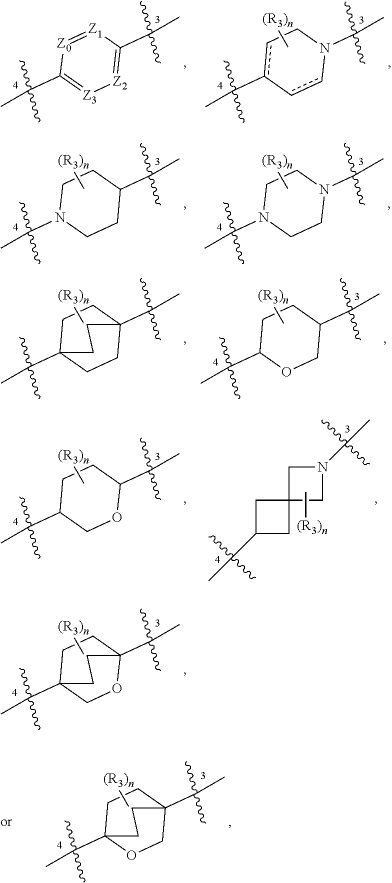

wherein
Z₀, Z₁, Z₂, and Z₃ are each independently CR₃₁, S, or N; each R₃₁ is independently H or R₃;
each R₃ is independently C₁-C₄ alkyl, halo-C₁-C₄ alkyl, C₁-C₄ alkoxy, halo-C₁-C₄ alkoxy, halogen, OH, CN, or NH₂; or
two geminal R₃ are taken together to form an oxo;
n is 0, 1, 2, 3, or 4;

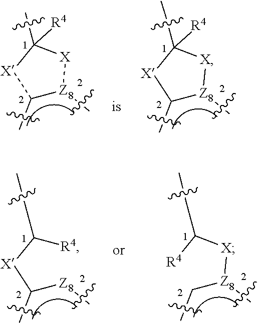

X and X′ are each independently O, NR₁₀, CR₁R₂, C(R₁₁)₂O or C(R₁₁)₂NR₁₀ wherein the carbon atom is bonded with the atom marked “1”;
R₁ and R₂ are each independently H, C₁-C₄ alkyl, halo-C₁-C₄ alkyl, C₁-C₄ alkoxy, halo-C₁-C₄ alkoxy, halogen, or OH, or R₁ and R₂, together with the carbon atom to which they are attached, form a C₃-C₆ carbocyclic ring or 4- to 6-membered heterocyclic ring comprising 1 or 2 heteroatoms selected from N, O, and S;
R₁₀ is H, C₁-C₄ alkyl, halo-C₁-C₄ alkyl, or C(O)Rₐ;
Rₐ is H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S;
each R₁₁ is independently H, C₁-C₄ alkyl, halo-C₁-C₄ alkyl, C₁-C₄ alkoxy, halo-C₁-C₄ alkoxy, halogen, or OH; or two R₁₁, together with carbon atom to which they are attached, form a C₃-C₆ carbocylyl or 4- to 6-membered heterocylyl comprising 1 or 2 heteroatoms selected from N, O, and S; or two Rn are taken together to form an oxo;
Z₄, Z₅, Z₆, and Z₇ are independently CR₆ or N;
Z₈ is C or N, wherein when Z₈ is N, then X is absent;
each R₆ is independently H, C₁-C₄ alkyl, halo-C₁-C₄ alkyl, C₁-C₄ alkoxy, halo-C₁-C₄ alkoxy, halogen, OH, CN, or NH₂;
R₄ is H, C₁-C₄ alkyl, halo-C₁-C₄ alkyl, or C₃-C₆ carbocyclyl;
each R₅ is independently C₁-C₄ alkyl, halo-C₁-C₄ alkyl, C₃-C₆ carbocyclyl, C₁-C₄ alkoxy, halo-C₁-C₄ alkoxy, halogen, OH, CN, or NH₂;
each R₇ is independently C₁-C₄ alkyl, halo-C₁-C₄ alkyl, C₁-C₄ alkoxy, halo-C₁-C₄ alkoxy, halogen, OH, CN, or NH₂; and
R₈′ is C(O)R₈, NHC(O)Rᵢᵢ, or 5- to 10-membered heteroaryl comprising one or two 5- or 6-membered rings and 1 to 4 heteroatoms selected from N, O, and S;
R₈ is ORᵢᵢ, N(Rᵢᵢ)₂, or NRᵢᵢSO₂Rᵢᵢᵢ, wherein
each Rᵢᵢ is independently H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S;
Rᵢᵢᵢ is C₁-C₄ alkyl, halo-C₁-C₄ alkyl, C₁-C₄ alkoxy, C₁-C₄ alkylamino, C₁-C₄ dialkylamino, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S; and
   provided that:
1) when Ring A is

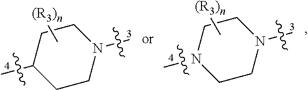

X and X′ are each O, and Z₄ and Z₅ are each CH, then R₄ is not H or methyl;
2) when

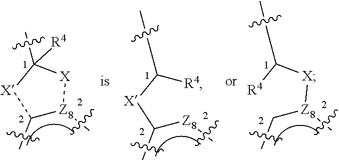

then Ring A is not

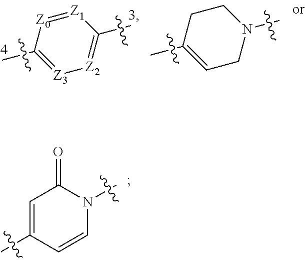

and
3) when

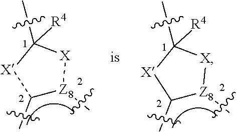

### Ring A is

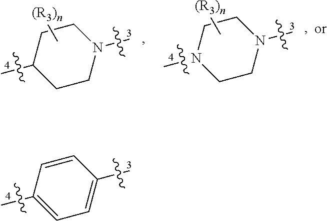

each of Z₄, Z₅, Z₆, Z₇, and Z₈ is CR₆, and X is C(R₁₁)₂O, then X′ is not O, NR₁₀, or CR₁R₂;
4) when

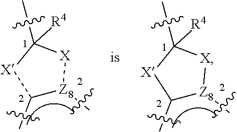

### Ring A is

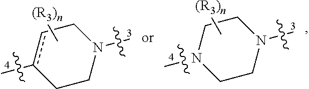

each of Z₄, Z₅, Z₇, and Z₈ is CR₆, Z₆ is CR₆ or N, and X is O, then X′ is not C(R₁₁)₂O;
5) when

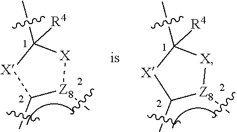

### Ring A is

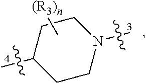

each of Z₄, Z₅, Z₆, Z₇, and Z₈ is CR₆, and X is C(R₁₁)₂O, then X′ is not O;
6) when

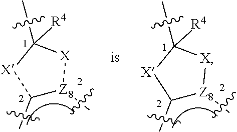

### Ring A is

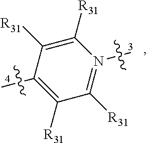

at least one R₃₁ is F, and X is O, NR₁₀ or CR₁R₂, then X′is not O, NR₁₀ or CR₁R₂; and
7) the compound is not
2-((4-(2-(hydroxymethyl)-2-phenylbenzo[d][1,3]dioxol-4-yl)piperidin-1-yl)methyl)-1-(2-methoxyethyl)-1H-benzo[d]imidazole-6-carboxylic acid;
2-((4-(2-(4-cyano-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid;
2-((4-(2-(4-chloro-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid;
2-((5-(2-(5-chloropyridin-2-yl)-2-methylbenzo[d][1,3]dioxol-4-yl)-3-fluoropyridin-2-yl)methyl)-1-(((S)-oxetan-2-yl)methyl)-1H-benzo[d]imidazole-6-carboxylic acid,
2-((4-(2-(5-chloropyridin-2-yl)-2-methylbenzo[d][1,3]dioxol-4-yl)-2-oxopyridin-1(2H)-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)pyridin-3-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)-2-methoxypyridin-3-yl)methyl)-1-(2-methoxyethyl)-1H-benzo[d]imidazole-6-carboxylic acid, or
2-((6-(2-(4-chloro-2-fluorophenyl)-2-methylbenzo[d][1,3]dioxol-4-yl)-2-oxo-1,2-dihydropyridin-3-yl)methyl)-1-(2-methoxyethyl)-1H-benzo[d]imidazole-6-carboxylic acid.

In certain embodiments, each Rᵢ is independently H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S.

In certain embodiments, two Rᵢ, together with the carbon to which they are attached, form a C₃-C₆ carbocyclyl or 4- to 6-membered heterocyclyl comprising 1 or 2 heteroatoms selected from N, O, and S.

In certain embodiments, two Rᵢ, together with the carbon to which they are attached, form a cyclopropyl.

In certain embodiments, one Rᵢ and X₁, together with the carbon to which they are attached, form a C₃-C₆ carbocyclyl or 4- to 6-membered heterocyclyl comprising 1 or 2 heteroatoms selected from N, O, and S.

In certain embodiments, one Rᵢ and X₁, together with the carbon to which they are attached, form an oxetanyl or a tetrahydrofuranyl.

In some embodiments, Ring A is

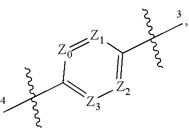

In some embodiments, Ring A is

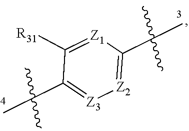

In some embodiments, Z₀, Z₁, Z₂, and Z₃ are each independently CR₃₁.

In some embodiments, only one of Z₀, Z₁, Z₂, and Z₃ is CR₃₁, and three of Z₀, Z₁, Z₂, and Z₃ are N.

In some embodiments, Z₀ is CR₃₁, and Z₁, Z₂, and Z₃ are N.

In some embodiments, Z₁ is CR₃₁, and Z₀, Z₂ and Z₃ are N.

In some embodiments, Z₂ is CR₃₁, and Z₀, Z₁ and Z₃ are N.

In some embodiments, Z₃ is CR₃₁, and Z₀, Z₁ and Z₂ are N.

In some embodiments, two of Z₀, Z₁, Z₂, and Z₃ are CR₃₁, and two of Z₀, Z₁, Z₂, and Z₃ are N.

In some embodiments, Z₀ and Z₁ are N; and Z₂ and Z₃ are CR₃₁.

In some embodiments, Z₀ and Z₂ are N; and Z₁ and Z₃ are CR₃₁.

In some embodiments, Z₀ and Z₃ are N; and Z₁ and Z₂ are CR₃₁.

In some embodiments, Z₁ and Z₂ are N; and Z₀ and Z₃ are CR₃₁.

In some embodiments, Z₁ and Z₃ are N; and Z₀ and Z₂ are CR₃₁.

In some embodiments, Z₂ and Z₃ are N; and Z₀ and Z₁ are CR₃₁.

In some embodiments, only one of Z₀, Z₁, Z₂, and Z₃ is N, and three of Z₀, Z₁, Z₂, and Z₃ are CR₃₁.

In some embodiments, Z₀ is N, and Z₁, Z₂ and Z₃ are CR₃₁.

In some embodiments, Z₁ is N, and Z₀, Z₂ and Z₃ are CR₃₁.

In some embodiments, Z₂ is N, and Z₀, Z₁ and Z₃ are CR₃₁.

In some embodiments, Z₃ is N, and Z₀, Z₁ and Z₂ are CR₃₁.

In some embodiments, Ring A is

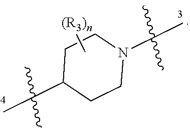

In some embodiments, Ring A is

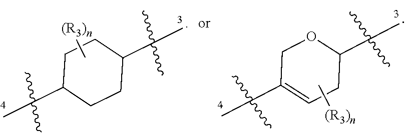

In some embodiments, Ring A is

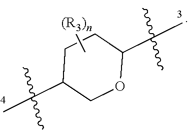

In some embodiments, Ring A is

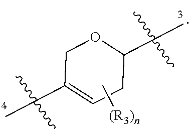

In some embodiments, Ring A is

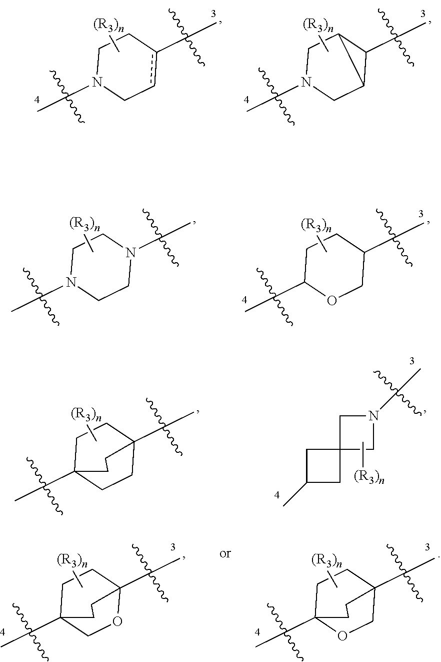

In some embodiments, RingA is

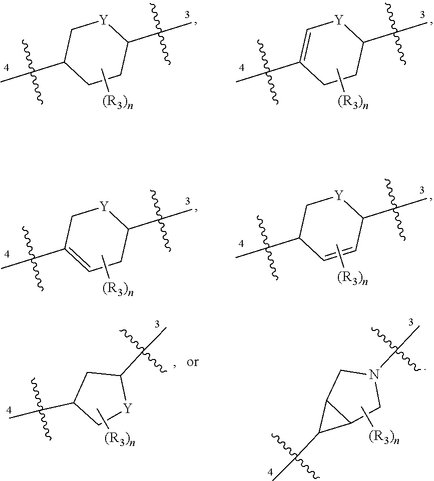

In some embodiments, Ring A is

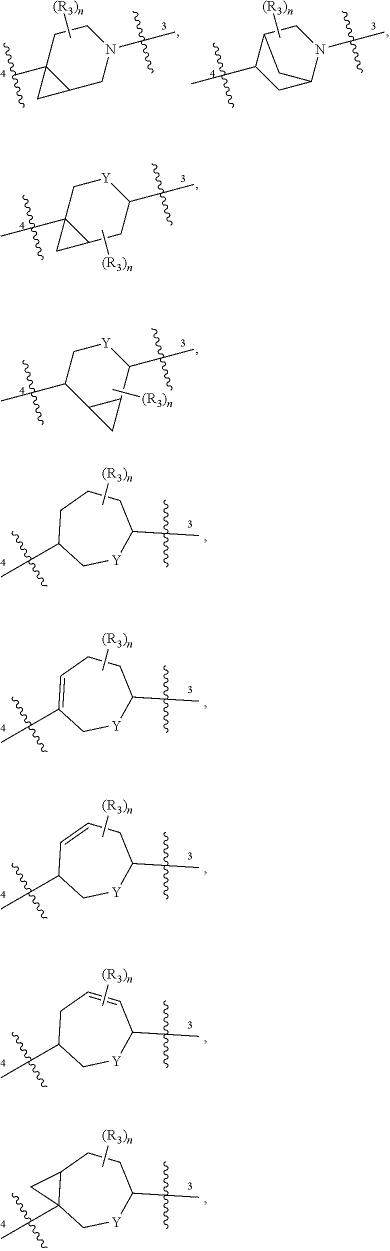

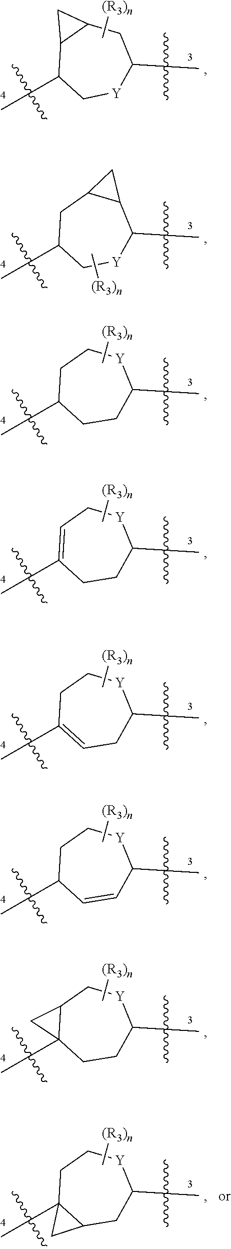

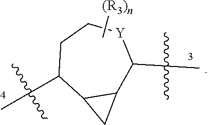

In some embodiments, Y is O, NR_{b}, or C(R_{c})₂.

In some embodiments, Y is O.

In some embodiments,

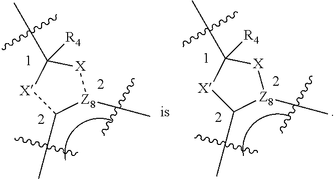

In some embodiments, X and X′ are each O.

In some embodiments, X is CR₁R₂ and X′ is O. In some embodiments, X is CHR₁₁O and X′ is O. In some embodiments, X is CHR₁₁NR₁₀ and X′ is O. In some embodiments, X is C(O)NR₁₀ and X′ is O. In some embodiments, X is C(R₁₁)₂NR₁₀ and X′ is O.

In some embodiments, X is O and X′ is CHR₁₁O. In some embodiments, X is O and X′ is CHR₁₁NR₁₀. In some embodiments, X is O and X′ is C(O)NR₁₀. In some embodiments, X is O and X′ is C(R₁₁)₂NR₁₀.

In some embodiments,

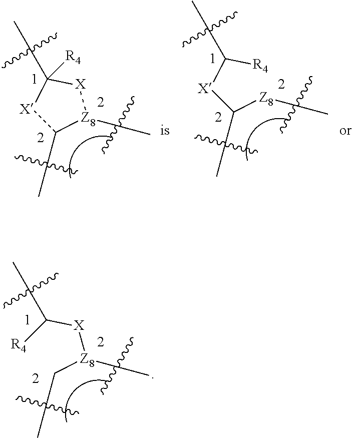

In some embodiments, X and X′ are each independently O, NR₁₀, CR₁R₂, or C(R₁₁)₂O or C(R₁₁)₂NR₁₀ wherein the carbon atom is bonded with the atom marked “1”.

In some embodiments, X and X′ are each independently O.

In some embodiments, each X₂ is N.

In some embodiments, each X₂ is CR₇.

In some embodiments, one X₂ is CR₇, and the other X₂ is N.

In some embodiments, Z₄, Z₅, and Z₇ are each CR₆.

In some embodiments, only one of Z₄, Z₅, and Z₇ is CR₆, and two of Z₄, Z₅, and Z₇ are N.

In some embodiments, Z₄ and Z₅ are each N, and Z₇ is CR₆.

In some embodiments, Z₄ and Z₇ are each N, and Z₅ is CR₆.

In some embodiments, Z₅ and Z₇ are each N, and Z₄ is CR₆.

In some embodiments, only one of Z₄, Z₅, and Z₇ is N, and two of Z₄, Z₅, and Z₇ are CR₆.

In some embodiments, Z₄ and Z₇ are each CR₆ and Z₅ is N.

In some embodiments, Z₅ and Z₇ are each CR₆ and Z₄ is N.

In some embodiments, Z₄ and Z₅ are each CR₆ and Z₇ is N.

In some embodiments, Z₄, Z₅, and Z₇ are each N.

In some embodiments, Z₈ is C. In some embodiments, Z₈ is N, wherein when Z₈ is N, X is absent.

In some embodiments, any Ring A described herein can be combined with any X, X′, and Z₈ described herein.

In some embodiments,

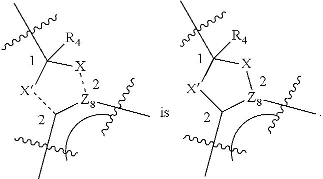

and Ring A, X, X′, and Z₈ can be combined as shown in the table below.

| Z₈ | X and X′ | Ring A |
|---|---|---|
| C | O | 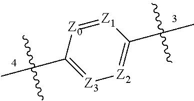 |
| | | 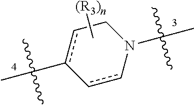 |
| | | 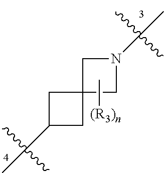 |
| | | 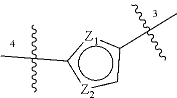 |
| | | 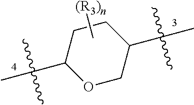 |
| | | 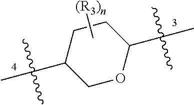 |
| | | 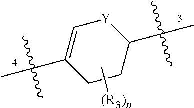 |
| | | 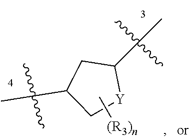 |
| | | 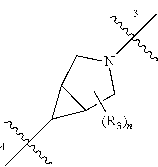 |
| C | CHR₁₁O, O | 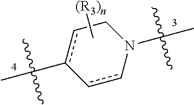 |
| | | 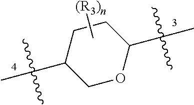 |
| | | 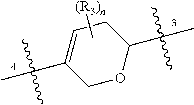 |
| | | 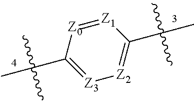 |
| | | 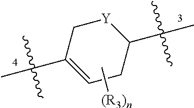 |
| C | C(R₁₁)₂NR₁₀, O | 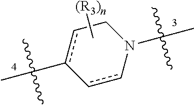 |
| | | 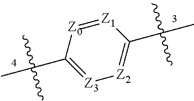 |
| C | CHR₁₁O | 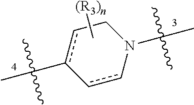 |
| | | 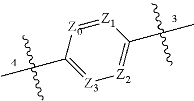 |
| C | CHR₁₁NR₁₀, O | 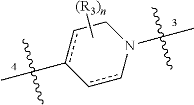 |

In some embodiments,

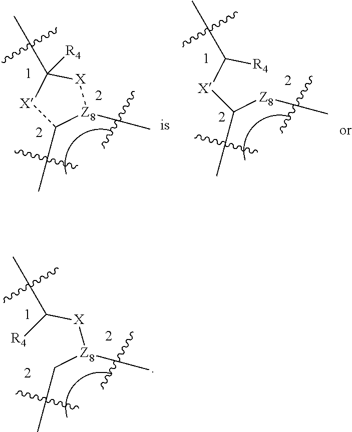

and Ring A, X, X′, and Z₈ can be combined as shown in the table below.

| Z₈ | X and X′ | Ring A |
|---|---|---|
| N | O | 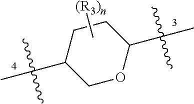 |
| | | 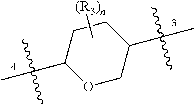 |
| | | 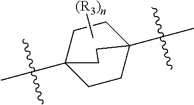 |
| | | 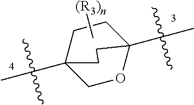 |
| | | 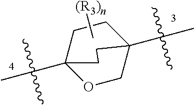 |

In some embodiments, R₈′ is C(O)R₈ or NHC(O)Rᵢᵢ.

In some embodiments, R₈′ is C(O)R₈.

In some embodiments, R₈′ is 5- to 10-membered heteroaryl comprising one or two 5- or 6-membered rings and 1 to 4 heteroatoms selected from N, O, and S.

In some embodiments, R₈ is ORᵢᵢ. In some embodiments, R₈ is N(Rᵢᵢ)₂. In some embodiments, R₈ is NRᵢᵢSO₂Rᵢᵢᵢ.

In some embodiments, each Rᵢᵢ is independently H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S.

In some embodiments, Rᵢᵢᵢ is C₁-C₄ alkyl, halo-C₁-C₄ alkyl, C₁-C₄ alkoxy, C₁-C₄ alkylamino, C₁-C₄ dialkylamino, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S.

In some embodiments, R₈ is OH or O—C₁-C₄ alkyl. In some embodiments, R₈ is OH.

In some embodiments, R₈ is NH₂, NH—C₁-C₄ alkyl, or N(C₁-C₄ alkyl)₂.

In some embodiments, R₈ is OH. In some embodiments, R₈ is O—C₁-C₄ alkyl (e.g., methyl, ethyl, propyl, i-propyl, n-butyl, i-butyl, s-butyl, or t-butyl). In some embodiments, R₈ is NH₂. In some embodiments, R₈ is NH—C₁-C₄ alkyl (e.g., methyl, ethyl, propyl, i-propyl, n-butyl, i-butyl, s-butyl, or t-butyl). In some embodiments, R₈ is N(C₁-C₄ alkyl)₂ (e.g., methyl, ethyl, propyl, i-propyl, n-butyl, i-butyl, s-butyl, or t-butyl).

In some embodiments, R₄, R₅, R₇, Z₄, Z₅, and X₁ can be any groups that are described herein for each variable.

In some embodiments, the remainder of the variables in Formula (Y) can be any of the substituent groups described herein, for example, for Formula (A).

In some embodiments, the compound of Formula (Y) is a compound of Formula (Y′):

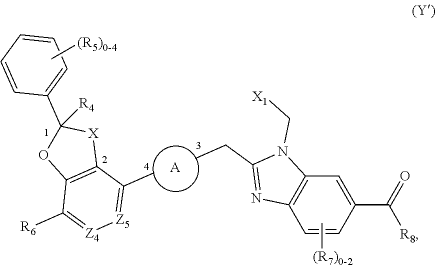

or a pharmaceutically acceptable salt, solvate, prodrug, stereoisomer, or tautomer thereof, wherein:
X is O, NR₁₀, CR₁R₂, or C(R₁₁)₂O or C(R₁₁)₂NR₁₀, C(R₁₁)₂O or C(R₁₁)₂NR₁₀ wherein the carbon atom is bonded with the atom marked “1”.

In one aspect of the application, a compound of Formula (A) is described:

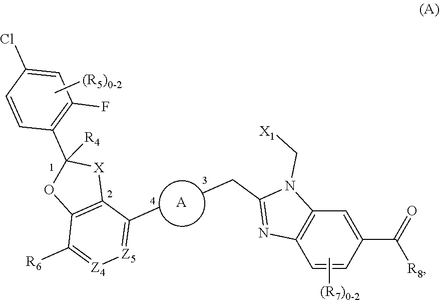

or a pharmaceutically acceptable salt, solvate, prodrug, stereoisomer, or tautomer thereof, wherein:

X¹ is H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S, wherein the carbocyclyl, heterocyclyl, aryl, or heteroaryl is optionally substituted with one or more substituents independently selected from C₁-C₄ alkyl, halo-C₁-C₄ alkyl, C₁-C₄ alkoxy, halo-C₁-C₄ alkoxy, halogen, OH, CN, and NH₂;

Ring A is

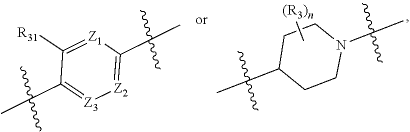

wherein
Z₁, Z₂, and Z₃ are each independently CR₃₁ or N;
each R₃₁ is independently H or R₃;
each R₃ is independently C₁-C₄ alkyl, halo-C₁-C₄ alkyl, C₁-C₄ alkoxy, halo-C₁-C₄ alkoxy, halogen, OH, CN, or NH₂;
n is 0, 1, 2, 3, or 4;

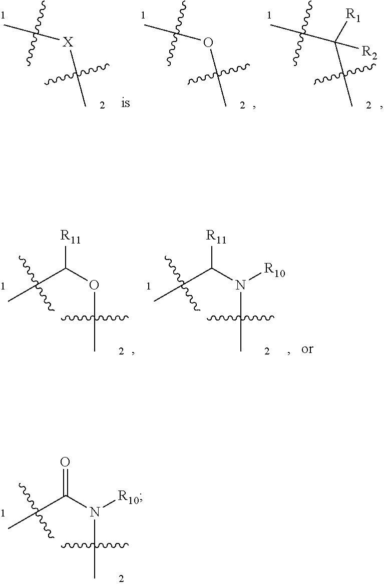

R₁ and R₂ are each independently H, C₁-C₄ alkyl, halo-C₁-C₄ alkyl, C₁-C₄ alkoxy, halo-C₁-C₄ alkoxy, halogen, or OH, or R₁ and R₂, together with the carbon atom to which they are attached, form a C₃-C₆ carbocyclic ring or 4- to 6-membered heterocyclic ring comprising 1 or 2 heteroatoms selected from N, O, and S;

R₁₀ is H, C₁-C₄ alkyl, or halo-C₁-C₄ alkyl;

R₁₁ is H, C₁-C₄ alkyl, halo-C₁-C₄ alkyl, C₁-C₄ alkoxy, halo-C₁-C₄ alkoxy, halogen, or OH;

Z₄ and Z₅ are each independently CR₆ or N;

each R₆ is independently H, C₁-C₄ alkyl, halo-C₁-C₄ alkyl, C₁-C₄ alkoxy, halo-C₁-C₄ alkoxy, halogen, OH, CN, or NH₂;

R₄ is H, C₁-C₄ alkyl, halo-C₁-C₄ alkyl, or C₃-C₆ carbocyclyl, provided that when Ring A is

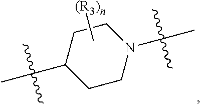

X is O, and Z₄ and Z₅ are each CR₆, then R₄ is not methyl;

each R₅ is independently C₁-C₄ alkyl, halo-C₁-C₄ alkyl, C₁-C₄ alkoxy, halo-C₁-C₄ alkoxy, C₃-C₆ carbocyclyl, halogen, OH, CN, or NH₂;

each R₇ is independently C₁-C₄ alkyl, halo-C₁-C₄ alkyl, C₁-C₄ alkoxy, halo-C₁-C₄ alkoxy, halogen, OH, CN, or NH₂; and

R₈ is OH, O—C₁-C₄ alkyl, NH₂, NH—C₁-C₄ alkyl, or N(C₁-C₄ alkyl)₂.

In some embodiments, a compound of Formula (A) is as described above, provided that:
3) when Ring A is

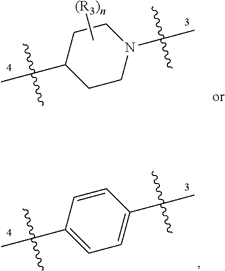

and each of Z₄ and Z₅ is CR₆, then

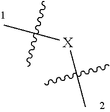

is not

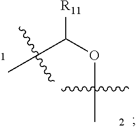

6) when Ring A is

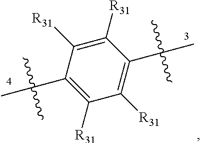

at least one R₃₁ is F, then

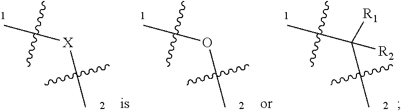

and
7) the compound is not
2-((4-(2-(hydroxymethyl)-2-phenylbenzo[d][1,3]dioxol-4-yl)piperidin-1-yl)methyl)-1-(2-methoxyethyl)-1H-benzo[d]imidazole-6-carboxylic acid;
2-((4-(2-(4-cyano-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid;
2-((4-(2-(4-chloro-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid,
2-((5-(2-(5-chloropyridin-2-yl)-2-methylbenzo[d][1,3]dioxol-4-yl)-3-fluoropyridin-2-yl)methyl)-1-(((S)-oxetan-2-yl)methyl)-1H-benzo[d]imidazole-6-carboxylic acid,
2-((4-(2-(5-chloropyridin-2-yl)-2-methylbenzo[d][1,3]dioxol-4-yl)-2-oxopyridin-1(2H)-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)pyridin-3-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)-2-methoxypyridin-3-yl)methyl)-1-(2-methoxyethyl)-1H-benzo[d]imidazole-6-carboxylic acid, or
2-((6-(2-(4-chloro-2-fluorophenyl)-2-methylbenzo[d][1,3]dioxol-4-yl)-2-oxo-1,2-dihydropyridin-3-yl)methyl)-1-(2-methoxyethyl)-1H-benzo[d]imidazole-6-carboxylic acid.

In some embodiments, a compound of Formula (A) is as described above, provided that:
1) when Ring A is

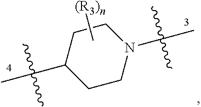

X is O, and each of Z₄ and Z₅ is CH, then R₄ is not H or methyl;
3) when Ring A is

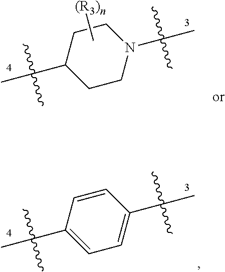

and each of Z₄ and Z₅ is CR₆, then

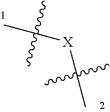

is not

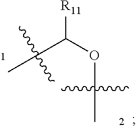

6) when Ring A is

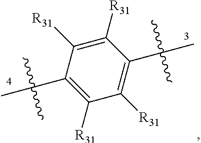

at least one R₃₁ is F, then

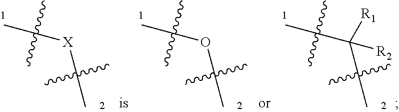

7) the compound is not
2-((4-(2-(hydroxymethyl)-2-phenylbenzo[d][1,3]dioxol-4-yl)piperidin-1-yl)methyl)-1-(2-methoxyethyl)-1H-benzo[d]imidazole-6-carboxylic acid;
2-((4-(2-(4-cyano-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid;
2-((4-(2-(4-chloro-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid,
2-((5-(2-(5-chloropyridin-2-yl)-2-methylbenzo[d][1,3]dioxol-4-yl)-3-fluoropyridin-2-yl)methyl)-1-(((S)-oxetan-2-yl)methyl)-1H-benzo[d]imidazole-6-carboxylic acid,
2-((4-(2-(5-chloropyridin-2-yl)-2-methylbenzo[d][1,3]dioxol-4-yl)-2-oxopyridin-1(2H)-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)pyridin-3-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)-2-methoxypyridin-3-yl)methyl)-1-(2-methoxyethyl)-1H-benzo[d]imidazole-6-carboxylic acid, or
2-((6-(2-(4-chloro-2-fluorophenyl)-2-methylbenzo[d][1,3]dioxol-4-yl)-2-oxo-1,2-dihydropyridin-3-yl)methyl)-1-(2-methoxyethyl)-1H-benzo[d]imidazole-6-carboxylic acid.

In some embodiments, the compound of Formula (A) is a compound of Formula (I):

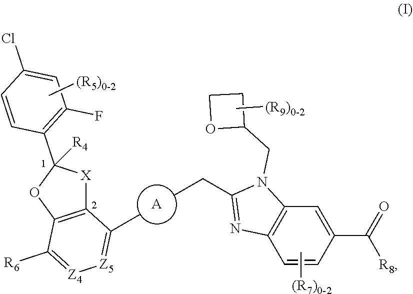

or a pharmaceutically acceptable salt, solvate, prodrug, stereoisomer, or tautomer thereof, wherein:

each R₉ is independently C₁-C₄ alkyl, halo-C₁-C₄ alkyl, C₁-C₄ alkoxy, halo-C₁-C₄ alkoxy, halogen, OH, CN, or NH₂.

In some embodiments, the compounds of Formula (A) are of Formula (Aa):

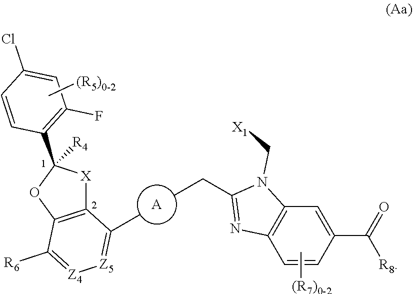

or a pharmaceutically acceptable salt, solvate, prodrug, stereoisomer, or tautomer thereof.

In some embodiments, the compounds of Formula (I) are of Formula (Ia) or Formula (Ia′):

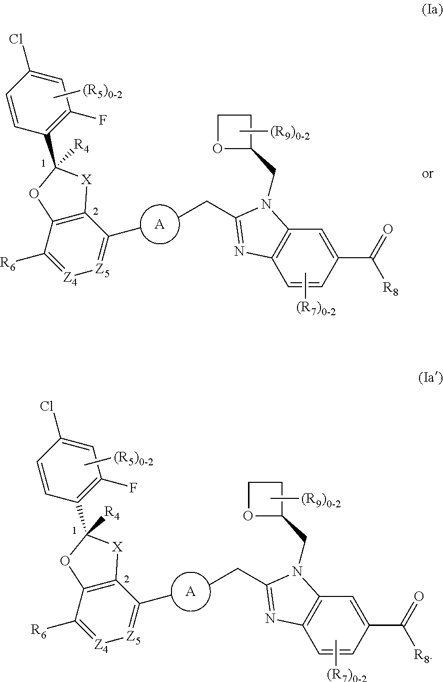

or a pharmaceutically acceptable salt, solvate, prodrug, stereoisomer, or tautomer thereof.

In some embodiments, the compounds of Formula (I) are of Formula (I1):

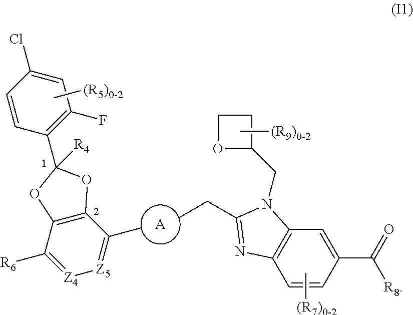

or a pharmaceutically acceptable salt, solvate, prodrug, stereoisomer, or tautomer thereof.

In some embodiments, the compounds of Formula (I) are of Formula (I1a) or Formula (I1a′):

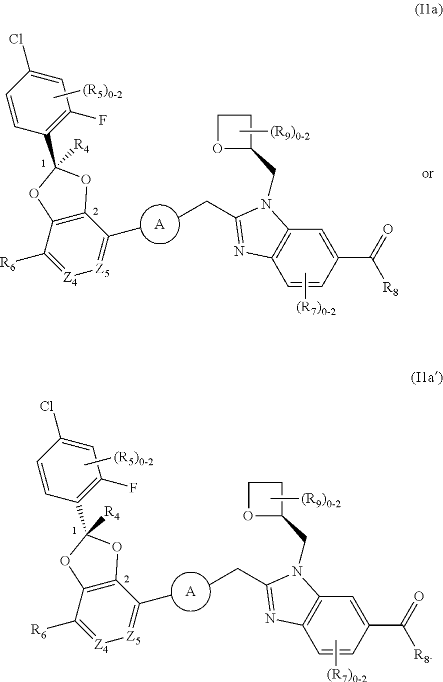

or a pharmaceutically acceptable salt, solvate, prodrug, stereoisomer, or tautomer thereof.

For compounds of Formula (Y), (Y′), (A), (Aa), (I), (Ia), (Ia′), (I1), or (I1a), (I1a′), where applicable:

In some embodiments, X₁ is H. In some embodiments, X₁ is C₁-C₄ alkyl (e.g., methyl, ethyl, propyl, i-propyl, n-butyl, i-butyl, s-butyl, or t-butyl). In some embodiments, X₁ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, Xi is C₁-C₄ alkoxy (e.g., methoxy, ethoxy, propoxy, i-propoxy, n-butoxy, i-butoxy, s-butoxy, or t-butoxy). In some embodiments, X₁ is halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, X₁ is halogen (e.g., F, Cl, Br, or I). In some embodiments, X₁ is OH.

In some embodiments, X₁ is C₃-C₆ carbocyclyl (e.g., cyclopropyl, cyclobutyl, cyclopentyl, or cyclohexyl). In some embodiments, X₁ is C₃-C₆ carbocyclyl substituted with one or more substituents independently selected from C₁-C₄ alkyl (e.g., methyl, ethyl, propyl, i-propyl, n-butyl, i-butyl, s-butyl, or t-butyl), halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, i-propoxy, n-butoxy, i-butoxy, s-butoxy, or t-butoxy), halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)), halogen (e.g., F, Cl, Br, or I), OH, CN, and NH₂. In some embodiments, X₁ is C₃-C₆ carbocyclyl substituted with one or more C₁-C₄ alkyl (e.g., methyl, ethyl, propyl, i-propyl, n-butyl, i-butyl, s-butyl, or t-butyl). In some embodiments, X₁ is C₃-C₆ carbocyclyl substituted with one or more halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, X₁ is C₃-C₆ carbocyclyl substituted with one or more C₁-C₄ alkoxy (e.g., methoxy, ethoxy, propoxy, i-propoxy, n-butoxy, i-butoxy, s-butoxy, or t-butoxy). In some embodiments, X₁ is C₃-C₆ carbocyclyl substituted with one or more halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, X₁ is C₃-C₆ carbocyclyl substituted with one or more halogen (e.g., F, Cl, Br, or I). In some embodiments, X₁ is C₃-C₆ carbocyclyl substituted with one or more OH. In some embodiments, X₁ is C₃-C₆ carbocyclyl substituted with one or more CN. In some embodiments, X₁ is C₃-C₆ carbocyclyl substituted with one or more NH₂.

In some embodiments, X₁ is 4- to 6-membered heterocyclyl comprising 1 or 2 heteroatoms selected from N, O, and S (e.g., azetidinyl, oxetanyl, thietanyl, pyrrolidinyl, tetrahydrofuranyl, tetrahydrothiophenyl, oxazolidinyl, isoxazolidinyl, thiazolidinyl, isothiazolidinyl, piperidinyl, piperazinyl, tetrahydropyranyl, tetrahydrothiapyranyl, dioxanyl, morpholinyl, oxazinanyl, thiazinanyl, or oxathianyl). In some embodiments, X₁ is 4- to 6-membered heterocyclyl comprising 1 or 2 heteroatoms selected from N, O, and S substituted with one or more substituents independently selected from C₁-C₄ alkyl (e.g., methyl, ethyl, propyl, i-propyl, n-butyl, i-butyl, s-butyl, or t-butyl), halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, i-propoxy, n-butoxy, i-butoxy, s-butoxy, or t-butoxy), halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)), halogen (e.g., F, Cl, Br, or I), OH, CN, and NH₂. In some embodiments, X₁ is 4- to 6-membered heterocyclyl comprising 1 or 2 heteroatoms selected from N, O, and S substituted with one or more C₁-C₄ alkyl (e.g., methyl, ethyl, propyl, i-propyl, n-butyl, i-butyl, s-butyl, or t-butyl). In some embodiments, X₁ is 4- to 6-membered heterocyclyl comprising 1 or 2 heteroatoms selected from N, O, and S substituted with one or more halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, X₁ is 4- to 6-membered heterocyclyl comprising 1 or 2 heteroatoms selected from N, O, and S substituted with one or more C₁-C₄ alkoxy (e.g., methoxy, ethoxy, propoxy, i-propoxy, n-butoxy, i-butoxy, s-butoxy, or t-butoxy). In some embodiments, X₁ is 4- to 6-membered heterocyclyl comprising 1 or 2 heteroatoms selected from N, O, and S substituted with one or more halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, X₁ is 4- to 6-membered heterocyclyl comprising 1 or 2 heteroatoms selected from N, O, and S substituted with one or more halogen (e.g., F, Cl, Br, or I). In some embodiments, X₁ is 4- to 6-membered heterocyclyl comprising 1 or 2 heteroatoms selected from N, O, and S substituted with one or more OH. In some embodiments, X₁ is 4- to 6-membered heterocyclyl comprising 1 or 2 heteroatoms selected from N, O, and S substituted with one or more CN. In some embodiments, X₁ is 4- to 6-membered heterocyclyl comprising 1 or 2 heteroatoms selected from N, O, and S with one or more NH₂.

In some embodiments, X₁ is C₆-C₁₀ aryl (e.g., phenyl or naphthyl). In some embodiments, X₁ is C₆-C₁₀ aryl substituted with one or more substituents independently selected from C₁-C₄ alkyl (e.g., methyl, ethyl, propyl, i-propyl, n-butyl, i-butyl, s-butyl, or t-butyl), halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, i-propoxy, n-butoxy, i-butoxy, s-butoxy, or t-butoxy), halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)), halogen (e.g., F, Cl, Br, or I), OH, CN, and NH₂. In some embodiments, X₁ is C₆-C₁₀ aryl substituted with one or more C₁-C₄ alkyl (e.g., methyl, ethyl, propyl, i-propyl, n-butyl, i-butyl, s-butyl, or t-butyl). In some embodiments, X₁ is 4- to 6-membered heterocyclyl comprising 1 or 2 heteroatoms selected from N, O, and S substituted with one or more halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, X₁ is C₆-C₁₀ aryl substituted with one or more C₁-C₄ alkoxy (e.g., methoxy, ethoxy, propoxy, i-propoxy, n-butoxy, i-butoxy, s-butoxy, or t-butoxy). In some embodiments, X₁ is C₆-C₁₀ aryl substituted with one or more halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, X₁ is C₆-C₁₀ aryl substituted with one or more halogen (e.g., F, Cl, Br, or I). In some embodiments, X₁ is C₆-C₁₀ aryl substituted with one or more OH. In some embodiments, X₁ is C₆-C₁₀ aryl substituted with one or more CN. In some embodiments, X₁ is C₆-C₁₀ aryl substituted with one or more NH₂.

In some embodiments, X₁ is 5- to 10-membered heteroaryl comprising one or two 5- or 6-membered rings and 1 to 4 heteroatoms selected from N, O, and S (e.g., pyrrolyl, furanyl, thiophenyl, pyrazolyl, imidazolyl, oxazolyl, isoxazolyl, isothiazolyl, thiazolyl, triazolyl, oxodiazolyl, thiadiazolyl, tetrazolyl, pyridinyl, pyrazinyl, pyrimidinyl, pyridazinyl, triazinyl, dioxinyl, oxazinyl, thiazinyl, dihydropyrrolopyrrolyl, furopyrrolyl, thienopyrrolyl, indolyl, isoindolyl, indolizinyl, indazolyl, azaindolyl, pyrazolopyrimidinyl, purinyl, benzofuranyl, isobenzofuranyl, benzothiophenyl, benzoisoxazolyl, benzoisothiazolyl, benzothiadiazolyl, phthalazinyl, cinnolinyl, naphthyridinyl, pyridopyrimidinyl, pyridopyrazinyl, pteridinyl, quinolinyl, isoquinolinyl, benzimidazolyl, benzooxazolyl, or quinoxalinyl). In some embodiments, X₁ is 5- to 10-membered heteroaryl comprising 1 or 2 heteroatoms selected from N, O, and S. In some embodiments, X₁ is 5- to 10-membered heteroaryl comprising one or two 5- or 6-membered rings and 1 to 4 heteroatoms selected from N, O, and S substituted with one or more substituents independently selected from C₁-C₄ alkyl (e.g., methyl, ethyl, propyl, i-propyl, n-butyl, i-butyl, s-butyl, or t-butyl), halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, i-propoxy, n-butoxy, i-butoxy, s-butoxy, or t-butoxy), halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)), halogen (e.g., F, Cl, Br, or I), OH, CN, and NH₂. In some embodiments, X₁ is 5- to 6-membered heteroaryl comprising 1 or 2 heteroatoms selected from N, O, and S substituted with one or more substituents independently selected from C₁-C₄ alkyl (e.g., methyl, ethyl, propyl, i-propyl, n-butyl, i-butyl, s-butyl, or t-butyl), halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)), C₁-C₄ alkoxy (e.g., methoxy, ethoxy, propoxy, i-propoxy, n-butoxy, i-butoxy, s-butoxy, or t-butoxy), halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)), halogen (e.g., F, Cl, Br, or I), OH, CN, and NH₂. In some embodiments, X₁ is 5- to 10-membered heteroaryl comprising one or two 5- or 6-membered rings and 1 to 4 heteroatoms selected from N, O, and S substituted with one or more C₁-C₄ alkyl (e.g., methyl, ethyl, propyl, i-propyl, n-butyl, i-butyl, s-butyl, or t-butyl). X₁ is 5- to 6-membered heteroaryl comprising 1 or 2 heteroatoms selected from N, O, and S substituted with one or more C₁-C₄ alkyl (e.g., methyl, ethyl, propyl, i-propyl, n-butyl, i-butyl, s-butyl, or t-butyl). In some embodiments, X₁ is 5- to 10-membered heteroaryl comprising one or two 5- or 6-membered rings and 1 to 4 heteroatoms selected from N, O, and S substituted with one or more halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, X₁ is 5- to 6-membered heteroaryl comprising 1 or 2 heteroatoms selected from N, O, and S substituted with one or more halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, X₁ is 5- to 10-membered heteroaryl comprising one or two 5- or 6-membered rings and 1 to 4 heteroatoms selected from N, O, and S substituted with one or more C₁-C₄ alkoxy (e.g., methoxy, ethoxy, propoxy, i-propoxy, n-butoxy, i-butoxy, s-butoxy, or t-butoxy). In some embodiments, X₁ is 5- to 6-membered heteroaryl comprising 1 or 2 heteroatoms selected from N, O, and S substituted with one or more C₁-C₄ alkoxy (e.g., methoxy, ethoxy, propoxy, i-propoxy, n-butoxy, i-butoxy, s-butoxy, or t-butoxy). In some embodiments, X₁ is 5- to 10-membered heteroaryl comprising one or two 5- or 6-membered rings and 1 to 4 heteroatoms selected from N, O, and S substituted with one or more halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, X₁ is 5- to 6-membered heteroaryl comprising 1 or 2 heteroatoms selected from N, O, and S substituted with one or more halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, X₁ is 5- to 10-membered heteroaryl comprising one or two 5- or 6-membered rings and 1 to 4 heteroatoms selected from N, O, and S substituted with one or more halogen (e.g., F, Cl, Br, or I). In some embodiments, X₁ is 5- to 6-membered heteroaryl comprising 1 or 2 heteroatoms selected from N, O, and S substituted with one or more halogen (e.g., F, Cl, Br, or I). In some embodiments, X₁ is 5- to 10-membered heteroaryl comprising one or two 5- or 6-membered rings and 1 to 4 heteroatoms selected from N, O, and S substituted with one or more OH. In some embodiments, X₁ is 5- to 6-membered heteroaryl comprising 1 or 2 heteroatoms selected from N, O, and S substituted with one or more OH. In some embodiments, X₁ is 5- to 10-membered heteroaryl comprising one or two 5- or 6-membered rings and 1 to 4 heteroatoms selected from N, O, and S substituted with one or more CN. In some embodiments, X₁ is 5- to 6-membered heteroaryl comprising 1 to 2 heteroatoms selected from N, O, and S substituted with one or more CN. In some embodiments, X₁ is 5- to 10-membered heteroaryl comprising one or two 5- or 6-membered rings and 1 to 4 heteroatoms selected from N, O, and S with one or more NH₂. In some embodiments, X₁ is 5- to 6-membered heteroaryl comprising 1 or 2 heteroatoms selected from N, O, and S substituted with one or more NH₂. In some embodiments, X₁ is 5-membered heteroaryl comprising 1 or 2 heteroatoms selected from N, O, and S. In some embodiments, X₁ is 5-membered heteroaryl comprising 1 or 2 heteroatoms selected from N, O, and S substituted as described herein. In some embodiments, X₁ is 5-membered heteroaryl comprising 1 or 2 heteroatoms selected from N and O. In some embodiments, X₁ is 5-membered heteroaryl comprising 1 or 2 heteroatoms selected from N and O substituted as described herein. In some embodiments, X₁ is 6-membered heteroaryl comprising 1 or 2 heteroatoms selected from N, O, and S. In some embodiments, X₁ is 6-membered heteroaryl comprising 1 or 2 heteroatoms selected from N, O, and S substituted as described herein. In some embodiments, X₁ is 6-membered heteroaryl comprising 1 or 2 heteroatoms selected from N and O. In some embodiments, X₁ is 6-membered heteroaryl comprising 1 or 2 heteroatoms selected from N and O substituted as described herein.

In some embodiments, X₁ is a 5- or 6-membered heteroaryl comprising 1 to 4 heteroatoms selected from N, O, and S (e.g., pyrrolyl, furanyl, thiophenyl, pyrazolyl, imidazolyl, oxazolyl, isoxazolyl, isothiazolyl, thiazolyl, triazolyl, oxodiazolyl, thiadiazolyl, tetrazolyl, pyridinyl, pyrazinyl, pyrimidinyl, pyridazinyl, triazinyl, dioxinyl, oxazinyl, or thiazinyl). In some embodiments, X₁ is a 5- or 6-membered heteroaryl comprising 1 to 4 heteroatoms selected from N, O, and S (e.g., pyrrolyl, furanyl, thiophenyl, pyrazolyl, imidazolyl, oxazolyl, isoxazolyl, isothiazolyl, thiazolyl, triazolyl, oxodiazolyl, thiadiazolyl, tetrazolyl, pyridinyl, pyrazinyl, pyrimidinyl, pyridazinyl, triazinyl, dioxinyl, oxazinyl, or thiazinyl) substituted as described herein.

In some embodiments, Ring A is

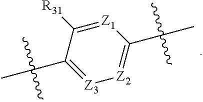

In some embodiments, Z₁, Z₂, and Z₃ are each independently CR₃₁.

In some embodiments, only one of Z₁, Z₂, and Z₃ is CR₃₁, and two of Z₁, Z₂, and Z₃ are N.

In some embodiments, Z₁ is CR₃₁, and Z₂ and Z₃ are N.

In some embodiments, Z₂ is CR₃₁, and Z₁ and Z₃ are N.

In some embodiments, Z₃ is CR₃₁, and Z₁ and Z₂ are N.

In some embodiments, only one of Z₁, Z₂, and Z₃ is N, and two of Z₁, Z₂, and Z₃ are CR₃₁.

In some embodiments, Z₁ is N, and Z₂ and Z₃ are CR₃₁.

In some embodiments, Z₂ is N, and Z₁ and Z₃ are CR₃₁.

In some embodiments, Z₃ is N, and Z₁ and Z₂ are CR₃₁.

In some embodiments, Ring A is

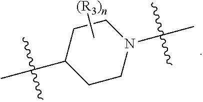

In some embodiments, the compounds of Formula (I) are of Formula (II1), (II2), (II3), (II4), (II5), (II6), (II7), (II8), (II9), (II10), (II11), (II12), (II13), (II14), (II15), (II16), (II17), or (II18):

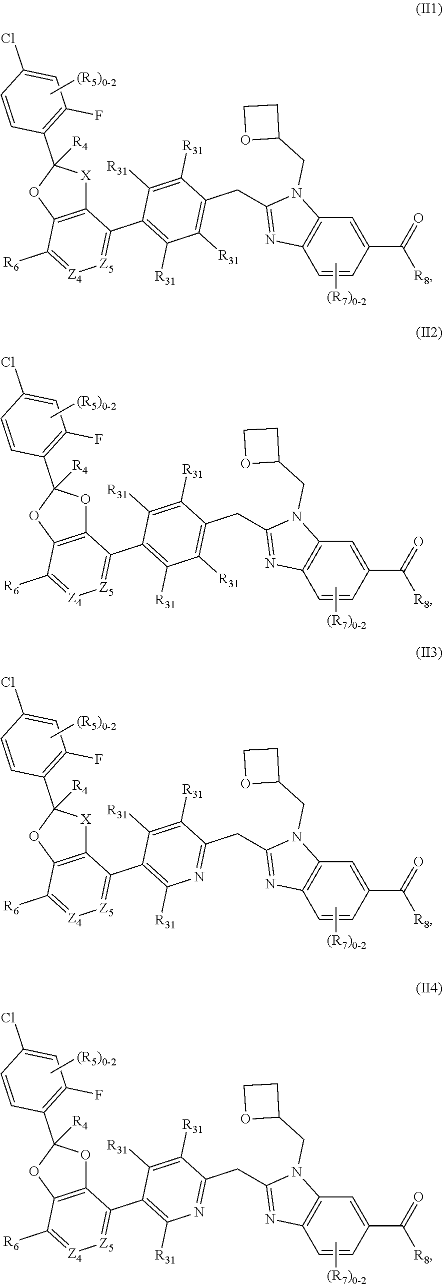

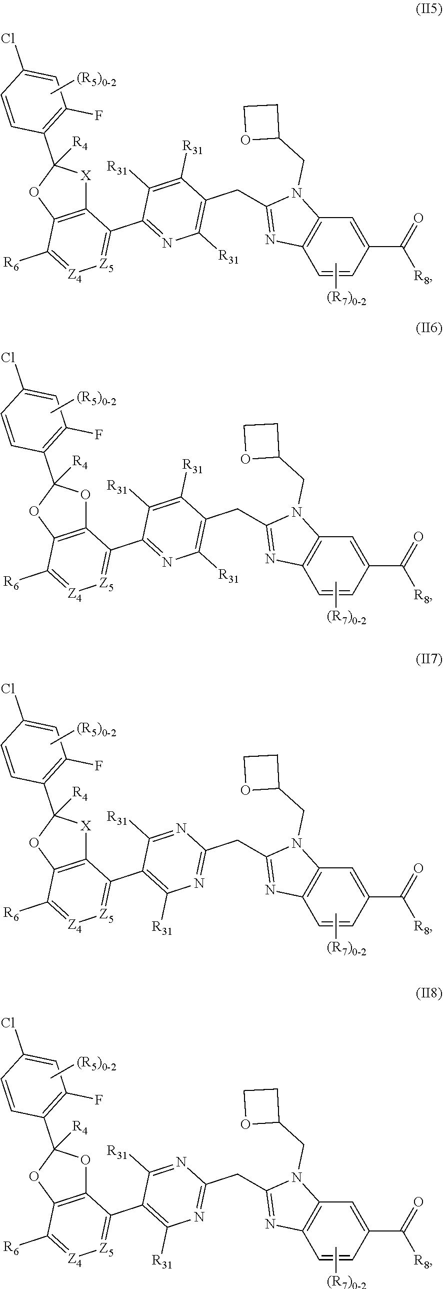

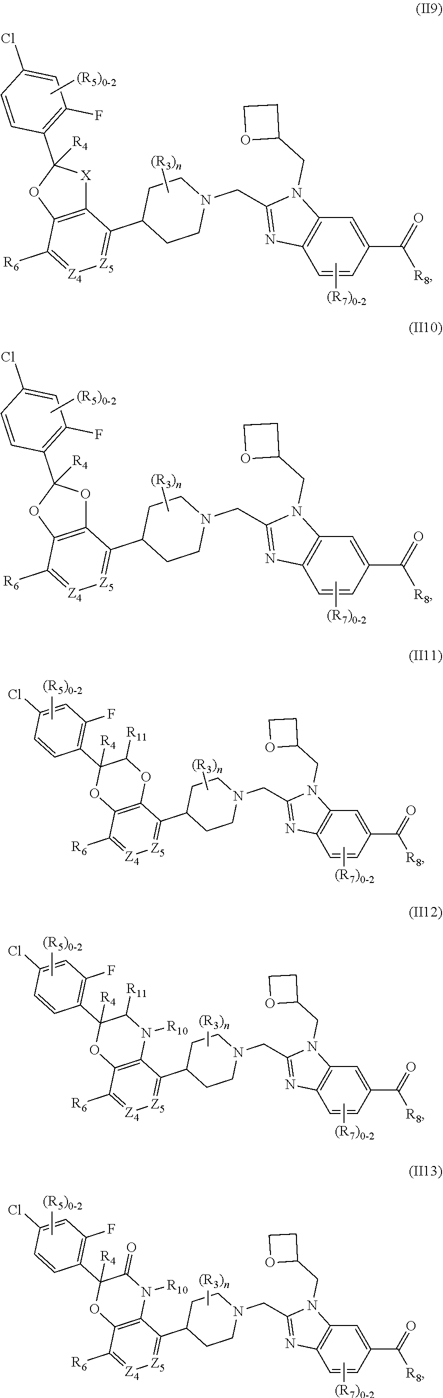

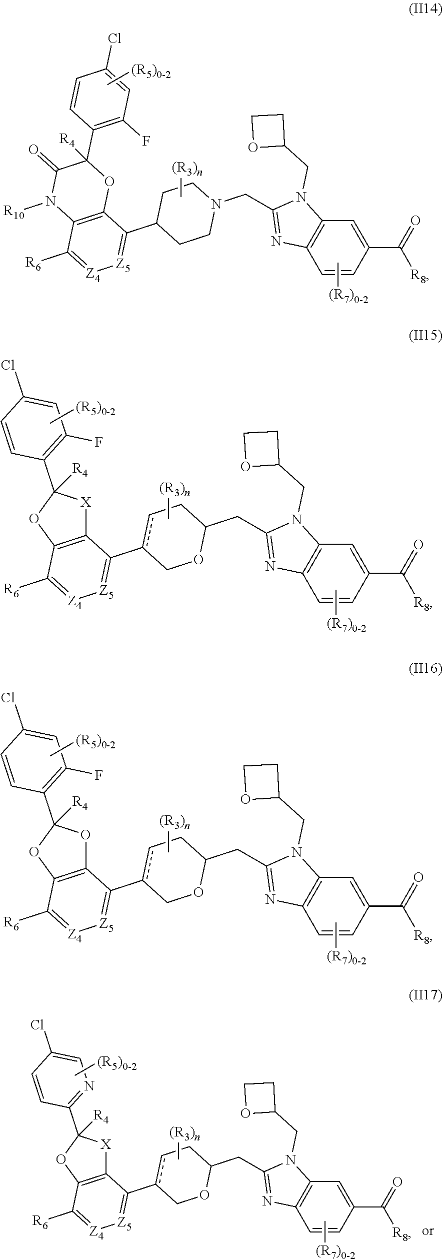

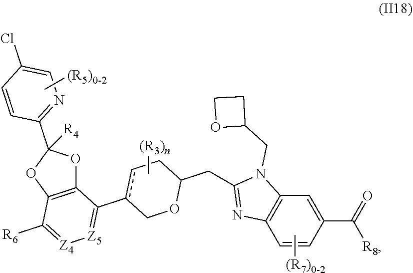

or a pharmaceutically acceptable salt, solvate, prodrug, stereoisomer, or tautomer thereof.

In some embodiments, the compounds of Formula (I) are of Formula ((II15), (II16), (II17), or (II18):

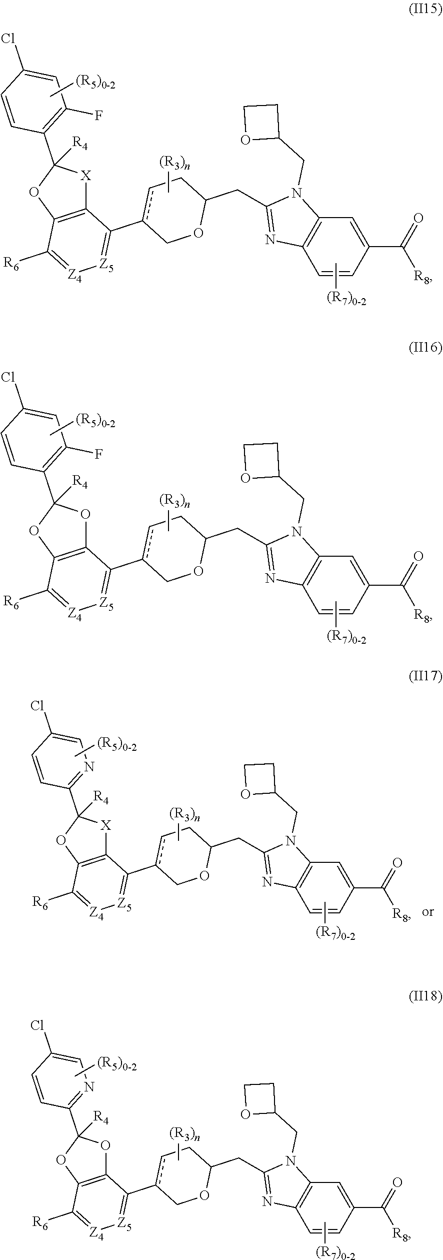

or a pharmaceutically acceptable salt, solvate, prodrug, stereoisomer, or tautomer thereof.

In some embodiments, the compounds of Formula (I) are of Formula (IIa1), (IIa2), (IIa3), (IIa4), (IIa5), (IIa6), (IIa7), (IIa8), (IIa9), (IIa10), (IIa11), (IIa12), or (IIa13):

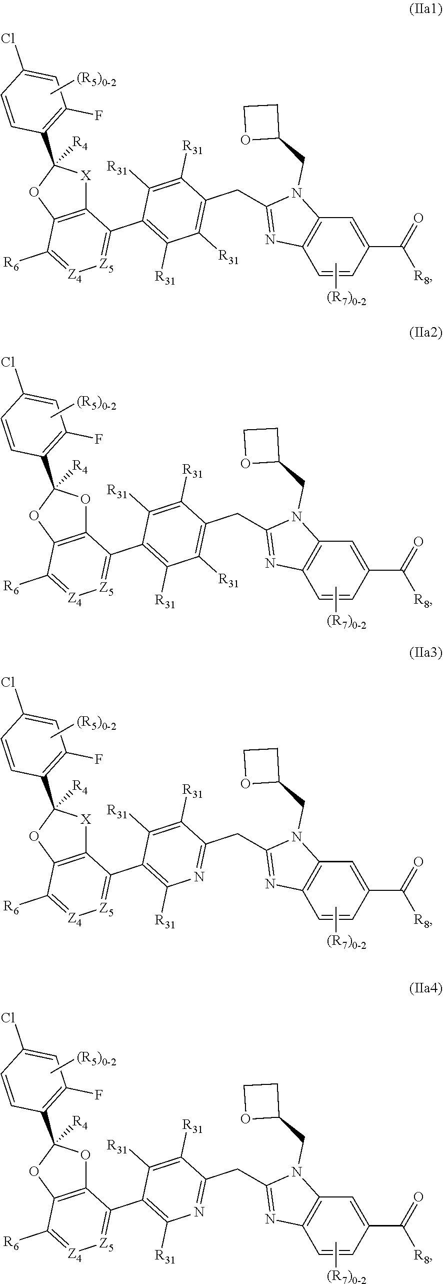

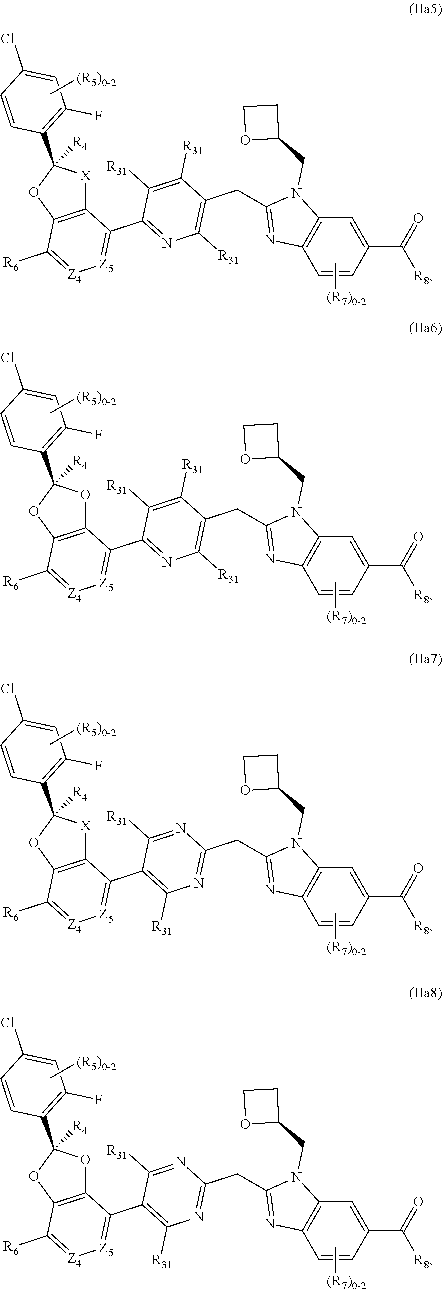

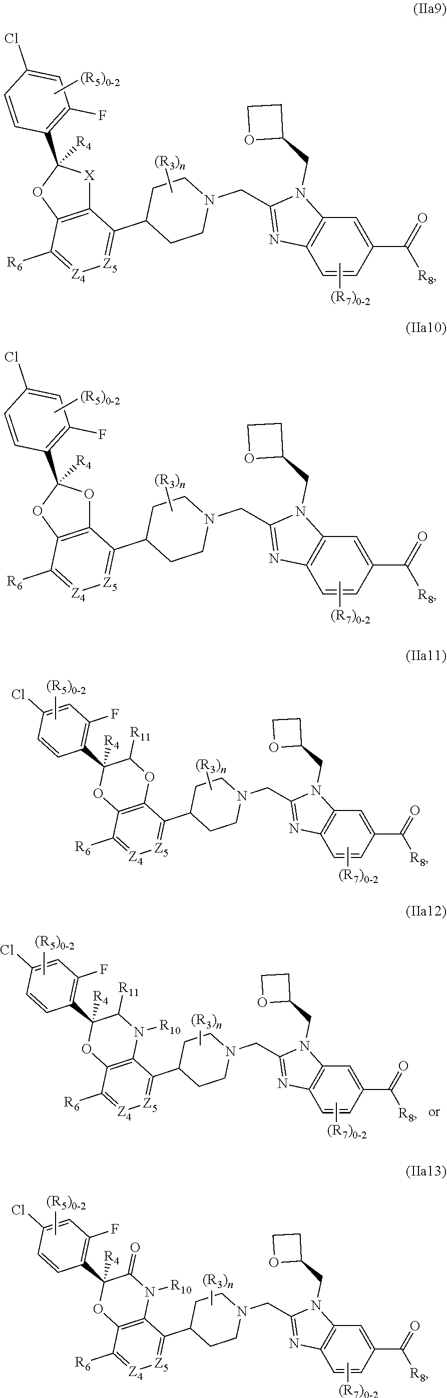

or a pharmaceutically acceptable salt, solvate, prodrug, stereoisomer, or tautomer thereof.

In some embodiments, the compounds of Formula (I) are of Formula (III1), (III2), (III3), (III4), (III5), (III6), (III7), (III8), (III9), (III10), (III11), (III12), (III13), (III14), or (III15):

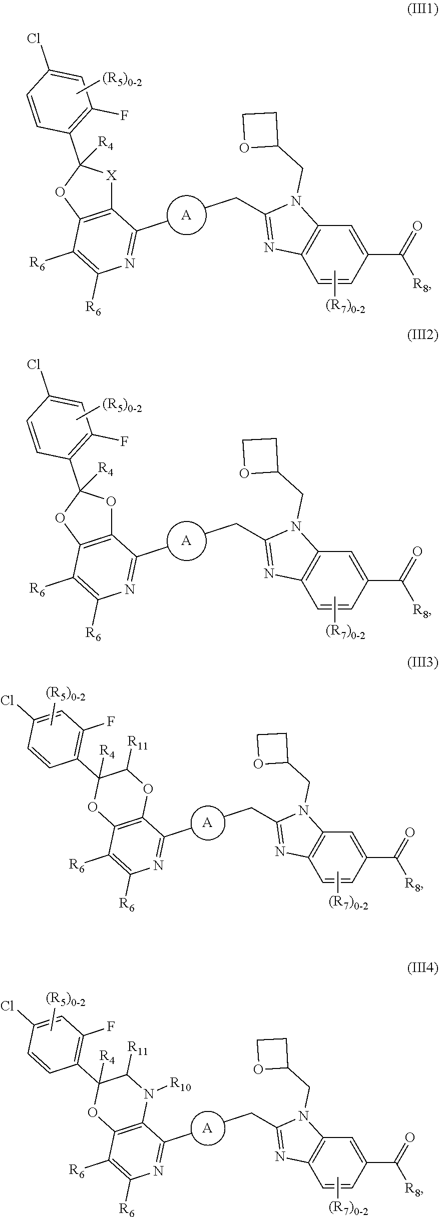

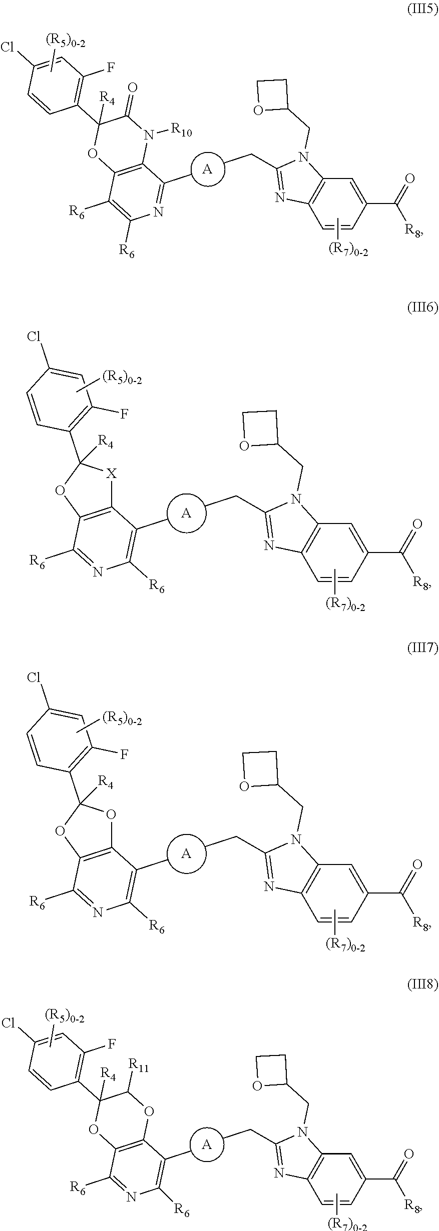

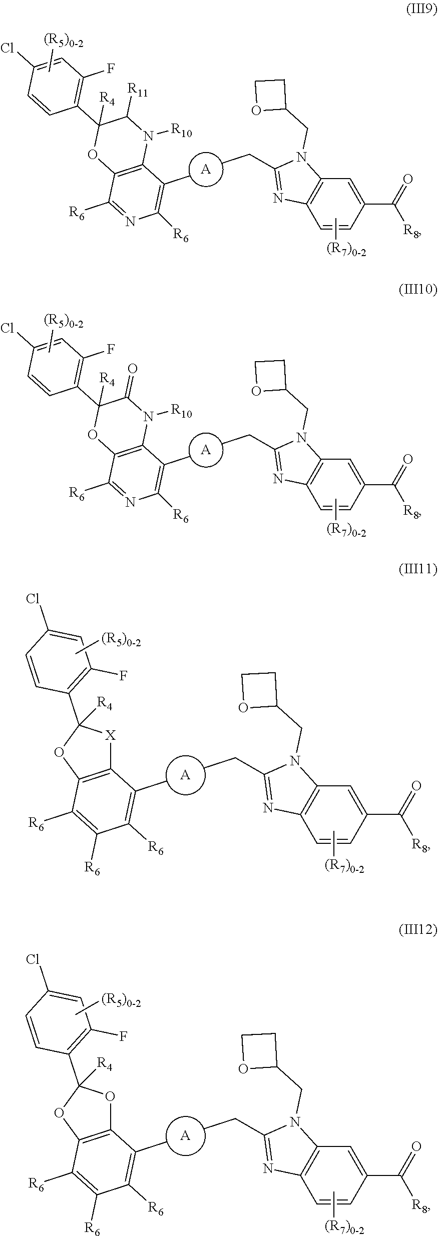

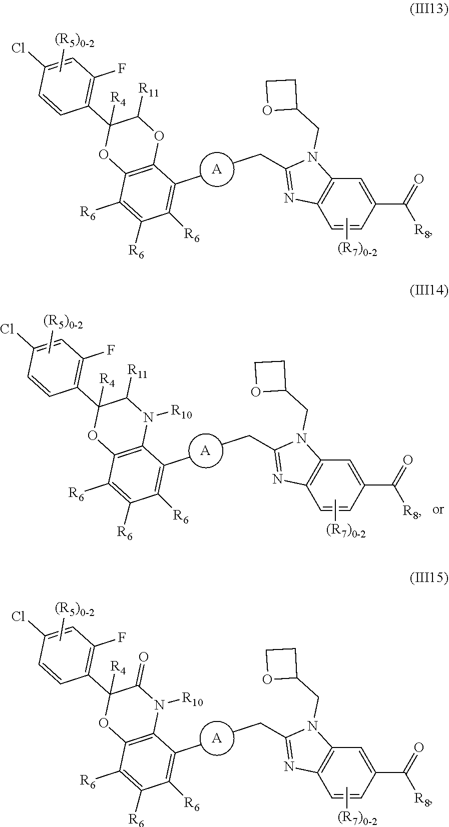

or a pharmaceutically acceptable salt, solvate, prodrug, stereoisomer, or tautomer thereof.

In some embodiments, the compounds of Formula (I) are of Formula (IIIa1), (IIIa2), (IIIa3), (IIIa4), (IIIa5), (IIIa6), (IIIa7), (IIIa8), (IIIa9), (IIIa10), (IIIa11), (IIIa12), (IIIa13), (IIIa14), or (IIIa15):

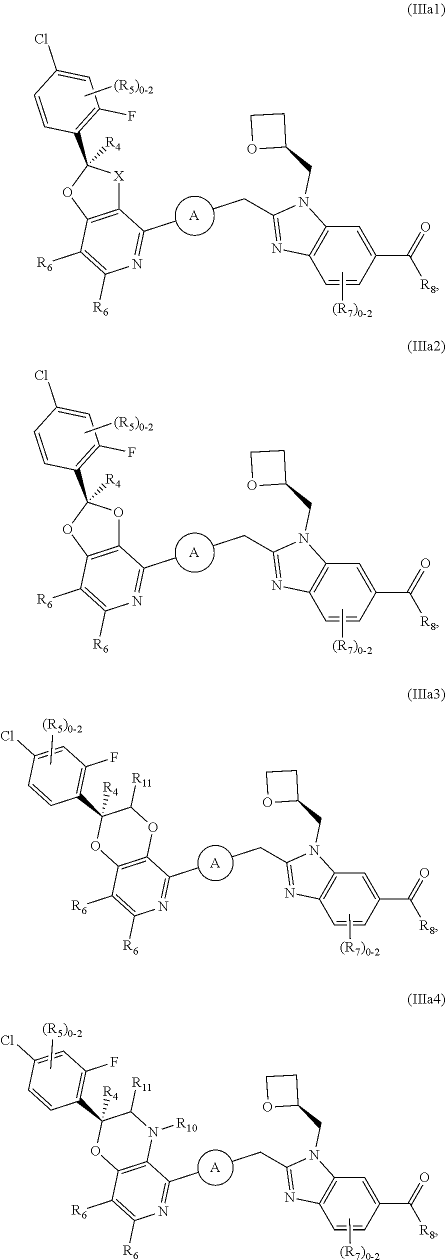

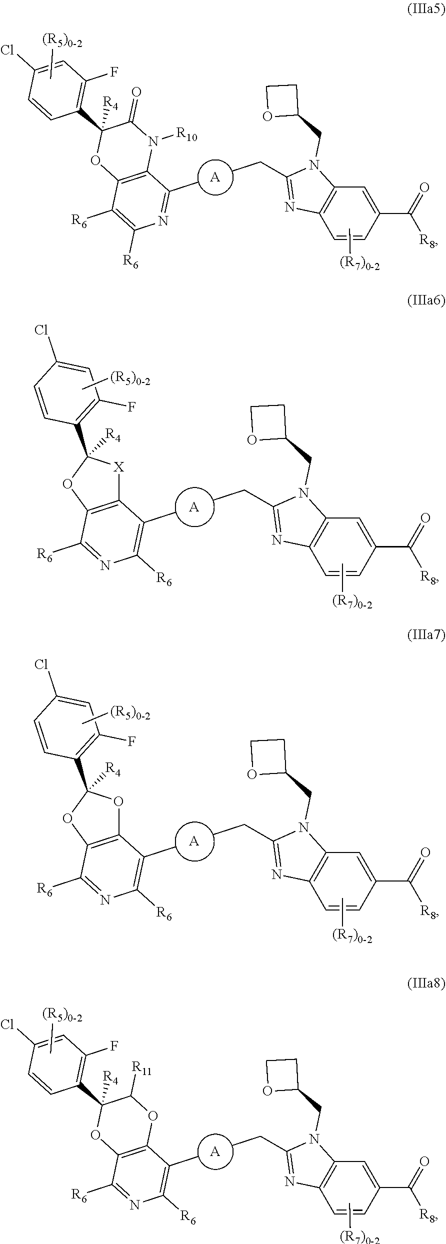

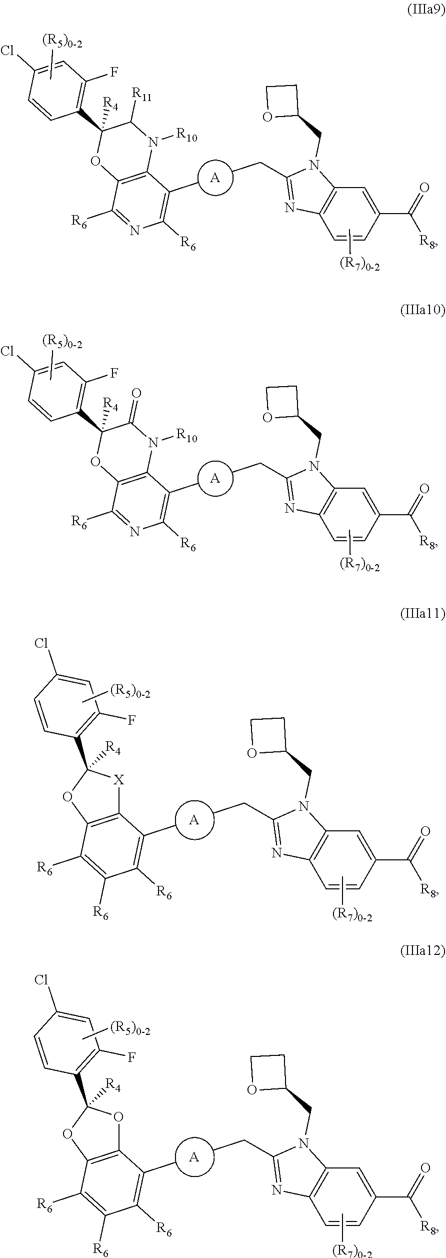

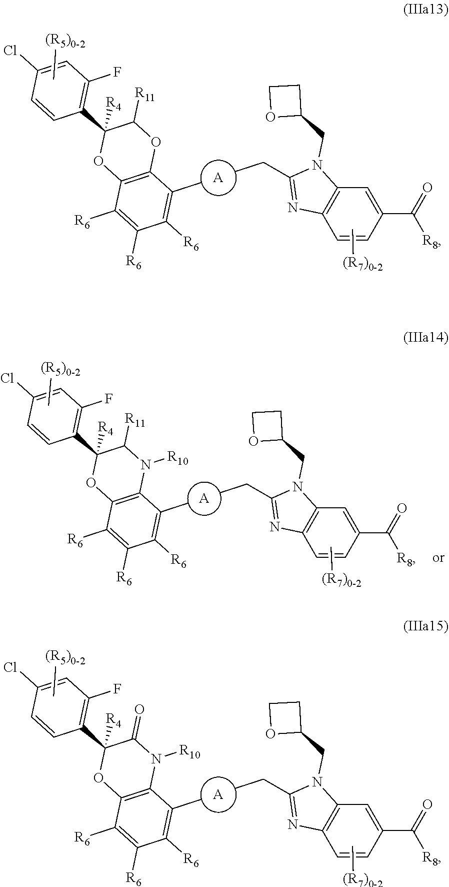

or a pharmaceutically acceptable salt, solvate, prodrug, stereoisomer, or tautomer thereof.

In some embodiments, the compounds of Formula (I) are of Formula (IV1), (IV2), (IV3), (IV4), (IV5), (IV6), (IV7), (IV8), (IV9), (IV10), (IV11), (IV12), (IV13), (IV14), (IV15), (IV16), (IV17), or (IV18):

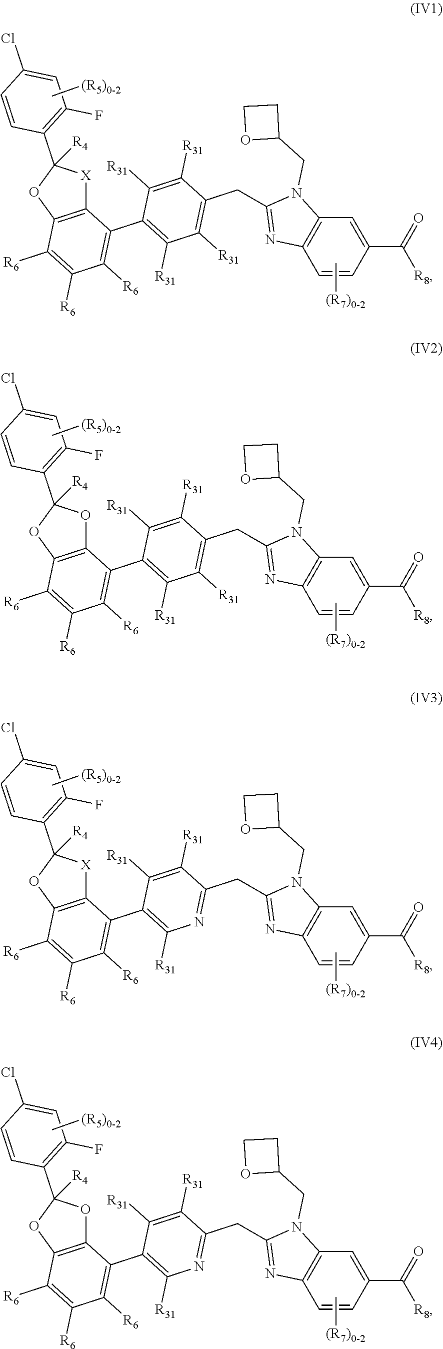

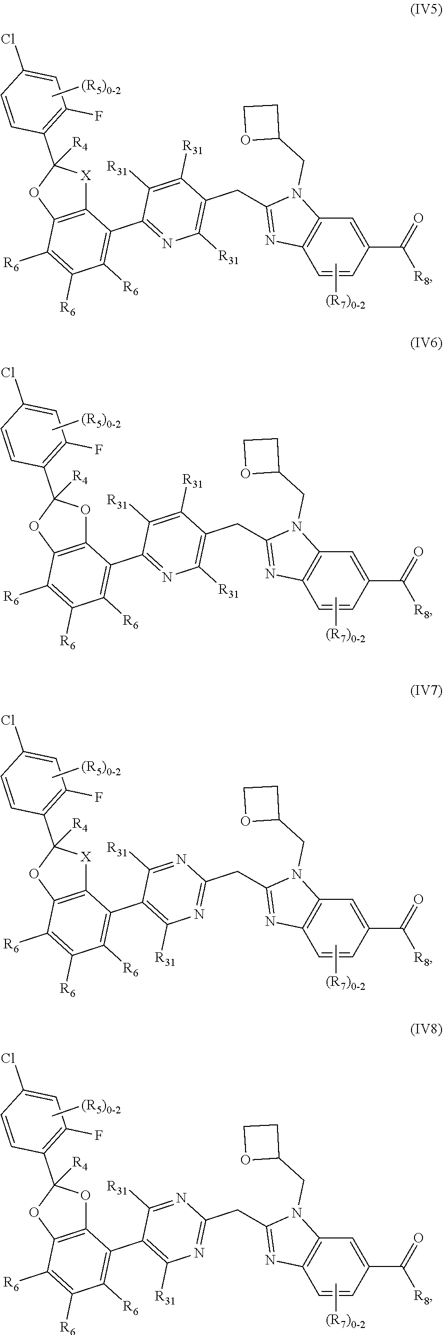

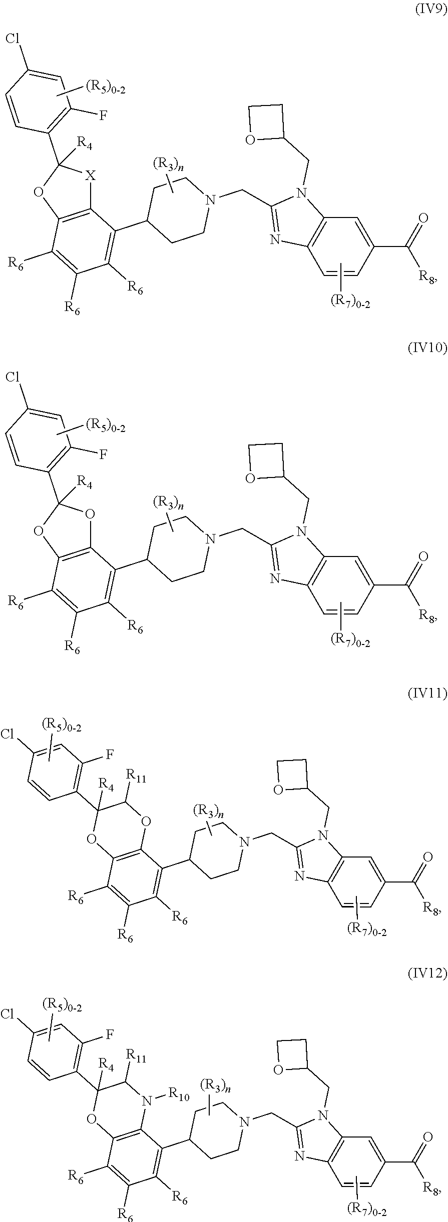

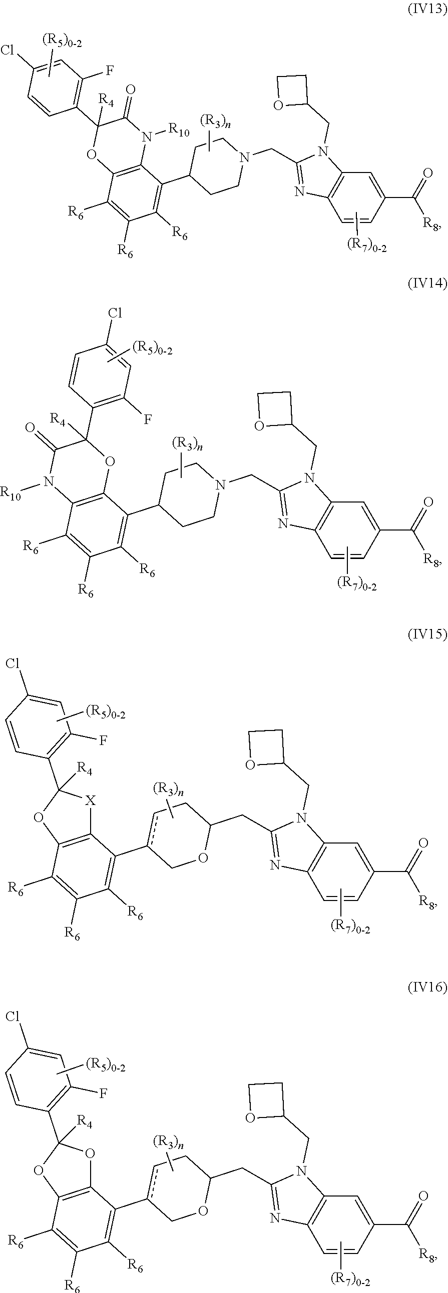

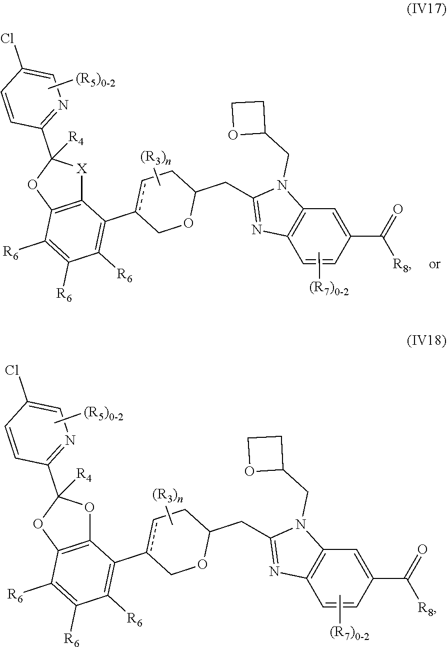

or a pharmaceutically acceptable salt, solvate, prodrug, stereoisomer, or tautomer thereof.

In some embodiments, the compounds of Formula (I) are of Formula (IV15), (IV16), (IV17), or (IV V18):

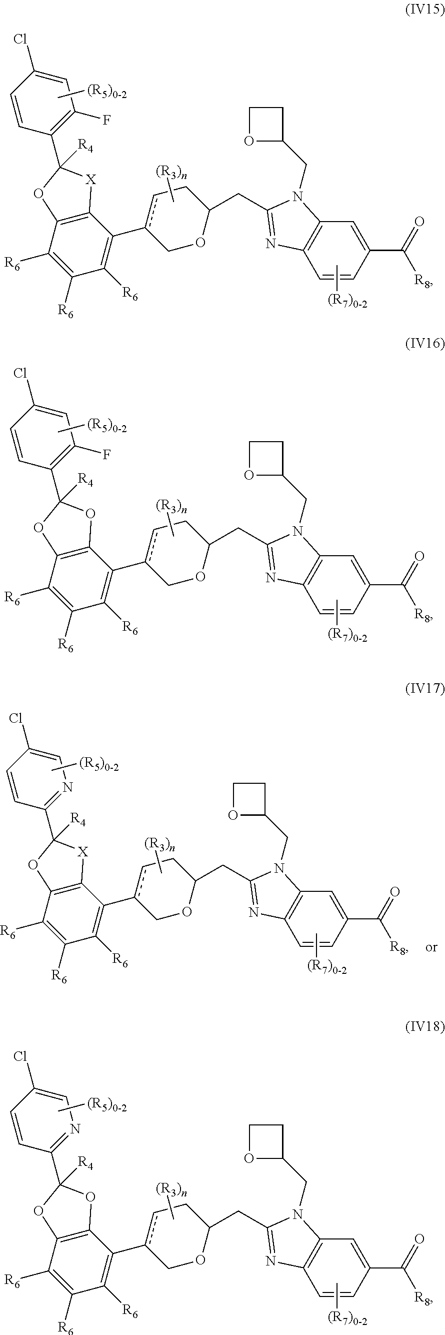

or a pharmaceutically acceptable salt, solvate, prodrug, stereoisomer, or tautomer thereof.

In some embodiments, the compounds of Formula (I) are of Formula (IVa1), (IVa2), (IVa3), (IVa4), (IVa5), (IVa6), (IVa7), (IVa8), (IVa9), (IVa10), (IVa11), (IVa12), or (IVa13):

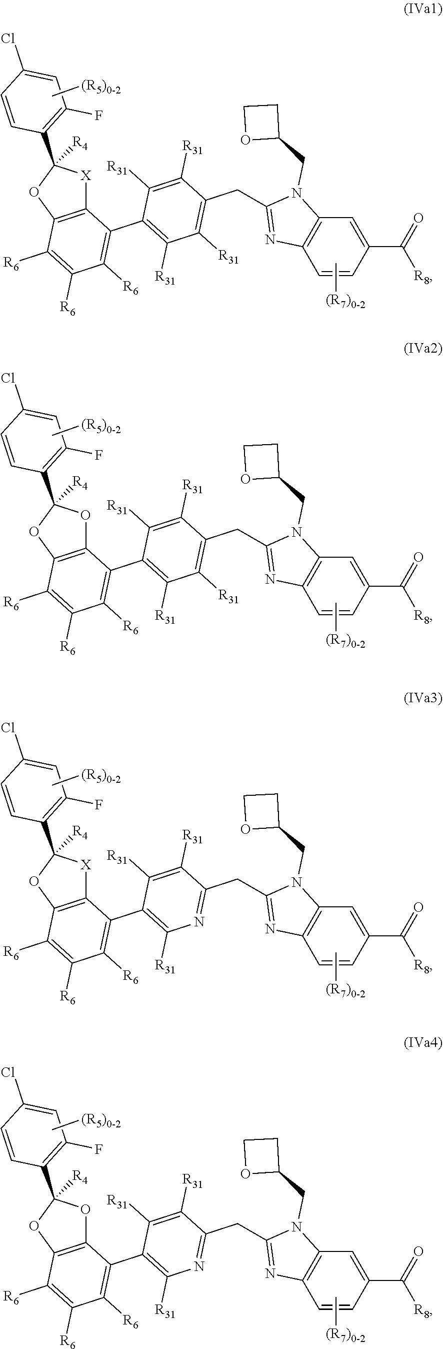

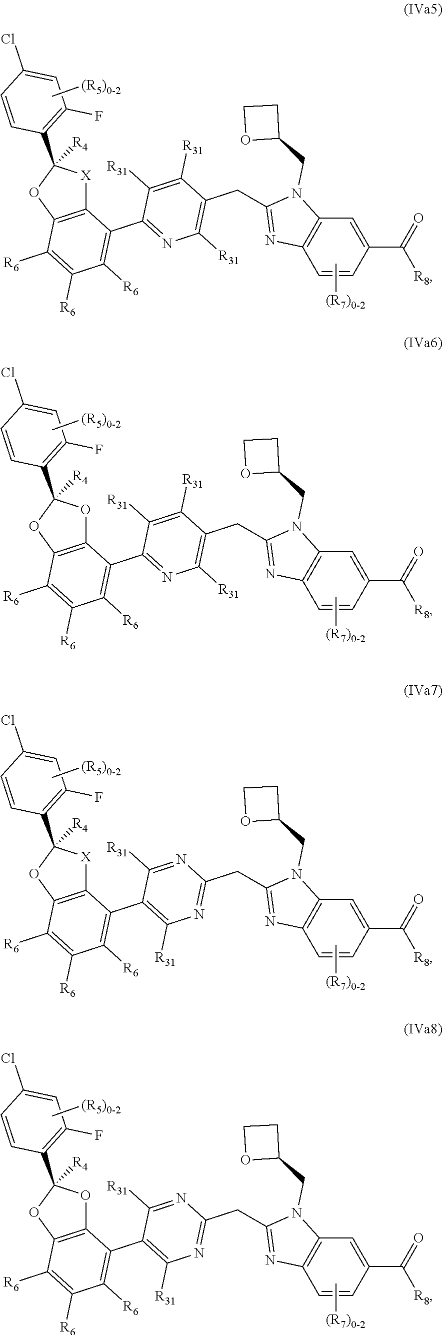

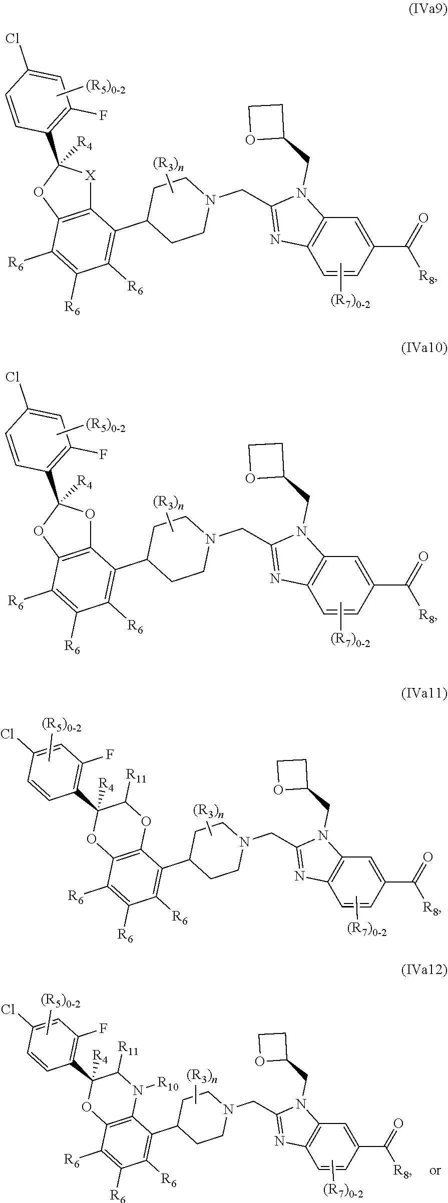

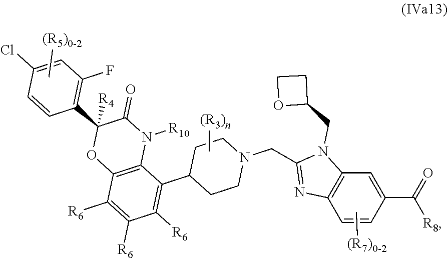

or a pharmaceutically acceptable salt, solvate, prodrug, stereoisomer, or tautomer thereof.

In some embodiments, the compounds of Formula (I) are of Formula (V1), (V2), (V3), (V4), (V5), (V6), (V7), (V8), (V9), (V10), (V11), (V12), or (V13):

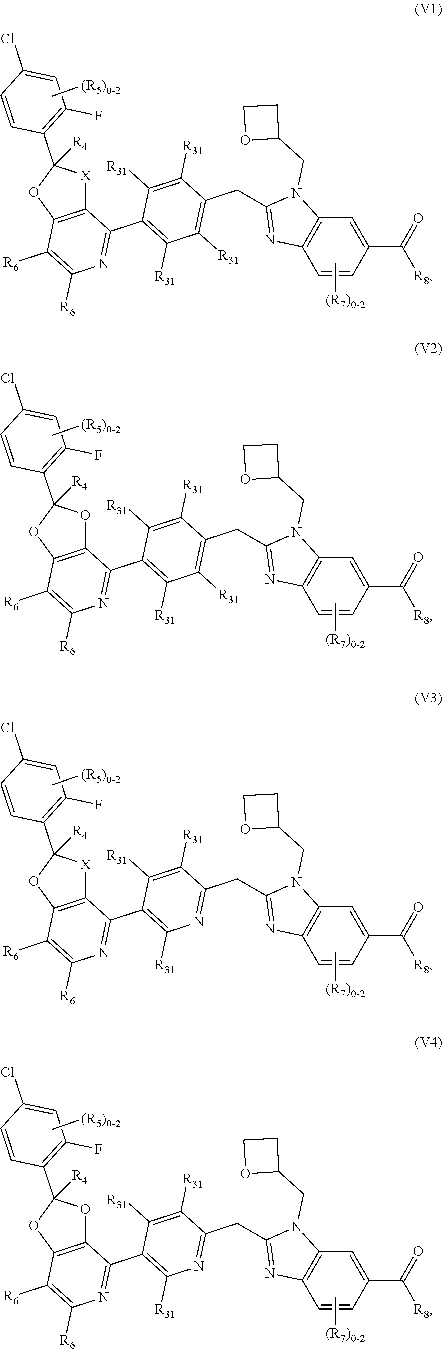

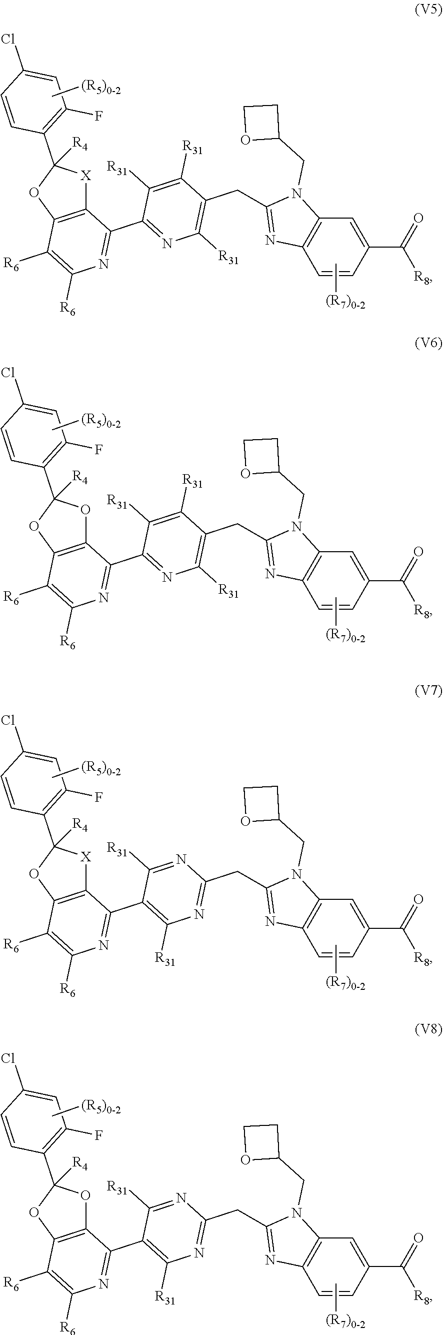

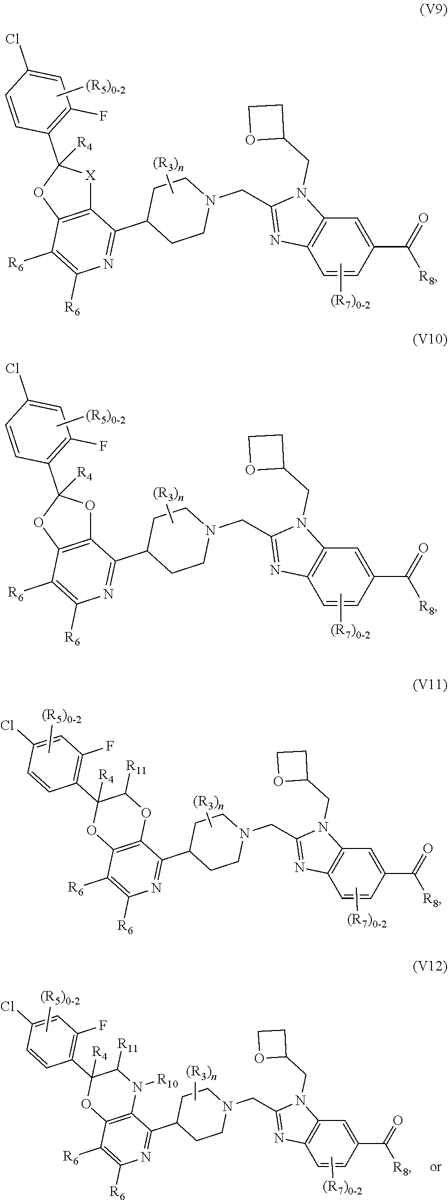

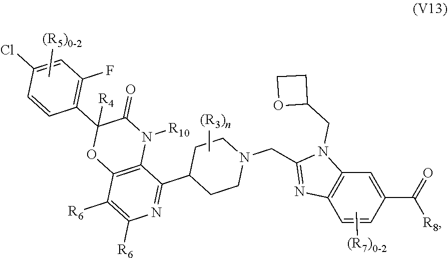

or a pharmaceutically acceptable salt, solvate, prodrug, stereoisomer, or tautomer thereof.

In some embodiments, the compounds of Formula (I) are of Formula (Va1), (Va2), (Va3), (Va4), (Va5), (Va6), (Va7), (Va8), (Va9), (Va10), (Va11), (Va12), or (Va13):

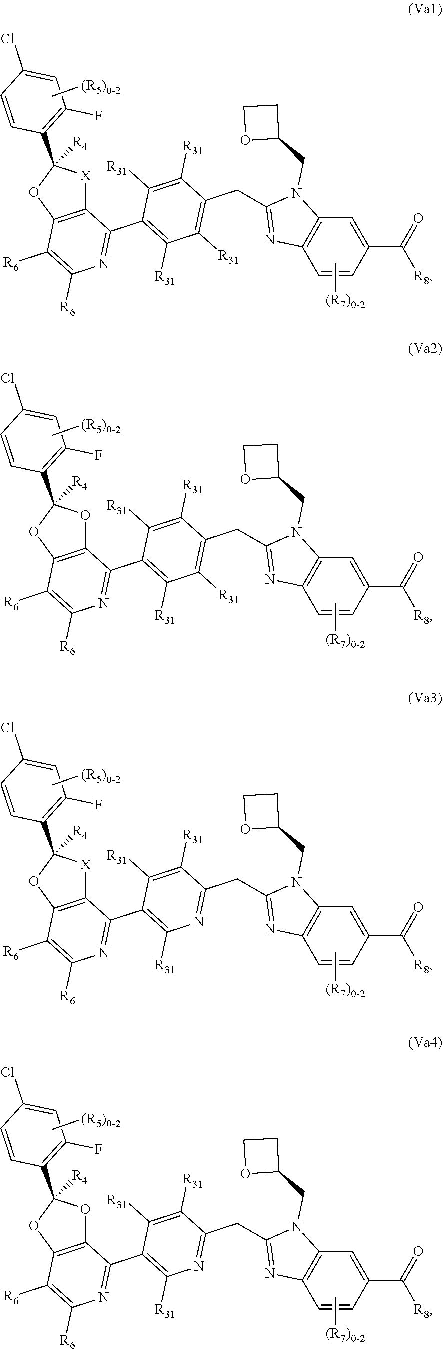

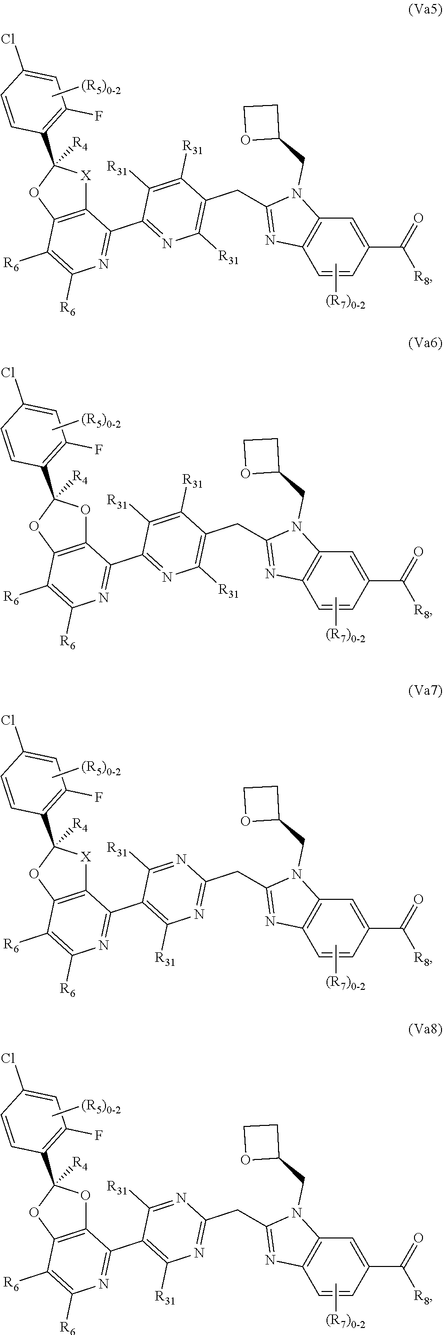

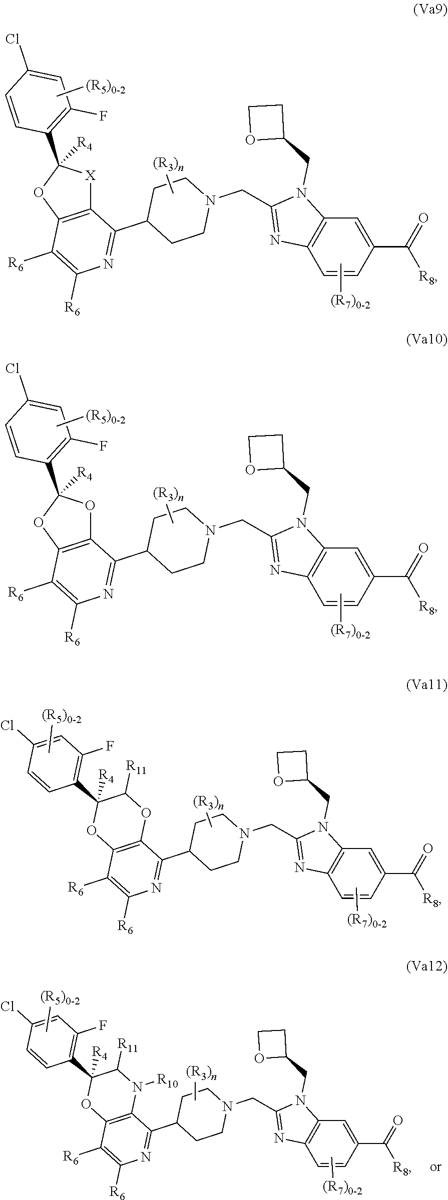

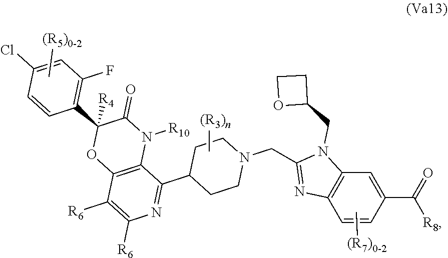

or a pharmaceutically acceptable salt, solvate, prodrug, stereoisomer, or tautomer thereof.

In some embodiments, the compounds of Formula (I) are of Formula (VI1), (VI2), (VI3), (VI4), (VI5), (VI6), (VI7), (VI8), (VI9), (VI10), (VI11), (VI12), or (VI13):

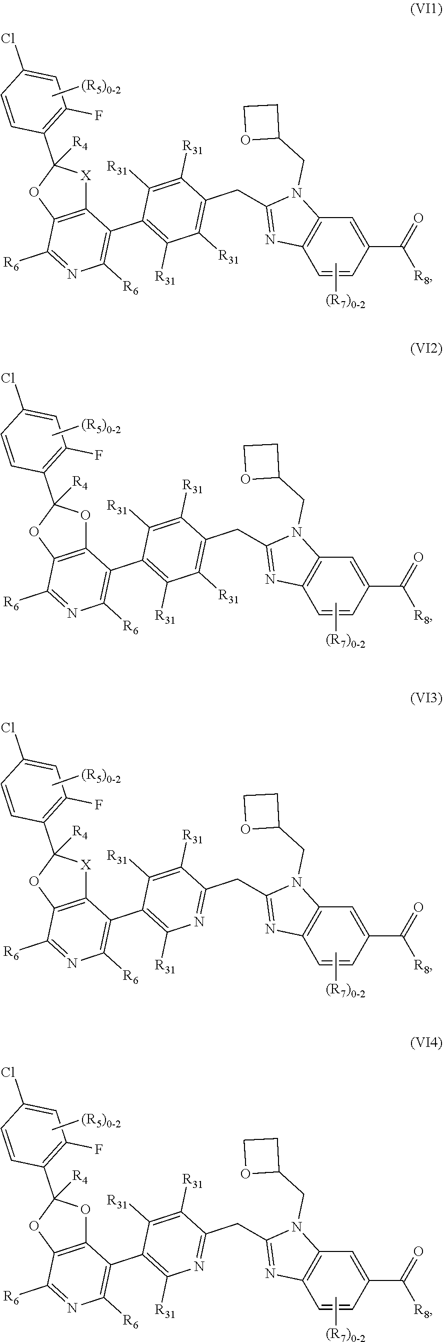

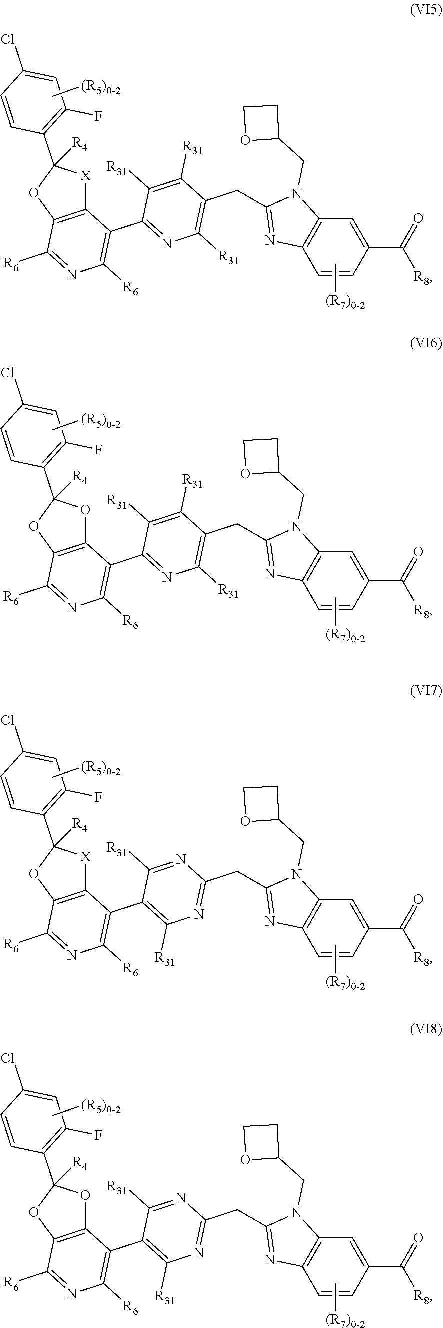

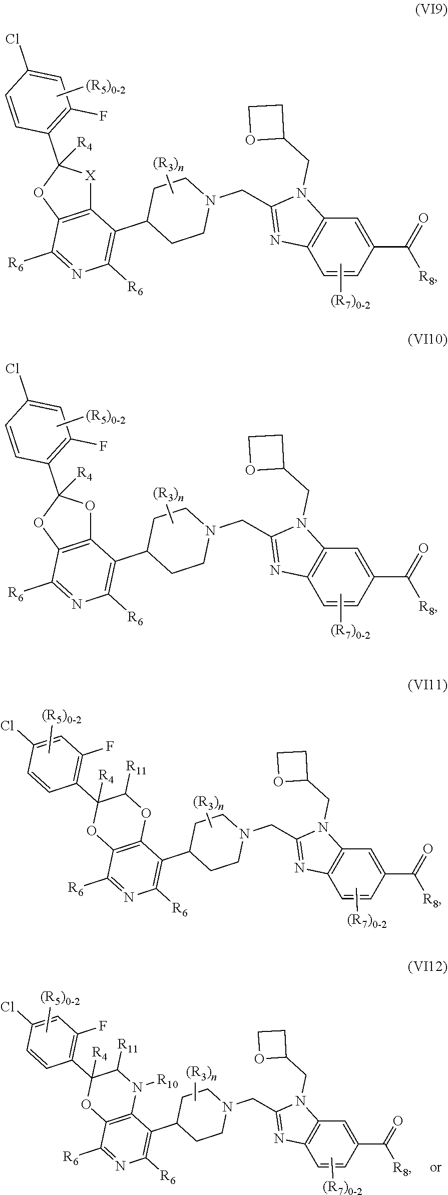

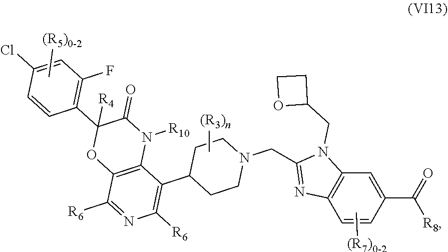

or a pharmaceutically acceptable salt, solvate, prodrug, stereoisomer, or tautomer thereof.

In some embodiments, the compounds of Formula (I) are of Formula (VIa1), (VIa2), (VIa3), (VIa4), (VIa5), (VIa6), (VIa7), (VIa8), (VIa9), (VIa10), (VIa11), (VIa12), or (VIa13):

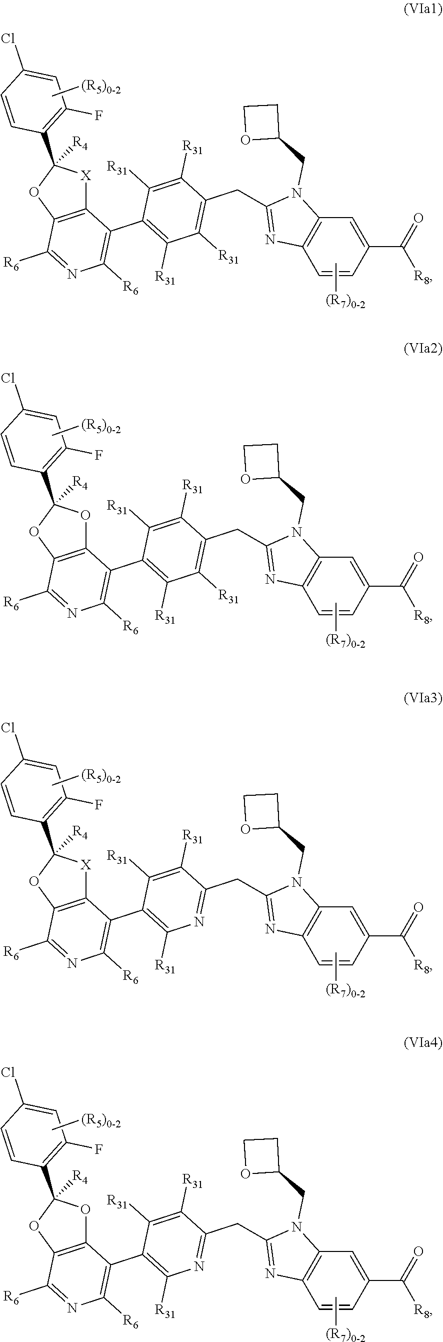

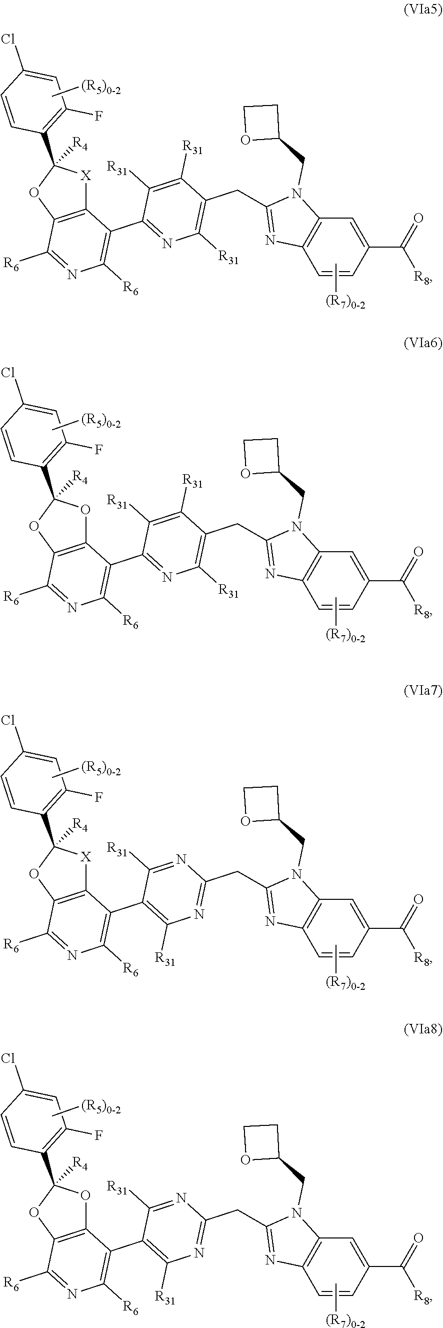

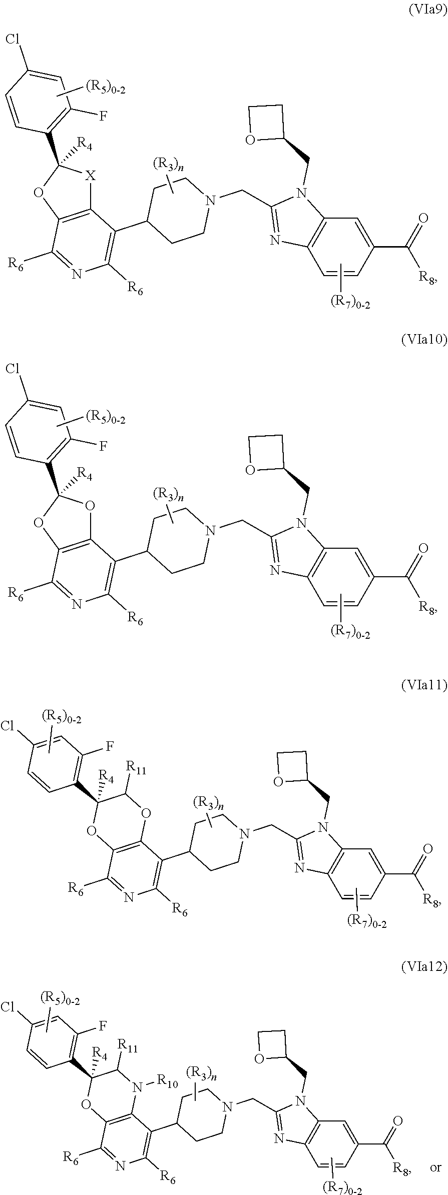

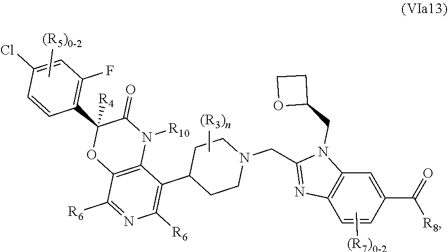

or a pharmaceutically acceptable salt, solvate, prodrug, stereoisomer, or tautomer thereof.

In some embodiments, the compound of Formula (Y) is a compound of Formula (A′):

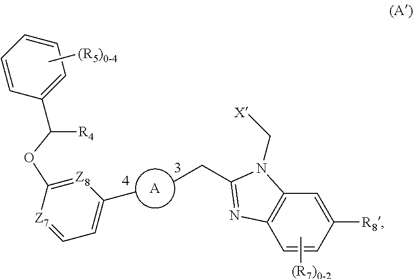

or a pharmaceutically acceptable salt, solvate, prodrug, stereoisomer, or tautomer thereof.

In some embodiments, the compound of Formula (Y) is a compound is of Formula (I′):

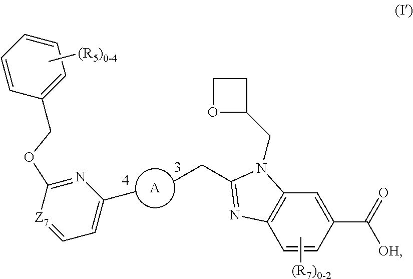

or a pharmaceutically acceptable salt, solvate, prodrug, stereoisomer, or tautomer thereof.

In some embodiments, the compound of Formula (Y) is a compound of the following formulas:

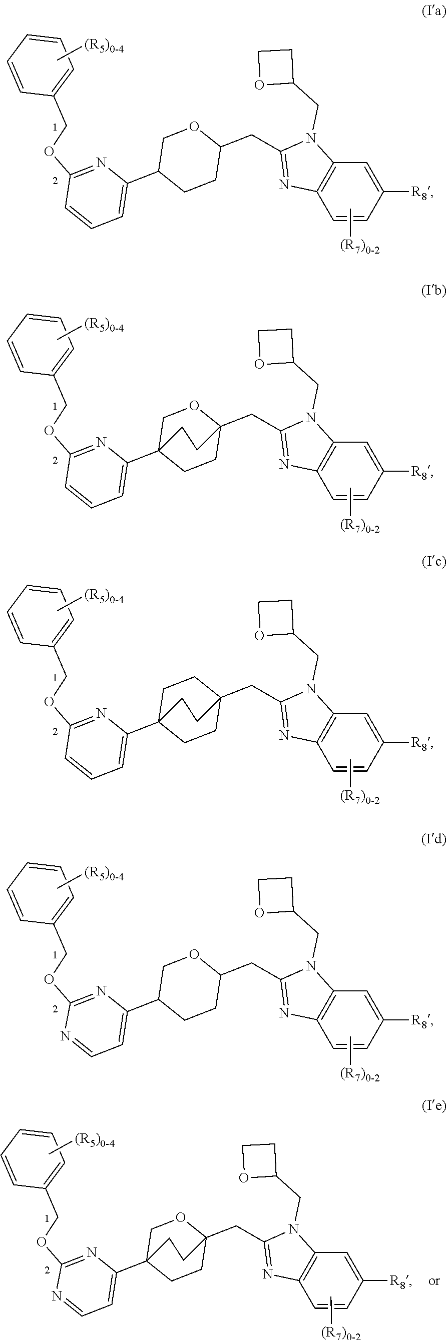

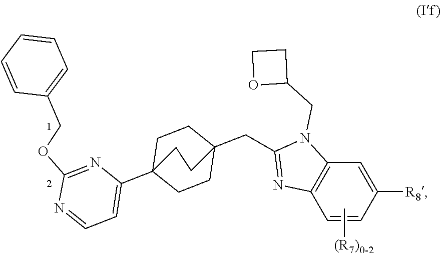

or a pharmaceutically acceptable salt, solvate, prodrug, stereoisomer, or tautomer thereof.

For compounds of any one of the formulae described herein, where applicable:

In some embodiments, Ring A is

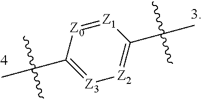

In some embodiments, Ring A is

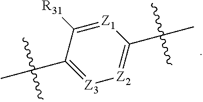

In some embodiments, Z₀, Z₁, Z₂, and Z₃ are each independently CR₃₁.

In some embodiments, only one of Z₀, Z₁, Z₂, and Z₃ is CR₃₁, and three of Z₀, Z₁, Z₂, and Z₃ are N.

In some embodiments, Z₀ is CR₃₁, and Z₁, Z₂, and Z₃ are N.

In some embodiments, Z₁ is CR₃₁, and Z₀, Z₂ and Z₃ are N.

In some embodiments, Z₂ is CR₃₁, and Z₀, Z₁ and Z₃ are N.

In some embodiments, Z₃ is CR₃₁, and Z₀, Z₁ and Z₂ are N.

In some embodiments, two of Z₀, Z₁, Z₂, and Z₃ are CR₃₁, and two of Z₀, Z₁, Z₂, and Z₃ are N.

In some embodiments, Z₀ and Z₁ are N; and Z₂ and Z₃ are CR₃₁.

In some embodiments, Z₀ and Z₂ are N; and Z₁ and Z₃ are CR₃₁.

In some embodiments, Z₀ and Z₃ are N; and Z₁ and Z₂ are CR₃₁.

In some embodiments, Z₁ and Z₂ are N; and Z₀ and Z₃ are CR₃₁.

In some embodiments, Z₁ and Z₃ are N; and Z₀ and Z₂ are CR₃₁.

In some embodiments, Z₂ and Z₃ are N; and Z₀ and Z₁ are CR₃₁.

In some embodiments, only one of Z₀, Z₁, Z₂, and Z₃ is N, and three of Z₀, Z₁, Z₂, and Z₃ are CR₃₁.

In some embodiments, Z₀ is N, and Z₁, Z₂ and Z₃ are CR₃₁.

In some embodiments, Z₁ is N, and Z₀, Z₂ and Z₃ are CR₃₁.

In some embodiments, Z₂ is N, and Z₀, Z₁ and Z₃ are CR₃₁.

In some embodiments, Z₃ is N, and Z₀, Z₁ and Z₂ are CR₃₁.

In some embodiments, Ring A is

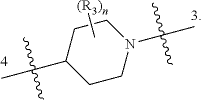

In some embodiments, Ring A is

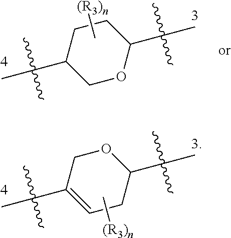

In some embodiments, Ring A is

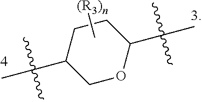

In some embodiments, Ring A is

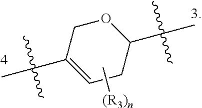

In some embodiments, Ring A is

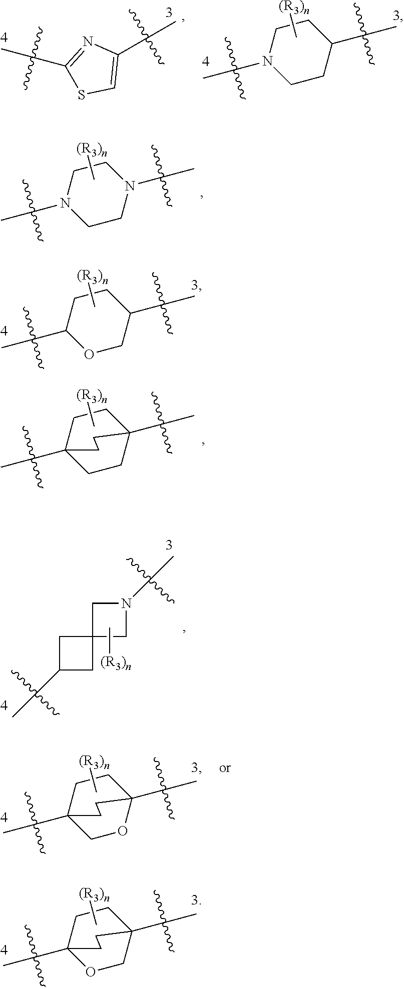

In some embodiments, each R₃₁ is H. In some embodiments, at least one R₃₁ is R₃.

In some embodiments, at least one R₃ is C₁-C₄ alkyl (e.g., methyl, ethyl, propyl, i-propyl, n-butyl, i-butyl, s-butyl, or t-butyl). In some embodiments, at least one R₃ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, at least one R₃ is C₁-C₄ alkoxy (e.g., methoxy, ethoxy, propoxy, i-propoxy, n-butoxy, i-butoxy, s-butoxy, or t-butoxy). In some embodiments, at least one R₃ is halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, at least one R₃ is halogen (e.g., F, Cl, Br, or I). In some embodiments, at least one R₃ is OH. In some embodiments, at least one R₃ is CN. In some embodiments, at least one R₃ is NH₂.

In some embodiments, n is 0. In some embodiments, n is 1. In some embodiments, n is 2. In some embodiments, n is 3. In some embodiments, n is 4.

In some embodiments

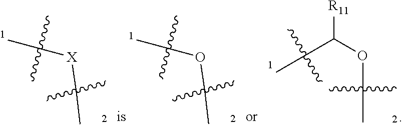

In some embodiments,

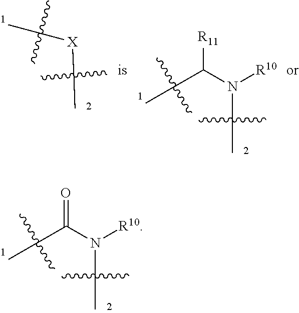

In some embodiments,

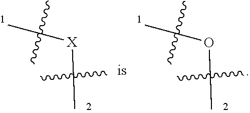

In some embodiments, R₁ and R₂ are each H. In some embodiments, at least one of R₁ and R₂ is C₁-C₄ alkyl (e.g., methyl, ethyl, propyl, i-propyl, n-butyl, i-butyl, s-butyl, or t-butyl). In some embodiments, at least one of R₁ and R₂ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, at least one of R₁ and R₂ is C₁-C₄ alkoxy (e.g., methoxy, ethoxy, propoxy, i-propoxy, n-butoxy, i-butoxy, s-butoxy, or t-butoxy). In some embodiments, at least one of R₁ and R₂ is halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, at least one of R₁ and R₂ is halogen (e.g., F, Cl, Br, or I). In some embodiments, at least one of R₁ and R₂ is OH.

In some embodiments, R₁ and R₂, together with the carbon atom to which they are attached, form a C₃-C₆ carbocyclic ring (e.g., cyclopropyl, cyclobutyl, cyclopentyl, or cyclohexyl). In some embodiments, R₁ and R₂, together with the carbon atom to which they are attached, form 4- to 6-membered heterocyclic ring comprising 1 or 2 heteroatoms selected from N, O, and S (e.g., azetidinyl, oxetanyl, thietanyl, pyrrolidinyl, tetrahydrofuranyl, tetrahydrothiophenyl, oxazolidinyl, isoxazolidinyl, thiazolidinyl, isothiazolidinyl, piperidinyl, piperazinyl, tetrahydropyranyl, tetrahydrothiapyranyl, dioxanyl, morpholinyl, oxazinanyl, thiazinanyl, or oxathianyl).

In some embodiments, R₁₀ is H. In some embodiments, R₁₀ is C₁-C₄ alkyl (e.g., methyl, ethyl, propyl, i-propyl, n-butyl, i-butyl, s-butyl, or t-butyl). In some embodiments, R₁₀ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, R₁₀ is C(O)Rₐ.

In some embodiments, R₁₁ is H. In some embodiments, R₁₁ is C₁-C₄ alkyl (e.g., methyl, ethyl, propyl, i-propyl, n-butyl, i-butyl, s-butyl, or t-butyl). In some embodiments, Ru is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, Ru is C₁-C₄ alkoxy (e.g., methoxy, ethoxy, propoxy, i-propoxy, n-butoxy, i-butoxy, s-butoxy, or t-butoxy). In some embodiments, Ru is halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, Ru is halogen (e.g., F, Cl, Br, or I). In some embodiments, Ru is OH. In some embodiments, two Ru, together with carbon atom to which they are attached, form a C₃-C₆ carbocylyl or 4- to 6-membered heterocylyl comprising 1 or 2 heteroatoms selected from N, O, and S. In some embodiments, two Ru are taken together to form an oxo.

In some embodiments, Z₄, Z₅, and Z₇ are each CR₆.

In some embodiments, only one of Z₄, Z₅, and Z₇ is CR₆, and two of Z₄, Z₅, and Z₇ are N.

In some embodiments, Z₄ and Z₅ are each N, and Z₇ is CR₆.

In some embodiments, Z₄ and Z₇ are each N, and Z₅ is CR₆.

In some embodiments, Z₅ and Z₇ are each N, and Z₄ is CR₆.

In some embodiments, only one of Z₄, Z₅, and Z₇ is N, and two of Z₄, Z₅, and Z₇ are CR₆.

In some embodiments, Z₄ and Z₇ are each CR₆ and Z₅ is N.

In some embodiments, Z₅ and Z₇ are each CR₆ and Z₄ is N.

In some embodiments, Z₄ and Z₅ are each CR₆ and Z₇ is N.

In some embodiments, Z₄, Z₅, and Z₇ are each N.

In some embodiments, Z₈ is C. In some embodiments, Z₈ is N, wherein when Z₈ is N, X is absent.

In some embodiments, each R₆ is H. In some embodiments, at least one R₆ is C₁-C₄ alkyl (e.g., methyl, ethyl, propyl, i-propyl, n-butyl, i-butyl, s-butyl, or t-butyl). In some embodiments, at least one R₆ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, at least one R₆ is C₁-C₄ alkoxy (e.g., methoxy, ethoxy, propoxy, i-propoxy, n-butoxy, i-butoxy, s-butoxy, or t-butoxy). In some embodiments, at least one R₆ is halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, at least one R₆ is halogen (e.g., F, Cl, Br, or I). In some embodiments, at least one R₆ is OH. In some embodiments, at least one R₆ is CN. In some embodiments, at least one R₆ is NH₂.

In some embodiments, R₄ is C₁-C₄ alkyl, halo-C₁-C₄ alkyl, or C₃-C₆ carbocyclyl.

In some embodiments, R₄ is H only when

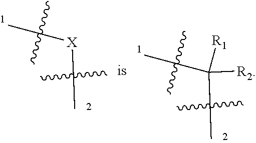

In some embodiments, R₄ is H.

In some embodiments, R₄ is C₁-C₄ alkyl (e.g., methyl, ethyl, propyl, i-propyl, n-butyl, i-butyl, s-butyl, or t-butyl). In some embodiments, R₄ is methyl. In some embodiments, R₄ is ethyl. In some embodiments, R₄ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, R₄ is C₃-C₆ carbocyclyl (e.g., cyclopropyl, cyclobutyl, cyclopentyl, or cyclohexyl). In some embodiments, R₄ is cyclopropyl.

In some embodiments, Ring A is

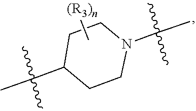

X is O, and Z₄ and Z₅ are each CR₆, and R₄ is C₂-C₄ alkyl (e.g., ethyl, propyl, i-propyl, n-butyl, i-butyl, s-butyl, or t-butyl).

In some embodiments, Ring A is

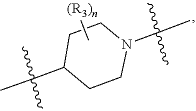

X is O, and Z₄ and Z₅ are each CR₆, and R₄ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)).

In some embodiments, Ring A is

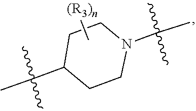

X is O, and Z₄ and Z₅ are each CR₆, and R₄ is C₃-C₆ carbocyclyl (e.g., cyclopropyl, cyclobutyl, cyclopentyl, or cyclohexyl).

In some embodiments, at least one R₅ is C₁-C₄ alkyl (e.g., methyl, ethyl, propyl, i-propyl, n-butyl, i-butyl, s-butyl, or t-butyl). In some embodiments, at least one R₅ is C₃-C₆ carbocyclyl (e.g., cyclopropyl, cyclobutyl, cyclopentyl, or cyclohexyl). In some embodiments, at least one R₅ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, at least one R₅ is C₁-C₄ alkoxy (e.g., methoxy, ethoxy, propoxy, i-propoxy, n-butoxy, i-butoxy, s-butoxy, or t-butoxy). In some embodiments, at least one R₅ is halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, at least one R₅ is halogen (e.g., F, Cl, Br, or I). In some embodiments, at least one R₅ is OH. In some embodiments, at least one R₅ is CN. In some embodiments, at least one R₅ is NH₂.

In some embodiments, at least one R₇ is C₁-C₄ alkyl (e.g., methyl, ethyl, propyl, i-propyl, n-butyl, i-butyl, s-butyl, or t-butyl). In some embodiments, at least one R₇ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, at least one R₇ is C₁-C₄ alkoxy (e.g., methoxy, ethoxy, propoxy, i-propoxy, n-butoxy, i-butoxy, s-butoxy, or t-butoxy). In some embodiments, at least one R₇ is halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, at least one R₇ is halogen (e.g., F, Cl, Br, or I). In some embodiments, at least one R₇ is OH. In some embodiments, at least one R₇ is CN. In some embodiments, at least one R₇ is NH₂.

In some embodiments, R₈′ is C(O)R₈ or NHC(O)Rᵢᵢ.

In some embodiments, R₈′ is C(O)R₈.

In some embodiments, R₈′ is 5- to 10-membered heteroaryl comprising one or two 5- or 6-membered rings and 1 to 4 heteroatoms selected from N, O, and S.

In some embodiments, R₈ is ORᵢᵢ.

In some embodiments, R₈ is N(Rᵢᵢ)₂.

In some embodiments, R₈ is NRᵢᵢSO₂Rᵢᵢᵢ.

In some embodiments, each Rᵢᵢ is independently H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S.

In some embodiments, Rᵢᵢᵢ is C₁-C₄ alkyl, halo-C₁-C₄ alkyl, C₁-C₄ alkoxy, C₁-C₄ alkylamino, C₁-C₄ dialkylamino, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S.

In some embodiments, R₈ is OH or O—C₁-C₄ alkyl. In some embodiments, R₈ is OH.

In some embodiments, R₈ is NH₂, NH—C₁-C₄ alkyl, or N(C₁-C₄ alkyl)₂.

In some embodiments, R₈ is OH. In some embodiments, R₈ is O—C₁-C₄ alkyl (e.g., methyl, ethyl, propyl, i-propyl, n-butyl, i-butyl, s-butyl, or t-butyl). In some embodiments, R₈ is NH₂. In some embodiments, R₈ is NH—C₁-C₄ alkyl (e.g., methyl, ethyl, propyl, i-propyl, n-butyl, i-butyl, s-butyl, or t-butyl). In some embodiments, R₈ is N(C₁-C₄ alkyl)₂ (e.g., methyl, ethyl, propyl, i-propyl, n-butyl, i-butyl, s-butyl, or t-butyl).

In some embodiments, at least one R₉ is C₁-C₄ alkyl (e.g., methyl, ethyl, propyl, i-propyl, n-butyl, i-butyl, s-butyl, or t-butyl). In some embodiments, at least one R₉ is halo-C₁-C₄ alkyl (e.g., methyl, ethyl, propyl, i-propyl, n-butyl, i-butyl, s-butyl, or t-butyl, each of which is substituted with one or more halogen (e.g., F, Cl, Br, or I)). In some embodiments, at least one R₉ is C₁-C₄ alkoxy (e.g., methoxy, ethoxy, propoxy, i-propoxy, n-butoxy, i-butoxy, s-butoxy, or t-butoxy). In some embodiments, at least one R₉ is halo-C₁-C₄ alkoxy (e.g., methoxy, ethoxy, propoxy, i-propoxy, n-butoxy, i-butoxy, s-butoxy, or t-butoxy, each of which is substituted with one or more halogen (e.g., F, Cl, Br, or I)). In some embodiments, at least one R₉ is halogen (e.g., F, Cl, Br, or I). In some embodiments, at least one R₉ is OH. In some embodiments, at least one R₉ is CN. In some embodiments, at least one R₉ is NH₂.

In some embodiments, the compounds of Formula (I) are of Formula (A1), (A1a), (A2), (A2a), (A3), (A3a), (A4), (A4a), (A5), (A5a), (A6), (A6a), (A7), (A7a), (A8), (A8a), (A9), (A9a), (A10), or (A10a):

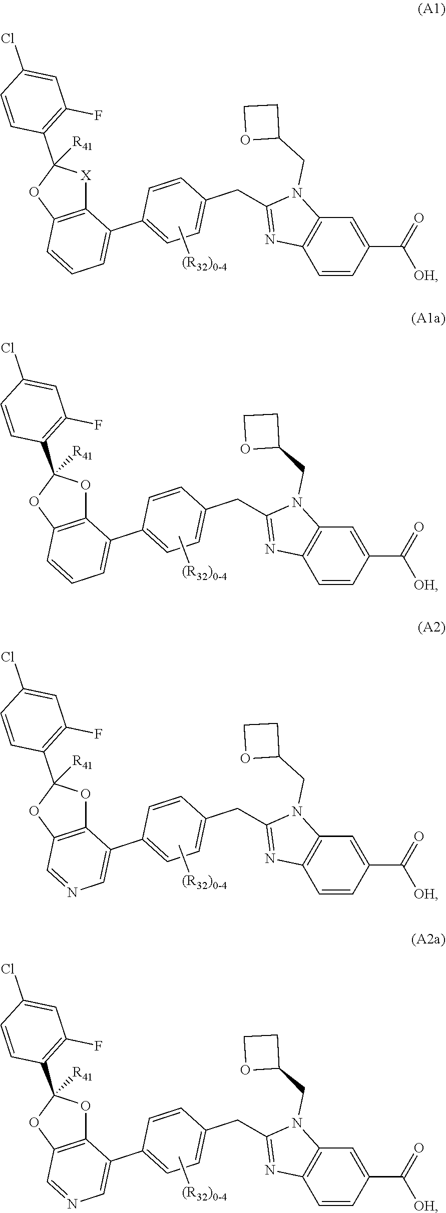

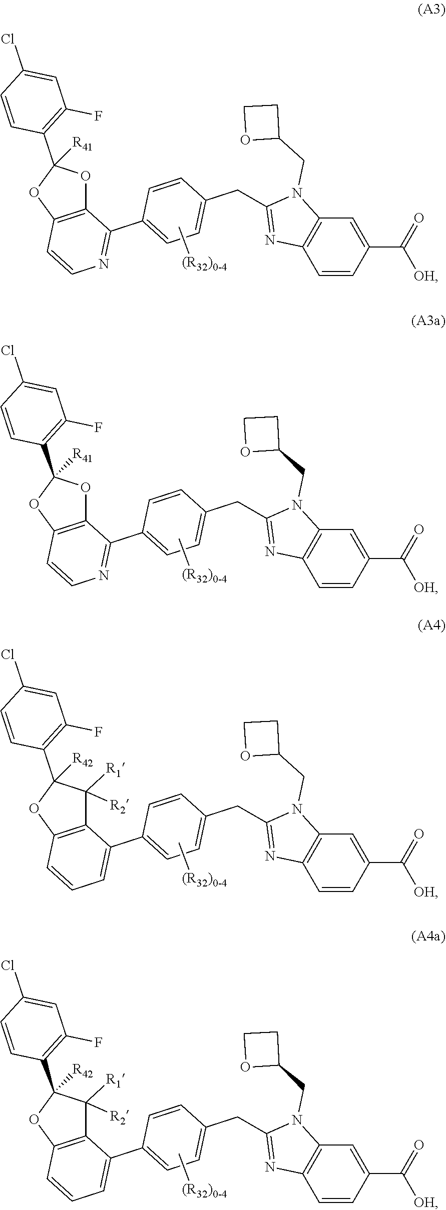

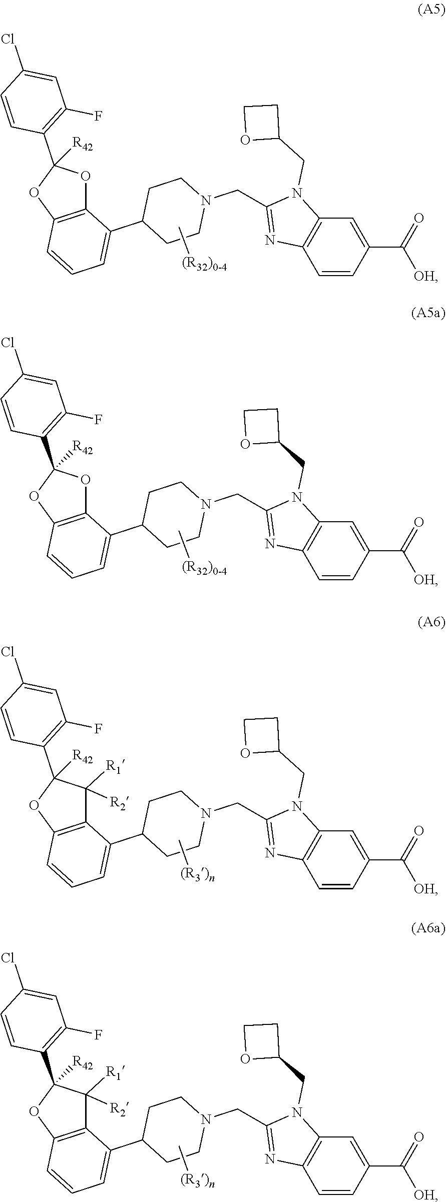

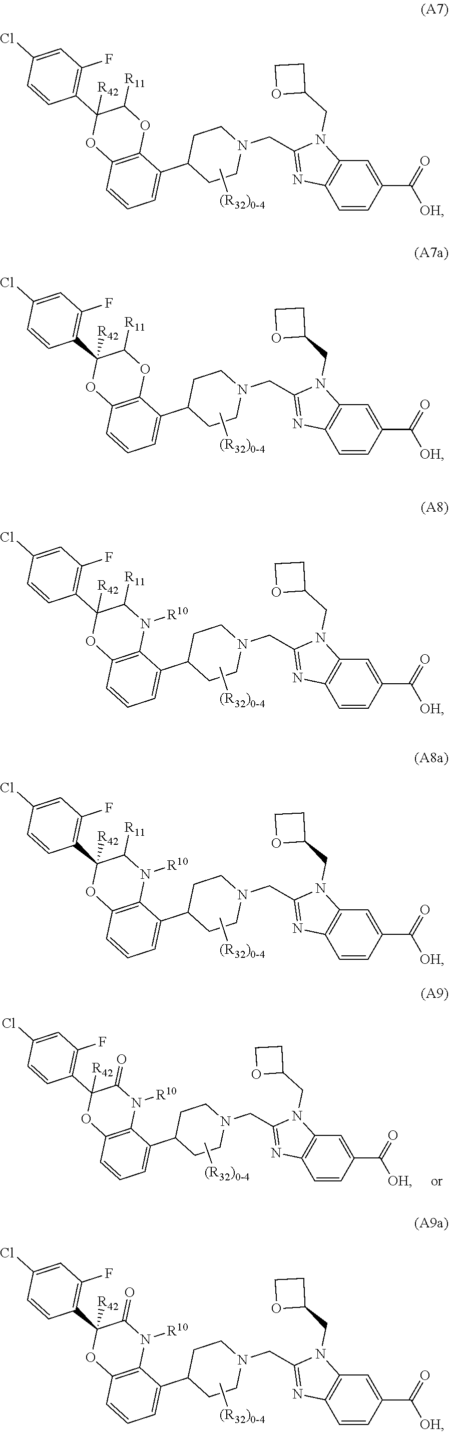

or a pharmaceutically acceptable salt, solvate, prodrug, stereoisomer, or tautomer thereof, wherein:

R₄₁ is methyl, ethyl, CF₃, or CH₂CF₃;

R₄₂ is H, methyl, ethyl, CF₃, or CH₂CF₃;

each R₃₂ is independently C₁-C₄ alkyl, CF₃, F, or CN;

R₁′ and R₂′ are each independently H, methyl, CF₃, or F, or R₁′ and R₂′, together with the carbon atom to which they are attached, form a C₃-C₆ carbocyclic ring or 4- to 6-membered heterocyclic ring comprising 1 or 2 heteroatoms selected from N, O, and S;

each R₃′ is independently C₁-C₄ alkyl, CF₃, F, or CN; and

n is 0, 1, 2, 3, or 4.

In some embodiments, R₄₁ is methyl or ethyl.

In some embodiments, R₄₂ is H.

In some embodiments, R₄₂ is methyl or ethyl.

In some embodiments, at least one R₃₂ is C₁-C₄ alkyl.

In some embodiments, at least one R₃₂ is CF₃, F, or CN.

In some embodiments, R₁′ and R₂′ are each H.

In some embodiments, at least one of R₁′ and R₂′ is methyl, CF₃, or F.

In some embodiments, R₁′ and R₂′, together with the carbon atom to which they are attached, form a C₃-C₆ carbocyclic ring or 4- to 6-membered heterocyclic ring comprising 1 or 2 heteroatoms selected from N, O, and S;

In some embodiments, at least one R₃′ is C₁-C₄ alkyl.

In some embodiments, at least one R₃′ is CF₃, F, or CN.

In some embodiments, n is 0.

In some embodiments, n is 1 or 2.

In one aspect, compounds selected from Table A are described.

In another aspect, pharmaceutical compositions comprising the compound described herein and a pharmaceutically acceptable salt, solvate, prodrug, stereoisomer, or tautomer thereof, and a pharmaceutically acceptable diluent, carrier, or excipient.

In yet another aspect, methods of treating or preventing a GLP-1 receptor-mediated disease or disorder or of modulating GLP-1 receptor, comprising administering to a subject in need thereof a therapeutically effective amount of the compound described herein or a pharmaceutically acceptable salt, solvate, prodrug, stereoisomer, or tautomer thereof, or of the pharmaceutical composition described herein.

In some embodiments, the disease or disorder is selected from diabetes, diabetic complication, obesity, impaired glucose tolerance, overweight condition, hyperlipidemia, hypercholesteremia, atherosclerosis, hypertension, coronary heart disease, congestive heart failure, cardiac arrhythmias, brain infarction, stroke, liver disease, nonalcoholic fatty liver disease (NAFLD), nonalcoholic steatohepatitis (NASH), dementia, Parkinson's disease, and diabetic kidney disease.

In one aspect, the compounds described herein or pharmaceutically acceptable salts, solvates, prodrugs, stereoisomers, or tautomers thereof, or pharmaceutical compositions described herein for use in methods of treating or preventing a GLP-1 receptor-mediated disease or disorder or of modulating GLP-1 receptor are described.

In another aspect, the compounds described herein or pharmaceutically acceptable salts, solvates, prodrugs, stereoisomers, or tautomers thereof, or pharmaceutical compositions of the compound described herein in the manufacture of a medicament for treating or preventing GLP-1 receptor-mediated diseases or disorders or for modulating GLP-1 receptor is described.

Non-limiting illustrative compounds of the application are listed in Table A. As shown in Table A, other tables of compounds, examples, schemes, and compounds throughout the present application, “or 1” (or “Or 1”) and “or 2” (or “Or 2”) indicate a single stereoisomeric configuration although the absolute stereochemistry of the indicated chiral carbon atom is not determined, and “&1” indicates a mixture of the stereoisomers of the indicated chiral carbon atom.

TABLE A

| Cmpd | | |
|---|---|---|
| No. | Structure | Chemical Name |
| 2 | 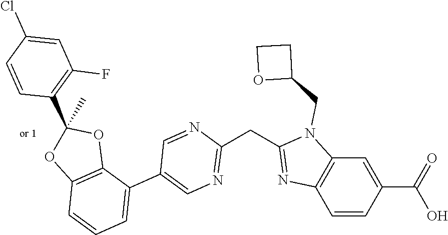 [SMILES: C[C@@]1(c2ccc(Cl)cc2F)Oc2cccc(-c3cnc(Cc4nc5ccc(C(=O)O)cc5n4C[C@@H]4CCO4)nc3)c2O1] | 2-((5-((R)-2-(4-chloro-2- fluorophenyl)-2- methylbenzo[d][1,3]dioxol-4- yl)pyrimidin-2-yl)methyl)-1- (((S)-oxetan-2-yl)methyl)-1H- benzo[d]imidazole-6-carboxylic acid |
| 3 | 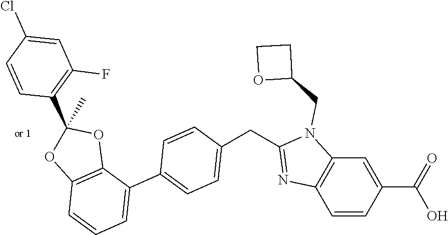 [SMILES: C[C@@]1(c2ccc(Cl)cc2F)Oc2cccc(-c3ccc(Cc4nc5ccc(C(=O)O)cc5n4C[C@@H]4CCO4)cc3)c2O1] | 2-(4-((R)-2-(4-chloro-2- fluorophenyl)-2- methylbenzo[d][1,3]dioxol-4- yl)benzyl)-1-(((S)-oxetan-2- yl)methyl)-1H- benzo[d]imidazole-6-carboxylic acid |
| 4 | 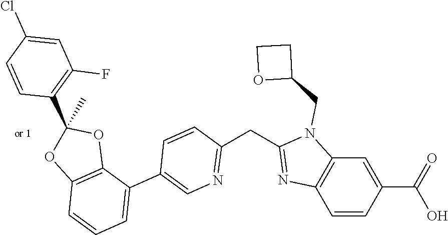 [SMILES: C[C@@]1(c2ccc(Cl)cc2F)Oc2cccc(-c3ccc(Cc4nc5ccc(C(=O)O)cc5n4C[C@@H]4CCO4)nc3)c2O1] | 2-((5-((R)-2-(4-chloro-2- fluorophenyl)-2- methylbenzo[d][1,3]dioxol-4- yl)pyridin-2-yl)methyl)-1-(((S)- oxetan-2-yl)methyl)-1H- benzo[d]imidazole-6-carboxylic acid |
| 5 | 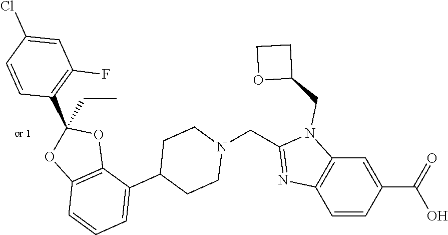 [SMILES: CC[C@@]1(c2ccc(Cl)cc2F)Oc2cccc(C3CCN(Cc4nc5ccc(C(=O)O)cc5n4C[C@@H]4CCO4)CC3)c2O1] | 2-((4-((R)-2-(4-chloro-2- fluorophenyl)-2- ethylbenzo[d][1,3]dioxol-4- yl)piperidin-1-yl)methyl)-1- (((S)-oxetan-2-yl)methyl)-1H- benzo[d]imidazole-6-carboxylic acid |
| 6 | 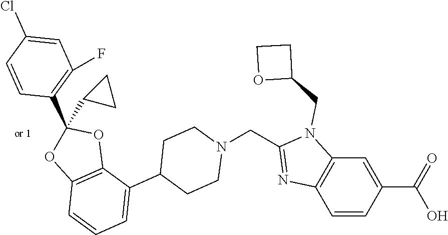 [SMILES: O=C(O)c1ccc2nc(CN3CCC(c4cccc5c4O[C@@](c4ccc(Cl)cc4F)(C4CC4)O5)CC3)n(C[C@@H]3CCO3)c2c1] | 2-((4-((R)-2-(4-chloro-2- fluorophenyl)-2- cyclopropylbenzo[d][1,3]dioxol- 4-yl)piperidin-1-yl)methyl)-1- (((S)-oxetan-2-yl)methyl)-1H- benzo[d]imidazole-6-carboxylic acid |
| 7 | 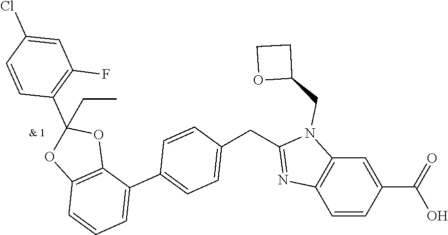 [SMILES: CCC1(c2ccc(Cl)cc2F)Oc2cccc(-c3ccc(Cc4nc5ccc(C(=O)O)cc5n4C[C@@H]4CCO4)cc3)c2O1] | 2-(4-(2-(4-chloro-2- fluorophenyl)-2- ethylbenzo[d][1,3]dioxol-4- yl)benzyl)-1-(((S)-oxetan-2- yl)methyl)-1H- benzo[d]imidazole-6-carboxylic acid |
| 8 | 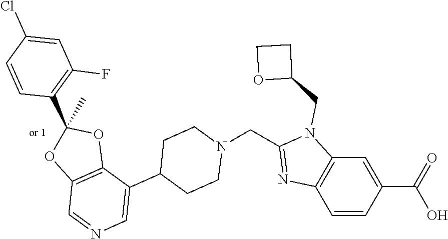 [SMILES: C[C@@]1(c2ccc(Cl)cc2F)Oc2cncc(C3CCN(Cc4nc5ccc(C(=O)O)cc5n4C[C@@H]4CCO4)CC3)c2O1] | 2-((4-((S)-2-(4-chloro-2- fluorophenyl)-2-methyl- [1,3]dioxolo[4,5-c]pyridin-7- yl)piperidin-1-yl)methyl)-1- (((S)-oxetan-2-yl)methyl)-1H- benzo[d]imidazole-6-carboxylic acid |
| 9 | 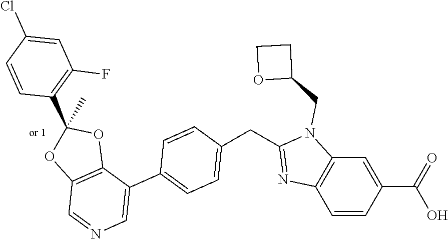 [SMILES: C[C@@]1(c2ccc(Cl)cc2F)Oc2cncc(-c3ccc(Cc4nc5ccc(C(=O)O)cc5n4C[C@@H]4CCO4)cc3)c2O1] | 2-(4-((S)-2-(4-chloro-2- fluorophenyl)-2-methyl- [1,3]dioxolo[4,5-c]pyridin-7- yl)benzyl)-1-(((S)-oxetan-2- yl)methyl)-1H- benzo[d]imidazole-6-carboxylic acid |
| 11 | 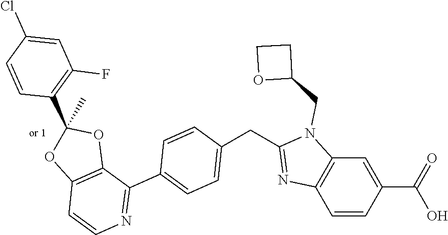 [SMILES: C[C@@]1(c2ccc(Cl)cc2F)Oc2ccnc(-c3ccc(Cc4nc5ccc(C(=O)O)cc5n4C[C@@H]4CCO4)cc3)c2O1] | 2-(4-((R)-2-(4-chloro-2- fluorophenyl)-2-methyl- [1,3]dioxolo[4,5-c]pyridin-4- yl)benzyl)-1-(((S)-oxetan-2- yl)methyl)-1H- benzo[d]imidazole-6-carboxylic acid |
| 12 | 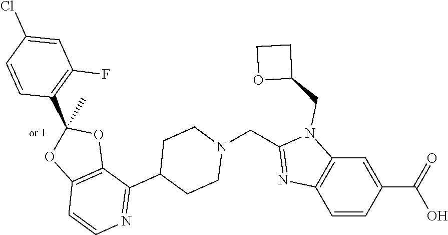 [SMILES: C[C@@]1(c2ccc(Cl)cc2F)Oc2ccnc(C3CCN(Cc4nc5ccc(C(=O)O)cc5n4C[C@@H]4CCO4)CC3)c2O1] | 2-((4-((R)-2-(4-chloro-2- fluorophenyl)-2-methyl- [1,3]dioxolo[4,5-c]pyridin-4- yl)piperidin-1-yl)methyl)-1- (((S)-oxetan-2-yl)methyl)-1H- benzo[d]imidazole-6-carboxylic acid |
| 13 | 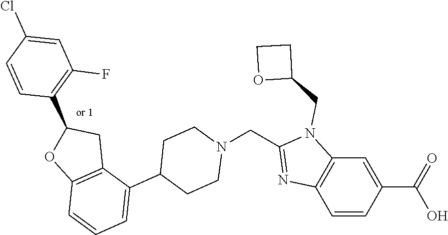 [SMILES: O=C(O)c1ccc2nc(CN3CCC(c4cccc5c4C[C@H](c4ccc(Cl)cc4F)O5)CC3)n(C[C@@H]3CCO3)c2c1] | 2-((4-((R)-2-(4-chloro-2- fluorophenyl)-2,3- dihydrobenzofuran-4- yl)piperidin-1-yl)methyl)-1- (((S)-oxetan-2-yl)methyl)-1H- benzo[d]imidazole-6-carboxylic acid |
| 14 | 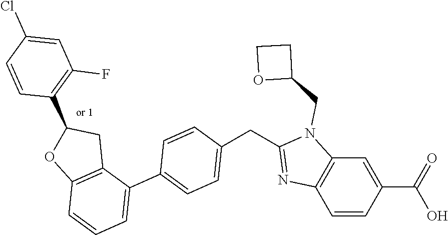 [SMILES: O=C(O)c1ccc2nc(Cc3ccc(-c4cccc5c4C[C@H](c4ccc(Cl)cc4F)O5)cc3)n(C[C@@H]3CCO3)c2c1] | 2-(4-((R)-2-(4-chloro-2- fluorophenyl)-2,3- dihydrobenzofuran-4-yl)benzyl)- 1-(((S)-oxetan-2-yl)methyl)-1H- benzo[d]imidazole-6-carboxylic acid |
| 15 | 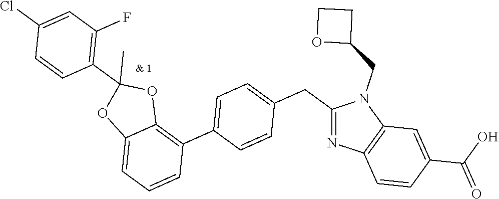 [SMILES: CC1(c2ccc(Cl)cc2F)Oc2cccc(-c3ccc(Cc4nc5ccc(C(=O)O)cc5n4C[C@@H]4CCO4)cc3)c2O1] | 2-(4-(2-(4-chloro-2- fluorophenyl)-2- methylbenzo[d][1,3]dioxol-4- yl)benzyl)-1-(((S)-oxetan-2- yl)methyl)-1H- benzo[d]imidazole-6-carboxylic acid |
| 16 | 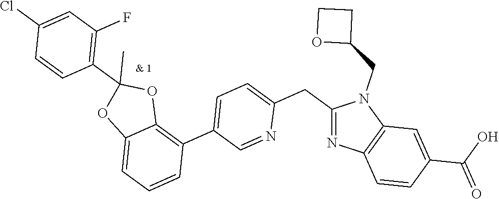 [SMILES: CC1(c2ccc(Cl)cc2F)Oc2cccc(-c3ccc(Cc4nc5ccc(C(=O)O)cc5n4C[C@@H]4CCO4)nc3)c2O1] | 2-((5-(2-(4-chloro-2- fluorophenyl)-2- methylbenzo[d][1,3]dioxol-4- yl)pyridin-2-yl)methyl)-1-(((S)- oxetan-2-yl)methyl)-1H- benzo[d]imidazole-6-carboxylic acid |
| 17 | 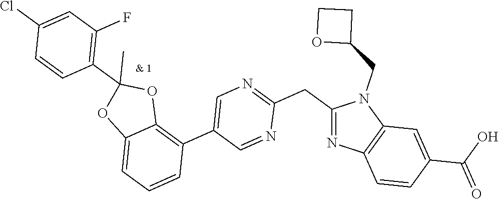 [SMILES: CC1(c2ccc(Cl)cc2F)Oc2cccc(-c3cnc(Cc4nc5ccc(C(=O)O)cc5n4C[C@@H]4CCO4)nc3)c2O1] | 2-((5-(2-(4-chloro-2- fluorophenyl)-2- methylbenzo[d][1,3]dioxol-4- yl)pyrimidin-2-yl)methyl)-1- (((S)-oxetan-2-yl)methyl)-1H- benzo[d]imidazole-6-carboxylic acid |
| 18 | 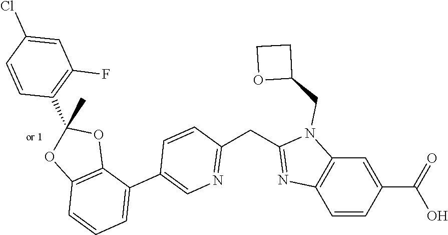 [SMILES: C[C@]1(c2ccc(Cl)cc2F)Oc2cccc(-c3ccc(Cc4nc5ccc(C(=O)O)cc5n4C[C@@H]4CCO4)nc3)c2O1] | 2-((5-((S)-2-(4-chloro-2- fluorophenyl)-2- methylbenzo[d][1,3]dioxol-4- yl)pyridin-2-yl)methyl)-1-(((S)- oxetan-2-yl)methyl)-1H- benzo[d]imidazole-6-carboxylic acid |
| 19 | 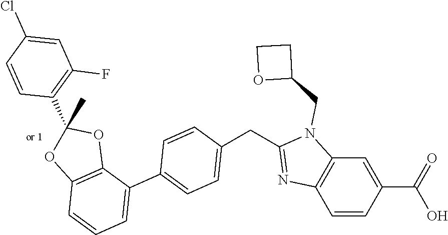 [SMILES: C[C@]1(c2ccc(Cl)cc2F)Oc2cccc(-c3ccc(Cc4nc5ccc(C(=O)O)cc5n4C[C@@H]4CCO4)cc3)c2O1] | 2-(4-((S)-2-(4-chloro-2- fluorophenyl)-2- methylbenzo[d][1,3]dioxol-4- yl)benzyl)-1-(((S)-oxetan-2- yl)methyl)-1H- benzo[d]imidazole-6-carboxylic acid |
| 21 | 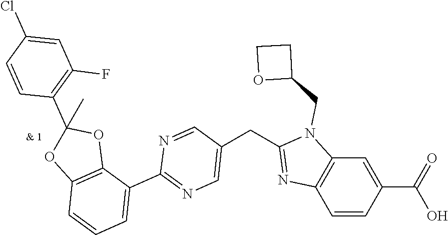 [SMILES: CC1(c2ccc(Cl)cc2F)Oc2cccc(-c3ncc(Cc4nc5ccc(C(=O)O)cc5n4C[C@@H]4CCO4)cn3)c2O1] | 2-((2-(2-(4-chloro-2- fluorophenyl)-2- methylbenzo[d][1,3]dioxol-4- yl)pyrimidin-5-yl)methyl)-1- (((S)-oxetan-2-yl)methyl)-1H- benzo[d]imidazole-6-carboxylic acid |
| 22 | 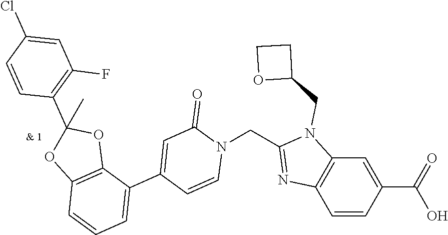 [SMILES: CC1(c2ccc(Cl)cc2F)Oc2cccc(-c3ccn(Cc4nc5ccc(C(=O)O)cc5n4C[C@@H]4CCO4)c(=O)c3)c2O1] | 2-((4-(2-(4-chloro-2- fluorophenyl)-2- methylbenzo[d][1,3]dioxol-4- yl)-2-oxopyridin-1(2H)- yl)methyl)-1-(((S)-oxetan-2- yl)methyl)-1H- benzo[d]imidazole-6-carboxylic acid |
| 23 | 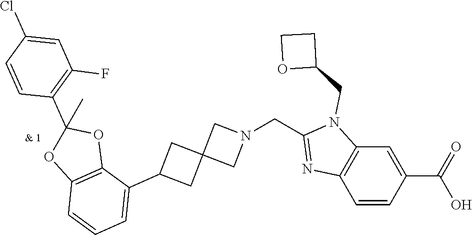 [SMILES: CC1(c2ccc(Cl)cc2F)Oc2cccc(C3CC4(C3)CN(Cc3nc5ccc(C(=O)O)cc5n3C[C@@H]3CCO3)C4)c2O1] | 2-((6-(2-(4-chloro-2- fluorophenyl)-2- methylbenzo[d][1,3]dioxol-4- yl)-2-azaspiro[3.3]heptan-2- yl)methyl)-1-(((S)-oxetan-2- yl)methyl)-1H- benzo[d]imidazole-6-carboxylic acid |
| 24 | 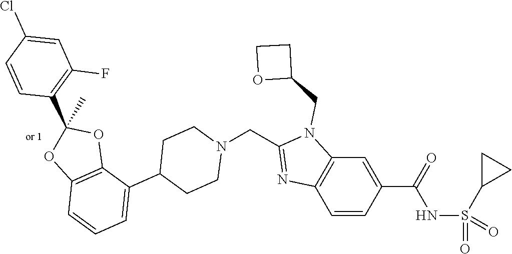 [SMILES: C[C@@]1(c2ccc(Cl)cc2F)Oc2cccc(C3CCN(Cc4nc5ccc(C(=O)NS(=O)(=O)C6CC6)cc5n4C[C@@H]4CCO4)CC3)c2O1] | 2-((4-((R)-2-(4-chloro-2- fluorophenyl)-2- methylbenzo[d][1,3]dioxol-4- yl)piperidin-1-yl)methyl)-N- (cyclopropylsulfonyl)-1-(((S)- oxetan-2-yl)methyl)-1H- benzo[d]imidazole-6- carboxamide |
| 25 | 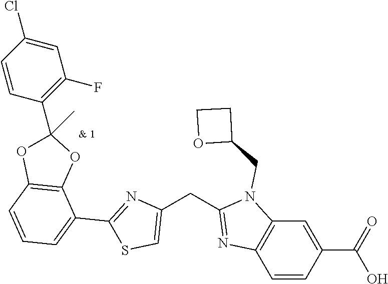 [SMILES: CC1(c2ccc(Cl)cc2F)Oc2cccc(-c3nc(Cc4nc5ccc(C(=O)O)cc5n4C[C@@H]4CCO4)cs3)c2O1] | 2-((2-(2-(4-chloro-2- fluorophenyl)-2- methylbenzo[d][1,3]dioxol-4- yl)thiazol-4-yl)methyl)-1-(((S)- oxetan-2-yl)methyl)-1H- benzo[d]imidazole-6-carboxylic acid |
| 26 | 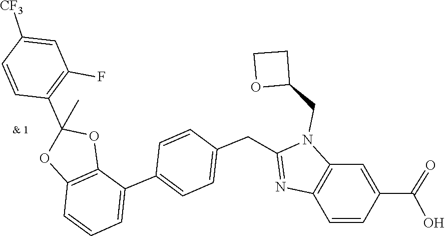 [SMILES: CC1(c2ccc(C(F)(F)F)cc2F)Oc2cccc(-c3ccc(Cc4nc5ccc(C(=O)O)cc5n4C[C@@H]4CCO4)cc3)c2O1] | 2-(4-(2-(2-fluoro-4- (trifluoromethyl)phenyl)-2- methylbenzo[d][1,3]dioxol-4- yl)benzyl)-1-(((S)-oxetan-2- yl)methyl)-1H- benzo[d]imidazole-6-carboxylic acid |
| 27 | 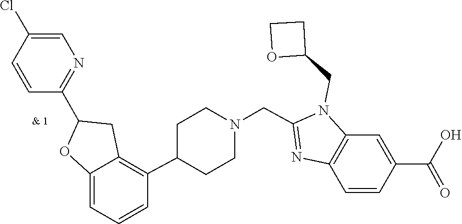 [SMILES: O=C(O)c1ccc2nc(CN3CCC(c4cccc5c4CC(c4ccc(Cl)cn4)O5)CC3)n(C[C@@H]3CCO3)c2c1] | 2-((4-(2-(5-chloropyridin-2-yl)- 2,3-dihydrobenzofuran-4- yl)piperidin-1-yl)methyl)-1- (((S)-oxetan-2-yl)methyl)-1H- benzo[d]imidazole-6-carboxylic acid |
| 28 | 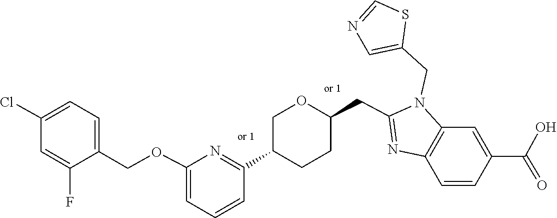 [SMILES: O=C(O)c1ccc2nc(C[C@H]3CC[C@H](c4cccc(OCc5ccc(Cl)cc5F)n4)CO3)n(Cc3cncs3)c2c1] | 2-(((2R,5R)-5-(6-((4-chloro-2- fluorobenzyl)oxy)pyridin-2- yl)tetrahydro-2H-pyran-2- yl)methyl)-1-(thiazol-5- ylmethyl)-1H- benzo[d]imidazole-6-carboxylic acid |
| 29 | 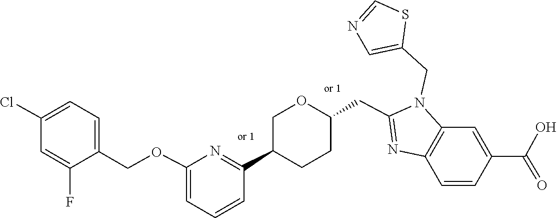 [SMILES: O=C(O)c1ccc2nc(C[C@@H]3CC[C@@H](c4cccc(OCc5ccc(Cl)cc5F)n4)CO3)n(Cc3cncs3)c2c1] | 2-(((2S,5S)-5-(6-((4-chloro-2- fluorobenzyl)oxy)pyridin-2- yl)tetrahydro-2H-pyran-2- yl)methyl)-1-(thiazol-5- ylmethyl)-1H- benzo[d]imidazole-6-carboxylic acid |
| 30 | 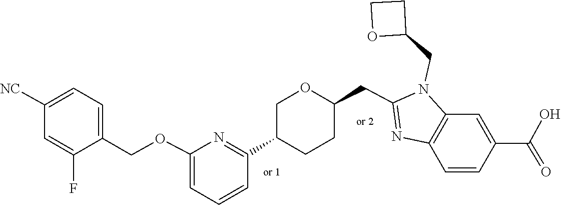 [SMILES: N#Cc1ccc(COc2cccc([C@H]3CC[C@H](Cc4nc5ccc(C(=O)O)cc5n4C[C@@H]4CCO4)OC3)n2)c(F)c1] | 2-(((2R,5R)-5-(6-((4-cyano-2- fluorobenzyl)oxy)pyridin-2- yl)tetrahydro-2H-pyran-2- yl)methyl)-1-(((S)-oxetan-2- yl)methyl)-1H- benzo[d]imidazole-6-carboxylic acid |
| 31 | 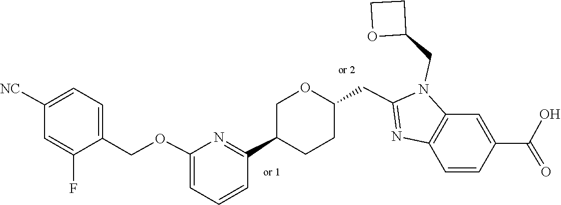 [SMILES: N#Cc1ccc(COc2cccc([C@@H]3CC[C@@H](Cc4nc5ccc(C(=O)O)cc5n4C[C@@H]4CCO4)OC3)n2)c(F)c1] | 2-(((2S,5S)-5-(6-((4-cyano-2- fluorobenzyl)oxy)pyridin-2- yl)tetrahydro-2H-pyran-2- yl)methyl)-1-(((S)-oxetan-2- yl)methyl)-1H- benzo[d]imidazole-6-carboxylic acid |
| 32 | 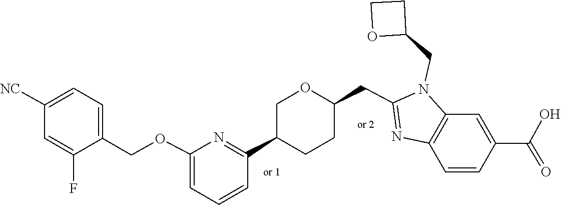 [SMILES: N#Cc1ccc(COc2cccc([C@@H]3CC[C@H](Cc4nc5ccc(C(=O)O)cc5n4C[C@@H]4CCO4)OC3)n2)c(F)c1] | 2-(((2R,5S)-5-(6-((4-cyano-2- fluorobenzyl)oxy)pyridin-2- yl)tetrahydro-2H-pyran-2- yl)methyl)-1-(((S)-oxetan-2- yl)methyl)-1H- benzo[d]imidazole-6-carboxylic acid |
| 33 | 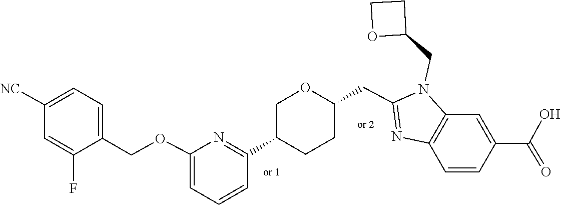 [SMILES: N#Cc1ccc(COc2cccc([C@H]3CC[C@@H](Cc4nc5ccc(C(=O)O)cc5n4C[C@@H]4CCO4)OC3)n2)c(F)c1] | 2-(((2S,5R)-5-(6-((4-cyano-2- fluorobenzyl)oxy)pyridin-2- yl)tetrahydro-2H-pyran-2- yl)methyl)-1-(((S)-oxetan-2- yl)methyl)-1H- benzo[d]imidazole-6-carboxylic acid |
| 34 | 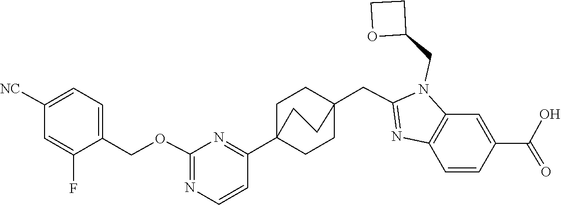 [SMILES: N#Cc1ccc(COc2nccc(C34CCC(Cc5nc6ccc(C(=O)O)cc6n5C[C@@H]5CCO5)(CC3)CC4)n2)c(F)c1] | (S)-2-((4-(2-((4-cyano-2- fluorobenzyl)oxy)pyrimidin-4- yl)bicyclo[2.2.2]octan-1- yl)methyl)-1-(oxetan-2- ylmethyl)-1H- benzo[d]imidazole-6-carboxylic acid |
| 35 | 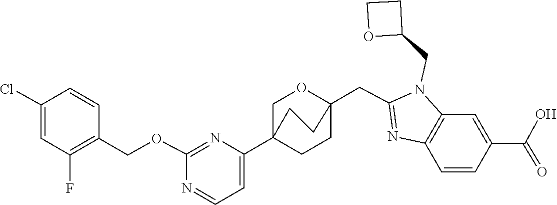 [SMILES: O=C(O)c1ccc2nc(CC34CCC(c5ccnc(OCc6ccc(Cl)cc6F)n5)(CC3)CO4)n(C[C@@H]3CCO3)c2c1] | (S)-2-((4-(2-((4-chloro-2- fluorobenzyl)oxy)pyrimidin-4- yl)-2-oxabicyclo[2.2.2]octan-1- yl)methyl)-1-(oxetan-2- ylmethyl)-1H- benzo[d]imidazole-6-carboxylic acid |
| 36 | 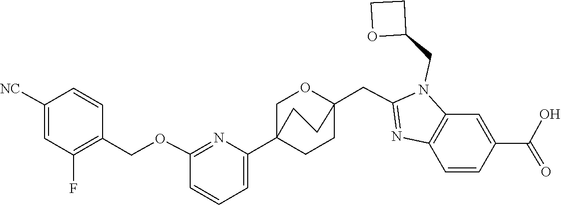 [SMILES: N#Cc1ccc(COc2cccc(C34CCC(Cc5nc6ccc(C(=O)O)cc6n5C[C@@H]5CCO5)(CC3)OC4)n2)c(F)c1] | (S)-2-((4-(6-((4-cyano-2- fluorobenzyl)oxy)pyridin-2-yl)- 2-oxabicyclo[2.2.2]octan-1- yl)methyl)-1-(oxetan-2- ylmethyl)-1H- benzo[d]imidazole-6-carboxylic acid |
| 37 | 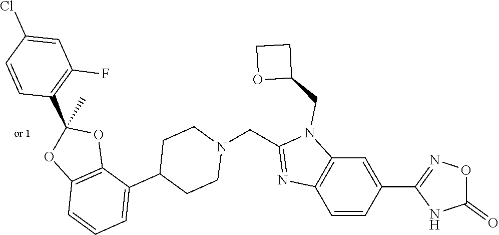 [SMILES: C[C@@]1(c2ccc(Cl)cc2F)Oc2cccc(C3CCN(Cc4nc5ccc(-c6noc(=O)[nH]6)cc5n4C[C@@H]4CCO4)CC3)c2O1] | 3-(2-((4-((R)-2-(4-chloro-2- fluorophenyl)-2- methylbenzo[d][1,3]dioxol-4- yl)piperidin-1-yl)methyl)-1- (((S)-oxetan-2-yl)methyl)-1H- benzo[d]imidazol-6-yl)-1,2,4- oxadiazol-5(4H)-one |
| 40 | 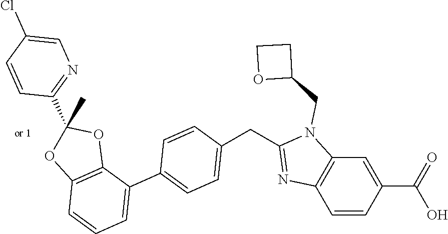 [SMILES: C[C@]1(c2ccc(Cl)cn2)Oc2cccc(-c3ccc(Cc4nc5ccc(C(=O)O)cc5n4C[C@@H]4CCO4)cc3)c2O1] | 2-(4-((S)-2-(5-chloropyridin-2- yl)-2- methylbenzo[d][1,3]dioxol-4- yl)benzyl)-1-(((S)-oxetan-2- yl)methyl)-1H- benzo[d]imidazole-6-carboxylic acid |
| 41 | 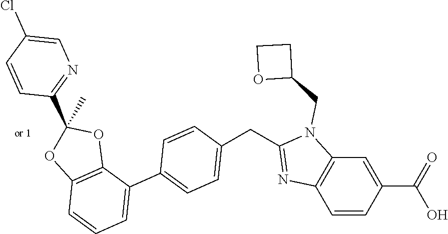 [SMILES: C[C@@]1(c2ccc(Cl)cn2)Oc2cccc(-c3ccc(Cc4nc5ccc(C(=O)O)cc5n4C[C@@H]4CCO4)cc3)c2O1] | 2-(4-((R)-2-(5-chloropyridin-2- yl)-2- methylbenzo[d][1,3]dioxol-4- yl)benzyl)-1-(((S)-oxetan-2- yl)methyl)-1H- benzo[d]imidazole-6-carboxylic acid |
| 42 | 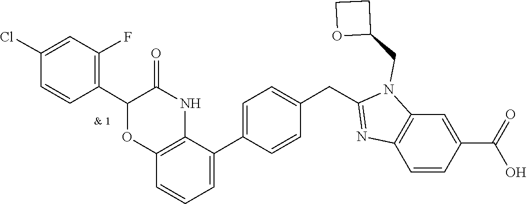 [SMILES: O=C(O)c1ccc2nc(Cc3ccc(-c4cccc5c4NC(=O)C(c4ccc(Cl)cc4F)O5)cc3)n(C[C@@H]3CCO3)c2c1] | 2-(4-(2-(4-chloro-2- fluorophenyl)-3-oxo-3,4- dihydro-2H- benzo[b][1,4]oxazin-5- yl)benzyl)-1-(((S)-oxetan-2- yl)methyl)-1H- benzo[d]imidazole-6-carboxylic acid |
| 43 | 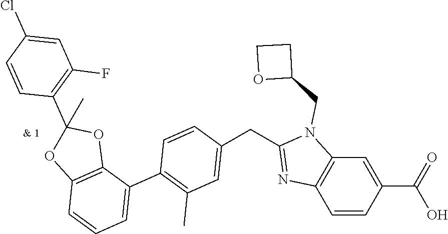 [SMILES: Cc1cc(Cc2nc3ccc(C(=O)O)cc3n2C[C@@H]2CCO2)ccc1-c1cccc2c1OC(C)(c1ccc(Cl)cc1F)O2] | 2-(4-(2-(4-chloro-2- fluorophenyl)-2- methylbenzo[d][1,3]dioxol-4- yl)-3-methylbenzyl)-1-(((S)- oxetan-2-yl)methyl)-1H- benzo[d]imidazole-6-carboxylic acid |
| 56 | 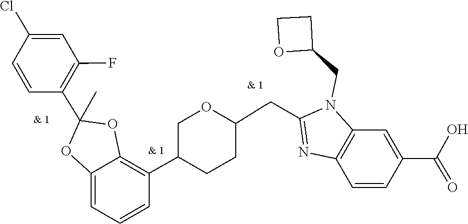 [SMILES: CC1(c2ccc(Cl)cc2F)Oc2cccc(C3CCC(CCc4nc5ccc(C(=O)O)cc5n4C[C@@H]4CCO4)OC3)c2O1] | 2-((5-(2-(4-chloro-2- fluorophenyl)-2- methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 57 | 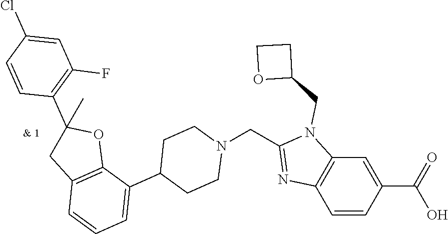 [SMILES: CC1(c2ccc(Cl)cc2F)Cc2cccc(C3CCN(Cc4nc5ccc(C(=O)O)cc5n4C[C@@H]4CCO4)CC3)c2O1] | 2-((4-(2-(4-chloro-2- fluorophenyl)-2-methyl-2,3- dihydrobenzofuran-7- yl)piperidin-1-yl)methyl)-1- (((S)-oxetan-2-yl)methyl)-1H- benzo[d]imidazole-6-carboxylic acid |
| 58 | 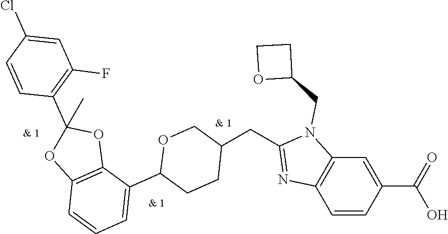 [SMILES: CC1(c2ccc(Cl)cc2F)Oc2cccc(C3CCC(Cc4nc5ccc(C(=O)O)cc5n4C[C@@H]4CCO4)CO3)c2O1] | 2-((6-(2-(4-chloro-2- fluorophenyl)-2- methylbenzo[d][1,3]dioxol-4- yl)tetrahydro-2H-pyran-3- yl)methyl)-1-(((S)-oxetan-2- yl)methyl)-1H- benzo[d]imidazole-6-carboxylic acid |
| 59 | 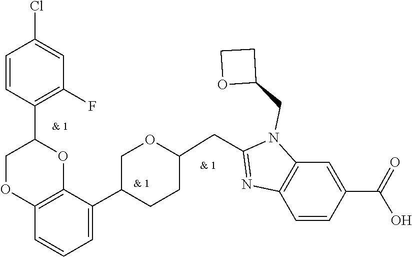 [SMILES: O=C(O)c1ccc2nc(CCC3CCC(c4cccc5c4OC(c4ccc(Cl)cc4F)CO5)CO3)n(C[C@@H]3CCO3)c2c1] | 2-((5-(3-(4-chloro-2- fluorophenyl)-2,3- dihydrobenzo[b][1,4]dioxin-5- yl)tetrahydro-2H-pyran-2- yl)methyl)-1-(((S)-oxetan-2- yl)methyl)-1H- benzo[d]imidazole-6-carboxylic acid |
| 60 | 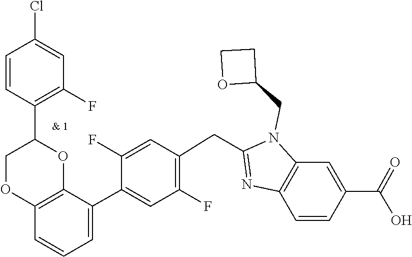 [SMILES: O=C(O)c1ccc2nc(CCc3cc(F)c(-c4cccc5c4OC(c4ccc(Cl)cc4F)CO5)cc3F)n(C[C@@H]3CCO3)c2c1] | 2-(4-(3-(4-chloro-2- fluorophenyl)-2,3- dihydrobenzo[b][1,4]dioxin-5- yl)-2,5-difluorobenzyl)-1-(((S)- oxetan-2-yl)methyl)-1H- benzo[d]imidazole-6-carboxylic acid |
| 61 | 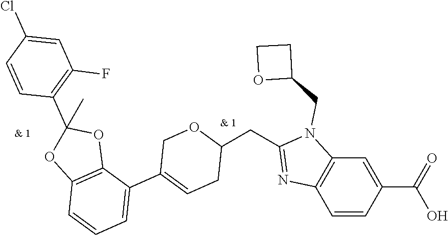 [SMILES: CC1(c2ccc(Cl)cc2F)Oc2cccc(C3=CCC(Cc4nc5ccc(C(=O)O)cc5n4C[C@@H]4CCO4)OC3)c2O1] | 2-((5-(2-(4-chloro-2- fluorophenyl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 62 | 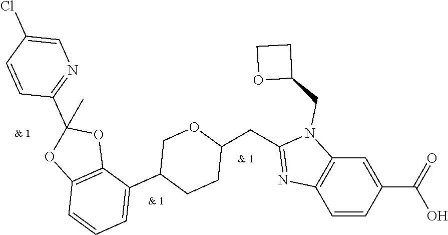 [SMILES: CC1(c2ccc(Cl)cn2)Oc2cccc(C3CCC(Cc4nc5ccc(C(=O)O)cc5n4C[C@@H]4CCO4)OC3)c2O1] | 2-((5-(2-(5-chloropyridin-2-yl)- 2-methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 63 | 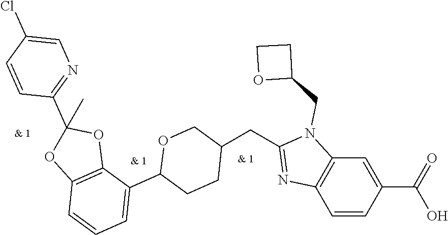 [SMILES: CC1(c2ccc(Cl)cn2)Oc2cccc(C3CCC(Cc4nc5ccc(C(=O)O)cc5n4C[C@@H]4CCO4)CO3)c2O1] | 2-((6-(2-(5-chloropyridin-2-yl)- 2-methylbenzo[d][1,3]dioxol-4- yl)tetrahydro-2H-pyran-3- yl)methyl)-1-(((S)-oxetan-2- yl)methyl)-1H- benzo[d]imidazole-6-carboxylic acid |
| 64 | 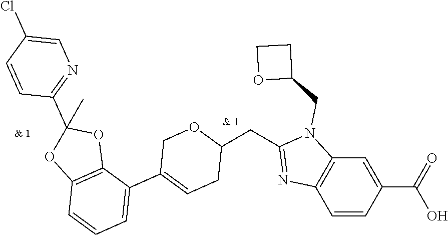 [SMILES: CC1(c2ccc(Cl)cn2)Oc2cccc(C3=CCC(Cc4nc5ccc(C(=O)O)cc5n4C[C@@H]4CCO4)OC3)c2O1] | 2-((5-(2-(5-chloropyridin-2-yl)- 2-methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 65 | 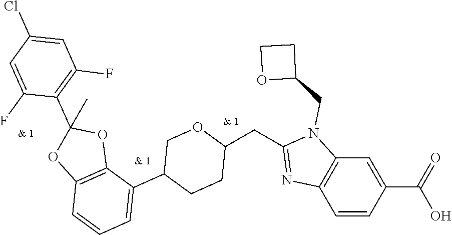 [SMILES: CC1(c2c(F)cc(Cl)cc2F)Oc2cccc(C3CCC(Cc4nc5ccc(C(=O)O)cc5n4C[C@@H]4CCO4)OC3)c2O1] | 2-((5-(2-(4-chloro-2,6- difluorophenyl)-2- methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 66 | 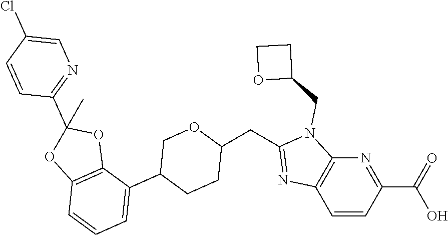 [SMILES: CC1(c2ccc(Cl)cn2)Oc2cccc(C3CCC(Cc4nc5ccc(C(=O)O)nc5n4C[C@@H]4CCO4)OC3)c2O1] | 2-((5-(2-(5-chloropyridin-2-yl)- 2-methylbenzo[d][1,3]dioxol-4- yl)tetrahydro-2H-pyran-2- yl)methyl)-3-(((S)-oxetan-2- yl)methyl)-3H-imidazo[4,5- b]pyridine-5-carboxylic acid |
| 67 | 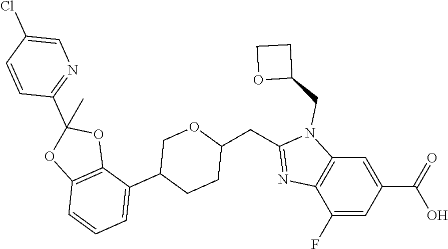 [SMILES: CC1(c2ccc(Cl)cn2)Oc2cccc(C3CCC(Cc4nc5c(F)cc(C(=O)O)cc5n4C[C@@H]4CCO4)OC3)c2O1] | 2-((5-(2-(5-chloropyridin-2-yl)- 2-methylbenzo[d][1,3]dioxol-4- yl)tetrahydro-2H-pyran-2- yl)methyl)-4-fluoro-1-(((S)- oxetan-2-y(methyl)-1H- benzo[d]imidazole-6-carboxylic acid |
| 68 | 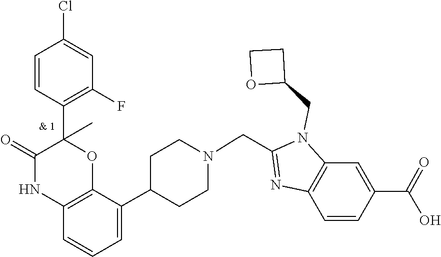 [SMILES: CC1(c2ccc(Cl)cc2F)Oc2c(cccc2C2CCN(CCc3nc4ccc(C(=O)O)cc4n3C[C@@H]3CCO3)CC2)NC1=O] | 2-((4-(2-(4-chloro-2- fluorophenyl)-2-methyl-3-oxo- 3,4-dihydro-2H- benzo[b][1,4]oxazin-8- yl)piperidin-1-yl)methyl)-1- (((S)-oxetan-2-yl)methyl)-1H- benzo[d]imidazole-6-carboxylic acid |
| 69 | 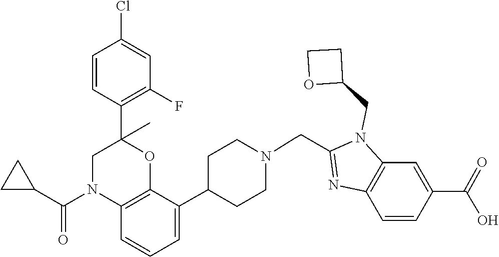 [SMILES: CC1(c2ccc(Cl)cc2F)CN(C(=O)C2CC2)c2cccc(C3CCN(CCc4nc5ccc(C(=O)O)cc5n4C[C@@H]4CCO4)CC3)c2O1] | 2-((4-(2-(4-chloro-2- fluorophenyl)-4- (cyclopropanecarbonyl)-2- methyl-3,4-dihydro-2H- benzo[b][1,4]oxazin-8- yl)piperidin-1-yl)methyl)-1- (((S)-oxetan-2-yl)methyl)-1H- benzo[d]imidazole-6-carboxylic acid |
| 70 | 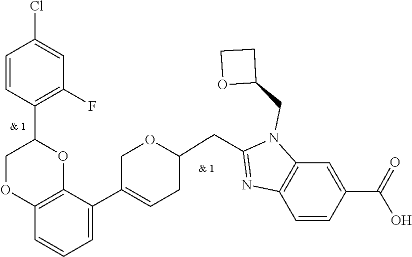 [SMILES: O=C(O)c1ccc2nc(CCC3CC=C(c4cccc5c4OC(c4ccc(Cl)cc4F)CO5)CO3)n(C[C@@H]3CCO3)c2c1] | 2-((5-(3-(4-chloro-2- fluorophenyl)-2,3- dihydrobenzo[b][1,4]dioxin-5- yl)-3,6-dihydro-2H-pyran-2- yl)methyl)-1-(((S)-oxetan-2- yl)methyl)-1H- benzo[d]imidazole-6-carboxylic acid |
| 71 | 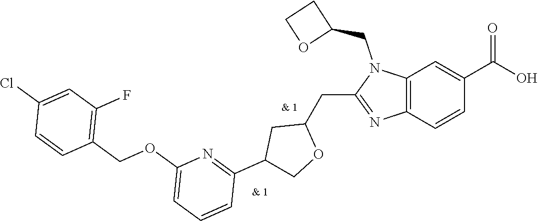 [SMILES: O=C(O)c1ccc2nc(CC3CC(c4cccc(OCc5ccc(Cl)cc5F)n4)CO3)n(C[C@@H]3CCO3)c2c1] | 2-((4-(6-((4-chloro-2- fluorobenzyl)oxy)pyridin-2- yl)tetrahydrofuran-2-yl)methyl)- 1-(((S)-oxetan-2-yl)methyl)-1H- benzo[d]imidazole-6-carboxylic acid |
| 72 | 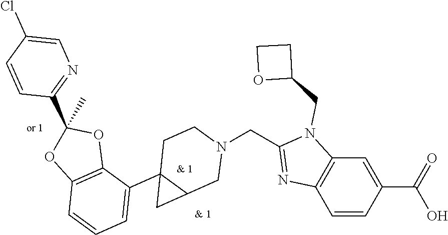 [SMILES: C[C@@]1(c2ccc(Cl)cn2)Oc2cccc(C34CCN(Cc5nc6ccc(C(=O)O)cc6n5C[C@@H]5CCO5)CC3C4)c2O1] | 2-((6-((S)-2-(5-chloropyridin-2- yl)-2- methylbenzo[d][1,3]dioxol-4- yl)-3-azabicyclo[4.1.0]heptan-3- yl)methyl)-1-(((S)-oxetan-2- yl)methyl)-1H- benzo[d]imidazole-6-carboxylic acid |
| 73 | 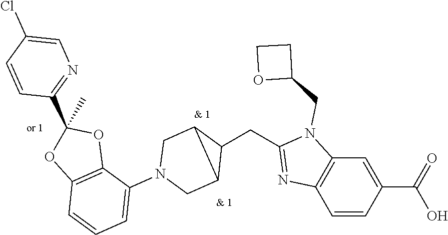 [SMILES: C[C@@]1(c2ccc(Cl)cn2)Oc2cccc(N3CC4C(Cc5nc6ccc(C(=O)O)cc6n5C[C@@H]5CCO5)C4C3)c2O1] | 2-((3-((R)-2-(5-chloropyridin-2- yl)-2- methylbenzo[d][1,3]dioxol-4- yl)-3-azabicyclo[3.1.0]hexan-6- yl)methyl)-1-(((S)-oxetan-2- yl)methyl)-1H- benzo[d]imidazole-6-carboxylic acid |
| 74 | 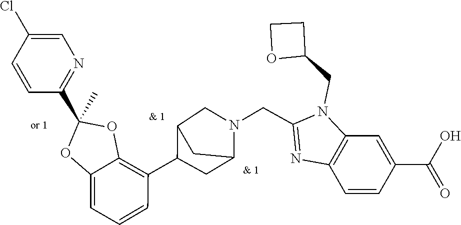 [SMILES: C[C@@]1(c2ccc(Cl)cn2)Oc2cccc(C3CC4CC3CN4Cc3nc4ccc(C(=O)O)cc4n3C[C@@H]3CCO3)c2O1] | 2-((5-((R)-2-(5-chloropyridin-2- yl)-2- methylbenzo[d][1,3]dioxol-4- yl)-2-azabicyclo[2.2.1]heptan-2- yl)methyl)-1-(((S)-oxetan-2- yl)methyl)-1H- benzo[d]imidazole-6-carboxylic acid |
| 75 | 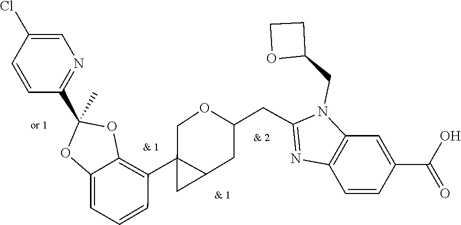 [SMILES: C[C@@]1(c2ccc(Cl)cn2)Oc2cccc(C34COC(Cc5nc6ccc(C(=O)O)cc6n5C[C@@H]5CCO5)CC3C4)c2O1] | 2-((1-((R)-2-(5-chloropyridin-2- yl)-2- methylbenzo[d][1,3]dioxol-4- yl)-3-oxabicyclo[4.1.0]heptan-4- yl)methyl)-1-(((S)-oxetan-2- yl)methyl)-1H- benzo[d]imidazole-6-carboxylic acid |
| 76 | 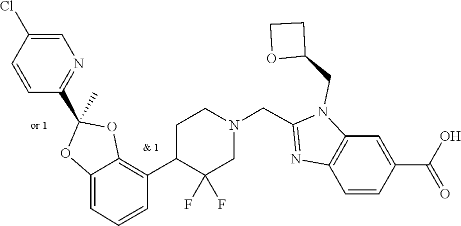 [SMILES: C[C@@]1(c2ccc(Cl)cn2)Oc2cccc(C3CCN(Cc4nc5ccc(C(=O)O)cc5n4C[C@@H]4CCO4)CC3(F)F)c2O1] | 2-((4-((R)-2-(5-chloropyridin-2- yl)-2- methylbenzo[d][1,3]dioxol-4- yl)-3,3-difluoropiperidin-1- yl)methyl)-1-(((S)-oxetan-2- yl)methyl)-1H- benzo[d]imidazole-6-carboxylic acid |
| 77 | 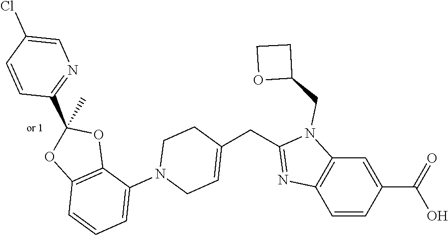 [SMILES: C[C@@]1(c2ccc(Cl)cn2)Oc2cccc(N3CC=C(Cc4nc5ccc(C(=O)O)cc5n4C[C@@H]4CCO4)CC3)c2O1] | 2-((1-((R)-2-(5-chloropyridin-2- yl)-2- methylbenzo[d][1,3]dioxol-4- yl)-1,2,3,6-tetrahydropyridin-4- yl)methyl)-1-(((S)-oxetan-2- yl)methyl)-1H- benzo[d]imidazole-6-carboxylic acid |
| 78 | 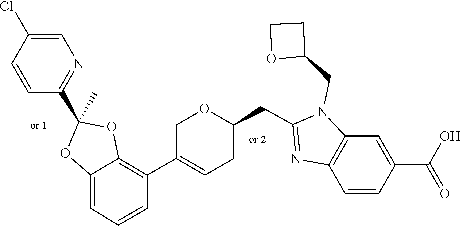 [SMILES: C[C@@]1(c2ccc(Cl)cn2)Oc2cccc(C3=CC[C@H](Cc4nc5ccc(C(=O)O)cc5n4C[C@@H]4CCO4)OC3)c2O1] | 2-(((R)-5-((R)-2-(5- chloropyridin-2-yl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 79 | 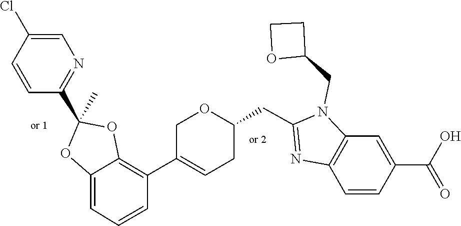 [SMILES: C[C@@]1(c2ccc(Cl)cn2)Oc2cccc(C3=CC[C@@H](Cc4nc5ccc(C(=O)O)cc5n4C[C@@H]4CCO4)OC3)c2O1] | 2-(((S)-5-((R)-2-(5- chloropyridin-2-yl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 80 | 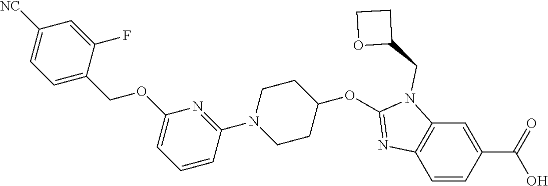 | (S)-2-((1-(6-((4-cyano-2- fluorobenzyl)oxy)pyridin-2- yl)piperidin-4-yl)oxy)-1-(oxetan- 2-ylmethyl)-1H- benzo[d]imidazole-6-carboxylic acid |
| 81 | 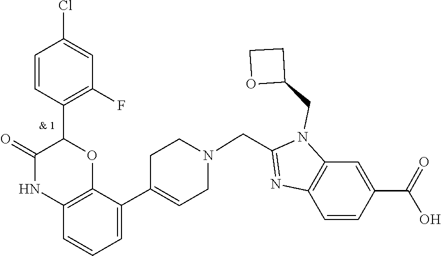 [SMILES: O=C(O)c1ccc2nc(CCN3CC=C(c4cccc5c4OC(c4ccc(Cl)cc4F)C(=O)N5)CC3)n(C[C@@H]3CCO3)c2c1] | 2-((4-(2-(4-chloro-2- fluorophenyl)-3-oxo-3,4- dihydro-2H- benzo[b][1,4]oxazin-8-yl)-3,6- dihydropyridin-1(2H)- yl)methyl)-1-(((S)-oxetan-2- yl)methyl)-1H- benzo[d]imidazole-6-carboxylic acid |
| 82 | 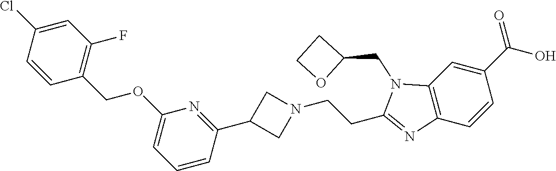 [SMILES: O=C(O)c1ccc2nc(CCN3CC(c4cccc(OCc5ccc(Cl)cc5F)n4)C3)n(C[C@@H]3CCO3)c2c1] | (S)-2-(2-(3-(6-((4-chloro-2- fluorobenzyl)oxy)pyridin-2- yl)azetidin-1-yl)ethyl)-1- (oxetan-2-ylmethyl)-1H- benzo[d]imidazole-6-carboxylic acid |
| 83 | 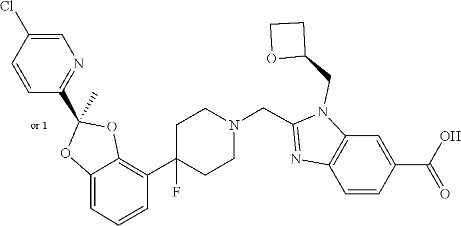 [SMILES: C[C@@]1(c2ccc(Cl)cn2)Oc2cccc(C3(F)CCN(Cc4nc5ccc(C(=O)O)cc5n4C[C@@H]4CCO4)CC3)c2O1] | 2-((4-((R)-2-(5-chloropyridin-2- yl)-2- methylbenzo[d][1,3]dioxol-4- yl)-4-fluoropiperidin-1- yl)methyl)-1-(((S)-oxetan-2- yl)methyl)-1H- benzo[d]imidazole-6-carboxylic acid |
| 84 | 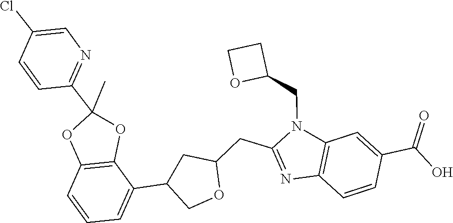 [SMILES: CC1(c2ccc(Cl)cn2)Oc2cccc(C3COC(Cc4nc5ccc(C(=O)O)cc5n4C[C@@H]4CCO4)C3)c2O1] | 2-((4-(2-(5-chloropyridin-2-yl)- 2-methylbenzo[d][1,3]dioxol-4- yl)tetrahydrofuran-2-yl)methyl)- 1-(((S)-oxetan-2-yl)methyl)-1H- benzo[d]imidazole-6-carboxylic acid |
| 85 | 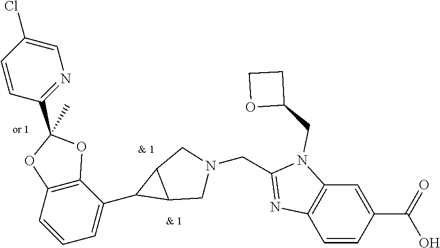 [SMILES: C[C@@]1(c2ccc(Cl)cn2)Oc2cccc(C3C4CN(Cc5nc6ccc(C(=O)O)cc6n5C[C@@H]5CCO5)CC43)c2O1] | 2-((6-((R)-2-(5-chloropyridin-2- yl)-2- methylbenzo[d][1,3]dioxol-4- yl)-3-azabicyclo[3.1.0]hexan-3- yl)methyl)-1-(((S)-oxetan-2- yl)methyl)-1H- benzo[d]imidazole-6-carboxylic acid |
| 86 | 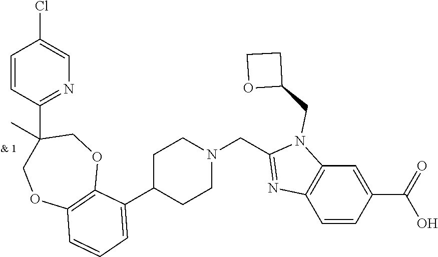 [SMILES: CC1(c2ccc(Cl)cn2)COc2cccc(C3CCN(Cc4nc5ccc(C(=O)O)cc5n4C[C@@H]4CCO4)CC3)c2OC1] | 2-((4-(3-(5-chloropyridin-2-yl)- 3-methyl-3,4-dihydro-2H- benzo[b][1,4]dioxepin-6- yl)piperidin-l-yl)methyl)-1- (((S)-oxetan-2-yl)methyl)-1H- benzo[d]imidazole-6-carboxylic acid |
| 87 | 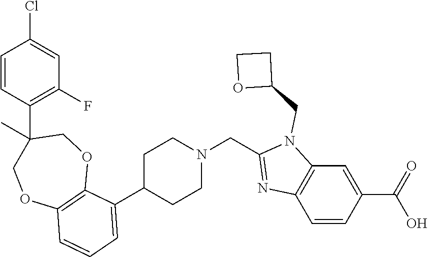 [SMILES: CC1(c2ccc(Cl)cc2F)COc2cccc(C3CCN(Cc4nc5ccc(C(=O)O)cc5n4C[C@@H]4CCO4)CC3)c2OC1] | 2-((4-(3-(4-chloro-2- fluorophenyl)-3-methyl-3,4- dihydro-2H- benzo[b][1,4]dioxepin-6- yl)piperidin-1-yl)methyl)-1- (((S)-oxetan-2-yl)methyl)-1H- benzo[d]imidazole-6-carboxylic acid |
| 88 | 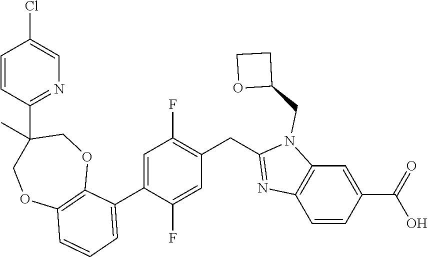 [SMILES: CC1(c2ccc(Cl)cn2)COc2cccc(-c3cc(F)c(Cc4nc5ccc(C(=O)O)cc5n4C[C@@H]4CCO4)cc3F)c2OC1] | 2-(4-(3-(5-chloropyridin-2-yl)-3- methyl-3,4-dihydro-2H- benzo[b][1,4]dioxepin-6-yl)-2,5- difluorobenzyl)-1-(((S)-oxetan- 2-yl)methyl)-1H- benzo[d]imidazole-6-carboxylic acid |
| 89 | 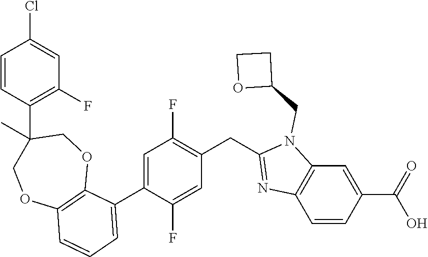 [SMILES: CC1(c2ccc(Cl)cc2F)COc2cccc(-c3cc(F)c(Cc4nc5ccc(C(=O)O)cc5n4C[C@@H]4CCO4)cc3F)c2OC1] | 2-(4-(3-(4-chloro-2- fluorophenyl)-3-methyl-3,4- dihydro-2H- benzo[b][1,4]dioxepin-6-yl)-2,5- difluorobenzyl)-1-(((S)-oxetan- 2-yl)methyl)-1H- benzo[d]imidazole-6-carboxylic acid |
| 90 | 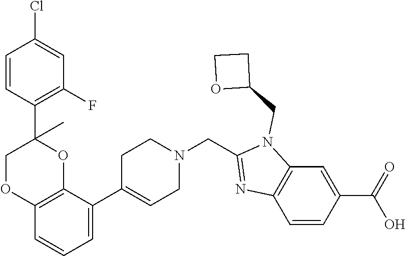 [SMILES: CC1(c2ccc(Cl)cc2F)COc2cccc(C3=CCN(Cc4nc5ccc(C(=O)O)cc5n4C[C@@H]4CCO4)CC3)c2O1] | 2-((4-(3-(4-chloro-2- fluorophenyl)-3-methyl-2,3- dihydrobenzo[b][1,4]dioxin-5- yl)-3,6-dihydropyridin-1(2H)- yl)methyl)-1-(((S)-oxetan-2- yl)methyl)-1H- benzo[d]imidazole-6-carboxylic acid |
| 91 | 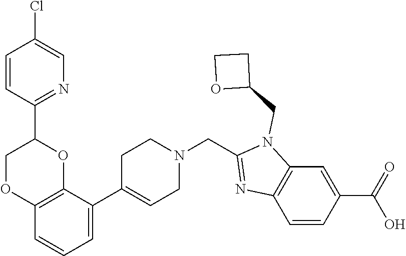 [SMILES: O=C(O)c1ccc2nc(CN3CC=C(c4cccc5c4OC(c4ccc(Cl)cn4)CO5)CC3)n(C[C@@H]3CCO3)c2c1] | 2-((4-(3-(5-chloropyridin-2-yl)- 2,3-dihydrobenzo[b][1,4]dioxin- 5-yl)-3,6-dihydropyridin-1(2H)- yl)methyl)-1-(((S)-oxetan-2- yl)methyl)-1H- benzo[d]imidazole-6-carboxylic acid |
| 92 | 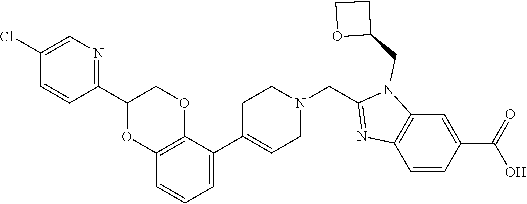 [SMILES: O=C(O)c1ccc2nc(CN3CC=C(c4cccc5c4OCC(c4ccc(Cl)cn4)O5)CC3)n(C[C@@H]3CCO3)c2c1] | 2-((4-(2-(5-chloropyridin-2-yl)- 2,3-dihydrobenzo[b][1,4]dioxin- 5-yl)-3,6-dihydropyridin-1(2H)- yl)methyl)-1-(((S)-oxetan-2- yl)methyl)-1H- benzo[d]imidazole-6-carboxylic acid |
| 93 | 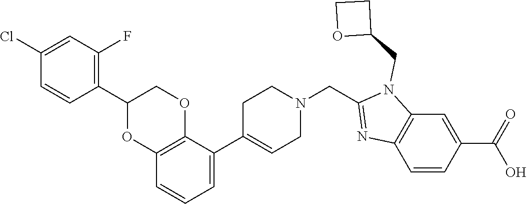 [SMILES: O=C(O)c1ccc2nc(CN3CC=C(c4cccc5c4OCC(c4ccc(Cl)cc4F)O5)CC3)n(C[C@@H]3CCO3)c2c1] | 2-((4-(2-(4-chloro-2- fluorophenyl)-2,3- dihydrobenzo[b][1,4]dioxin-5- yl)-3,6-dihydropyridin-1(2H)- yl)methyl)-1-(((S)-oxetan-2- yl)methyl)-1H- benzo[d]imidazole-6-carboxylic acid |
| 94 | 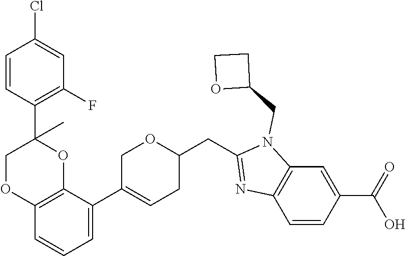 [SMILES: CC1(c2ccc(Cl)cc2F)COc2cccc(C3=CCC(Cc4nc5ccc(C(=O)O)cc5n4C[C@@H]4CCO4)OC3)c2O1] | 2-((5-(3-(4-chloro-2- fluorophenyl)-3-methyl-2,3- dihydrobenzo[b][1,4]dioxin-5- yl)-3,6-dihydro-2H-pyran-2- yl)methyl)-1-(((S)-oxetan-2- yl)methyl)-1H- benzo[d]imidazole-6-carboxylic acid |
| 95 | 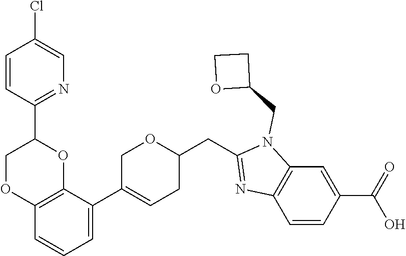 [SMILES: O=C(O)c1ccc2nc(CC3CC=C(c4cccc5c4OC(c4ccc(Cl)cn4)CO5)CO3)n(C[C@@H]3CCO3)c2c1] | 2-((5-(3-(5-chloropyridin-2-yl)- 2,3-dihydrobenzo[b][1,4]dioxin- 5-yl)-3,6-dihydro-2H-pyran-2- yl)methyl)-1-(((S)-oxetan-2- yl)methyl)-1H- benzo[d]imidazole-6-carboxylic acid |
| 96 | 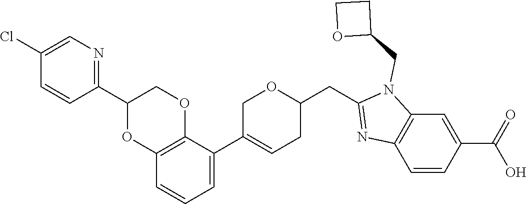 [SMILES: O=C(O)c1ccc2nc(CC3CC=C(c4cccc5c4OCC(c4ccc(Cl)cn4)O5)CO3)n(C[C@@H]3CCO3)c2c1] | 2-((5-(2-(5-chloropyridin-2-yl)- 2,3-dihydrobenzo[b][1,4]dioxin- 5-yl)-3,6-dihydro-2H-pyran-2- yl)methyl)-1-(((S)-oxetan-2- yl)methyl)-1H- benzo[d]imidazole-6-carboxylic |
| 97 | 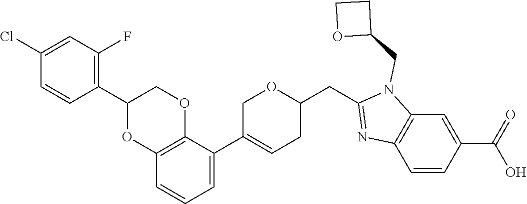 [SMILES: O=C(O)c1ccc2nc(CC3CC=C(c4cccc5c4OCC(c4ccc(Cl)cc4F)O5)CO3)n(C[C@@H]3CCO3)c2c1] | 2-((5-(2-(4-chloro-2- fluorophenyl)-2,3- dihydrobenzo[b][1,4]dioxin-5- yl)-3,6-dihydro-2H-pyran-2- yl)methyl)-1-(((S)-oxetan-2- yl)methyl)-1H- benzo[d]imidazole-6-carboxylic acid |
| 98 | 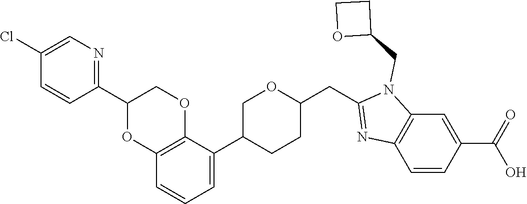 [SMILES: O=C(O)c1ccc2nc(CC3CCC(c4cccc5c4OCC(c4ccc(Cl)cn4)O5)CO3)n(C[C@@H]3CCO3)c2c1] | 2-((5-(2-(5-chloropyridin-2-yl)- 2,3-dihydrobenzo[b][1,4]dioxin- 5-yl)tetrahydro-2H-pyran-2- yl)methyl)-1-(((S)-oxetan-2- yl)methyl)-1H- benzo[d]imidazole-6-carboxylic acid |
| 99 | 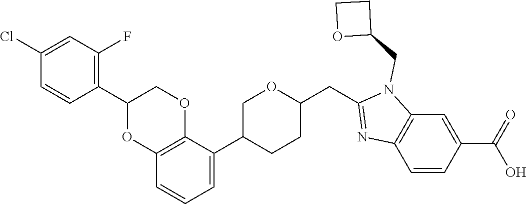 [SMILES: O=C(O)c1ccc2nc(CC3CCC(c4cccc5c4OCC(c4ccc(Cl)cc4F)O5)CO3)n(C[C@@H]3CCO3)c2c1] | 2-((5-(2-(4-chloro-2- fluorophenyl)-2,3- dihydrobenzo[b][1,4]dioxin-5- yl)tetrahydro-2H-pyran-2- yl)methyl)-1-(((S)-oxetan-2- yl)methyl)-1H- benzo[d]imidazole-6-carboxylic acid |
| 100 | 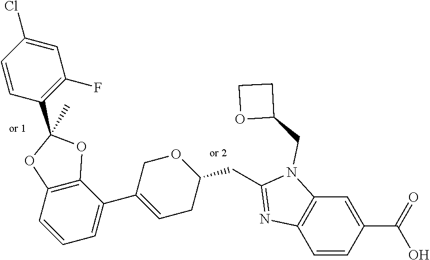 [SMILES: C[C@@]1(c2ccc(Cl)cc2F)Oc2cccc(C3=CC[C@@H](Cc4nc5ccc(C(=O)O)cc5n4C[C@@H]4CCO4)OC3)c2O1] | 2-(((S)-5-((S)-2-(4-chloro-2- fluorophenyl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 101 | 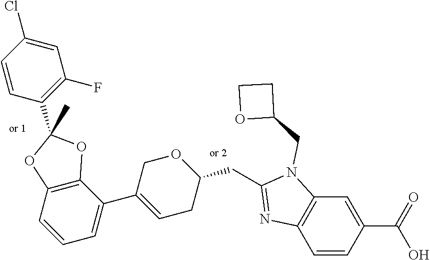 [SMILES: C[C@]1(c2ccc(Cl)cc2F)Oc2cccc(C3=CC[C@@H](Cc4nc5ccc(C(=O)O)cc5n4C[C@@H]4CCO4)OC3)c2O1] | 2-(((S)-5-((S)-2-(4-chloro-2- fluorophenyl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 102 | 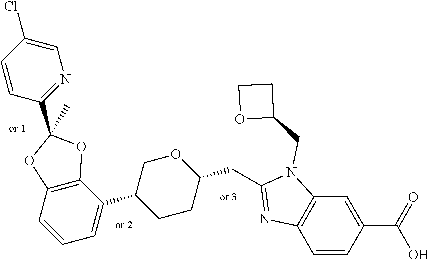 [SMILES: C[C@@]1(c2ccc(Cl)cn2)Oc2cccc([C@H]3CC[C@@H](Cc4nc5ccc(C(=O)O)cc5n4C[C@@H]4CCO4)OC3)c2O1] | 2-(((2S,5R)-5-((R)-2-(5- chloropyridin-2-yl)-2- methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 103 | 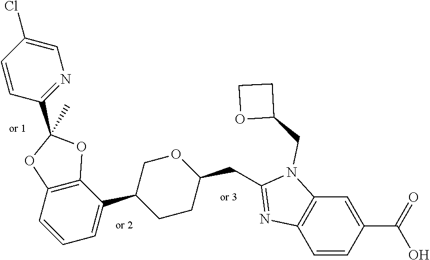 [SMILES: C[C@@]1(c2ccc(Cl)cn2)Oc2cccc([C@@H]3CC[C@H](Cc4nc5ccc(C(=O)O)cc5n4C[C@@H]4CCO4)OC3)c2O1] | 2-(((2R,5S)-5-((R)-2-(5- chloropyridin-2-yl)-2- methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 104 | 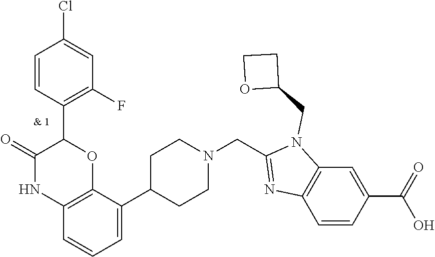 [SMILES: O=C(O)c1ccc2nc(CN3CCC(c4cccc5c4OC(c4ccc(Cl)cc4F)C(=O)N5)CC3)n(C[C@@H]3CCO3)c2c1] | 2-((4-(2-(4-chloro-2- fluorophenyl)-3-oxo-3,4- dihydro-2H- benzo[b][1,4]oxazin-8- yl)piperidin-1-yl)methyl)-1- (((S)-oxetan-2-yl)methyl)-1H- benzo[d]imidazole-6-carboxylic acid |
| 105 | 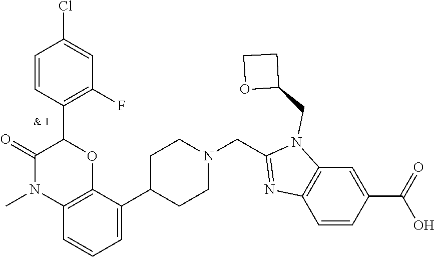 [SMILES: CN1C(=O)C(c2ccc(Cl)cc2F)Oc2c(C3CCN(Cc4nc5ccc(C(=O)O)cc5n4C[C@@H]4CCO4)CC3)cccc21] | 2-((4-(2-(4-chloro-2- fluorophenyl)-4-methyl-3-oxo- 3,4-dihydro-2H- benzo[b][1,4]oxazin-8- yl)piperidin-1-yl)methyl)-1- (((S)-oxetan-2-yl)methyl)-1H- benzo[d]imidazole-6-carboxylic acid |
| 106 | 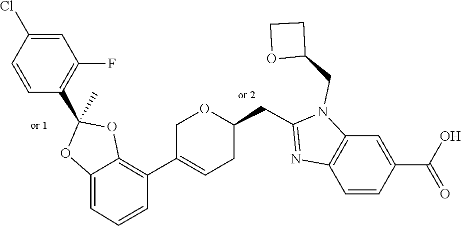 [SMILES: C[C@@]1(c2ccc(Cl)cc2F)Oc2cccc(C3=CC[C@H](Cc4nc5ccc(C(=O)O)cc5n4C[C@@H]4CCO4)OC3)c2O1] | 2-(((R)-5-((R)-2-(4-chloro-2- fluorophenyl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 107 | 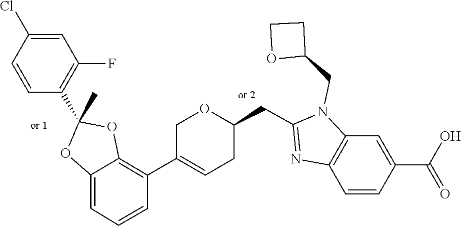 [SMILES: C[C@]1(c2ccc(Cl)cc2F)Oc2cccc(C3=CC[C@H](Cc4nc5ccc(C(=O)O)cc5n4C[C@@H]4CCO4)OC3)c2O1] | 2-(((R)-5-((S)-2-(4-chloro-2- fluorophenyl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 108 | 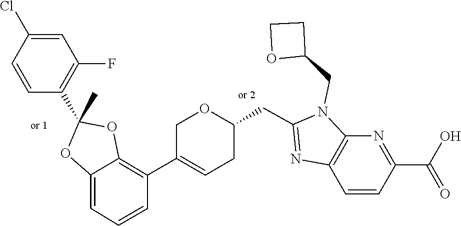 [SMILES: C[C@]1(c2ccc(Cl)cc2F)Oc2cccc(C3=CC[C@@H](Cc4nc5ccc(C(=O)O)nc5n4C[C@@H]4CCO4)OC3)c2O1] | 2-(((S)-5-((S)-2-(4-chloro-2- fluorophenyl)-2- methylbenzo[d][1,3]dioxol-4- yl)-3,6-dihydro-2H-pyran-2- yl)methyl)-3-(((S)-oxetan-2- yl)methyl)-3H-imidazo[4,5- b]pyridine-5-carboxylic acid |
| 109 | 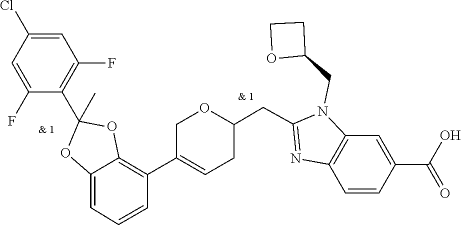 [SMILES: CC1(c2c(F)cc(Cl)cc2F)Oc2cccc(C3=CCC(Cc4nc5ccc(C(=O)O)cc5n4C[C@@H]4CCO4)OC3)c2O1] | 2-((5-(2-(4-chloro-2,6- difluorophenyl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 110 | 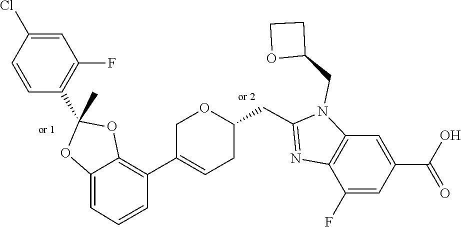 [SMILES: C[C@]1(c2ccc(Cl)cc2F)Oc2cccc(C3=CC[C@@H](Cc4nc5c(F)cc(C(=O)O)cc5n4C[C@@H]4CCO4)OC3)c2O1] | 2-(((S)-5-((S)-2-(4-chloro-2- fluorophenyl)-2- methylbenzo[d][1,3]dioxol-4- yl)-3,6-dihydro-2H-pyran-2- yl)methyl)-4-fluoro-1-(((S)- oxetan-2-yl)methyl)-1H- benzo[d]imidazole-6-carboxylic acid |
| 111 | 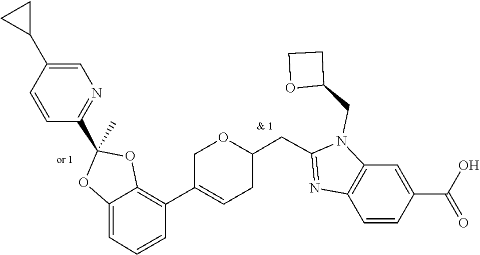 [SMILES: C[C@@]1(c2ccc(C3CC3)cn2)Oc2cccc(C3=CCC(Cc4nc5ccc(C(=O)O)cc5n4C[C@@H]4CCO4)OC3)c2O1] | 2-((5-((R)-2-(5- cyclopropylpyridin-2-yl)-2- methylbenzo[d][1,3]dioxol-4- yl)-3,6-dihydro-2H-pyran-2- yl)methyl)-1-(((S)-oxetan-2- yl)methyl)-1H- benzo[d]imidazole-6-carboxylic acid |
| 112 | 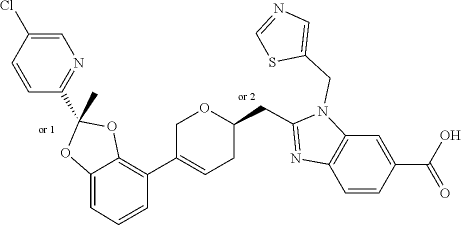 [SMILES: C[C@]1(c2ccc(Cl)cn2)Oc2cccc(C3=CC[C@H](Cc4nc5ccc(C(=O)O)cc5n4Cc4cncs4)OC3)c2O1] | 2-(((R)-5-((S)-2-(5- chloropyridin-2-yl)-2- methylbenzo[d][1,3]dioxol-4- yl)-3,6-dihydro-2H-pyran-2- yl)methyl)-1-(thiazol-5- ylmethyl)-1H- benzo[d]imidazole-6-carboxylic acid |
| 113 | 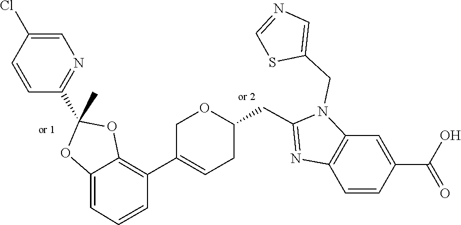 [SMILES: C[C@]1(c2ccc(Cl)cn2)Oc2cccc(C3=CC[C@@H](Cc4nc5ccc(C(=O)O)cc5n4Cc4cncs4)OC3)c2O1] | 2-(((S)-5-((S)-2-(5- chloropyridin-2-yl)-2- methylbenzo[d][1,3]dioxol-4- yl)-3,6-dihydro-2H-pyran-2- yl)methyl)-1-(thiazol-5- ylmethyl)-1H- benzo[d]imidazole-6-carboxylic acid |
| 114 | 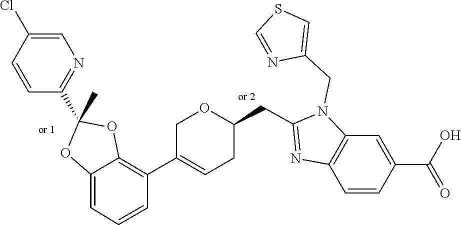 [SMILES: C[C@]1(c2ccc(Cl)cn2)Oc2cccc(C3=CC[C@H](Cc4nc5ccc(C(=O)O)cc5n4Cc4cscn4)OC3)c2O1] | 2-(((R)-5-((S)-2-(5- chloropyridin-2-yl)-2- methylbenzo[d][1,3]dioxol-4- yl)-3,6-dihydro-2H-pyran-2- yl)methyl)-1-(thiazol-4- ylmethyl)-1H- benzo[d]imidazole-6-carboxylic acid |
| 115 | 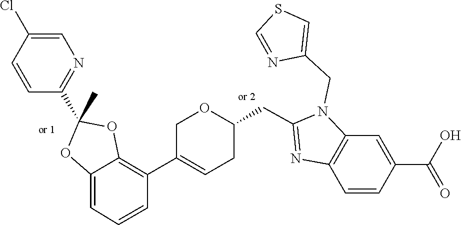 [SMILES: C[C@]1(c2ccc(Cl)cn2)Oc2cccc(C3=CC[C@@H](Cc4nc5ccc(C(=O)O)cc5n4Cc4cscn4)OC3)c2O1] | 2-(((S)-5-((S)-2-(5- chloropyridin-2-yl)-2- methylbenzo[d][1,3]dioxol-4- yl)-3,6-dihydro-2H-pyran-2- yl)methyl)-1-(thiazol-4- ylmethyl)-1H- benzo[d]imidazole-6-carboxylic acid |
| 116 | 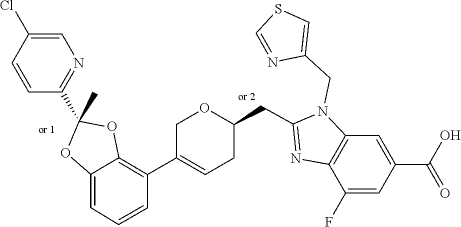 [SMILES: C[C@]1(c2ccc(Cl)cn2)Oc2cccc(C3=CC[C@H](Cc4nc5c(F)cc(C(=O)O)cc5n4Cc4cscn4)OC3)c2O1] | 2-(((R)-5-((S)-2-(5- chloropyridin-2-yl)-2- methylbenzo[d][1,3]dioxol-4- yl)-3,6-dihydro-2H-pyran-2- yl)methyl)-4-fluoro-1-(thiazol-4- ylmethyl)-1H- benzo[d]imidazole-6-carboxylic acid |
| 117 | 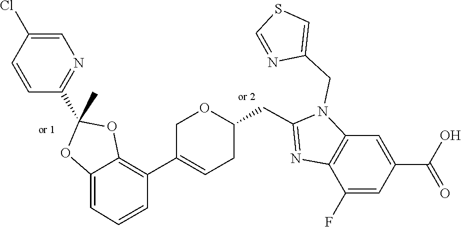 [SMILES: C[C@]1(c2ccc(Cl)cn2)Oc2cccc(C3=CC[C@@H](Cc4nc5c(F)cc(C(=O)O)cc5n4Cc4cscn4)OC3)c2O1] | 2-(((S)-5-((S)-2-(5- chloropyridin-2-yl)-2- methylbenzo[d][1,3]dioxol-4- yl)-3,6-dihydro-2H-pyran-2- yl)methyl)-4-fluoro-1-(thiazol-4- ylmethyl)-1H- benzo[d]imidazole-6-carboxylic acid |
| 118 | 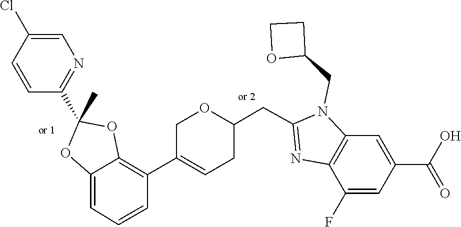 [SMILES: C[C@]1(c2ccc(Cl)cn2)Oc2cccc(C3=CCC(Cc4nc5c(F)cc(C(=O)O)cc5n4C[C@@H]4CCO4)OC3)c2O1] | 2-(((R)-5-((S)-2-(5- chloropyridin-2-yl)-2- methylbenzo[d][1,3]dioxol-4- yl)-3,6-dihydro-2H-pyran-2- yl)methyl)-4-fluoro-1-(((S)- oxetan-2-yl)methyl)-1H- benzo[d]imidazole-6-carboxylic acid |
| 119 | 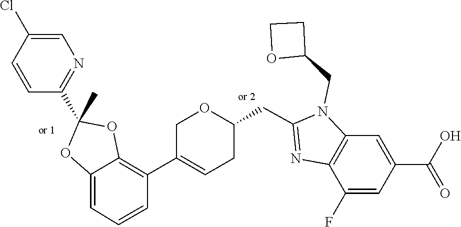 [SMILES: C[C@]1(c2ccc(Cl)cn2)Oc2cccc(C3=CC[C@@H](Cc4nc5c(F)cc(C(=O)O)cc5n4C[C@@H]4CCO4)OC3)c2O1] | 2-(((S)-5-((S)-2-(5- chloropyridin-2-yl)-2- methylbenzo[d][1,3]dioxol-4- yl)-3,6-dihydro-2H-pyran-2- yl)methyl)-4-fluoro-1-(((S)- oxetan-2-yl)methyl)-1H- benzo[d]imidazole-6-carboxylic acid |
| 120 | 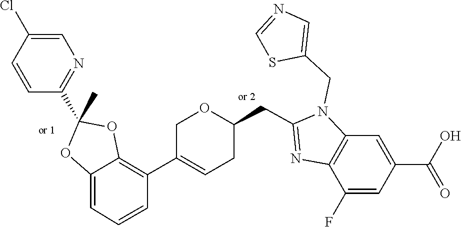 [SMILES: C[C@]1(c2ccc(Cl)cn2)Oc2cccc(C3=CC[C@H](Cc4nc5c(F)cc(C(=O)O)cc5n4Cc4cncs4)OC3)c2O1] | 2-(((R)-5-((S)-2-(5- chloropyridin-2-yl)-2- methylbenzo[d][1,3]dioxol-4- yl)-3,6-dihydro-2H-pyran-2- yl)methyl)-4-fluoro-1-(thiazol-5- ylmethyl)-1H- benzo[d]imidazole-6-carboxylic acid |
| 121 | 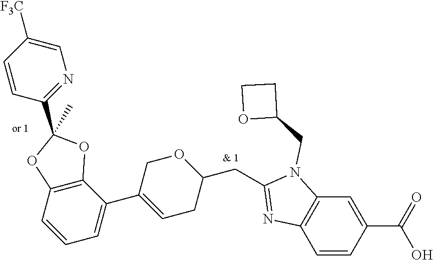 [SMILES: C[C@@]1(c2ccc(C(F)(F)F)cn2)Oc2cccc(C3=CCC(Cc4nc5ccc(C(=O)O)cc5n4C[C@@H]4CCO4)OC3)c2O1] | 2-((5-((R)-2-methyl-2-(5- (trifluoromethyl)pyridin-2- yl)benzo[d][1,3]dioxol-4-yl)-3,6- dihydro-2H-pyran-2-yl)methyl)- 1-(((S)-oxetan-2-yl)methyl)-1H- benzo[d]imidazole-6-carboxylic acid |
| 122 | 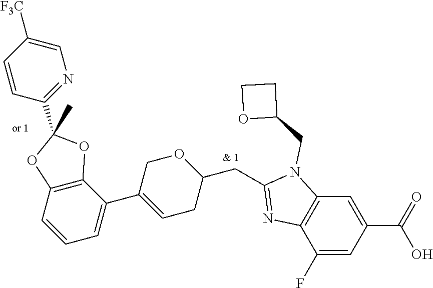 [SMILES: C[C@]1(c2ccc(C(F)(F)F)cn2)Oc2cccc(C3=CCC(Cc4nc5c(F)cc(C(=O)O)cc5n4C[C@@H]4CCO4)OC3)c2O1] | 4-fluoro-2-((5-((S)-2-methyl-2- (5-(trifluoromethyl)pyridin-2- yl)benzo[d][1,3]dioxol-4-yl)-3,6- dihydro-2H-pyran-2-yl)methyl)- 1-(((S)-oxetan-2-yl)methyl)-1H- benzo[d]imidazole-6-carboxylic acid |
| 123 | 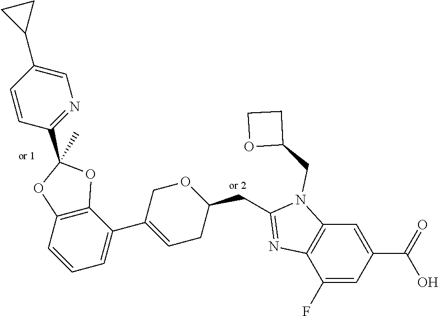 [SMILES: C[C@@]1(c2ccc(C3CC3)cn2)Oc2cccc(C3=CC[C@H](Cc4nc5c(F)cc(C(=O)O)cc5n4C[C@@H]4CCO4)OC3)c2O1] | 2-(((R)-5-((R)-2-(5- cyclopropylpyridin-2-yl)-2- methylbenzo[d][1,3]dioxol-4- yl)-3,6-dihydro-2H-pyran-2- yl)methyl)-4-fluoro-1-(((S)- oxetan-2-yl)methyl)-1H- benzo[d]imidazole-6-carboxylic acid |

The compounds of the present application possess advantageous characteristics, as compared to known compounds, such as known GLP-1 agonists. For example, the compounds of the present application display more potent GLP-1 agonistic activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present application can be measured according to methods commonly available in the art, such as methods exemplified herein.

Due to the existence of double bonds, the compounds of the present application may be in cis or trans, or Z or E, configuration. It is understood that although one configuration may be depicted in the structure of the compounds or formulae of the present application, the present application also encompasses the other configuration. For example, the compounds or formulae of the present application may be depicted in cis or trans, or Z or E, configuration.

In one embodiment, a compound of the present application (e.g., a compound of any of the formulae or any individual compounds disclosed herein) is a pharmaceutically acceptable salt. In another embodiment, a compound of the present application (e.g., a compound of any of the formulae or any individual compounds disclosed herein) is a solvate. In another embodiment, a compound of the present application (e.g., a compound of any of the formulae or any individual compounds disclosed herein) is a hydrate.

The details of the application are set forth in the accompanying description below. Although methods and materials similar or equivalent to those described herein can be used in the practice or testing of the present application, illustrative methods and materials are now described. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. All patents and publications cited in this specification are incorporated herein by reference in their entireties.

### Definitions

The articles “a” and “an” are used in this application to refer to one or more than one (i.e., at least one) of the grammatical object of the article. By way of example, “an element” means one element or more than one element.

The term “and/or” is used in this application to mean either “and” or “or” unless indicated otherwise.

The application also includes pharmaceutical compositions comprising an effective amount of a compound of the present application (e.g., a compound of any of the formulae or any individual compounds disclosed herein) and a pharmaceutically acceptable carrier.

The term “alkyl,” as used herein, refers to saturated, straight or branched-chain hydrocarbon radicals containing, in some embodiments, between one and six carbon atoms. Examples of C₁-C₈ alkyl radicals include, but are not limited to, methyl, ethyl, propyl, isopropyl, n-butyl, tert-butyl, neopentyl, n-hexyl, n-heptyl, and n-octyl radicals. Examples of C₁-C₆ alkyl radicals include, but are not limited to, methyl, ethyl, propyl, isopropyl, n-butyl, tert-butyl, neopentyl, and n-hexyl radicals.

The term “alkenyl,” as used herein, denotes a monovalent group derived from a hydrocarbon moiety containing, in some embodiments, from two to six carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl and the like.

The term “alkoxy” refers to an —O-alkyl radical.

The terms “hal,” “halo,” and “halogen,” as used herein, refer to an atom selected from fluorine, chlorine, bromine and iodine.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, indenyl and the like.

The term “aralkyl,” as used herein, refers to an alkyl residue attached to an aryl ring. Examples include, but are not limited to, benzyl, phenethyl and the like.

The term “cycloalkyl,” as used herein, denotes a monovalent group derived from a monocyclic or polycyclic saturated or partially unsaturated carbocyclic ring compound (fused, bridged, or spiro rings). Examples of C₃-C₈ cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1] heptyl, and bicyclo [2.2.2] octyl. Also contemplated is a monovalent group derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heteroaryl,” as used herein, refers to a mono- or poly-cyclic (e.g., bi-, or tri-cyclic or more) fused or non-fused, radical or ring system having at least one aromatic ring, having from five to ten ring atoms of which one ring atoms is selected from S, O, and N; zero, one, or two ring atoms are additional heteroatoms independently selected from S, O, and N; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “heteroaralkyl” as used herein, refers to an alkyl residue attached to a heteroaryl ring. Examples include, but are not limited to, pyridinylmethyl, pyrimidinylethyl and the like.

The term “heterocyclyl” or “heterocycloalkyl,” as used herein, refers to a saturated or unsaturated non-aromatic 3-, 4-, 5-, 6-, 7-, or 8-membered monocyclic, 7-, 8-, 9-, 10-, 11-, or 12-membered bicyclic (fused, bridged, or spiro rings), or 11-, 12, 13, or 14-membered tricyclic ring system (fused, bridged, or spiro rings), where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, and (iv) the nitrogen heteroatom may optionally be quaternized. Representative heterocycloalkyl groups include, but are not limited to, [1,3]dioxolanyl, pyrrolidinyl, pyrazolidinyl, pyrazolinyl, imidazolinyl, imidazolidinyl, piperidinyl, piperazinyl, 2-pyridone, oxazolidinyl, isoxazolidinyl, morpholinyl, tetrahydropyranyl, thiazolidinyl, isothiazolidinyl, tetrahydrofuryl, dioxanyl, oxetanyl, azetidinyl, thietanyl, oxiranyl, aziridinyl, thiiranyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 2-azaspiro[3.3]heptan-5-amine, 1-azaspiro[3.3]heptan-5-amine, 1-oxa-6-azaspiro[3.3]heptan-3-amine, 2-azaspiro[3.3]heptan-6-amine, 1-azaspiro[3.3]heptan-6-amine, 6-azaspiro[3.4]octan-2-amine, 5-azaspiro[3.4]octan-2-amine, 6-azaspiro[3.4]octan-1-amine, 5-azaspiro[3.4]octan-1-amine, 5-oxa-2-azaspiro[3.4]octan-7-amine, 7-amino-5-thia-2-azaspiro[3.4]octane 5,5-dioxide, 5-oxa-2-azaspiro[3.4]octan-8-amine, 8-amino-5-thia-2-azaspiro[3.4]octane 5,5-dioxide, and the like.

The term “alkylamino” refers to a group having the structure, e.g., NH(C₁-C₆ alkyl), where C₁-C₆ alkyl is as previously defined.

The term “dialkylamino” refers to a group having the structure, e.g., N(C₁-C₆ alkyl)₂, where C₁-C₆ alkyl is as previously defined.

In accordance with the application, any of the aryls, substituted aryls, heteroaryls and substituted heteroaryls described herein, can be any aromatic group. Aromatic groups can be substituted or unsubstituted.

As described herein, compounds of the application may optionally be substituted with one or more substituents, such as are illustrated generally above, or as exemplified by particular classes, subclasses, and species of the application. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted alkenyl,” “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocyclyl,” and any other optionally substituted group as used herein, refer to groups that are substituted or unsubstituted by independent replacement of one, two, or three or more of the hydrogen atoms thereon with substituents including, but not limited to: —F, —Cl, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —C(O)NH₂, —C(O)NH—C₁-C₁₂-alkyl, —C(O)NH—C₂-C₁₂-alkenyl, —C(O)NH—C₂-C₁₂-alkenyl, —C(O)NH—C₃-C₁₂-cycloalkyl, —C(O)NH-aryl, —C(O)NH-heteroaryl, —C(O)NH-heterocycloalkyl, —OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OC(O)NH₂, —OC(O)NH—C₁-C₁₂-alkyl, —OC(O)NH— C₂-C₁₂-alkenyl, —OC(O)NH— C₂-C₁₂-alkenyl, —OC(O)NH—C₃-C₁₂-cycloalkyl, —OC(O)NH-aryl, —OC(O)NH-heteroaryl, —OC(O)NH— heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, NHC(O)NH-heterocycloalkyl, —NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NHheterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, C(NH)NH—C₃-C₁₂-cycloalkyl, —C(NH)NH-aryl, —C(NH)NH-heteroaryl, —C(NH)NHheterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl.

The term “carrier”, as used in this application, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The compounds of the present application may form salts which are also within the scope of this application. Reference to a compound of the Formulae herein is understood to include reference to salts thereof, unless otherwise indicated.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The compounds of the present application, for example, including the pharmaceutically acceptable salts, solvates, prodrugs, stereoisomers, or tautomers of the compounds, can exist in a solvated form with other solvent molecules or in an unsolvated form.

“Solvate” means solvent addition forms that contain either stoichiometric or non stoichiometric amounts of solvent. Some compounds or salts have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this application, as are positional isomers (such as, for example, 4-pyridyl and 3-pyridyl). For example, if a compound of Formula (Y), (Y′), (A), or (I) incorporates a double bond or a fused ring, both the cis- and trans-forms, as well as mixtures, are embraced within the scope of the application. Individual stereoisomers of the compound of the application may, for example, be substantially free of other isomers, or may be admixed, for example, as racemates or with all other, or other selected, stereoisomers. The chiral centers of the present application can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of the present application (e.g., a compound of any of the formulae or any individual compounds disclosed herein) may have one or more asymmetric carbon atom and may occur as racemates, racemic mixtures or as individual enantiomers or diastereomers.

In the present specification, the structural formula of the compound represents a certain isomer for convenience in some cases, but the present application includes all isomers, such as geometrical isomers, optical isomers based on an asymmetrical carbon, stereoisomers, tautomers, and the like.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

The compounds of the application may contain asymmetric or chiral centers, and, therefore, exist in different stereoisomeric forms. It is intended that all stereoisomeric forms of the compounds of the application as well as mixtures thereof, including racemic mixtures, form part of the present application. In addition, the present application embraces all geometric and positional isomers. For example, if a compound of the application incorporates a double bond or a fused ring, both the cis- and trans-forms, as well as mixtures, are embraced within the scope of the application. Each compound herein disclosed includes all the enantiomers that conform to the general structure of the compound. The compound may be in a racemic or enantiomerically pure form, or any other form in terms of stereochemistry. The assay results may reflect the data collected for the racemic form, the enantiomerically pure form, or any other form in terms of stereochemistry.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

In another embodiment of the application, the compound of the present application (e.g., a compound of any of the formulae or any individual compounds disclosed herein) is an enantiomer. In some embodiments the compound is the (S)-enantiomer. In other embodiments the compound is the (R)-enantiomer. In yet other embodiments, the compounds of the present application (e.g., a compound of any of the formulae or any individual compounds disclosed herein) may be (+) or (−) enantiomers. The compound may contain more than one stereocenter.

In another embodiment of the application, the compounds of the present application (e.g., a compound of any of the formulae or any individual compounds disclosed herein) are diastereomers. In some embodiments, the compounds are the syn diastereomer. In other embodiments, the compounds are the anti diastereomer.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column.

It is also possible that the compounds of the application may exist in different tautomeric forms, and all such forms are embraced within the scope of the application. Also, for example, all keto-enol and imine-enamine forms of the compounds are included in the application.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-imine.

The present application relates to a compound of the present application (e.g., a compound of any of the formulae or any individual compounds disclosed herein) or pharmaceutically acceptable salts, solvates, prodrugs, stereoisomers, or tautomers thereof, capable of modulating (e.g., activating or stimulating) GLP-1 receptor, which are useful for the treatment of diseases and disorders associated with modulation of GLP-1 receptor. The application further relates to compounds of the present application (e.g., a compound of any of the formulae or any individual compounds disclosed herein), or pharmaceutically acceptable salts, solvates, prodrugs, stereoisomers, or tautomers thereof, which are useful for modulating (e.g., activating or stimulating) GLP-1 receptor. In some embodiments, the GLP-1 receptor is wild-type GLP-1 receptor. In other embodiments, the GLP-1 receptor is a mutant GLP-1 receptor.

In some embodiments, the application provides a compound of the present application (e.g., a compound of any of the formulae or any individual compounds disclosed herein), wherein the compound possesses advantageous characteristics, such as increased potency, improved oral bioavailability, or desirable pharmacodynamic/pharmacokinetic profile, compared to one or more known GLP-1 receptor ligands (e.g., incretin or small molecule GLP-1 receptor agonists).

Potency of the agonist/activator/stimulator can be determined by EC₅₀ value. A compound with a lower EC₅₀ value, as determined under substantially similar conditions, is a more potent agonist/activator/stimulator relative to a compound with a higher EC₅₀ value.

The compounds of the present application can be converted to N-oxides by treatment with an oxidizing agent (e.g., 3-chloroperoxybenzoic acid (m-CPBA) and/or hydrogen peroxides) to afford other compounds of the present application. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present application can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compounds as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

The term “prodrug,” as used in this application, means a compound which is convertible in vivo by metabolic means (e.g., by hydrolysis) to a disclosed compound.

Since prodrugs are known to enhance numerous desirable qualities of pharmaceuticals (e.g., solubility, bioavailability, manufacturing, etc.) the compounds of the present application (e.g., a compound of any of the formulae or any individual compounds disclosed herein), or pharmaceutically acceptable salts, solvates, prodrugs, stereoisomers, or tautomers thereof can be delivered in prodrug form. Thus, the present application is intended to cover prodrugs of a compound of the present application (e.g., a compound of any of the formulae or any individual compounds disclosed herein), or a pharmaceutically acceptable salt, solvate, prodrug, stereoisomer, or tautomer thereof, methods of delivering the same and compositions containing the same. “Prodrugs” are intended to include any covalently bonded carriers that release an active parent drug of the present application in vivo when such prodrug is administered to a mammalian subject. Prodrugs are prepared by modifying functional groups present in the compound in such a way that the modifications are cleaved, either in routine manipulation or in vivo, to the parent compound. Prodrugs include compounds of the application wherein a hydroxyl or amino, group is bonded to any group that, when the prodrug of the present application is administered to a mammalian subject, it cleaves to form a free hydroxyl or free amino group, respectively. Examples of prodrugs include, but are not limited to, acetate, formate, and benzoate derivatives of alcohol and amine functional groups in the compounds of each of the formulae described herein or a pharmaceutically acceptable salt, solvate, prodrug, stereoisomer, or tautomer thereof.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate.

As used herein, the term “analog” refers to a compound that is structurally similar to another compound but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

The application also comprehends isotopically-labeled compounds, which are identical to those recited in the each of the formulae described herein, but for the fact that one or more atoms are replaced by an atom having an atomic mass or mass number different from the atomic mass or mass number most commonly found in nature. Examples of isotopes that can be incorporated into compounds of the application include isotopes of hydrogen, carbon, nitrogen, fluorine, such as ³H, ¹¹C, ¹⁴C, ²H and ¹⁸F.

Compounds of the present application (e.g., a compound of any of the formulae or any individual compounds disclosed herein), or pharmaceutically acceptable salts, solvates, prodrugs, stereoisomers, or tautomers thereof, that contain the aforementioned isotopes and/or other isotopes of other atoms are within the scope of the present application. Isotopically-labeled compounds of the present application, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are useful for their ease of preparation and detectability. ¹¹C and ¹⁸F isotopes are useful in PET (positron emission tomography). PET is useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances, isotopically labeled compounds of the present application (e.g., a compound of any of the formulae or any individual compounds disclosed herein), or pharmaceutically acceptable salts, solvates, prodrugs, stereoisomers, or tautomers thereof, can generally be prepared by carrying out the procedures disclosed in the Schemes and/or in the Examples described herein, by substituting a readily available isotopically labeled reagent for a non-isotopically labeled reagent. In one embodiment, the compound of the present application (e.g., a compound of any of the formulae or any individual compounds disclosed herein) or pharmaceutically acceptable salts, solvates, prodrugs, stereoisomers, or tautomers thereof, is not isotopically labelled.

The term “administer”, “administering”, or “administration” as used in this application refers to either directly administering a disclosed compound or pharmaceutically acceptable salt of the disclosed compound or a composition to a subject, or administering a prodrug, derivative or analog of the compound or pharmaceutically acceptable salt of the compound or a composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” or “therapeutically effective amount” when used in connection with a compound or pharmaceutical composition is an amount effective for treating or preventing a disease in a subject as described herein.

The term “treating” with regard to a subject, refers to improving at least one symptom of the subject's disorder. Treating includes curing, improving, or at least partially ameliorating the disorder.

The compounds of the present application, or a pharmaceutically acceptable salt, solvate, prodrug, stereoisomer, or tautomer thereof, can also be used to prevent a disease, condition or disorder. As used herein, “preventing” or “prevent” describes reducing or eliminating the onset of the symptoms or complications of the disease, condition or disorder.

The term “disorder” is used in this application to mean, and is used interchangeably with, the terms disease, condition, or illness, unless otherwise indicated.

As used herein, the term “GLP-1 receptor-mediated” diseases or disorders means any disease or other deleterious condition in which GLP-1 receptor, or a mutant thereof, is known to play a role. Accordingly, another embodiment of the present application relates to treating or lessening the severity of one or more diseases in which GLP-1 receptor, or a mutant thereof, is known to play a role. Specifically, the present application relates to a method of treating or lessening the severity of a disease or condition as described herein, wherein said method comprises administering to a subject in need thereof a compounds of the present application (e.g., a compound of any of the formulae or any individual compounds disclosed herein), or pharmaceutically acceptable salts, solvates, prodrugs, stereoisomers, or tautomers thereof, or a composition according to the present application.

### Methods for Preparing the Compounds

The compounds of the present application may be made by a variety of methods, including standard chemistry. Suitable synthetic routes are depicted in the Schemes given below.

The compounds of the present application (e.g., a compound of any of the formulae or any individual compounds disclosed herein) may be prepared by methods known in the art of organic synthesis as set forth in part by the following synthetic schemes. In the scheme described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of the compounds of the present application.

Those skilled in the art will recognize if a stereocenter exists in the compounds of the present application (e.g., a compound of any of the formulae or any individual compounds disclosed herein). Accordingly, the present application includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

The compounds of the present application can be prepared in a number of ways well known to those skilled in the art of organic synthesis. By way of example, the compounds of the present application can be synthesized using the methods described below, together with synthetic methods known in the art of synthetic organic chemistry, or variations thereon as appreciated by those skilled in the art. Preferred methods include but are not limited to those methods described below. The compounds of the present application (i.e., a compound of the present application (e.g., a compound of any of the formulae or any individual compounds disclosed herein)) can be synthesized by following the steps outlined in the examples, schemes, procedures, and/or synthesis described herein (e.g., Examples). Starting materials are either commercially available or made by known procedures in the reported literature or as illustrated.

A mixture of enantiomers, diastereomers, cis trans isomers resulting from the processes described above can be separated into their single components by chiral salt technique, chromatography using normal phase, reverse phase or chiral column, depending on the nature of the separation.

The present application relates to methods of synthesizing a compound of the application or a pharmaceutically acceptable salt, solvate, prodrug, stereoisomer, or tautomer thereof. The schemes and description below depict general routes for the preparation of a compound of the application. For example, compounds of the present application can be synthesized by following the steps outlined in Schemes 1 or 2, which comprises different sequences of assembling intermediates (A), (A)-1, (A)-2, (A)-3, and (A)-4. Starting materials are either commercially available or made by known procedures in the reported literature or as illustrated.

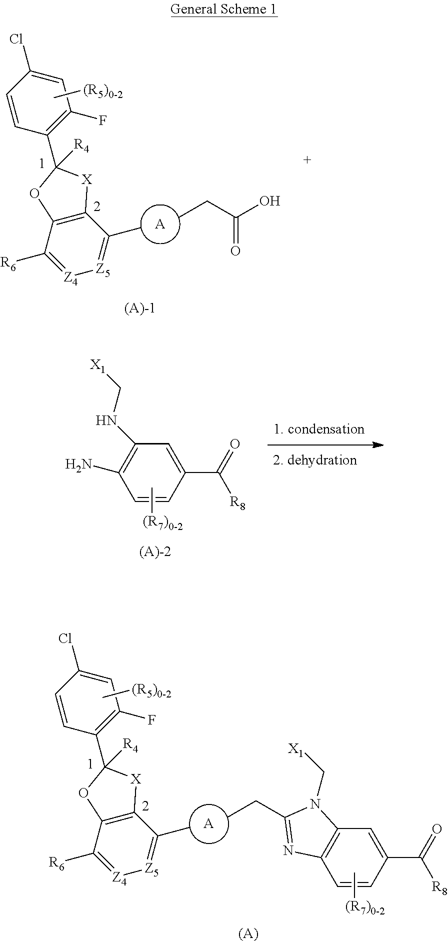

Scheme 1 depicts a generic synthesis of compounds of Formula (A). Specifically, the acid group of compound (A)-1 can be condensed with a suitable amine, typically under condensation conditions, which can be dehydrated to give compounds of Formula (A).

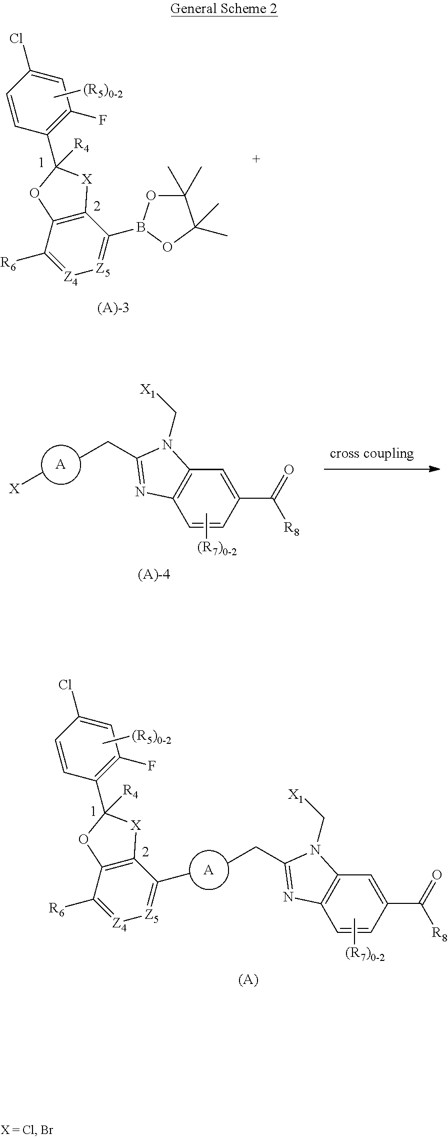

Scheme 2 depicts another generic synthesis of compounds of Formula (A). (A)-3 can be coupled with (A)-4, typically under Suzuki coupling conditions, to give compounds of Formula (A).

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

Abbreviations used in the following examples and elsewhere herein are:
DIEA N,N-diisopropylethylamine
DMF N,N-dimethylformamide
DMA N,N-dimethylacetamide
DMSO dimethylsulfoxide
DEAD DiethylAzodicarboxylate
EA ethyl acetate
IPA iso-propyl alcohol
IPE di-isopropyl ether
MeCN acetonitrile
THF tetrahydrofuran
m-CPBA 3-chlorobenzenecarboperoxoic acid
DCM dichloromethane
LC/MS liquid chromatography-mass spectrometry
MeOH methanol
MS mass spectrometry
PE petroleum ether
NMP N-methyl pyrrolidinone
NMR nuclear magnetic resonance
ppm parts per million
TEA triethylamine

### Biological Assays

The biological activities of the compounds of the present application can be assessed with methods and assays known in the art. Exemplary methods are described in the Examples, such as GLP1R cAMP assay and human GLP-1 activity assay.

The compounds of the present application also possess favorable pharmacokinetic properties and/or activity profile against hepatic drug transporters (e.g., OATP1B1, OATP1B3), compared to known small molecule GLP-1 receptor agonists. These properties can be evaluated with methods and assays available in the art, such as those described and/or exemplified herein.

### Methods of Using the Compounds

The compounds of the present application are useful for modulating (e.g., activating or stimulating) GLP-1 receptor. As such, the compounds of the present application are useful for the treatment of a disease or disorder associated with the GLP-1 receptor, including metabolic diseases such as diabetes and obesity, cardiovascular diseases, liver diseases such as NASH, kidney diseases, neurodegenerative diseases, and other diseases or disorders associated with the modulation of GLP-1 receptor. For example, a disease or disorder associated with the GLP-1 receptor includes, but is not limited to, diabetes (non-insulin-dependent diabetes mellitus (Type 2 diabetes) or insulin-dependent diabetes mellitus (Type 1 diabetes)), diabetic complication, obesity, impaired glucose tolerance, overweight condition, hyperlipidemia, hypercholesteremia, atherosclerosis, hypertension, coronary heart disease such as myocardial infarction and angina pectoris, congestive heart failure, cardiac arrhythmias, brain infarction, stroke, liver diseases such as nonalcoholic fatty liver disease (NAFLD) and nonalcoholic steatohepatitis (NASH), dementia, Parkinson's disease, and diabetic kidney disease.

“Diabetes” is a state or a disease in which the metabolism for generating and using glucose becomes deficient due to a failure in maintaining an appropriate blood glucose level in the body, and encompasses insulin-dependent diabetes mellitus (Type 1 diabetes) and non-insulin-dependent diabetes mellitus (Type 2 diabetes).

“Dementia” includes, for example, Alzheimer's disease, vascular dementia, and diabetic dementia.

“Diabetic complication” is a complication caused by diabetes or hyperglycemia, including ketoacidosis, infectious disease (e.g., skin infection, soft tissue infection, biliary system infection, respiratory system infection, urinary tract infection), microangiopathy (e.g., nephropathy, retinopathy), neuropathy (e.g., sensory nerve disorder, motor nerve disorder, autonomic nerve disorder), and gangrene. Major diabetes complexes include diabetic retinopathy, diabetic nephropathy, and diabetic neuropathy.

A liver disease (e.g., a liver disease associated with GLP-1 receptor) includes, but is not limited to, NASH, NAFLD, liver inflammation, liver fibrosis, cirrhosis, liver autoimmune diseases, autoimmune hepatitis, primary biliary cirrhosis, sclerosing cholangitis, autoimmune cholangitis, and alcoholic liver disease.

Another aspect of the application relates to a method of treating, preventing, inhibiting, or eliminating a disease or disorder associated with modulation of GLP-1 receptor (e.g., activation or stimulation of GLP-1 receptor). The method comprises administering to a subject in need of a treatment for diseases or disorders associated with modulation of GLP-1 receptor an effective amount a compound of the present application (e.g., a compound of any of the formulae or any individual compounds disclosed herein) or a pharmaceutically acceptable salt, solvate, prodrug, stereoisomer, or tautomer thereof or a pharmaceutical composition of a compound of the present application (e.g., a compound of any of the formulae or any individual compounds disclosed herein). In one embodiment, the GLP-1 receptor-mediated disorder is a disease or disorder described herein. In some embodiments, the GLP-1 receptor is wild-type GLP-1 receptor. In other embodiments, the GLP-1 receptor is mutant GLP-1 receptor.

Another aspect of the application relates to a method of modulating GLP-1 receptor, the method comprising administering to a subject in need thereof a therapeutically effective amount of a compound of the present application (e.g., a compound of any of the formulae or any individual compounds disclosed herein), or a pharmaceutically acceptable salt, solvate, prodrug, stereoisomer, or tautomer thereof, or a pharmaceutical composition of a compound of the present application (e.g., a compound of any of the formulae or any individual compounds disclosed herein). In one embodiment, modulating GLP-1 receptor is activating GLP-1 receptor. In some embodiments, the GLP-1 receptor is wild-type GLP-1 receptor. In other embodiments, the GLP-1 receptor is mutant GLP-1 receptor.

Another aspect of the application relates to a compound of the present application (e.g., a compound of any of the formulae or any individual compounds disclosed herein), or a pharmaceutically acceptable salt, solvate, prodrug, stereoisomer, or tautomer thereof, for use in a method of treating a GLP-1 receptor-mediated disease or disorder. In one embodiment, the GLP-1 receptor-mediated disorder is a disease or disorder described herein. In some embodiments, the GLP-1 receptor is wild-type GLP-1 receptor. In other embodiments, the GLP-1 receptor is mutant GLP-1 receptor.

In another aspect, the present application relates to a pharmaceutical composition of a compound of the present application (e.g., a compound of any of the formulae or any individual compounds disclosed herein), or a pharmaceutically acceptable salt, solvate, prodrug, stereoisomer, or tautomer thereof, for use in a method of treating a GLP-1 receptor-mediated disease or disorder. In one embodiment, the GLP-1 receptor-mediated disorder is a disease or disorder described herein. In some embodiments, the GLP-1 receptor is wild-type GLP-1 receptor. In other embodiments, the GLP-1 receptor is mutant GLP-1 receptor.

Another aspect of the application relates to a compound of the present application (e.g., a compound of any of the formulae or any individual compounds disclosed herein), or a pharmaceutically acceptable salt, solvate, prodrug, stereoisomer, or tautomer thereof, for use in modulating GLP-1 receptor. In one embodiment, modulating GLP-1 receptor is activating or stimulating GLP-1 receptor. In some embodiments, the GLP-1 receptor is wild-type GLP-1 receptor. In other embodiments, the GLP-1 receptor is mutant GLP-1 receptor.

In another aspect, the present application relates to a pharmaceutical composition of a compound of the present application (e.g., a compound of any of the formulae or any individual compounds disclosed herein), or a pharmaceutically acceptable salt, solvate, prodrug, stereoisomer, or tautomer thereof, for use in modulating GLP-1 receptor. In one embodiment, modulating GLP-1 receptor is activating or stimulating GLP-1 receptor. In some embodiments, the GLP-1 receptor is wild-type GLP-1 receptor. In other embodiments, the GLP-1 receptor is mutant GLP-1 receptor.

Another aspect of the application relates to the use of a compound of the present application (e.g., a compound of any of the formulae or any individual compounds disclosed herein), or a pharmaceutically acceptable salt, solvate, prodrug, stereoisomer, or tautomer thereof, in the manufacture of a medicament for treating a GLP-1 receptor-mediated disease or disorder. In one embodiment, the GLP-1 receptor-mediated disorder is a disease or disorder described herein. In some embodiments, the GLP-1 receptor is wild-type GLP-1 receptor. In other embodiments, the GLP-1 receptor is mutant GLP-1 receptor.

In another aspect, the present application relates to the use of a pharmaceutical composition of a compound of the present application (e.g., a compound of any of the formulae or any individual compounds disclosed herein), or a pharmaceutically acceptable salt, solvate, prodrug, stereoisomer, or tautomer thereof, in the manufacture of a medicament for treating a GLP-1 receptor-mediated disease or disorder. In one embodiment, the GLP-1 receptor-mediated disorder is a disease or disorder described herein. In some embodiments, the GLP-1 receptor is wild-type GLP-1 receptor. In other embodiments, the GLP-1 receptor is mutant GLP-1 receptor.

Another aspect of the application relates to the use of a compound of the present application (e.g., a compound of any of the formulae or any individual compounds disclosed herein), or a pharmaceutically acceptable salt, solvate, prodrug, stereoisomer, or tautomer thereof, in the manufacture of a medicament for modulating GLP-1 receptor. In one embodiment, modulating GLP-1 receptor is activating or stimulating GLP-1 receptor. In some embodiments, the GLP-1 receptor is wild-type GLP-1 receptor. In other embodiments, the GLP-1 receptor is mutant GLP-1 receptor.

In another aspect, the present application relates to the use of a pharmaceutical composition of a compound of the present application (e.g., a compound of any of the formulae or any individual compounds disclosed herein), or a pharmaceutically acceptable salt, solvate, prodrug, stereoisomer, or tautomer thereof, in the manufacture of a medicament for modulating GLP-1 receptor. In one embodiment, modulating GLP-1 receptor is activating or stimulating GLP-1 receptor. In some embodiments, the GLP-1 receptor is wild-type GLP-1 receptor. In other embodiments, the GLP-1 receptor is mutant GLP-1 receptor.

The disclosed compound of the application can be administered in effective amounts to treat or prevent a disorder and/or prevent the development thereof in subjects.

The compound of the application can be administered in therapeutically effective amounts in a combinational therapy with one or more therapeutic agents (pharmaceutical combinations) or modalities, e.g., non-drug therapies. For example, synergistic effects can occur with other anti-proliferative, anti-cancer, immunomodulatory or anti-inflammatory substances. In some embodiments, a compound of the present application (e.g., a compound of any of the formulae or any individual compounds disclosed herein) is administered in combination with an additional therapeutic agent selected from an anti-inflammatory agent, an immunomodulatory agent, a chemotherapeutic agent, an agent for treating cardiovascular disease, an agent for treating liver disease, an agent for treating lung disease, an agent for treating kidney disease, an agent for treating ocular disease, an agent for treating skin disease, an anti-viral agent, an agent for treating blood disorders, an agent for treating diabetes, and an agent for treating immunodeficiency disorders. Where the compound of the application is administered in conjunction with other therapies, dosages of the co-administered compounds will of course vary depending on the type of co-drug employed, on the specific drug employed, on the condition being treated and so forth.

Combination therapy includes the administration of the subject compound in further combination with other biologically active ingredients (such as, but not limited to, an anti-inflammatory agent, an immunomodulatory agent, chemotherapeutic agent, an agent for treating cardiovascular disease, an agent for treating liver disease, an anti-viral agent, an agent for treating blood disorders, an agent for treating diabetes, an agent for treating immunodeficiency disorders, and an agent for treating pain) and non-drug therapies (such as, but not limited to, surgery or radiation treatment). For instance, the compound of the application can be used in combination with other pharmaceutically active compounds, preferably compounds that are able to enhance the effect of the compound of the application. The compound of the application can be administered simultaneously (as a single preparation or separate preparation) or sequentially to the other drug therapy or treatment modality. In general, a combination therapy envisions administration of two or more drugs during a single cycle or course of therapy.

### Pharmaceutical Compositions

The present application also provides pharmaceutical compositions comprising a compound of the present application (e.g., a compound of any of the formulae or any individual compounds disclosed herein), or a pharmaceutically acceptable salt, solvate, prodrug, stereoisomer, or tautomer thereof, in combination with at least one pharmaceutically acceptable excipient or carrier.

A “pharmaceutical composition” is a formulation containing the compound of the present application in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or a pharmaceutically acceptable salt, solvate, prodrug, stereoisomer, or tautomer thereof thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical compositions of the application are formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the application can be administered to a subject in many of the well-known methods currently used for chemotherapeutic treatment. The dose chosen should be sufficient to constitute effective treatment but not as high as to cause unacceptable side effects. The state of the disease condition and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or modulatory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In one embodiment, the disease or disorder is a disease or disorder described herein.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compound (i.e., a compound of the present application (e.g., a compound of any of the formulae or any individual compounds disclosed herein)) of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compound into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compound is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compound is formulated into ointments, salves, gels, or creams as generally known in the art.

The active compound can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the application are dictated by and directly dependent on the unique characteristics of the active compound and the particular therapeutic effect to be achieved.

In therapeutic applications, the dosages of the pharmaceutical compositions used in accordance with the application vary depending on the agent, the age, weight, and clinical condition of the recipient patient, and the experience and judgment of the clinician or practitioner administering the therapy, among other factors affecting the selected dosage. Dosages can range from about 0.01 mg/kg per day to about 5000 mg/kg per day. An effective amount of a pharmaceutical agent is that which provides an objectively identifiable improvement as noted by the clinician or other qualified observer. As used herein, the term “dosage effective manner” refers to amount of an active compound to produce the desired biological effect in a subject or cell.

The pharmaceutical compositions can be included in a container, pack, or dispenser together with instructions for administration.

As used herein, “pharmaceutically acceptable salts” refer to derivatives of the compound of the present application wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present application also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compound of the present application can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The compound of the present application can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compound of the present application can be delivered in prodrug form. Thus, the present application is intended to cover prodrugs of the presently claimed compound, methods of delivering the same and compositions containing the same. “Prodrugs” are intended to include any covalently bonded carriers that release an active parent drug of the present application in vivo when such prodrug is administered to a subject. Prodrugs in the present application are prepared by modifying functional groups present in the compound in such a way that the modifications are cleaved, either in routine manipulation or in vivo, to the parent compound. Prodrugs include the compound of the present application wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in the compound of the application, and the like, See Bundegaard, H., Design of Prodrugs, p 1-92, Elsevier, New York-Oxford (1985).

The compound, or pharmaceutically acceptable salts, tautomers, prodrugs, solvates, metabolites, polymorphs, analogs or derivatives thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound or a pharmaceutically acceptable salt, solvate, prodrug, stereoisomer, or tautomer thereof is administered orally. One skilled in the art will recognize the advantages of certain routes of administration.

The dosage regimen utilizing the compound is selected in accordance with a variety of factors including type, species, age, weight, sex and medical condition of the patient; the severity of the condition to be treated; the route of administration; the renal and hepatic function of the patient; and the particular compound or pharmaceutically acceptable salt, solvate, prodrug, stereoisomer, or tautomer thereof employed. An ordinarily skilled physician or veterinarian can readily determine and prescribe the effective amount of the drug required to prevent, counter or arrest the progress of the condition.

Techniques for formulation and administration of the disclosed compound of the application can be found in Remington: the Science and Practice of Pharmacy, 19^{th} edition, Mack Publishing Co., Easton, Pa. (1995). In an embodiment, the compound described herein, and the pharmaceutically acceptable salts, solvates, prodrugs, stereoisomers, or tautomers thereof, are used in pharmaceutical preparations in combination with a pharmaceutically acceptable carrier or diluent. Suitable pharmaceutically acceptable carriers include inert solid fillers or diluents and sterile aqueous or organic solutions. The compound or pharmaceutically acceptable salts, solvates, prodrugs, stereoisomers, or tautomers thereof will be present in such pharmaceutical compositions in amounts sufficient to provide the desired dosage amount in the range described herein.

All percentages and ratios used herein, unless otherwise indicated, are by weight. Other features and advantages of the present application are apparent from the different examples. The provided examples illustrate different components and methodology useful in practicing the present application. The examples do not limit the claimed application. Based on the present application the skilled artisan can identify and employ other components and methodology useful for practicing the present application.

### Examples

The application is further illustrated by the following examples and synthesis schemes, which are not to be construed as limiting this application in scope or spirit to the specific procedures herein described. It is to be understood that the examples are provided to illustrate certain embodiments and that no limitation to the scope of the application is intended thereby. It is to be further understood that resort may be had to various other embodiments, modifications, and equivalents thereof which may suggest themselves to those skilled in the art without departing from the spirit of the present application and/or scope of the appended claims.

### Intermediate A:

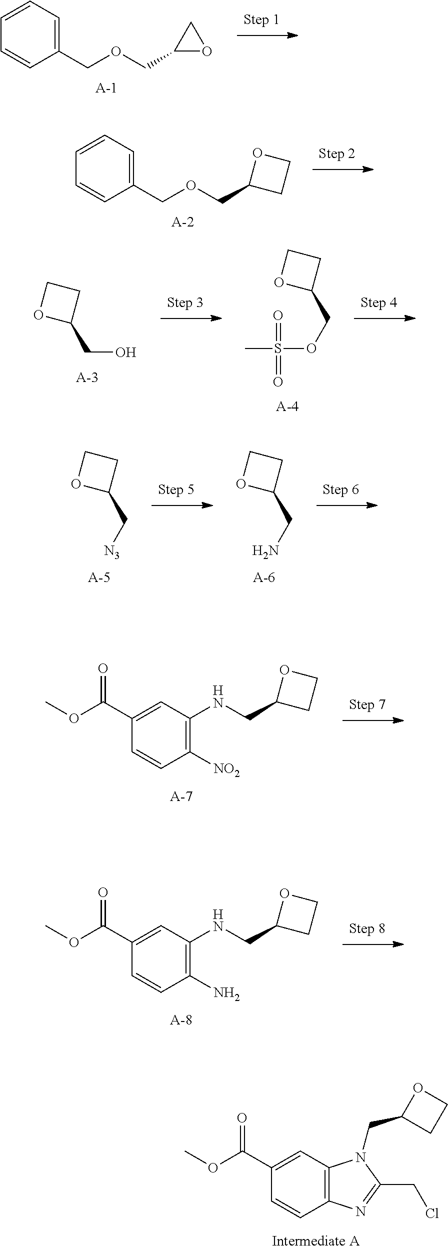

### Step 1: A-2

To a solution of t-BuOK (152.05 g, 1.36 mol) in t-BuOH (900 mL) was added iodo-trimethyl-oxo-sulfane (298.21 g, 1.36 mol) at 25° C., and the mixture was stirred at 60° C. for 30 minutes, before A-1 (89 g, 542.0 mmol) was added slowly. The mixture was stirred at 80° C. for 2 hrs. The resulting mixture was cooled, filtered through celite, and washed with PE. The filtrate was poured into water (1500 mL) and extracted with PE (800 mL×3). The organic layers were dried over Na₂SO₄ and concentrated to give a residue, which was purified by silica gel column (PE:EA=20:1-10:1) to give A-2 (54 g, 55.9% yield). ¹H NMR (300 MHz, CDCl₃): δ 7.49-7.22 (m, 5H), 4.97 (m, 1H), 4.71-4.53 (m, 4H), 3.70-3.57 (m, 2H), 2.72-2.49 (m, 2H) ppm.

### Step 2: A-3

To a solution of A-2 (18 g, 100.99 mmol) in MeOH (200 mL) was added Pd/C (10%, 3.6 g). The reaction mixture was stirred at 25° C. under H2 atmosphere for 20 hrs. The resulting mixture was filtered and the filtrate was concentrated under reduced pressure to give A-3 (7.2 g, 81.1% yield). ¹H NMR (300 MHz, CDCl₃): δ 4.96-4.89 (m, 1H), 4.75-4.62 (m, 1H), 4.55-4.49 (m, 1H), 3.77-3.54 (m, 2H), 2.69-2.54 (m, 2H) ppm.

### Step 3: A-4

To a solution of A-3 (14.90 g, 169.06 mmol, from several batches) in THF (150 mL) was added TEA (51.32 g, 507.18 mmol, 70.69 mL) at 25° C. Then methylsulfonyl methanesulfonate (44.17 g, 253.59 mmol) was added into the reaction mixture dropwise at 10° C. After addition, the reaction mixture was warmed up to 25° C. and stirred for 2 hrs. The resulting mixture was poured into water (300 mL) and the organic layer was separated. The aqueous phase was extracted with DCM (200 mL×3), and the combined organic layers were dried over Na₂SO₄ and concentrated under reduced pressure to give A-4 (25.30 g, 90.1% yield). ¹H NMR (300 MHz, CDCl₃): δ 5.03-4.90 (m, 1H), 4.74-4.51 (m, 2H), 4.36 (d, J=3.7 Hz, 2H), 3.11 (s, 3H), 2.85-2.54 (m, 2H) ppm.

### Step 4: A-5

To a solution of A-4 (12.65 g, 76.12 mmol) in DMF (100 mL) was added NaN₃ (7.5 g, 115.37 mmol), the mixture was stirred at 80° C. for 16 hrs. The mixture was filtered and the filter cake was washed with THF (10 mL). A-5 was used directly to next step as a solution in THF and DMF.

### Step 5: A-6

To a solution of A-5 in THE and DMF from last step was added Pd/C (2.46 g) and the reaction mixture was stirred under H2 atmosphere for 48 hrs. The resulting mixture was filtered and the filtrate was used directly to the next step as A-6 solution in THE and DMF.

### Step 6: A-7

To a solution of A-6 in THE and DMF (10 mL) from last step was added TEA (1.22 g, 12.05 mmol) and methyl 3-fluoro-4-nitro-benzoate (800 mg, 4.02 mmol). The reaction mixture was stirred at 25° C. for 16 hrs, poured into water (50 mL) and extracted with EA (40 mL×3). The combined organic layers were washed with brine (20 mL×3), dried over Na₂SO₄ and concentrated to give a residue, which was purified by silica gel column (PE:EA=3:1) to give A-7 (710 mg).

### Step 7: A-8

To a solution of A-7 (710 mg, 2.67 mmol) in THF (10 mL) was added Pd/C (10%, 140 mg). The reaction mixture was stirred at 25° C. under H2 atmosphere for 4 hrs. The resulting mixture was filtered and the filtrate was concentrated under reduced pressure to give A-8 (630 mg, 99.99% yield). ¹H NMR (300 MHz, CDCl₃): δ 7.46 (dd, J=8.1, 1.8 Hz, 1H), 7.36 (d, J=1.8 Hz, 1H), 6.67 (d, J=8.1 Hz, 1H), 5.19-5.01 (m, 1H), 4.81-4.69 (m, 1H), 4.62-4.55 (m, 1H), 3.85 (s, 3H), 3.45-3.32 (m, 2H), 2.86-2.68 (m, 1H), 2.58 (m, 1H) ppm; MS: m/z=237 (M+1).

### Step 8: Intermediate A

To a solution of methyl A-8 (630 mg, 2.67 mmol) and p-TSA (45.92 mg, 266.65 μmol) in CH₃CN (20 mL) was added 2-chloro-1,1,1-trimethoxyethane (494.66 mg, 3.20 mmol). The reaction mixture was stirred at 60° C. for 1 hr. The resulting mixture was poured into water (80 mL) and extracted with EA (40 mL×3). The combined organic layers were washed with brine (20 mL×3), dried over Na₂SO₄ and concentrated to give a residue, which was purified by silica gel column (PE:EA=3:1) to give methyl Intermediate A (720 mg, 91.6% yield). ¹H NMR (300 MHz, CDCl₃): δ 8.10 (s, 1H), 7.98 (dd, J=8.5, 1.3 Hz, 1H), 7.77 (d, J=8.5 Hz, 1H), 5.30-5.13 (m, 1H), 5.01 (s, 2H), 4.68-4.50 (m, 3H), 4.32-4.25 (m, 1H), 3.93 (s, 3H), 2.83-2.59 (m, 1H), 2.50-2.29 (m, 1H) ppm; MS: m/z=295 (M+1).

### Intermediate B:

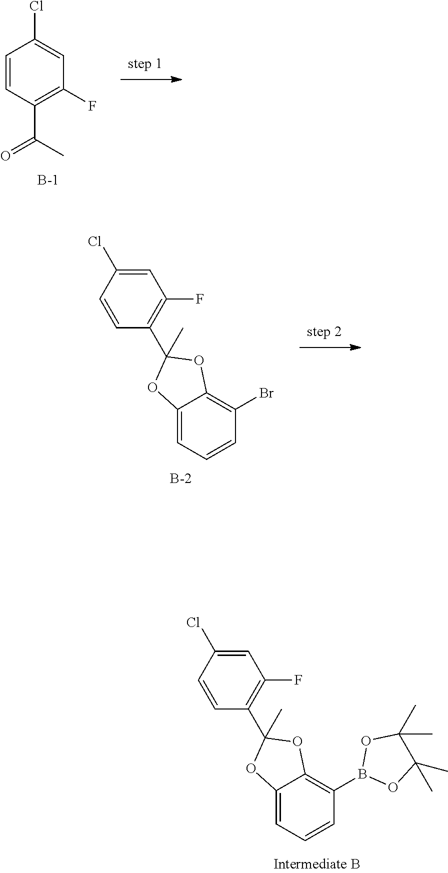

### Step 1: B-2

To a solution of 3-bromobenzene-1,2-diol (15 g, 79.36 mmol) in Toluene (300 mL) was added B-1 (13.70 g, 79.36 mmol) and p-TSA (6.83 g, 39.68 mmol). The reaction apparatus was fitted with a Dean-Stark trap, and the mixture was heated at 140° C. for 14 hrs. The solution was concentrated to give a residue, which was purified by silica gel chromatography (PE) to give B-2 (1.7 g, 4.95 mmol).

### Step 2: Intermediate B

To a mixture of B-2 (700 mg, 2.04 mmol), 4,4,5,5-tetramethyl-2-(4,4,5,5-tetramethyl-1,3,2-dioxaborolan-2-yl)-1,3,2-dioxaborolane (672.59 mg, 2.65 mmol) in dioxane (3 mL) was added KOAc (599.86 mg, 6.11 mmol) and Pd(dppf)Cl₂ (298.16 mg, 407.48 μmol) at 30° C. The mixture was stirred for 2 hrs at 100° C. The mixture was cooled and concentrated to give a residue, which was purified by silica gel chromatography (PE/EA=10/1-1:1, v/v) to give Intermediate B (710 mg, 1.82 mmol).

The compound in Table B was made according to the procedure of Compound B-2.

TABLE B

| | | ¹H NMR |
|---|---|---|
| | | and/or |
| | | LC/MS |
| Name | Structure | data |
| Inter- medi- ate C | 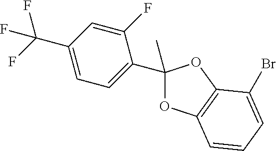 [SMILES: CC1(c2ccc(C(F)(F)F)cc2F)Oc2cccc(Br)c2O1] | MS: m/z = 377.1 (M + 1, ESI). |

### Intermediate D:

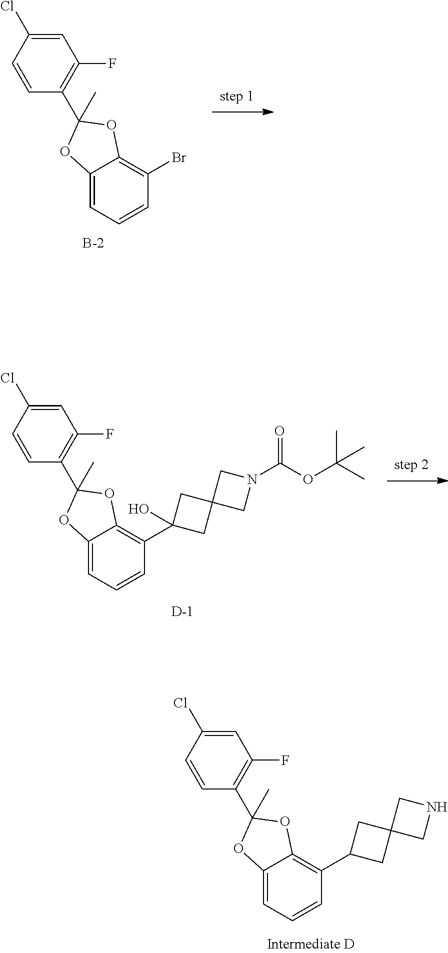

### Step 1: D-1

To a mixture of B-2 (400 mg, 1.16 mmol) in THF (10 mL) was added n-Butyllithium (2.5 M, 698.54 μL) at −60° C. for 0.2 hr. Tert-butyl 6-oxo-2-azaspiro[3.3]heptane-2-carboxylate (368.93 mg, 1.75 mmol) in THF (2 mL) was then added. The reaction solution was stirred for 0.8 hr at −60° C., and quenched with MeOH (6 mL) at −50° C. The resulted mixture was concentrated, charged with H₂O (30 mL) and extracted by EA (30 mL×2). The combined organic layers were washed with brine (30 mL), dried over Na₂SO₄, and concentrated to give a residue, which was purified by silica gel chromatography (PE/EA=3/1) to give D-1 (300 mg, 630.34 μmol, 54.1% yield). MS: m/z=420.1 (M+1-56).

### Step 2: Intermediate D

To a mixture of D-1 (285 mg, 598.82 μmol) in DCM (4 mL) was added CF₃COOH (1.64 g, 14.37 mmol) and Triethylsilane (1.39 g, 11.98 mmol, 1.91 mL) at 0° C. The reaction mixture was stirred for 24 hrs at 25° C. The mixture was charged with EA (10 mL) at 25° C., and concentrated with a rotary evaporator to give Intermediate D (160 mg, crude). MS: m/z=360.1 (M+1).

### Intermediate E:

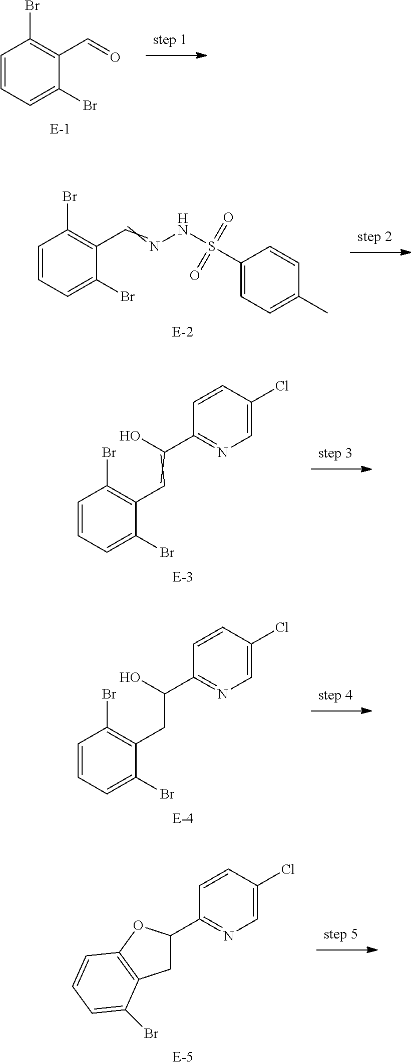

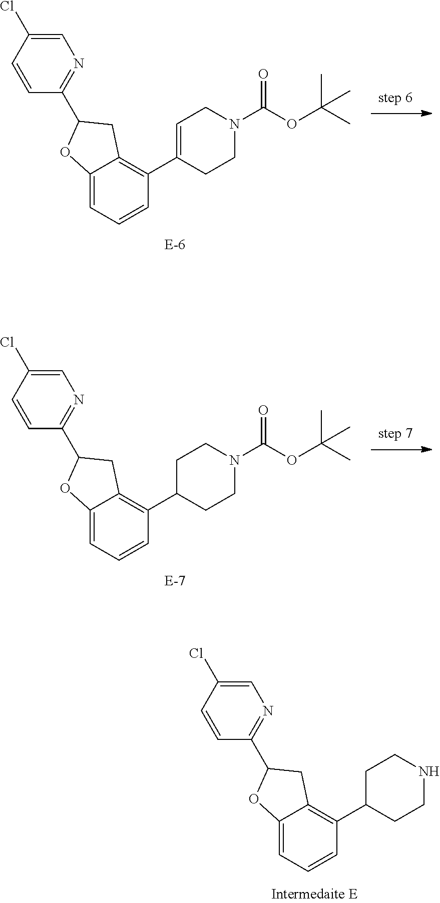

### Step 1: E-2

To a solution of E-1 (5 g, 18.95 mmol) in MeOH (50 mL) was added 4-methylbenzenesulfonohydrazide (5.29 g, 28.42 mmol), the mixture was stirred at 20° C. for 2 hrs. Solvent was then removed to give a residue, which was treated with MeOH (20 mL) and filtered. The cake was washed with toluene (10 mL) and dried in vacuum to give E-2 (7.5 g, 17.36 mmol, 91.6% yield). MS: m/z=430.9 (M+1).

### Step 2: E-3

To a suspension of E-2 (7.5 g, 17.36 mmol) in MeOH (100 mL) was added MeONa (937 mg, 17.36 mmol), and stirred at 20° C. for 1 hr. Then, 5-chloropyridine-2-carbaldehyde (1.64 g, 11.57 mmol) was added, and the mixture was stirred at 45° C. for 16 hrs. Solvent was removed, the residue was diluted with water (10 mL), and extracted with EA (100 mL×2). The combined organic layers were washed with brine (20 mL), dried over Na₂SO₄, concentrated in vacuum to give a residue, which was purified by silica gel column (PE/EA=50/1) to give E-3 (2.1 g, 46.6% yield). MS: m/z=387.9 (M+1).

### Step 3: E-4

A solution of E-3 (300 mg, 770.28 μmol) in i-PrOH (5 mL) was stirred at 45° C., treated with a solution of NaBH₄ (32 mg, 847.31 μmol) in H₂O (0.5 mL), and heated at reflux for 3 hrs. The mixture was quenched with acetone (0.5 mL), diluted with H₂O and extracted with EA (50 mL×3). The combined organic layers were washed with brine (20 mL), dried over Na₂SO₄, concentrated in vacuum to give a residue, which was purified by silica gel column (PE/EA=10/1) to give E-4 (180 mg, 59.7% yield). MS: m/z=389.9 (M+1).

### Step 4: E-5

To a solution of E-4 (180 mg, 459.79 μmol) in DMF (3 mL) was added K₂CO₃ (190 mg, 1.38 mmol), CuI (43.78 mg, 229.89 μmol), (1R,2R)-N,N′-Dimethyl-1,2-cyclohexanediamine (62 mg, 459.79 μmol), and the mixture was stirred at 100° C. for 10 hrs. The mixture was diluted with H₂O (20 mL) and extracted with EA (50 mL×3). The combined organic layers were washed with brine (20 mL), dried over Na₂SO₄, concentrated in vacuum to give a residue, which was purified by silica gel column (PE/EA=5/1) to give E-5 (60 mg, 42% yield). MS: m/z=310.0 (M+1).

### Step 5: E-6

A mixture of E-5 (60 mg, 193.19 μmol), tert-butyl 4-(4,4,5,5-tetramethyl-1,3,2-dioxaborolan-2-yl)-3,6-dihydro-2H-pyridine-1-carboxylate (89 mg, 289.79 μmol), K₂CO₃ (80 mg, 579.57 μmol) and Pd(dppf)Cl₂ (14 mg, 19.32 μmol) in dioxane/H₂O (1 mL, v/v=5:1) was stirred at 90° C. for 3 hrs. The mixture was filtered, the filtrate was charged with water (10 mL) and extracted with EA (10 mL×3). The combined organic layers were washed with brine (5 mL), dried over Na₂SO₄ and concentrated to give a residue, which was purified by silica gel column (PE/EA=5/1) to give E-6 (60 mg, 75.2% yield). MS: m/z=413.1 (M+1).

### Step 6: E-7

A solution of E-6 (60 mg, 145.31 μmol) and Pd/C (60 mg, 10%, 50% wet) in EA (5 mL) was degasses and then back-filled with hydrogen, this evacuation-purge cycle was carried out a total of three times. The reaction mixture was then stirred at 25° C. under hydrogen balloon for 16 hrs. The mixture was filtered, and concentrated to give E-7 (60 mg, 144.60 μmol, 99.5% yield). MS: m/z=415.2 (M+1).

### Step 7: Intermediate E

To a solution of E-7 (60 mg, 144.60 μmol) in MeOH (1 mL) was added HCl/dioxane (4 M, 1 mL) and stirred for 1 hr. The solution was then concentrated to give Intermediate E (40 mg, 87.9% yield). MS: m/z=315.0 (M+1).

### Intermediate F:

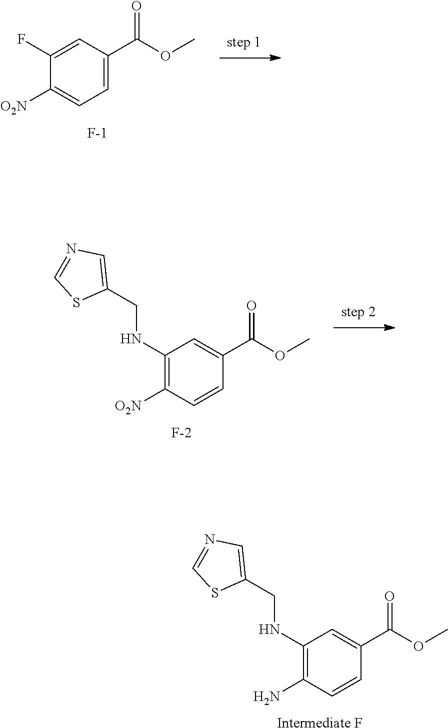

### Step 1: F-2

A mixture of F-1 (7.5 g, 37.66 mmol), thiazol-5-ylmethanamine (6.24 g, 41.43 mmol, 1.18 mL, HCl salt) and DIPEA (14.60 g, 112.99 mmol, 19.68 mL) in MeCN (150 mL) was stirred at 70° C. for 8 hrs. The mixture was concentrated, re-dissolved in EA (150 mL) and washed with HCl (1 M, 80 mL). The separated organic layer was washed with brine (30 mL), dried over Na₂SO₄, filtered, and concentrated to give F-2 (8 g, 27.28 mmol, 72.4% yield). MS: m/z=294.2 (M+1).

### Step 2: Intermediate F

A mixture of F-2 (8 g, 27.28 mmol), zinc (17.84 g, 272.76 mmol) and NH₄Cl (5.84 g, 109.10 mmol) in Methanol (50 mL) and Water (20 mL) was stirred at 25° C. for 1 hr. The resulting mixture was filtered, concentrated, and extracted with DCM (100 mL). The separated organic layer was washed with brine (30 mL), dried over Na₂SO₄, filtered, and concentrated to give Intermediate F (6.7 g, 25.44 mmol, 93.3% yield). MS: m/z=264.2 (M+1).

### Intermediate G:

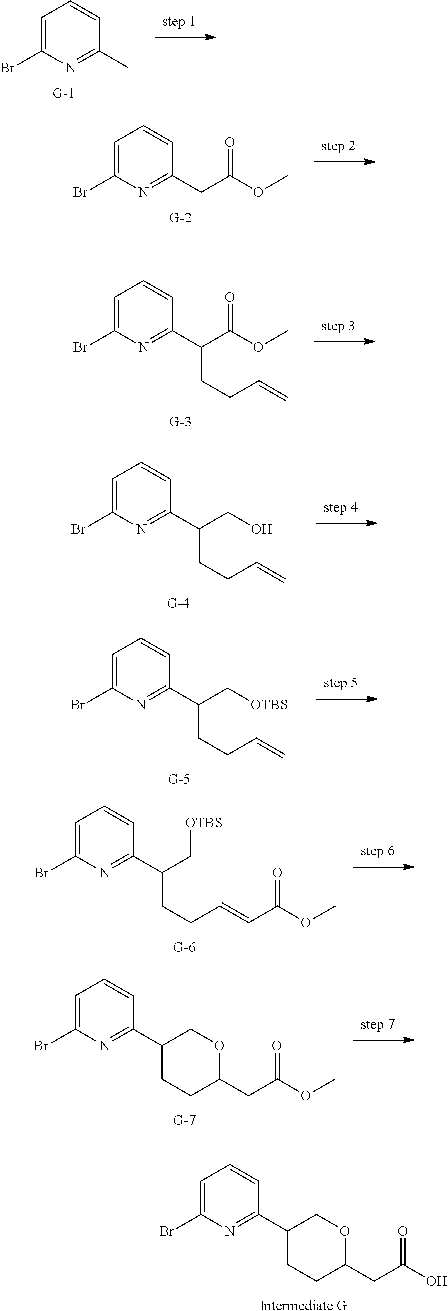

### Step 1: G-2

To a solution of N-isopropylpropan-2-amine (15.29 g, 151.14 mmol, 21.30 mL) in THF (100 mL) was added n-BuLi (2.5 M, 51.16 mL) at −78° C. slowly. After addition, the resulting solution was stirred at 0° C. for 1 hr and re-cooled to −78° C. B-1 (20 g, 116.26 mmol, 13.25 mL) and DMPU (16.39 g, 127.89 mmol, 15.46 mL) was added. After addition, the solution was stirred at −78° C. for 0.5 hr, to which dimethyl carbonate (62.84 g, 697.58 mmol) was added. After addition, the solution was slowly warmed to room temperature and stirred for additional 4 hrs. The mixture was quenched with aqueous NH₄Cl (100 mL) and extracted with EA (150 mL). The separated organic layer was concentrated and purified by FCC (silica gel, PE/EA=10/1 to 3/1) to give G-2 (9.9 g, 43.03 mmol, 37.0% yield). MS: m/z=230.1 (M+1).

### Step 2: G-3

To a solution of G-2 (9.9 g, 43.03 mmol) in DMF (50 mL) was added NaH (1.73 g, 45.18 mmol, 60% purity in mineral oil) at 0° C. After addition, the mixture was stirred at 0° C. for 0.5 hr, before 4-bromobut-1-ene (6.39 g, 47.34 mmol, 4.80 mL) was added. The mixture was stirred at 25° C. overnight, diluted with H₂O (100 mL) and extracted with EA (100 mL). The separated organic layer was washed with H₂O (100 mL×3) and brine, dried over Na₂SO₄, filtered and concentrated. The residue was purified by FCC (silica gel, PE/EA=10/1 to 5/1) to give G-3 (5.6 g, 19.71 mmol, 45.8% yield). MS: m/z=284.1 (M+1).

### Step 3: G-4

To a solution of G-3 (5.6 g, 19.71 mmol) in THF (100 mL) was added lithium;alumanuide (1.00 g, 26.35 mmol) slowly at 0° C. After addition, the mixture was stirred at 25° C. for 2 hrs. The resulting mixture was quenched with H₂O (1 mL), aqueous NaOH (1 mL) and H₂O (3 mL), dried over Na₂SO₄, filtered, and concentrated to give G-4 (5 g, 19.52 mmol, 99.0% yield). MS: m/z=256.1 (M+1).

### Step 4: G-5

A mixture of G-4 (5 g, 19.52 mmol), imidazole (2.67 g, 39.17 mmol) and TBSCl (1.93 g, 23.48 mmol) in DCM (20 mL) was stirred at 25° C. for 2 hrs. The resulting mixture was washed with H₂O, concentrated, and purified by FCC (silica gel, PE/EA=20/1 to 10/1) to give G-5 (4 g, 10.80 mmol, 55.3% yield). MS: m/z=370.2 (M+1).

### Step 5: G-6

To a solution of G-5 (4 g, 10.80 mmol) and methyl prop-2-enoate (2.80 g, 32.52 mmol, 2.93 mL) in DCM (10 mL) was added Grubbs II (916.82 mg, 1.08 mmol). After addition, the mixture was stirred at 25° C. overnight. The resulting mixture was concentrated and purified by FCC (silica gel, PE/EA=10/1 to 5/1) to give G-6 (3.4 g, 7.94 mmol, 73.5% yield). MS: m/z=428.2 (M+1).

### Step 6: G-7

A solution of G-6 (3.3 g, 7.70 mmol) and TBAF (2.05 g, 7.83 mmol, 2.27 mL) in THF (30 mL) was stirred at 25° C. for 4 hrs. The solution was charged with H₂O (20 mL) and extracted with EA (20 mL). The separated organic layer was concentrated and purified by FCC (silica gel, PE/EA=10/1 to 5/1) to give G-7 (2.0 g, 6.37 mmol, 82.6% yield). MS: m/z=314.1 (M+1).

### Step 7: Intermediate G

A mixture of G-7 (2.0 g, 6.37 mmol) and lithium hydroxide monohydrate (801.41 mg, 19.10 mmol) in THF (20 mL) and water (20 mL) was stirred at 25° C. for 4 hrs. The resulting mixture was concentrated, acidified with 1 M HCl to pH=5˜6, and extracted with EA (30 mL). The separated organic layer was washed with brine (30 mL), dried over Na₂SO₄, and concentrated to give Intermediate G (1.5 g, 5.00 mmol, 78.5% yield). MS: m/z=300.1 (M+1).

### Intermediate H:

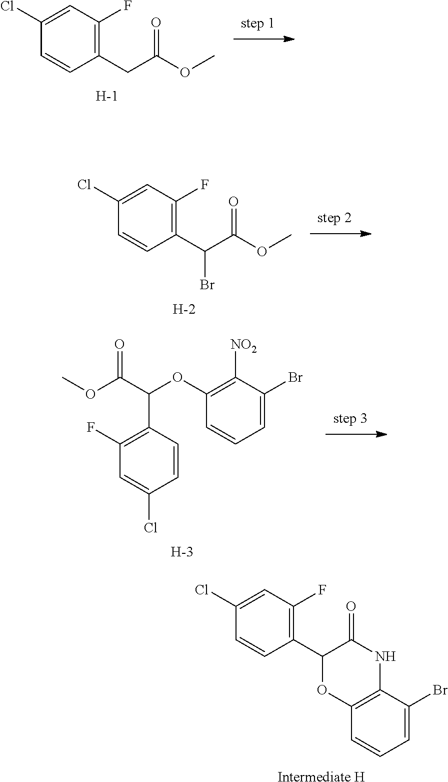

### Step 1: H-2

To a mixture of H-1 (9 g, 44.42 mmol) in THF (40 mL) at −78° C. was added LiHMDS (1 M, 88.84 mL) dropwise and stirred at this temperature for 30 minutes, a solution of TMSCl (7.72 g, 71.07 mmol) in THF (20 mL) was added dropwise and stirred for 15 minutes, then a solution of NBS (8.70 g, 48.86 mmol) in THF (20 mL) was added and stirred for 2 hrs at −78° C. The reaction mixture was quenched with aqueous NH₄Cl (50 mL), extracted with EA (100 mL×3), dried over Na₂SO₄, filtered, and concentrated to give H-2 (10 g, 35.52 mmol, 80.0% yield).

### Step 2: H-3

A mixture of 3-bromo-2-nitro-phenol (1 g, 4.59 mmol), H-2 (1.42 g, 5.05 mmol) and K₂CO₃ (1.90 g, 13.76 mmol) in MeCN (100 mL) was stirred at 30° C. for 2 hrs. The mixture was filtered and concentrated to give a residue, which was purified by column chromatography on silical gel (PE/EA=50/1 to 10/1) to give H-3 (1.4 g, 3.34 mmol, 72.9% yield). MS: m/z=418.1 (M+1).

### Step 3: Intermediate H

A mixture of H-3 (1 g, 2.39 mmol), Zn (468.64 mg, 7.17 mmol) and NH₄Cl (1.28 g, 23.89 mmol) in Methanol (100 mL) and Water (25 mL) was stirred at 25° C. for 2 hrs. The resulting mixture was filtered, concentrated, and extracted with EA (50 mL). The separated organic layer was washed with brine (40 mL), dried over Na₂SO₄, filtered, and concentrated to give Intermediate H (600 mg, 1.62 mmol, 67.8% yield). MS: m/z=356.1 (M+1).

### Intermediate I:

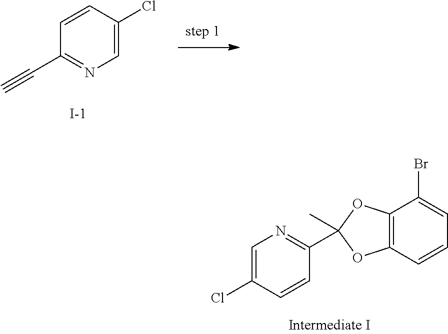

### Step 1: Intermediate I

To a mixture of 3-bromobenzene-1,2-diol (5.22 g, 27.62 mmol), I-1 (3.8 g, 27.62 mmol) in toluene (30 mL) was added carbon monoxide;ruthenium (883.01 mg, 1.38 mmol) at 0° C. The reaction was stirred for 16 hrs at 100° C. Then, the mixture was concentrated to give a residue, which was purified by silica gel chromatography (PE/EA=10/1 to 4/1) to give Intermediate I (3.1 g, 9.49 mmol, 34.4% yield). MS: m/z=326.0 (M+1).

The compounds in Table C below were made according to the procedure of Compound 15-2.

TABLE C

| | | ¹H NMR |
|---|---|---|
| | | and/or |
| | | LC/MS |
| Name | Structure | data |
| Inter- medi- ate J | 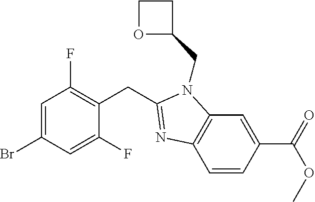 [SMILES: COC(=O)c1ccc2nc(Cc3c(F)cc(Br)cc3F)n(C[C@@H]3CCO3)c2c1] | MS: m/z = 451.1 (M + 1). |
| Inter- medi- ate K | 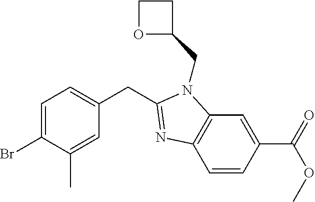 [SMILES: COC(=O)c1ccc2nc(Cc3ccc(Br)c(C)c3)n(C[C@@H]3CCO3)c2c1] | MS: m/z = 429.1 (M + 1). |
| Inter- medi- ate L | 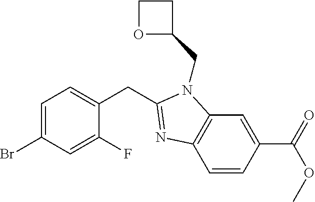 [SMILES: COC(=O)c1ccc2nc(Cc3ccc(Br)cc3F)n(C[C@@H]3CCO3)c2c1] | MS: m/z = 433.1 (M + 1). |
| Inter- medi- ate M | 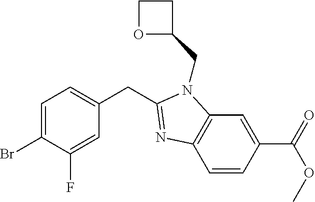 [SMILES: COC(=O)c1ccc2nc(Cc3ccc(Br)c(F)c3)n(C[C@@H]3CCO3)c2c1] | MS: m/z = 433.1 (M + 1). |
| Inter- medi- ate N | 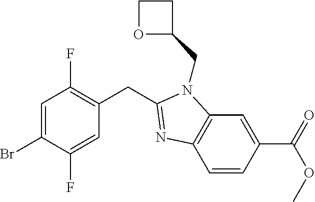 [SMILES: COC(=O)c1ccc2nc(Cc3cc(F)c(Br)cc3F)n(C[C@@H]3CCO3)c2c1] | MS: m/z = 451.1 (M + 1). |

### Intermediate O:

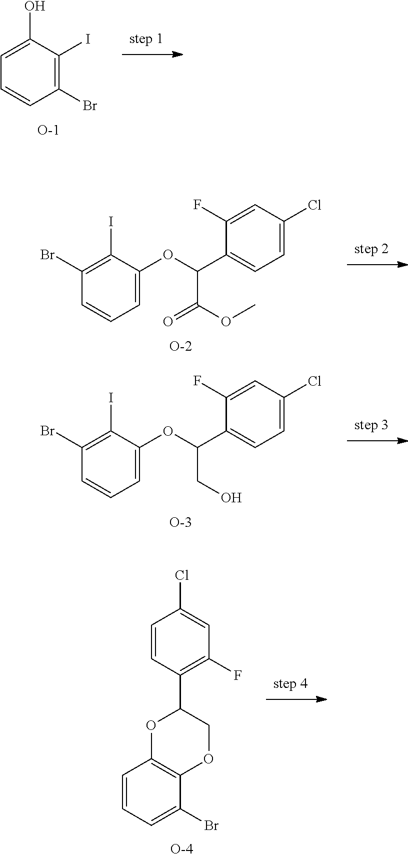

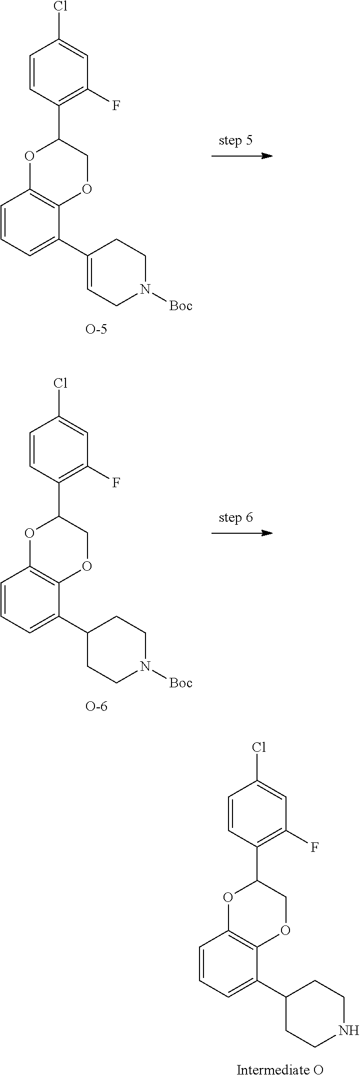

### Step 1: O-2

A solution of H-2 (1.04 g, 3.68 mmol), O-1 (1.0 g, 3.35 mmol) and K₂CO₃ (1.39 g, 10.04 mmol) in DMF (30 mL) was stirred at 25° C. for 2 hrs. The resulting solution was concentrated and purified by FCC (silica gel, PE/EA=10/1 to 5/1) to give O-2 (1.4 g, 2.80 mmol, 83.8% yield). MS: m/z=500.9 (M+1).

### Step 2: O-3

To a solution of O-2 (1.2 g, 2.40 mmol) in THF (30 mL) was added Lithium borohydride (2 M in THF, 2.4 mL) slowly at 0° C. After addition, the mixture was stirred at 25° C. for 4 hrs. The resulting mixture was quenched with NH₄Cl (saturated), extracted with EA (200 mL), dried over Na₂SO₄, filtered, and concentrated to give O-3 (1.1 g, 2.33 mmol, 97.100 yield).

### Step 3: O-4

A mixture of O-3 (1.1 g, 2.33 mmol), 1,10-phenanthroline (63.07 mg, 349.96 mol), CuI (22.22 mg, 116.65 mol) and Cs₂CO₃ (1.52 g, 4.67 mmol) in 1,4-dioxane (150 mL) was stirred at 100° C. for 4 hrs. The resulting mixture was cooled to room temperature and filtered. The filtrate was concentrated and purified by FCC (silica gel, PE/EA=20/1) to give O-4 (500 mg, 1.46 mmol, 62.4% yield).

### Step 4: O-5

A mixture of O-4 (500 mg, 1.46 mmol), tert-butyl 4-(4,4,5,5-tetramethyl-1,3,2-dioxaborolan-2-yl)-3,6-dihydro-2H-pyridine-1-carboxylate (674.98 mg, 2.18 mmol), cyclopentyl(diphenyl)phosphane;dichloromethane;dichloropalladium;iron (59.42 mg, 72.76 mol) and K₂CO₃ (402.26 mg, 2.91 mmol) in 1,4-dioxane (8 mL) and water (2 mL) was stirred at 120° C. (under microwave) for 40 minutes. The resulting mixture was cooled and filtered. The filtrate was concentrated and purified by FCC (silica gel, PE/EA=3/1) to give O-5 (500 mg, 1.12 mmol, 77.0% yield). MS: m/z=390.1 (M+1).

### Step 5: O-6

A mixture of O-5 (500 mg, 1.12 mmol) and dioxoplatinum (50 mg, 220.19 mol) in Methanol (20 mL) was stirred at 25° C. under H₂ atmosphere for 1 hr. The resulting mixture was filtered and concentrated to give O-6 (500 mg, 1.12 mmol, 99.5% yield). MS: m/z=392.1 (M+1).

### Step 6: Intermediate O

A mixture of O-6 (500 mg, 1.12 mmol) and HCl/dioxane (4 M, 5 mL) in 1,4-dioxane (3 mL) was stirred at 25° C. for 2 hrs. The resulting solution was concentrated to give Intermediate O (400 mg, 1.04 mmol, 93.2% yield, HCl salt). MS: m/z=348.1 (M+1).

The compound in Table D below was made according to the procedure of Intermediate O.

TABLE D

| | | ¹H NMR |
|---|---|---|
| | | and/or |
| | | LC/MS |
| Name | Structure | data |
| Inter- medi- ate P | 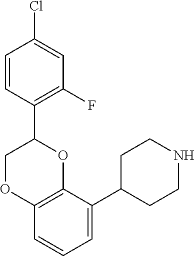 [SMILES: Fc1cc(Cl)ccc1C1COc2cccc(C3CCNCC3)c2O1] | MS: m/z = 348.1 (M + 1). |

### Intermediate Q:

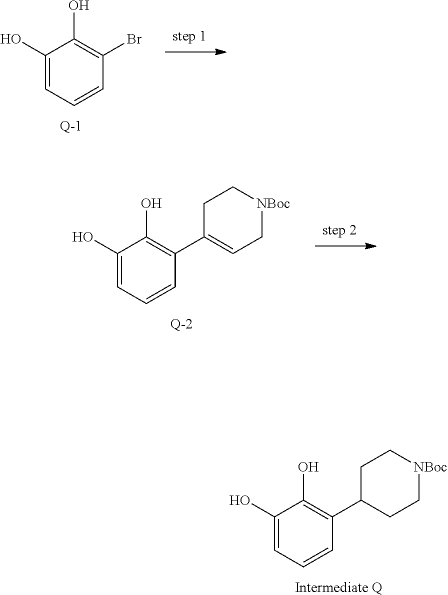

### Step 1: Q-2

A solution of Q-1 (1 g, 5.29 mmol), tert-butyl 4-(4,4,5,5-tetramethyl-1,3,2-dioxaborolan-2-yl)-3,6-dihydro-2H-pyridine-1-carboxylate (1.64 g, 5.29 mmol), Pd(t-Bu₃P)₂ (135.19 mg, 264.54 μmol) and K₂CO₃ (2.19 g, 15.87 mmol) in THF (10 mL) and H₂O (8 mL) was stirred at 70° C. for 3 hrs. The resulting solution was quenched with aq. NH₄Cl (30 mL), and the mixture was extracted with EA (30 mL×2), dried over Na₂SO₄, filtered and concentrated to give Q-2 (1.5 g, 5.15 mmol, 97.3% yield).

### Step 2: Intermediate Q

A mixture of Q-2 (1.35 g, 4.63 mmol) and Pd/C (5%, 800 mg) in MeOH (15 mL) was stirred under H2 atmosphere at 50° C. for 12 hrs. The mixture was cooled to room temperature, filtered and concentrated to give Intermediate Q (1.35 g, 4.60 mmol, 99.3% yield). MS: m/z=238.2 (M−55).

### Intermediate R:

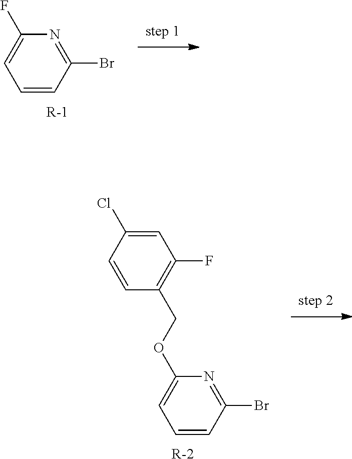

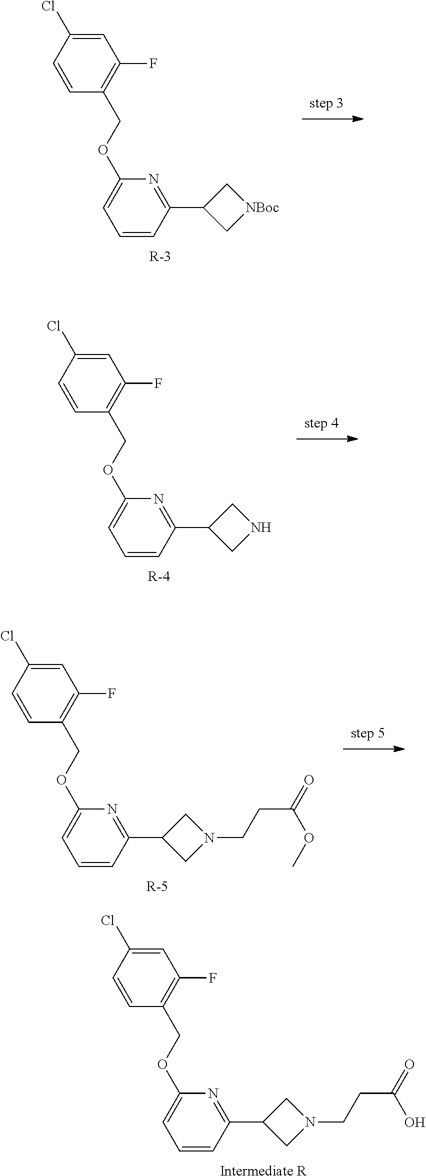

### Step 1: R-2

A solution of (4-chloro-2-fluoro-phenyl)methanol (4 g, 24.91 mmol), R-1 (4.38 g, 24.91 mmol), 18-Crown-6 (329.22 mg, 1.25 mmol), KOH (4.19 g, 74.73 mmol) in Toluene (70 mL) was degassed three times with nitrogen and stirred overnight. The mixture was diluted with H₂O (20 mL) and extracted with EA (20 mL×3). The combined organic layers were washed with brine (30 mL×3), dried over anhydrous Na₂SO₄, filtered and concentrated to give a crude, which was purified by flash chromatography (EA in petroleum ether: 0˜15%) to give R-2 (5.2 g, 66% yield). MS: m/z=316 (M+1).

### Step 2: R-3

A mixture of Zinc (4.13 g, 63.18 mmol) and 1,2-dibromoethane (1.19 g, 6.32 mmol) in THF (25 mL) was heated to reflux for 1 hr. After cooling to room temperature, the reaction mixture was treated with chloro(trimethyl)silane (686.39 mg, 6.32 mmol) and stirred for 1 hr. Then, a solution of tert-butyl 3-iodoazetidine-1-carboxylate (8.94 g, 31.59 mmol) in THF (5 mL) was added dropwise. The reaction was stirred for 1 hr at 60° C. and cooled to room temperature, to which R-2 (2.0 g, 6.32 mmol) and cyclopentyl(diphenyl)phosphane;dichloromethane;dichloropalladium;iron (2.58 g, 3.16 mmol) were added, and the mixture was stirred at 70° C. for 2 hrs. The mixture was filtered, concentrated, and treated with EA (50 mL) and saturated aqueous sodium carbonate. The resulting precipitate was removed by filtration and the filter cake was washed with EA (20 mL). The combined filtrates were washed with brine, dried over MgSO₄, filtered and concentrated to give a crude, which was purified by prep-TLC (petroleum ether:EA=7:1) to give R-3 (500 mg).

### Step 3: R-4

To a solution of R-3 (600 mg, 1.53 mmol) in 2,2,2-trifluoroethanol (10 mL) was added chloro(trimethyl)silane (165.93 mg, 1.53 mmol), and the mixture was stirred at room temperature for 4 hrs. The solution was concentrated to give a crude, which was triturated with n-pentane and MTBE (5.0 mL) to afford R-4 (500 mg, crude). MS: m/z=293 (M+H).

### Step 4: R-5

A solution of R-4 (500 mg, 1.71 mmol), methyl prop-2-enoate (147.04 mg, 1.71 mmol) and DBU (430.18 mg, 1.71 mmol) in MeCN (15 mL) was stirred at room temperature overnight. The mixture was diluted with H₂O (20 mL), and extracted with EA (20 mL×3). The combined organic layers were washed with brine (30 mL×3), dried over anhydrous Na₂SO₄, filtered and concentrated to give a crude, which was purified by flash chromatography (petroleum ether:EA=1:1) to give R-5 (400 mg, 61.8% yield). MS: m/z=379 (M+1).

### Step 5: Intermediate R

A solution of R-5 (400 mg, 1.06 mmol), LiOH (75.87 mg, 3.17 mmol) in THF (10 mL) and Water (2 mL) was stirred at room temperature overnight. The resulting solution was acidified with 1 M HCl till pH=5-6 and extracted with EA (30 mL). The organic layer was washed with brine (10 mL), dried over anhydrous Na₂SO₄, filtered and concentrated to give a crude, which was purified by prep-HPLC to give Intermediate R (270 mg, 70.1% yield). MS: m/z=365 (M+1).

### Intermediate S:

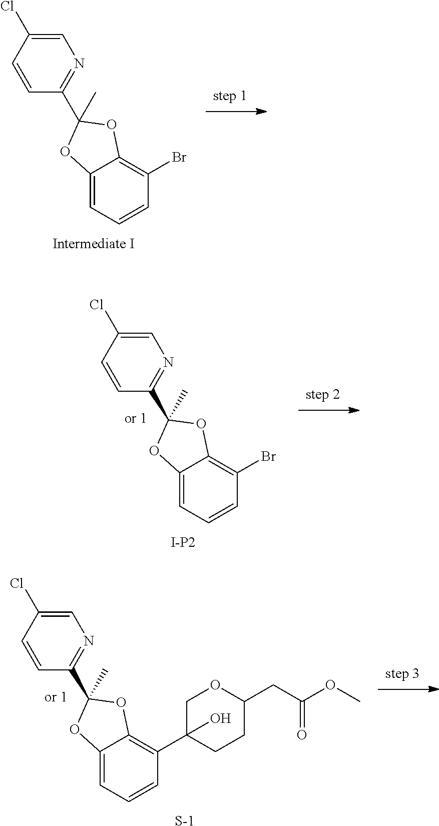

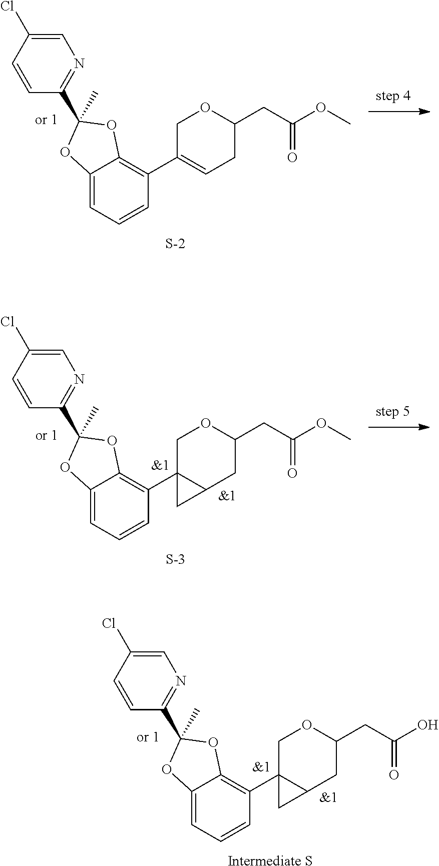

### Step 1: I-P2

Intermediate I (40 g) was purified by SFC (Column: Daicel ChiralPak AY-H 250 mm*30 mm I.D., 5 μm; Mobile Phase: CO₂/EtOH=90/10; Flowrate: 50 g/min; Wavelength: 254 nm; Temperature: 40° C.) to give I-P1 (peak 1) (19 g, 58.18 mmol, 24.25% yield) and I-P2 (peak 2) (18 g, 55.12 mmol, 22.98% yield).

### Step 2: S-1

To a mixture of I-P2 (2 g, 6.12 mmol) in THF (10 mL) was added butyllithium (2.5 M, 3.18 mL) dropwise at −78° C. under N₂ atmosphere, and the mixture was stirred −78° C. for 30 minutes. Methyl 2-(5-oxotetrahydropyran-2-yl)acetate (1.05 g, 6.12 mmol) was added to the mixture, and stirred for another 30 minutes. The mixture was quenched by aqueous NH₄Cl (10 mL) and extracted with EA (50 mL). The organic phase was concentrated and purified by column chromatography (EA/petroleum ether=1/10-1/3) to give S-1 (1 g, 2.38 mmol, 38.9% yield). MS: m/z=420.1 (M+1).

### Step 3: S-2

To a mixture of S-1 (0.6 g, 1.43 mmol) in DCM (10 mL) was added diethyloxonio(trifluoro)boranuide (567.04 mg, 1.86 mmol, 46.5% purity) at 0° C. The reaction solution was stirred for 6 hrs at 25° C. Then, the solution was concentrated to give a crude, which was purified by silica gel chromatography (petroleum ether/ethyl acetate=10/1-4/1, v/v) to give S-2 (0.4 g, 69.7% yield). MS: m/z=402.2 (M+1).

### Step 4: S-3

To a stirred solution of S-2 (374.76 mg, 932.60 μmol) in DCM (20 mL) at −78° C. was added Diethylzine (1 M, 4.66 mL), followed by diiodomethane (2.50 g, 9.33 mmol, 750.10 μL). After stirring for 20 minutes, the reaction mixture was warmed to 25° C. and stirred for 1 hr. The mixture was poured into saturated aqueous ammonium chloride and extracted with DCM. The organic phase was washed with brine, dried over magnesium sulfate, filtered and concentrated to give S-3 (55 mg, 14.2% yield). MS: m/z=416.3 (M+1).

### Step 5: Intermediate S

A mixture of S-3 (55 mg, 132.25 μmol) and NaOH (21.16 mg, 529.02 μmol) in Water (2 mL), THF (4 mL) and Methanol (4 mL) was stirred at 25° C. overnight. The resulting mixture was acidified with 1 M HCl till pH=5-6 and extracted with EA (30 mL). The organic layer was washed with brine (10 mL), dried over Na₂SO₄, filtered and concentrated to give Intermediate S (50 mg, crude). MS: m/z=402.1 (M+1).

### Intermediate T:

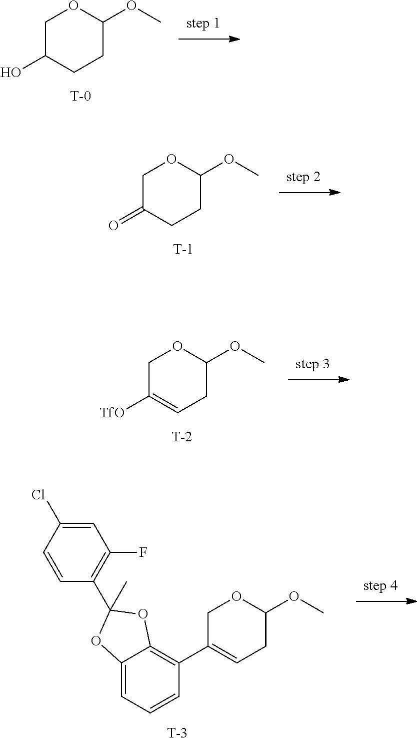

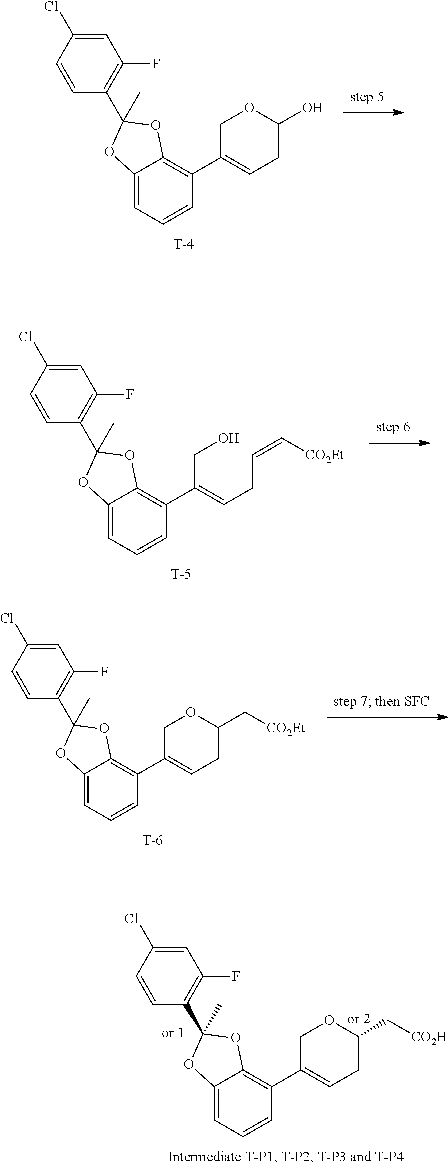

### Step 1: T-1

To a solution of T-0 (30 g, 227.0 mmol) in DCM (350 mL) was added (1,1-diacetoxy-3-oxo-1,2-benziodoxol-1-yl) acetate (144.42 g, 340.50 mmol) and NaHCO₃ (95.35 g, 1.14 mol, 44.14 mL) at 0° C. The mixture was then warmed to room temperature and stirred for 18 hrs. The mixture was quenched with water (500 mL) and extracted with DCM (200 mL×2). The combined organic layers were washed with brine (300 mL), dried over N₂SO₄, filtered and concentrated. The residue was purified by silica gel (EA in petroleum ether from 0% to 15%) to give T-1 (25 g, 192.10 mmol, 84.6% yield). ¹H NMR (400 MHz, Chloroform-d) δ 4.88 (td, J=4.3, 1.5 Hz, 1H), 4.15 (d, J=16.9 Hz, 1H), 3.92 (d, J=16.9, 1H), 3.44 (s, 3H), 2.66-2.36 (m, 2H), 2.30-2.20 (m, 1H), 2.10-1.90 (m, 1H).

### Step 2: T-2

To a solution of T-1 (10 g, 76.84 mmol) in THF (100 mL) was added KHMDS (1.0 M, 115.26 mL) at −78° C. under N2 atmosphere. The mixture was stirred for 1 hr, to which, a solution of N-(5-chloro-2-pyridyl)-1,1,1-trifluoro-N-(trifluoromethylsulfonyl)methanesulfonamide (36.21 g, 92.21 mmol) in THF (50 mL) was added. The mixture was stirred for 2 hrs at −78° C. before quenched with aq. NH₄Cl (300 mL) and extracted with EA (150 mL×2). The combined organic layers were washed with brine (150 mL), dried over Na₂SO₄, filtered and concentrated. The residue was purified by silica gel (EA in petroleum ether from 0% to 15%) to give T-2 (5.5 g, 20.98 mmol, 27.3% yield). ¹H NMR (500 MHz, Chloroform-d) δ 5.60 (dt, J=3.2, 1.7 Hz, 1H), 4.62 (dd, J=4.2, 2.0 Hz, 1H), 4.13-4.02 (m, 1H), 3.99-3.88 (m, 1H), 3.34-3.27 (m, 4H), 2.41 (dp, J=17.7, 3.4 Hz, 1H), 2.22-2.09 (m, 1H).

### Step 3: T-3

A mixture of Intermediate B (12 g, 30.72 mmol), T-2 (9.67 g, 36.86 mmol, multiple batches), K₂CO₃ (8.49 g, 61.44 mmol) and cyclopentyl(diphenyl)phosphane;dichloropalladium;iron (2.25 g, 3.07 mmol) in dioxane (30 mL) and Water (5 mL) was stirred at 100° C. under N2 atmosphere for 4 hrs. The resulting mixture was cooled to room temperature and filtered. The filtrate was concentrated and purified by FCC (silica gel, PE/EA=10/1-1/1) to give T-3 (9.2 g, 24.42 mmol, 79.5% yield). MS: m/z=345.1 (M−31).

### Step 4: T-4

A solution of T-3 (9.2 g, 24.42 mmol) in AcOH (20 mL) and Water (4 mL) was stirred at 100° C. for 4 hrs. The resulting solution was cooled to room temperature and concentrated. The residue was purified by FCC (silica gel, PE/EA=10/1 to 1/1) to give T-4 (8.1 g, 22.33 mmol, 91.4% yield). MS: m/z=345.1 (M−17).

### Step 5: T-5

A mixture of T-4 (8 g, 22.05 mmol) and ethyl 2-(triphenyl-phosphanylidene)acetate (11.52 g, 33.08 mmol) in Toluene (100 mL) was heated at 110° C. for 2 hrs. The mixture was concentrated to give a residue, which was purified by column chromatography on silical gel (EA/petroleum ether=1/10-10/1) to give T-5 (5 g, 11.55 mmol, 52.4% yield).

### Step 6: T-6

To a mixture of T-5 (5 g, 11.55 mmol) in THF (50 mL) was added NaH (923.98 mg, 23.10 mmol, 60% purity) at 0° C. The reaction solution was stirred for 3 hrs at 73° C. To the mixture, saturated aqueous ammonium chloride (100 mL) and EA (100 mL) was added at 0° C. The organic layer was separated and the aqueous layer was further extracted with EA (50 ml×3). The combined organic extracts were dried over sodium sulfate, filtered, and concentrated to give T-6 (5 g, crude).

### Step 7: Intermediate T-P1, T-P2, T-P3 and T-P4

A mixture of T-6 (5 g, 11.55 mmol) and NaH (2.31 g, 60.29 mmol, 60% purity) in THF (10 mL), Methanol (10 mL) and Water (4 mL) was stirred at 40° C. for 2 hrs. The resulting solution was acidified with 1 M HCl till pH=5-6 and extracted with EA (100 mL×3), the organic phases were dried over Na₂SO₄, filtered and concentrated to give a residue, which was purified by column chromatography on silica gel (EA/PE=1/10-1/4) to give Intermediate T (3.1 g, 7.66 mmol, 66.3% yield). The product was further separated by SFC (Column: Daicel ChiralPak G-11250 mm×30 mm I.D., 5 μm; Mobile Phase: CO₂/MeOH (0.1% DEA)=80:20; Flowrate: 50 g/min; Wavelength: 254 nm; Temperature: 40° C.) to give Intermediate T-PI (peak 1, 388 mg), Intermediate T-P2 (peak 2, 393 mg), Intermediate T-P3 (peak 3, 640 mg) and Intermediate T-P4 (peak 4, 600 mg).

### Intermediate U:

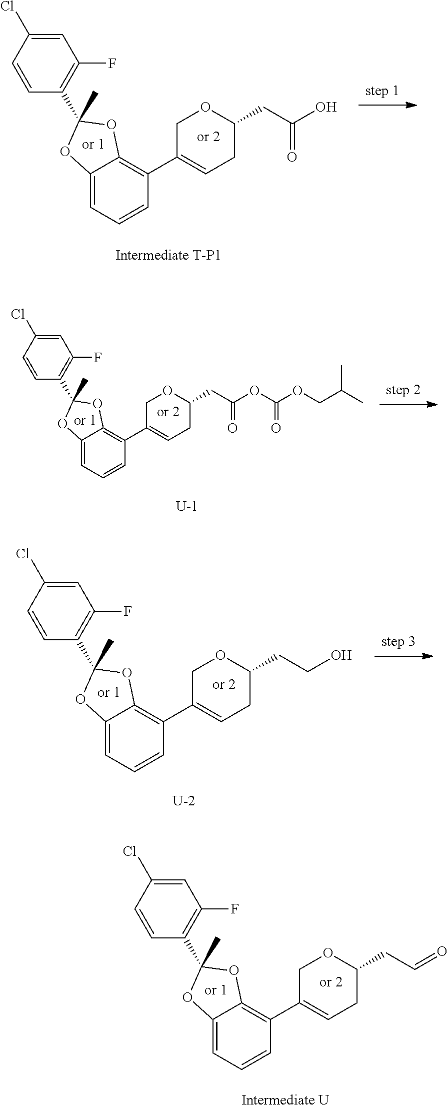

### Step 1: U-1

To a solution of Intermediate T-P1 (100 mg, 247.03 μmol) and TEA (49.99 mg, 68.86 μL) in DCM (10 mL) was added isobutyl carbonochloridate (50.61 mg, 370.54 μmol) at 0° C., and the mixture was stirred at 0° C. for 1 hr. The resulting mixture was quenched with water. The separated organic layer was washed with brine, dried over Na₂SO₄, filtered and concentrated to give U-1 (124 mg, crude).

### Step 2: U-2

To a solution of U-1 (120 mg, 237.66 μmol) in THF (10 mL) was added NaBH₄ (17.98 mg, 475.31 umol) at 25° C. and stirred for 1 hr. The resulting mixture was quenched with MeOH and concentrated to give a residue, which was purified by FCC (silica gel, petroleum ether/EA=10/1 to 5/1) to give U-2 (80 mg, 204.69 μmol, 86.1% yield). MS: m/z=391 (M+1).

### Step 3: Intermediate U

To a solution of U-2 (80 mg, 204.69 μmol) in DCM (10 mL) was added Dess-Martin Periodinane (173.64 mg, 409.38 μmol) at 0° C. After addition, the mixture was stirred at 25° C. for 1 hr before quenched with saturated aqueous NaHCO₃. The separated organic layer was concentrated and purified by FCC (silica gel, petroleum ether/EA=50/1 to 20/1) to give Intermediate U (65 mg, 167.17 μmol, 81.7% yield). MS: m/z=389 (M+1).

### Intermediate V:

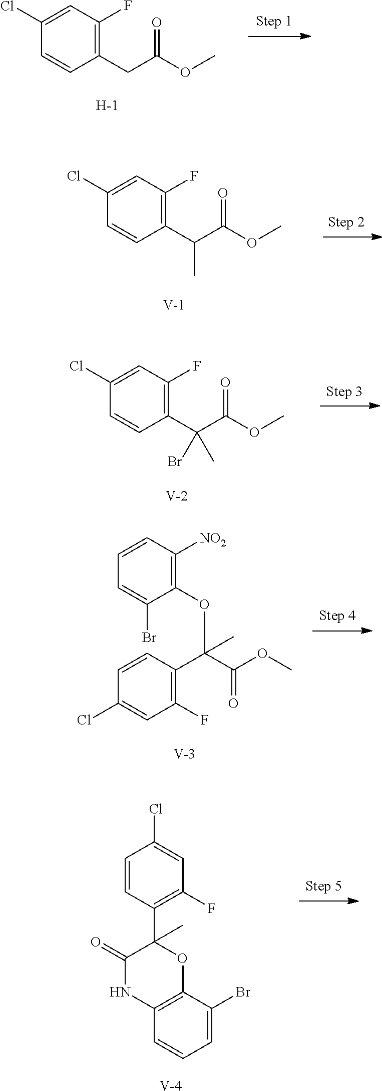

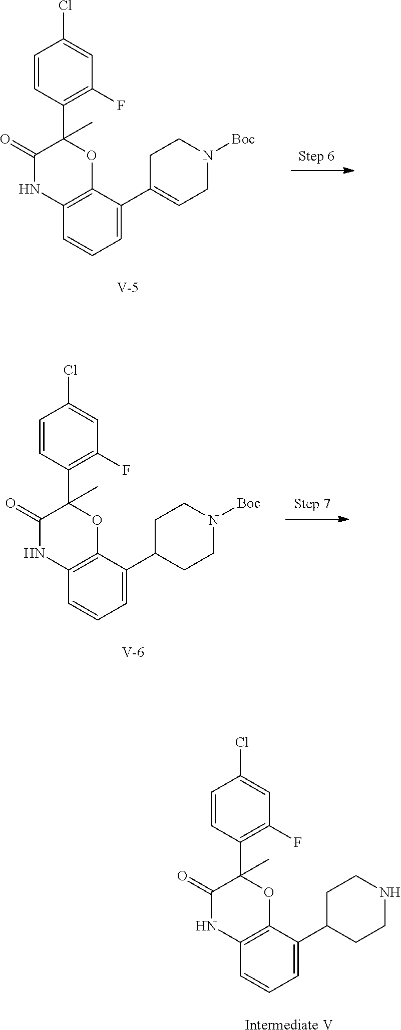

### Step 1: V-1

To a mixture of H-1 (5 g, 24.68 mmol) in THF (50 mL) at −78° C. was added LiHMDS (1 M, 32.08 mL) dropwise and stirred at this temperature for 30 minutes, iodomethane (5.25 g, 37.02 mmol, 2.30 mL) was then added dropwise and stirred for 2 hrs. The mixture was quenched with saturated ammonium chloride (30 mL) and extracted with EA (60 mL×2). The organic layers were dried over Na₂SO₄, filtered and concentrated to give V-1 (3.6 g, 16.62 mmol, 67.3% yield).

### Step 2: V-2

To a mixture of V-1 (2.5 g, 11.54 mmol) in THF (50 mL) was added LiHMDS (1 M, 12.12 mL) dropwise at −78° C. and stirred for 30 minutes, chloro(trimethyl)silane (1.32 g, 12.12 mmol, 1.54 mL) was then added dropwise and stirred for 15 minutes, before 1-bromopyrrolidine-2,5-dione (3.08 g, 17.31 mmol) was added. The mixture was stirred for 2 hrs at 25° C. and quenched with saturated ammonium chloride (20 mL). The mixture was extracted with EA (30 mL×3), dried over Na₂SO₄, filtered and concentrated to give V-2 (3 g, 10.15 mmol, 88.0% yield).

### Step 3: V-3

A solution of V-2 (3 g, 10.15 mmol), 2-bromo-6-nitro-phenol (2.21 g, 10.15 mmol) and K₂CO₃ (2.81 g, 20.3 mmol) in DMF (30 mL) was stirred at 25° C. for 2 hrs. The mixture was diluted with EA (80 mL), and the mixture was washed with water (80 mL×3), dried over Na₂SO₄, filtered, and concentrated to give a residue, which was purified by FCC (Gradient: 0-20% EA in petroleum ether) to give V-3 (1.6 g, 3.70 mmol, 36.4% yield).

### Step 4: V-4

A solution of V-3 (1.6 g, 3.70 mmol), aqueous NH₄Cl (7 M, 2 mL) and Zinc (1.21 g, 18.49 mmol) in MeOH (20 mL) was stirred at 25° C. for 2 hrs. The resulting solution was filtered, concentrated and purified by FCC (Gradient: 0-30% EA in petroleum ether) to give V-4 (1.3 g, 3.51 mmol, 94.8% yield). MS: m/z=370.0 (M+1).

### Step 5: V-5

A solution of tert-butyl 4-(4,4,5,5-tetramethyl-1,3,2-dioxaborolan-2-yl)-3,6-dihydro-2H-pyridine-1-carboxylate (1.25 g, 4.05 mmol), V-4 (1.25 g, 3.37 mmol), potassium carbonate (932.31 mg, 6.75 mmol) and [1,1′-Bis(diphenylphosphino)ferrocene]dichloropalladium(II) (246.80 mg, 337.29 μmol) in Dioxane (15 mL) and water (3 mL) was stirred under microwave at 100° C. for 1 hr. The mixture was quenched with water (20 mL), extracted with EA (60 mL×2), dried over Na₂SO₄, filtered and concentrated to give a residue, which was purified by FCC (Gradient: 0-5 0% EA in petroleum ether) to give V-5 (1.35 g, 2.85 mmol, 84.6% yield). MS: m/z=473.2 (M+1).

### Step 6: V-6

To a mixture of V-5 (1.3 g, 2.75 mmol) in Methanol (20 mL) was added platinum dioxide (62.42 mg, 274.88 μmol) and stirred at 25° C. under hydrogen atmosphere for 12 hrs. The mixture was filtered and concentrated to give V-6 (1.3 g, 2.74 mmol, 99.6% yield). MS: m/z=497.1 (M+23).

### Step 7: Intermediate V

A solution of V-6 (100 mg, 210.55 μmol) in HCl (4 M, 5 mL) was stirred at 25° C. for 2 hrs. The mixture was concentrated to give Intermediate V (78 mg, 208.09 μmol, 98.8% yield). MS: m/z=375.2 (M+1).

The compound in Table E below was made according to procedure of Intermediate G.

TABLE E

| | | ¹H NMR |
|---|---|---|
| | | and/or |
| | | LC/MS |
| Name | structure | data |
| Inter- medi- ate W | 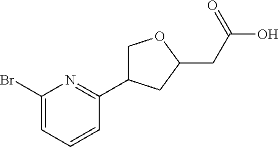 [SMILES: O=C(O)CC1CC(c2cccc(Br)n2)CO1] | MS: m/z = 486.1 (M + 1). |

### Intermediate X:

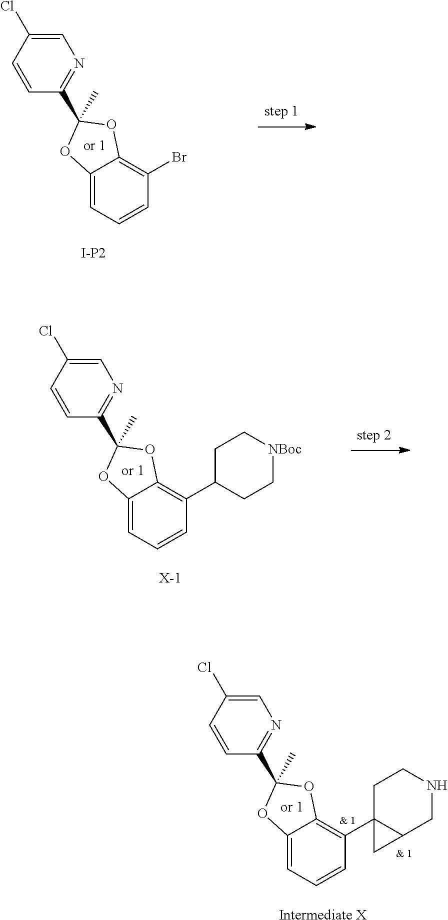

### Step 1: X-1

To a mixture of tert-butyl 4-(4,4,5,5-tetramethyl-1,3,2-dioxaborolan-2-yl)-3,6-dihydro-2H-pyridine-1-carboxylate (473.41 mg, 1.53 mmol) and I-P2 (500 mg, 1.53 mmol) in Water (5 mL) and dioxane (20 mL) was added cyclopentyl(diphenyl)phosphane;dichloropalladium;iron (112.03 mg, 153.11 μmol) and K₂CO₃ (634.80 mg, 4.59 mmol) at 30° C. The mixture was stirred for 4 hrs at 100° C. under N2 atmosphere. The mixture was concentrated and purified by column chromatography on silical gel (EA/petroleum ether=1/10-1/3) to give X-1 (590 mg, 1.38 mmol, 89.8% yield).

### Step 2: Intermediate X

To a stirred solution of X-1 (200 mg, 466.3 μmol) in DCM (20 mL) at −78° C. was added Diethylzinc (1 M, 2.33 mL) followed by diiodomethane (1.25 g, 4.66 mmol) under N2 atmosphere. The mixture was warmed to 25° C. and stirred for 1 hr before poured into saturated aqueous NH₄Cl and extracted with DCM. The combined organic layers were washed with brine, dried over MgSO₄, filtered and concentrated to give Intermediate X (120 mg, crude). MS: m/z=343.2 (M+1).

### Intermediate Y:

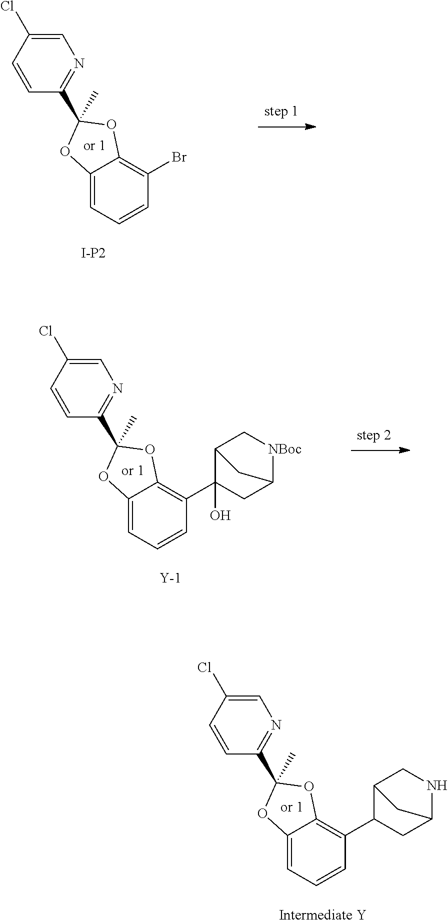

### Step 1: Y-1

To a solution of I-P2 (200 mg, 612.42 μmol) in THF (8 mL) was added butyllithium (2.5 M, 367.45 μL) at −70° C., and the mixture was stirred at −70° C. for 0.5 hr. To which, tert-butyl 5-oxo-2-azabicyclo[2.2.1]heptane-2-carboxylate (155.25 mg, 734.91 μmol) in THF (2 mL) was added at −70° C. and stirred for 1.5 hrs. The reaction was quenched by aqueous NH₄Cl (40 mL) extracted with EA (50 mL×2). The combined organic layers were washed with brine (30 mL), dried over Na₂SO₄ and concentrated to give a residue, which was purified by silica gel chromatography (petroleum ether/EA=5/1) to give Y-1 (110 mg, 239.69 μmol, 39.1% yield). MS: m/z=459.2 (M+1).

### Step 2: Intermediate Y

A solution of Y-1 (110 mg, 239.69 μmol), Triethylsilane (27.87 mg, 239.69 μmol) in TFA (5 mL) was stirred at 100° C. for 4 hrs. The reaction mixture was filtered and concentrated to give a residue, which was purified by silica gel chromatography (EA in petroleum ether: 0˜100%) to give Intermediate Y (20 mg, 58.34 μmol, 24.3% yield). MS: m/z=343.1 (M+1).

### Intermediate Z:

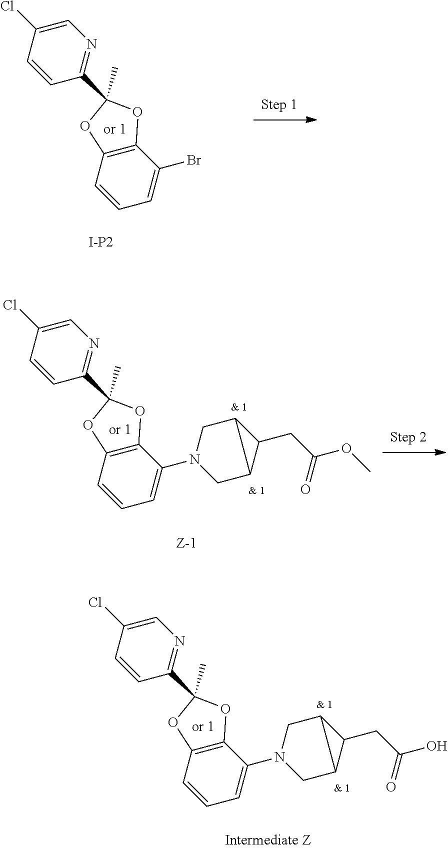 [SMILES: C[C@@]1(c2ccc(Cl)cn2)Oc2cccc(Br)c2O1 ; "I-P2" ; COC(=O)CC1C2CN(c3cccc4c3O[C@](C)(c3ccc(Cl)cn3)O4)CC12 ; C[C@@]1(c2ccc(Cl)cn2)Oc2cccc(N3CC4C(CC(=O)O)C4C3)c2O1]

### Step 1: Z-1

A mixture of I-P2 (100 mg, 306.21 μmol), methyl 2-(3-azabicyclo[3.1.0]hexan-6-yl)acetate (64.56 mg, 336.83 μmol, HCl salt), BINAP (29.89 mg, 45.93 μmol), tris(dibenzylideneacetone)dipalladium (28.04 mg, 30.62 μmol), and Cs₂CO₃ (199.54 mg, 612.42 mol) in Toluene (2 mL) was stirred at 100° C. for 12 hrs. The mixture was concentrated and purified by FCC (Gradient: 0-20% EA in petroleum ether) to give Z-1 (80 mg, 199.57 μmol, 65.2% yield). MS: m/z=401.1 (M+1).

### Step 2: Intermediate Z

To a solution of Z-1 (80 mg, 199.57 μmol) in THF (2 mL), H₂O (1 mL) and Methanol (1 mL) was added NaOH (40 mg, 1.00 mmol). The mixture was stirred at 50° C. for 1 hr. The mixture was concentrated and adjusted to pH˜5 and extracted with EA (10 mL×2). The organic layers were dried over Na₂SO₄, filtered and concentrated to give Intermediate Z (70 mg, 180.96 μmol, 90.7% yield). MS: m/z=387.1 (M+1).

### Intermediate AA:

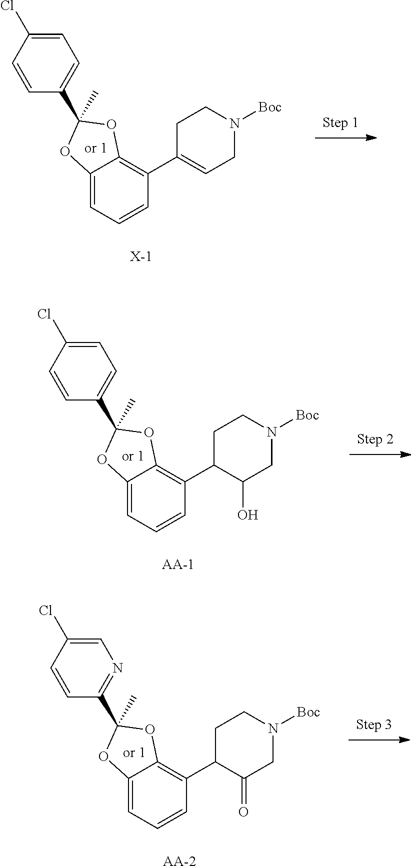

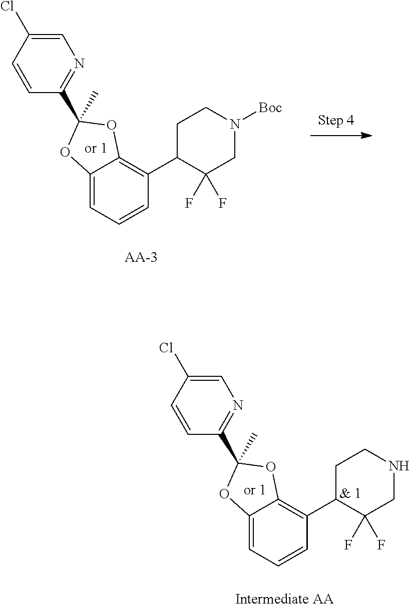

### Step 1: AA-1

To a solution of X-1 (586 mg, 1.37 mmol) in THF (10 mL) was added borane;tetrahydrofuran (1.0 M, 1.50 mL) at 0° C. under N2 atmosphere and stirred for 1 hr before sodium hydroxide (136.62 mg, 3.42 mmol) and hydrogen peroxide (774.44 mg, 6.83 mmol, 704.04 μL, 30% purity) were added. The mixture was warmed to room temperature and stirred for 16 hrs. The resulting mixture was quenched with water (50 mL) and extracted with EA (30 mL×2). The combined organic layers were washed with brine (50 mL), dried over Na₂SO₄, filtered and concentrated to give a residue, which was purified by FCC (silica gel, EA in petroleum ether from 0% to 50%) to give AA-1 (380 mg, 850.26 μmol, 62.2% yield). MS: m/z=447 (M+1).

### Step 2: AA-2

To a mixture of DMSO (199.30 mg, 2.55 mmol) in DCM (10 mL) was added oxalyl dichloride (215.84 mg, 1.70 mmol, 147.84 μL) slowly at −78° C. under N2 atmosphere. The mixture was stirred for 15 minutes, to which, a solution of AA-1 (380 mg, 850.26 μmol) in DCM (3 mL) was added slowly. The mixture was stirred for 1 hr at −78° C. before N,N-diethylethanamine (344.15 mg, 3.40 mmol, 474.04 μL) was added, and the mixture was stirred for further 2 hrs. The mixture was quenched with water (50 mL) and extracted with EA (30 mL×2). The combined organic layers were washed with brine (50 mL), dried over Na₂SO₄, filtered and concentrated to give a residue, which was purified by silica gel (EA in petroleum ether from 0% to 50%) to give AA-2 (30 mg, 67.43 μmol, 7.9% yield). MS: m/z=445 (M+1). Step 3: AA-3

To a solution of AA-2 (30 mg, 67.43 μmol) in DCM (5 mL) was added DAST (44.75 mg, 202.29 μmol), and the mixture was stirred for 12 hrs. The reaction was quenched with 5% aqueous sodium bicarbonate solution, stirred for 15 minutes, and the resulting mixture was extracted twice using DCM. The organic phases were combined, dried over MgSO₄, filtered and concentrated to give a residue, which was purified by silica gel flash chromatography (EA in petroleum ether: 0˜40%) to give AA-3 (20 mg, 42.84 μmol, 63.5% yield). MS: m/z=467.2 (M+1).

### Step 4: Intermediate AA

To a solution of AA-3 (20 mg, 42.84 μmol) in DCM (5 mL) was added HCl (4 M, 53.54 μL) and stirred at 25° C. for 4 hrs. The reaction mixture was concentrated to give Intermediate AA (18 mg, crude, HC salt). MS: m/z=367.1 (M+1).

### Intermediate AB:

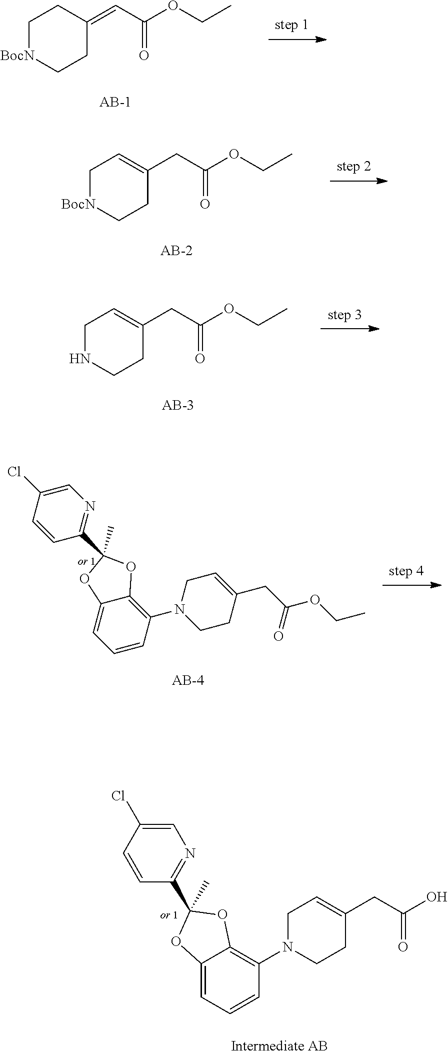

### Step 1: AB-2

A solution of AB-1 (5 g, 18.56 mmol) in THF (100 mL) was cooled to −78° C., LDA (2 M, 18.56 mL) was added under stirring, and the mixture was stirred for 30 minutes at −78° C. before 2.4 nL of AcOH in THF (20 mL) was added. The mixture was stirred for 20 minutes at this temperature before warmed up to room temperature. To the mixture was added 200 mL of saturated aqueous NaHCO₃ and 70 mL of EA, the organic layer was separated, and the aqueous layer was extracted with 100 mL of FA. The combined organic phases were washed with brine, dried over Na₂SO₄ and concentrated to give AB-2 (4 g, 14.85 mmol, 80.0% yield).

### Step 2: AB-3

To a mixture of AB-2 (3 g, 11.14 mmol) in DCM (10 mL) was added HCl/dioxane (4 M, 15 mL) and stirred at 20° C. for 1 hr. The mixture was concentrated to give AB-3 (2.29 g, crude, HCl salt).

### Step 3: AB-4

A solution of AB-3 (211.27 mg, 1.10 mmol, HCl salt), I-P2 (300 mg, 918.63 mol) and Tris(dibenzylideneacetone)dipalladium (84.12 mg, 91.86 μmol) in Toluene (20 mL) was stirred at 120° C. for 4 hrs. The mixture was cooled to room temperature, concentrated and purified by FCC (silica gel, EA in petroleum ether: 0˜40%) to give AB-4 (200 mg, 498.93 mol, 54.3% yield).

### Step 4: Intermediate AB

AB-4 (200 mg, 498.93 μmol) in Methanol (10 mL) and THF (10 mL) at 25° C. was treated with NaOH/H₂O (4 M, 498.93 μL), and the mixture was stirred for 35 minutes at 50° C. The mixture was then cooled to 10° C. and pH was adjusted to ˜2 by addition of 1 M HCl (4 mL) over 25 minutes while continuing to cool to 5° C., after which a solid precipitate was formed. The slurry was diluted with water (100 mL) and stirred for 40 minutes, and the solid was collected by filtration. The solid was dissolved in EA (100 mL), dried over Na₂SO₄, filtered and concentrated to give Intermediate AB (150 mg, 387.77 μmol, 77.7% yield). MS: m/z=387.2 (M+1).

### Intermediate AC:

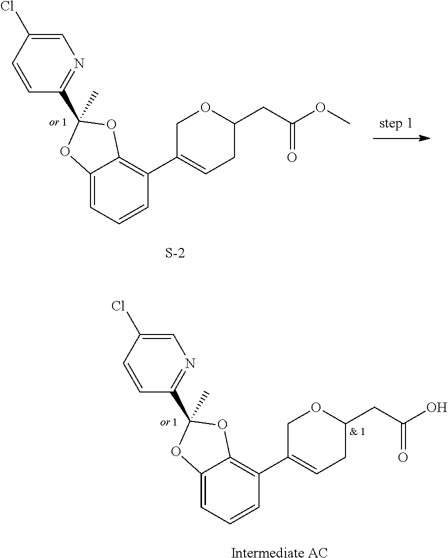

### Step 1: Intermediate AC

A mixture of S-2 (200 mg, 497.71 μmol) and NaOH (19.91 mg, 497.71 mol) in Methanol (3 mL), THF (5 mL) and Water (2 mL) was stirred at 40° C. for 2 hrs. The resulting mixture was acidified with 1 M HCl to pH=5-6 and extracted with EA (30 mL). The separated organic layer was washed with brine, dried over Na₂SO₄, filtered and concentrated to give Intermediate AC (160 mg, 412.57 μmol, 82.9% yield). MS: m/z=388.1 (M+1).

### Intermediate AD:

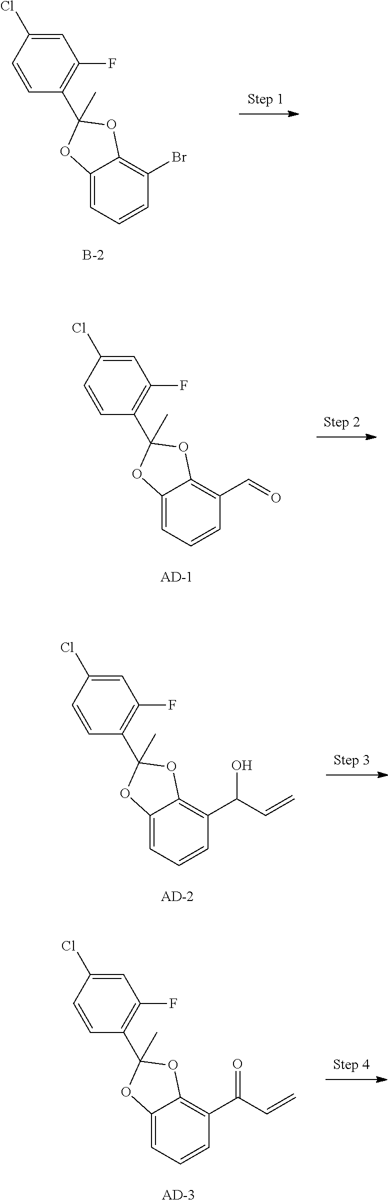

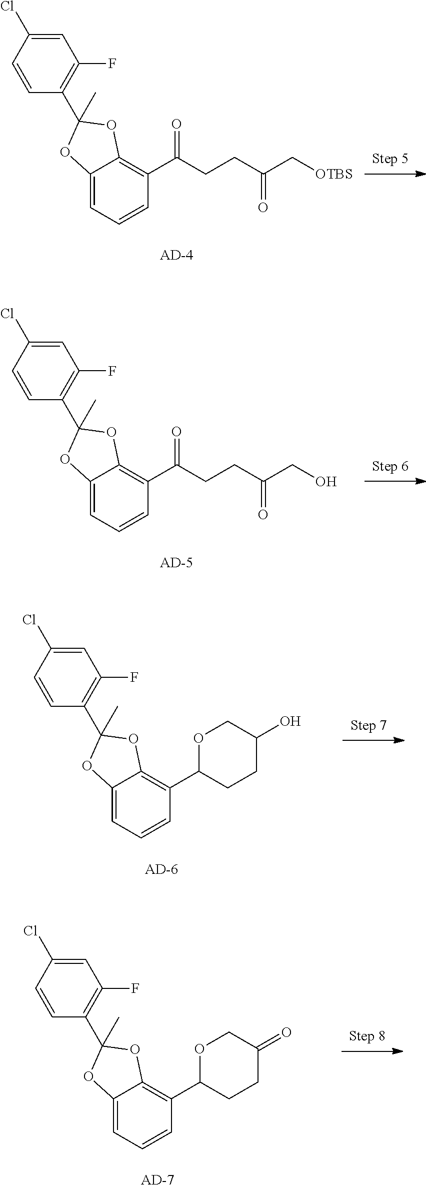

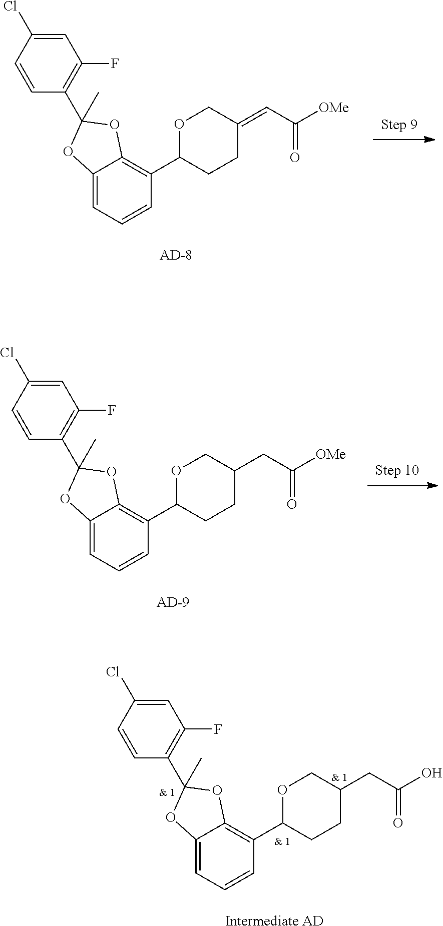

### Step 1: AD-1

To a solution of B-2 (6.59 g, 19.18 mmol) in THF (60 mL) was added butyllithium (2.5 M, 8.44 mL) at −78° C., the mixture was stirred for 1 hr before DMF (1.54 g, 21.10 mmol, 1.63 mL) was added. The mixture was stirred for further 1 hr. The reaction was quenched by aqueous NH₄Cl (150 mL) and extracted with EA (80 mL×3). The combined organic layers were washed with brine (120 mL), dried over Na₂SO₄, filtered and concentrated to give a residue, which was purified by silica gel (EA in petroleum ether from 0% to 15%) to give AD-1 (4.17 g, 14.25 mmol, 74.3% yield). MS: m/z=293 (M+1). ¹H NMR (400 MHz, DMSO-d₆) δ 10.30 (s, 1H), 7.83 (t, J=8.2 Hz, 1H), 7.51 (dd, J=8.5, 1.2 Hz, 1H), 6.98 (dd, J=5.7, 3.3 Hz, 2H), 6.87 (dd, J=5.7, 3.3 Hz, 2H), 2.06 (s, 3H).

### Step 2: AD-2

To a solution of AD-1 (3.77 g, 12.88 mmol) in TH (60 mL) was added Vinylmagnesium chloride (1.0 M, 15.46 mL) at −78° C. and the mixture was stirred for 1 hr before quenched with aqueous NH₄Cl (50 mL) and extracted with EA (50 mL×2). The combined organic layers were washed with brine (50 mL), dried over Na₂SO₄, filtered and concentrated to give a residue, which was purified by silica gel (EA in petroleum ether from 0% to 15%) to give AD-2 (3.56 g, 11.10 mmol, 86.2% yield). MS: m/z=321 (M+1, ESI).

### Step 3: AD-3

To a solution of AD-2 (3.56 g, 11.10 mmol) in DCM (35 mL) was added dioxomanganese (9.65 g, 110.99 mmol) and stirred for 18 hrs at 25° C. The mixture was filtered and washed with THF (30 mL×3). The combined organic phases were concentrated to give a residue, which was purified by silica gel (EA in petroleum ether from 0% to 20%) to give AD-3 (2.38 g, 7.47 mmol, 67.3% yield). MS: m/z=319 (M+1).

### Step 4: AD-4

To a solution of AD-3 (2.38 g, 7.47 mmol) and 2-[tert-butyl(dimethyl)silyl]oxyacetaldehyde (1.30 g, 7.47 mmol) in Dioxane (30 mL) was added 2-(3-benzyl-4-methyl-thiazol-3-ium-5-yl)ethanol;chloride (201.46 mg, 746.72 μmol) and N,N-diethylethanamine (75.56 mg, 746.72 umol), the mixture was stirred for 18 hrs at 80° C. The mixture was concentrated to give a residue, which was purified by silica gel (EA in petroleum ether from 0% to 15%) to give AD-4 (2.0 g, 4.06 mmol, 54.3% yield). MS: m/z=493 (M+1).

### Step 5: AD-5

To a solution of AD-4 (2.0 g, 4.06 mmol) in DCM (10 mL) was added HCl/Dioxane (4.0 M, 10.14 mL) and stirred for 2 hrs at 25° C. The mixture was concentrated and re-dissolved with EA (50 mL), the organic phase was washed with aqueous NaHCO₃ (50 mL×2) and brine (50 mL), dried over Na₂SO₄, filtered and concentrated to give a residue, which was purified by silica gel (EA in petroleum ether from 0% to 50%) to give AD-5 (884 mg, 2.33 mmol, 57.5% yield). MS: m/z=379 (M+1).

### Step 6: AD-6

To a mixture of AD-5 (884 mg, 2.33 mmol) in DCM (50 mL) was added diethyloxonio(trifluoro)boranuide (794.97 mg, 5.60 mmol) and triethylsilane (651.30 mg, 5.60 mmol) at 0° C. The reaction was stirred for 1.5 hrs at 0° C. before quenched with aqueous NaHCO₃ (50 mL) and extracted with DCM (30 mL×2). The combined organic layers were washed with brine (50 mL), dried over Na₂SO₄, filtered and concentrated to give a residue, which was purified by silica gel (EA in petroleum ether from 0% to 15%) to give AD-6 (485 mg, 1.33 mmol, 57.0% yield). MS: m/z=365 (M+1). ¹H NMR (400 MHz, Chloroform-d) δ 7.52-7.40 (m, 1H), 7.18-7.09 (m, 1H), 6.92-6.70 (m, 4H), 5.64-5.35 (m, 1H), 4.59-4.14 (m, 1H), 3.90-3.78 (m, 1H), 3.75-3.55 (m, 1H), 2.41-2.08 (m, 4H), 2.06 (dd, J=4.1, 1.2 Hz, 3H).

### Step 7: AD-7

To a mixture of AD-6 (485 mg, 1.33 mmol) in DCM (10 mL) was added (1,1-diacetoxy-3-oxo-1,2-benziodoxol-1-yl) acetate (676.68 mg, 1.60 mmol) and stirred for 0.5 hr at 25° C. The reaction was quenched with water (50 mL) and extracted with EA (30 mL×3). The combined organic layers were washed with brine (50 mL), dried over Na₂SO₄, filtered and concentrated to give a residue, which was purified by silica gel (EA in petroleum ether from 0% to 15%) to give AD-7 (427 mg, 1.18 mmol, 88.5% yield). MS: m/z=363 (M+1). ¹H NMR (400 MHz, Chloroform-d) δ 10.06-9.64 (m, 1H), 7.55-7.45 (m, 1H), 7.22-7.12 (m, 1H), 6.82 (s, 4H), 5.73-5.54 (m, 1H), 4.79-4.30 (m, 1H), 2.52-2.13 (m, 4H), 2.05 (d, J=1.3 Hz, 3H).

### Step 8: AD-8

To a mixture of methyl 2-dimethoxyphosphorylacetate (421.67 mg, 2.32 mmol) in THF (10 mL) was added Sodium hydride (115.76 mg, 2.89 mmol, 60% purity) at 0° C. and stirred for 2 hrs. Then, AD-7 (420 mg, 1.16 mmol) was added and stirred for another 3 hrs. The reaction was quenched with aqueous NH₄Cl (50 mL) and extracted with EA (30 mL×3). The combined organic layers were washed with brine (50 mL), dried over Na₂SO₄, filtered and concentrated to give a residue, which was purified by silica gel chromatography (EA in petroleum ether from 0% to 25%) to give AD-8 (330 mg, 787.89 μmol, 68.0% yield). MS: m/z=419 (M+1).

### Step 9: AD-9

To a mixture of AD-8 (330 mg, 787.89 μmol) in MeOH (6 mL) was added dioxoplatinum (17.89 mg, 78.79 μmol) and stirred for 1 hr under H2 atmosphere. The reaction was filtered and concentrated to give AD-9 (314 mg, 746.10 μmol, 94.7% yield).

### Step 10: Intermediate AD

To a mixture of AD-9 (314 mg, 746.10 μmol) in MeOH (2 mL), THF (2 mL) and water (2 mL) was added lithium;hydroxide;hydrate (78.27 mg, 1.87 mmol) and stirred for 1 hr. The reaction was quenched with water (50 mL) and extracted with EA (50 mL). The aqueous phase was separated and acidified with 1 M HCl to pH=3-4 and extracted with EA (50 mL×3). The combined organic layers were washed with brine (80 mL), dried over Na₂SO₄, filtered and concentrated to give Intermediate AD (218 mg, 535.85 μmol, 71.8% yield). MS: m/z=407 (M+1).

### Intermediate AE:

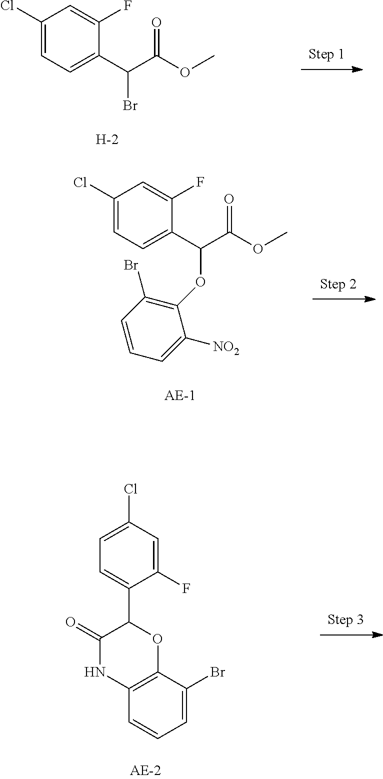

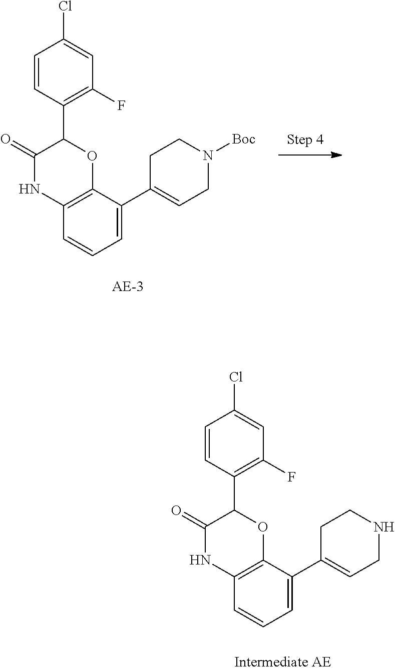 [SMILES: CC(C)(C)OC(=O)N1CC=C(c2cccc3c2OC(c2ccc(Cl)cc2F)C(=O)N3)CC1 ; O=C1Nc2cccc(C3=CCNCC3)c2OC1c1ccc(Cl)cc1F]

### Step 1: AE-1

A solution of H-2 (700 mg, 2.49 mmol), 2-bromo-6-nitro-phenol (542.10 mg, 2.49 mmol) and K₂CO₃ (1.03 g, 7.46 mmol) in DMF (30 mL) was stirred at 25° C. for 2 hrs. The mixture was concentrated and purified by FCC (silica gel, petroleum ether/EA=10/1 to 5/1) to give AE-1 (700 mg, 1.67 mmol, 67.2% yield).

### Step 2: AE-2

A solution of AE-1 (700 mg, 1.67 mmol), iron (933.87 mg, 16.72 mmol) and NH₄Cl (1.79 g, 33.44 mmol, 1.17 mL) in methanol (30 mL) and water (20 mL) was stirred at 60° C. for 2 hrs. The resulting solution was concentrated and purified by FCC (silica gel, petroleum ether/EA=10/1 to 5/1) to give AE-2 (400 mg, 1.12 mmol, 67.1% yield). MS: m/z=356 (M+1).

### Step 3: AE-3

A mixture of AE-2 (100 mg, 280.45 μmol), tert-butyl 4-(4,4,5,5-tetramethyl-1,3,2-dioxaborolan-2-yl)-3,6-dihydro-2H-pyridine-1-carboxylate (130.07 mg, 420.67 μmol), cyclopentyl(diphenyl)phosphane;dichloromethane;dichloropalladium;iron (11.45 mg, 14.02 mol) and K₂CO₃ (77.52 mg, 560.89 μmol) in water (2 mL) and dioxane (8 mL) was heated at 120° C. under microwave for 40 minutes. The mixture was cooled to room temperature, filtered, concentrated and purified by FCC (silica gel, petroleum ether/EA=3/1) to give AE-3 (120 mg, 261.49 μmol, 93.2% yield). MS: m/z=403 (M−55).

### Step 4: Intermediate AE

A mixture of AE-3 (60 mg, 130.74 μmol) and HCl/dioxane (4 M, 585.64 μL) in dioxane (3 mL) was stirred at 25° C. for 2 hrs. The resulting mixture was concentrated to give Intermediate AE (50 mg, crude, HCl salt). MS: m/z=359 (M+1).

### Intermediate AF:

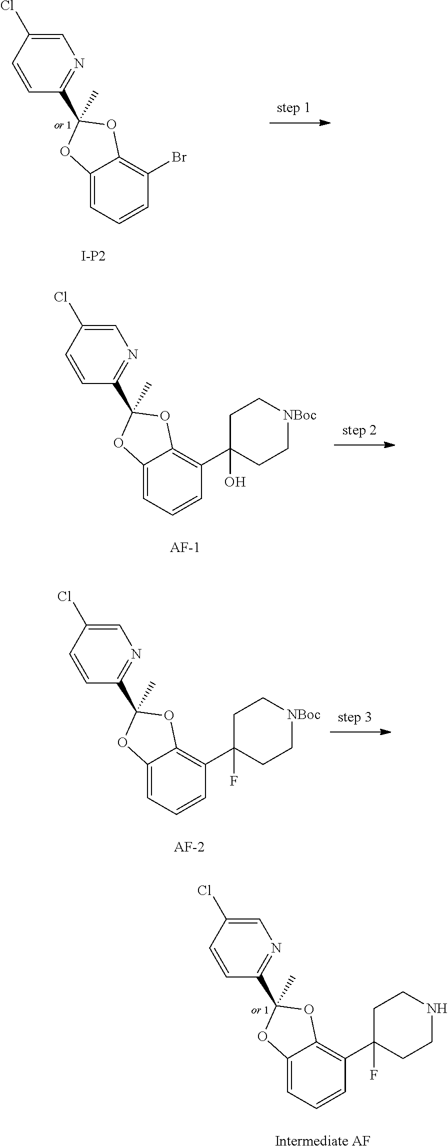

### Step 1: AF-1

To a mixture of I-P2 (500 mg, 1.53 mmol) in THF (30 mL) at −78° C. was added butyllithium (2.5 M, 673.66 μL) dropwise, and the mixture was stirred at −78° C. for 30 minutes before tert-butyl 4-oxopiperidine-1-carboxylate (305.06 mg, 1.53 mmol) in THF (5 mL) was added. The mixture was stirred for 30 minutes, quenched by saturated aqueous NH₄Cl (10 mL) and extracted with EA (50 mL). The organic phase was concentrated and purified by column chromatography on silical gel (EA/petroleum ether=1/10-1/3) to give AF-1 (450 mg, 1.01 mmol, 65.8% yield).

### Step 2: AF-2

To a solution of AF-1 (200 mg, 447.50 μmol) and 2,3,4,6,7,8,9,10-octahydropyrimido[1,2-a]azepine (102.19 mg, 671.26 μmol) in DCM (50 mL) was added XtalFluor-E (153.71 mg, 671.26 μmol), the mixture was stirred for 30 minutes at 0° C. The reaction mixture was allowed to warm to 25° C. and stirred for another 12 hrs before quenched by 5% aqueous sodium bicarbonate and extracted twice with DCM. The organic phases were combined, dried over MgSO₄, filtered and concentrated to give AF-2 (200 mg, crude).

### Step 3: Intermediate AF

To a mixture of AF-2 (200 mg, 445.52 μmol) in DCM (10 mL) was added HCl/dioxane (4 M, 4 mL) and stirred at 20° C. for 1 hr. The mixture was concentrated to give Intermediate AF (175 mg, crude, HCl salt). MS: m/z=349.1 (M+1).

### Intermediate AG:

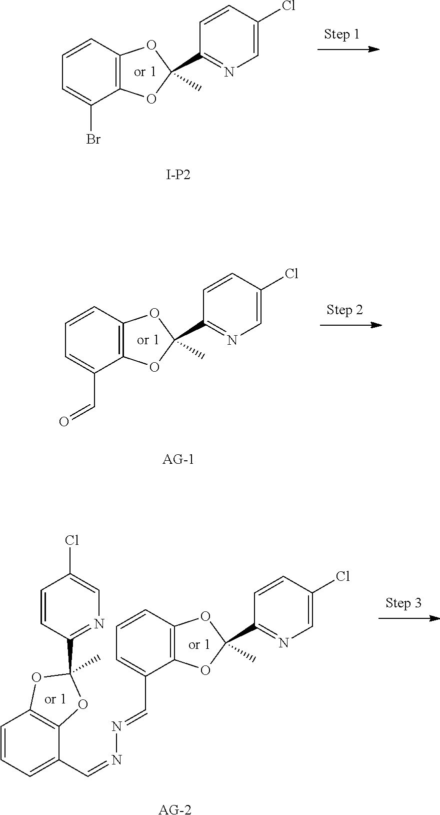 [SMILES: C[C@@]1(c2ccc(Cl)cn2)Oc2cccc(Br)c2O1 ; "I-P2" ; C[C@@]1(c2ccc(Cl)cn2)Oc2cccc(C=O)c2O1 ; "AG-1" ; C[C@@]1(c2ccc(Cl)cn2)Oc2cccc(/C=N\N=C\c3cccc4c3O[C@](C)(c3ccc(Cl)cn3)O4)c2O1 ; "AG-2"]

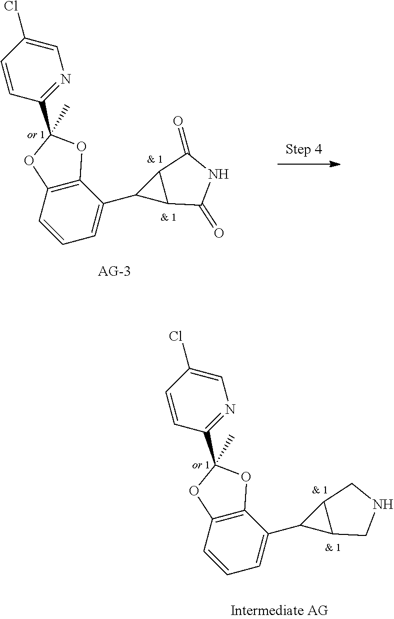 [SMILES: C[C@@]1(c2ccc(Cl)cn2)Oc2cccc(C3C4C(=O)NC(=O)C43)c2O1 ; C[C@@]1(c2ccc(Cl)cn2)Oc2cccc(C3C4CNCC43)c2O1]

### Step 1: AG-1

To a mixture of I-P2 (1.25 g, 3.83 mmol) in THF (10 mL) was added butyllithium (2.5 M, 1.84 mL) at −78° C. and stirred for 0.5 hr before DMF (559.52 mg, 7.66 mmol, 592.72 μL) was added. The mixture was stirred at −78° C. for another 1 hr, quenched by H₂O (30 mL) and extracted with EA (20 mL×3). The combined organic phases were dried over Na₂SO₄, filtered, concentrated and purified by FCC (Gradient: 0-20% EA in petroleum ether) to give AG-1 (0.9 g, 3.26 mmol, 85.3% yield). MS: m/z=276.0 (M+1).

### Step 2: AG-2

To a mixture of hydrazine (156.92 mg, 4.90 mmol) in Methanol (10 mL) was added AG-1 (0.9 g, 3.26 mmol) and stirred for 2 hrs at 80° C. The mixture was concentrated and purified by FCC (Gradient: 0-30% EA in petroleum ether) to give AG-2 (0.75 g, 1.37 mmol). MS: m/z=547.2 (M+1).

### Step 3: AG-3

To a solution of AG-2 (650 mg, 1.19 mmol) in Dioxane (6 mL) was added manganese dioxide (825.90 mg, 9.50 mmol) and the reaction mixture stirred for 40 minutes. The slurry was filtered through celite and the celite pad was washed with dioxane (2 ml×3). The filtrate was returned to a pot and to which pyrrole-2,5-dione (461.08 mg, 4.75 mmol) was added portion wise over a period of 20 minutes. The reaction mixture was stirred at room temperature for 4 hrs before being heated at 105° C. for 20 hrs. The mixture was concentrated and purified by FCC (Gradient: 0-20% EA in petroleum ether) to give AG-3 (90 mg, 252.27 μmol, 21.2% yield). MS: m/z=357.1 (M+1).

### Step 4: Intermediate AG

Borane;tetrahydrofuran (130.08 mg, 1.51 mmol) was added to a solution of AG-3 (90 mg, 252.27 μmol) in THF (5 mL), and the mixture was stirred at 60° C. for 2 hrs. The reaction mixture was cooled, treated with saturated aqueous NaHCO₃ (20 mL) and extracted with EA (15 mL×3). The organic layers were dried over Na₂SO₄, filtered, and concentrated to give Intermediate AG (85 mg, crude). MS: m/z=329.1 (M+1).

### Intermediate AH:

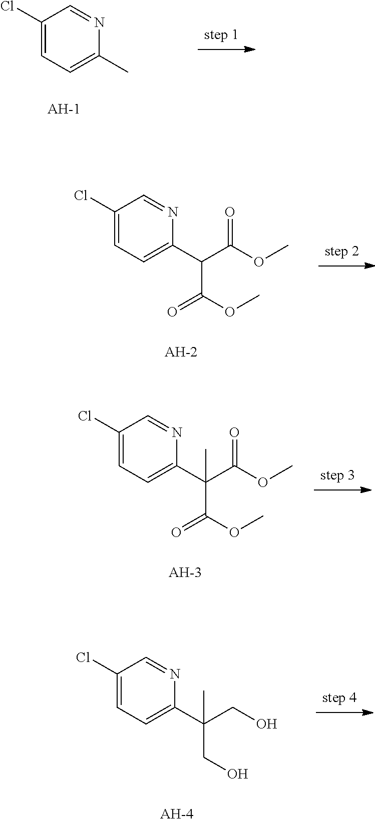

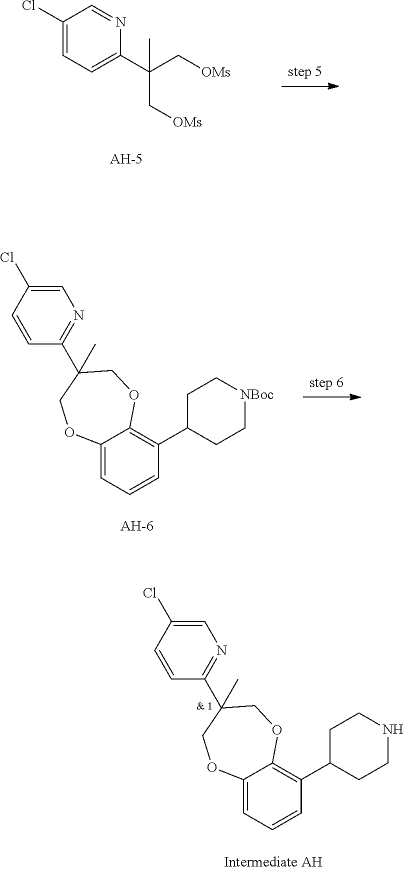 [SMILES: CC(COS(C)(=O)=O)(COS(C)(=O)=O)c1ccc(Cl)cn1 ; CC(C)(C)OC(=O)N1CCC(c2cccc3c2OCC(C)(c2ccc(Cl)cn2)CO3)CC1 ; CC1(c2ccc(Cl)cn2)COc2cccc(C3CCNCC3)c2OC1]

### Step 1: AH-2

To a solution of AH-1 (10 g, 78.39 mmol) in THF (50 mL) was added LDA (2 M, 117.58 mL) at −78° C. and stirred for 1 hr before dimethyl carbonate (7.06 g, 78.39 mmol, 6.60 mL) was added. After addition, the solution was slowly warmed to room temperature and stirred for 4 hrs. The reaction was quenched by aqueous NH₄Cl (100 mL) and extracted with EA (250 mL). The separated organic layer was concentrated and purified by FCC (silica gel, PE/EA=10/1 to 3/1) to give AH-2 (3 g, 12.31 mmol, 15.7% yield).

### Step 2: AH-3

To a solution of AH-2 (1 g, 4.10 mmol) in DMF (30 mL) was added K₂CO₃ (1.70 g, 12.31 mmol) and Mel (1.17 g, 8.21 mmol, 511.03 μL). The mixture was heated at 45° C. for 4 hrs. The reaction mixture was partitioned between EA (50 ml) and water (50 ml). The organic layer was dried over Na₂SO₄ and concentrated to give a residue, which was purified by chromatographed on silica gel (EA/petroleum ether=1/60-1/30) to give AH-3 (1 g, 3.88 mmol, 94.6% yield).

### Step 3: AH-4

To a mixture of AH-3 (1 g, 3.88 mmol) in Methanol (20 mL) was added NaBH₄ (146.83 mg, 3.88 mmol) and stirred at 25° C. for 2 hrs. The mixture was concentrated and purified by column chromatography on silica gel (EA/petroleum ether=1/10-1/1) to give AH-4 (400 mg, 1.98 mmol, 51.1% yield).

### Step 4: AH-5

To a solution of AH-4 (200 mg, 991.82 μmol) in DCM (10 mL) was added N,N-diethylethanamine (301.09 mg, 2.98 mmol) and Ms₂O (207.33 mg, 1.19 mmol), the mixture was stirred at 25° C. for 15 hrs. To the mixture, saturated aqueous NaHCO₃ (30 mL) was added, and the aqueous phase was extracted with DCM. The organic phase was dried over MgSO₄, filtered, and concentrated to give AH-5 (354 mg, crude).

### Step 5: AH-6

A mixture of tert-butyl 4-(2,3-dihydroxyphenyl)piperidine-1-carboxylate (81.98 mg, 279.46 μmol), AH-5 (100 mg, 279.46 μmol), KI (92.78 mg, 558.92 μmol), and Cs₂CO₃ (273.16 mg, 838.39 μmol) in MeCN (30 mL) was stirred at 80° C. for 8 hrs. To the mixture, EA (80 mL) was added and filtered, which was concentrated to give a residue, which was purified by column chromatography on silica gel (EA/petroleum ether=1/10-1/1) to give AH-6 (80 mg, 174.30 mol, 62.4% yield).

### Step 6: Intermediate AH

To a mixture of AH-6 (80 mg, 174.30 μmol) in DCM (20 mL) was added HCl/dioxane (4 M, 2 mL) and stirred at 20° C. for 1 hr. The mixture was concentrated to give Intermediate AH (65 mg, crude, HCl salt). MS: m/z=359.2 (M+1).

### Intermediate AI:

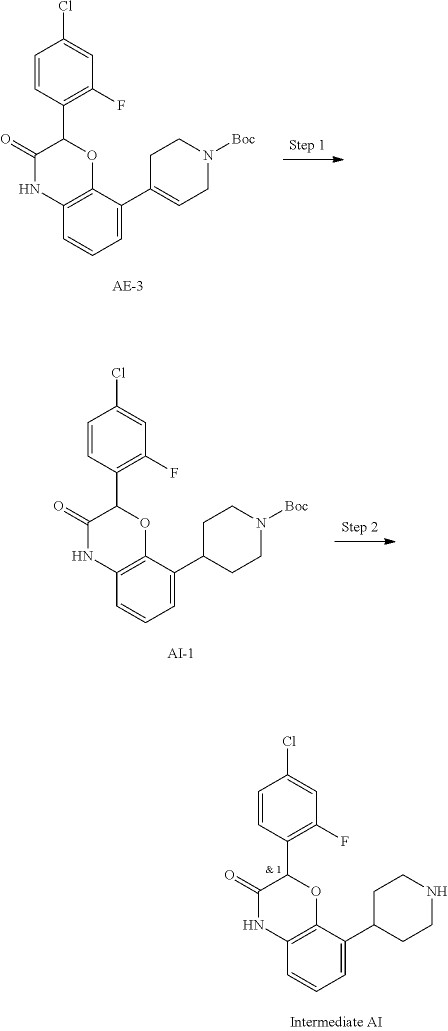 [SMILES: CC(C)(C)OC(=O)N1CC=C(c2cccc3c2OC(c2ccc(Cl)cc2F)C(=O)N3)CC1 ; CC(C)(C)OC(=O)N1CCC(c2cccc3c2OC(c2ccc(Cl)cc2F)C(=O)N3)CC1 ; O=C1Nc2cccc(C3CCNCC3)c2OC1c1ccc(Cl)cc1F]

Step 1: A-1

To a mixture of AE-3 (70 mg, 152.54 μmol) in methanol (20 mL) was added PtO2 (3.46 mg, 15.25 umol), and the reaction stirred at 25° C. for 12 hrs under H2 atmosphere. The mixture was filtered and concentrated to give AI-1 (70 mg, crude). MS: m/z=405 (M−55).

### Step 2: Intermediate AI

A mixture of AI-1 (55 mg, 119.33 μmol) and HCl/dioxane (4 M, 534.49 μL) in dioxane (3 mL) was stirred at 25° C. for 2 hrs. The resulting mixture was concentrated to give Intermediate AT (40 mg, 110.86 μmol, 92.9% yield, HCl salt). MS: m/z=361 (M+1).

### Intermediate AJ:

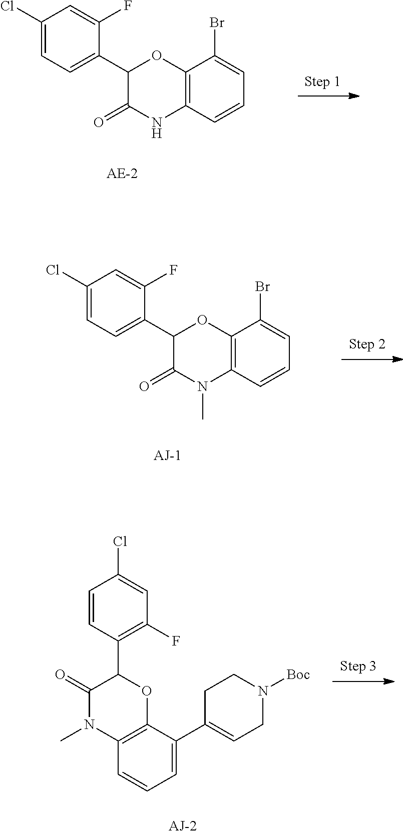 [SMILES: O=C1Nc2cccc(Br)c2OC1c1ccc(Cl)cc1F ; CN1C(=O)C(c2ccc(Cl)cc2F)Oc2c(Br)cccc21 ; CN1C(=O)C(c2ccc(Cl)cc2F)Oc2c(C3=CCN(C(=O)OC(C)(C)C)CC3)cccc21]

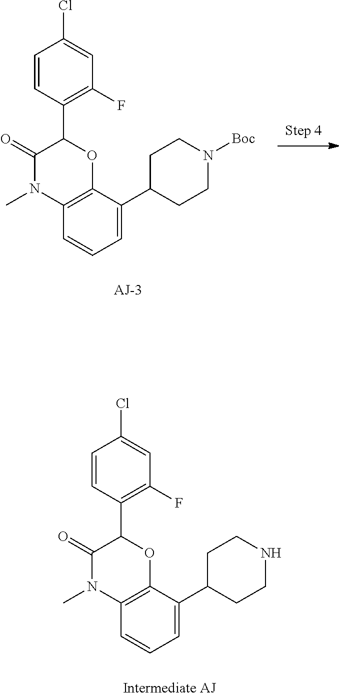 [SMILES: CN1C(=O)C(c2ccc(Cl)cc2F)Oc2c(C3CCN(C(=O)OC(C)(C)C)CC3)cccc21 ; CN1C(=O)C(c2ccc(Cl)cc2F)Oc2c(C3CCNCC3)cccc21]

### Step 1: AJ-1

A mixture of AE-2 (100 mg, 280.45 μmol), Mel (199.03 mg, 1.40 mmol) and K₂CO₃ (116.28 mg, 841.34 μmol) in DMF (15 mL) was stirred at 25° C. for 4 hrs. The resulting mixture was diluted with water and extracted with EA (20 mL). The separated organic layer was washed with water (20 mL) and brine, dried over Na₂SO₄, filtered and concentrated to give a residue, which was purified by FCC (silica gel, petroleum ether/EA=5/1 to 3/1) to give AJ-1 (100 mg, 269.83 μmol, 96.2% yield). MS: m/z=370 (M+1).

### Step 2: AJ-2

A mixture of AJ-1 (100 mg, 269.83 μmol), tert-butyl 4-(4,4,5,5-tetramethyl-1,3,2-dioxaborolan-2-yl)-3,6-dihydro-2H-pyridine-1-carboxylate (125.15 mg, 404.75 μmol), cyclopentyl(diphenyl)phosphane;dichloromethane;dichloropalladium;iron (11.02 mg, 13.49 mol) and K₂CO₃ (74.58 mg, 539.66 μmol) in water (2 mL) and dioxane (8 mL) was stirred at 120° C. under microwave for 40 minutes. The resulting mixture was cooled to room temperature and filtered. The filtrate was concentrated and purified by FCC (silica gel, petroleum ether/EA=3/1) to give AJ-2 (120 mg, 253.73 μmol, 94.0% yield). MS: m/z=417 (M−55).

### Step 3: AJ-3

To a mixture of AJ-2 (120 mg, 253.73 μmol) in methanol (20 mL) at 25° C. was added dioxoplatinum (12 mg, 52.84 μmol) and stirred for 12 hrs under H2 atmosphere. The mixture was filtered and concentrated to give AJ-3 (120 mg, 252.66 μmol, 99.6% yield). MS: m/z=419 (M−55).

### Step 4: Intermediate AJ

A mixture of AJ-3 (120 mg, 252.66 μmol) and HCl/dioxane (4 M, 1.13 mL) in dioxane (3 mL) was stirred at 25° C. for 2 hrs. The resulting mixture was concentrated to give Intermediate AJ (90 mg, crude, HCl salt). MS: m/z=375 (M+1).

### Intermediate AK:

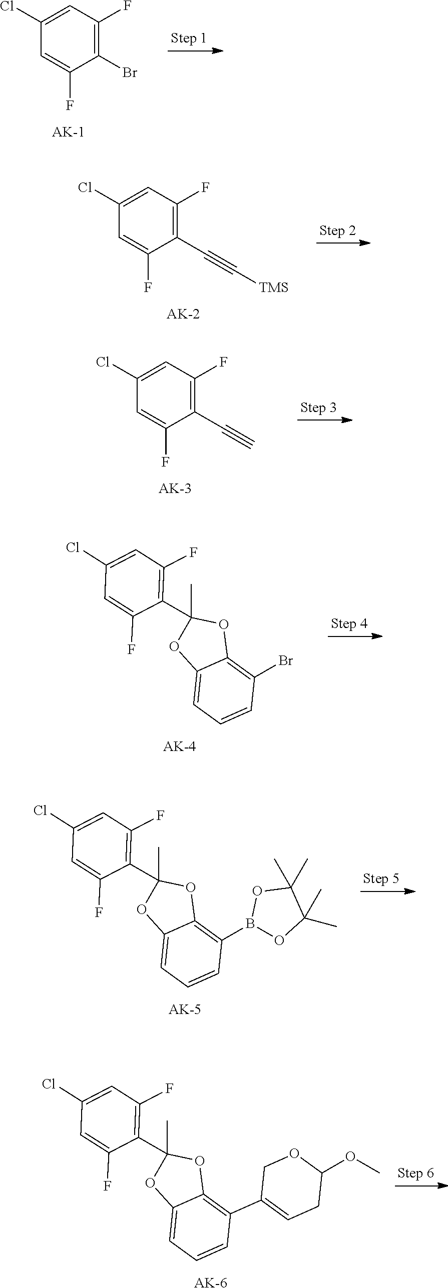

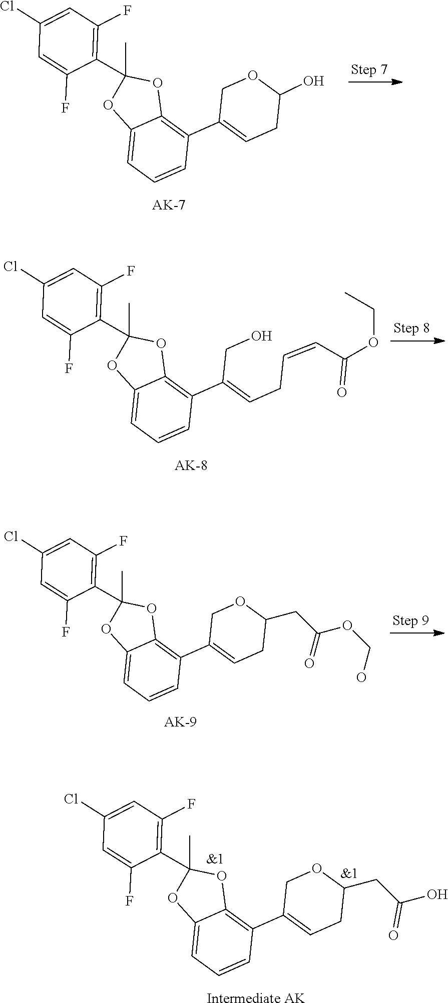

### Step 1: AK-2

A solution of AK-1 (25 g, 109.92 mmol), ethynyl(trimethyl) silane (12.96 g, 131.91 mmol, 18.64 mL), DIPEA (33.37 g, 329.77 mmol, 45.96 mL), Bis(triphenylphosphine)palladium(II) chloride (3.86 g, 5.50 mmol) and cuprous iodide (2.09 g, 10.99 mmol) in THF (150 mL) was stirred at 25° C. for 3 hrs. The mixture was concentrated and purified by FCC (Gradient: 0-2% EA in petroleum ether) to give AK-2 (23 g, 93.98 mmol, 85.5% yield).

### Step 2: AK-3

To a solution of AK-2 (15 g, 61.29 mmol) in THF (100 mL), DMF (10 mL) and THE (100 mL) was added KF (10.68 g, 183.87 mmol), and the mixture was stirred for 1 hr at 25° C. The mixture was diluted with EA (200 mL), washed by water (150 mL×3). The combined organic phases were dried over Na₂SO₄, filtered and concentrated to give a residue, which was purified by FCC (Gradient: 0-5% EA in petroleum ether) to give AK-3 (3 g, 17.39 mmol, 28.4% yield).

### Step 3: AK-4

To a mixture of AK-3 (1.2 g, 5.01 mmol) and 3-bromobenzene-1,2-diol (946.35 mg, 5.01 mmol) in Water (2 mL) and Toluene (15 mL) was added triruthenium dodecacarbonyl (160.06 mg, 250.35 μmol), and the mixture was stirred for 16 hrs at 100° C. The mixture was concentrated and purified by FCC (Gradient: 0-5% EA in petroleum ether) to give AK-4 (850 mg, 2.35 mmol, 46.9% yield).

### Step 4: AK-5

To a mixture of AK-4 (850 mg, 2.35 mmol), 4,4,5,5-tetramethyl-2-(4,4,5,5-tetramethyl-1,3,2-dioxaborolan-2-yl)-1,3,2-dioxa borolane (656.68 mg, 2.59 mmol) in Dioxane (10 mL) was added [1,1′-Bis(diphenylphosphino) ferrocene]dichloropalladium(II) (172.02 mg, 235.09 μmol) and KOAc (692.16 mg, 7.05 mmol). The mixture was stirred for 16 hrs at 100° C. under microwave. The reaction was cooled and quenched with water (10 mL), and the mixture was extracted with EA (20 mL×2). The combined organic phase was dried over Na₂SO₄, filtered and concentrated to give AK-5 (900 mg, 2.20 mmol, 93.7% yield).

### Step 5: AK-6

To a mixture of (2-methoxy-3,6-dihydro-2H-pyran-5-yl) trifluoromethanesulfonate (519.75 mg, 1.98 mmol), AK-5 (900 mg, 2.20 mmol) in Dioxane (10 mL) and H₂O (2.5 mL) was added Tetrakis(triphenylphosphine)palladium (254.51 mg, 220.25 μmol) and KOAc (608.79 mg, 4.40 mmol). The mixture was stirred for 1 hr at 100° C. under microwave. The reaction was cooled and quenched with water (10 mL), and the mixture was extracted with EA (20 mL×2). The combined organic phase was dried over Na₂SO₄, filtered, concentrated and purified by FCC (Gradient: 0-20% EA in petroleum ether) to give AK-6 (300 mg, 759.89 μmol, 34.5% yield). MS: m/z=417.1 (M+23).

### Step 6: AK-7

A solution of AK-6 (210 mg, 531.92 μmol) in H₂O (3 mL) and AcOH (6 mL) was stirred for 12 hrs at 80° C. The mixture was concentrated to give AK-7 (200 mg, 525.25 μmol, 98.7% yield). MS: m/z=363.1 (M−17).

### Step 7: AK-8

A mixture of ethyl 2-(triphenyl-phosphanylidene)acetate (274.48 mg, 787.88 mol) and AK-7 (200 mg, 525.25 μmol) in Toluene (10 mL) was stirred for 2 hrs at 100° C. The mixture was concentrated and purified by FCC (Gradient: 0-50% EA in petroleum ether) to give AK-8 (100 mg, 221.80 μmol, 42.2% yield). MS: m/z=473.2 (M+23).

### Step 8: AK-9

To a solution of AK-8 (100 mg, 211.8 μmol) in THF (6 mL) was added NaH (16.94 mg, 423.6 μmol, 60% purity) at 0° C., the mixture was stirred at 70° C. for 1 hr. The reaction was concentrated to give a residue, which was purified by FCC (Gradient: 0-20% EA in petroleum ether) to give AK-9 (80 mg, 177.44 μmol, 76.2% yield). MS: m/z=451.1 (M+1).

### Step 9: Intermediate AK

To a solution of AK-9 (80 mg, 177.44 μmol) in THF (1 mL), Water (1 mL) and Methanol (2 mL) was added NaOH (80 mg, 2.00 mmol). The mixture was stirred at 50° C. for 2 hrs. The mixture was concentrated, adjusted to pH˜5, and extracted with EA (10 mL×2). The combined organic phase was dried over Na₂SO₄, filtered and concentrated to give Intermediate AK (60 mg, 141.91 μmol, 80.0% yield). MS: m/z=423.1 (M+1).

### Intermediate AL:

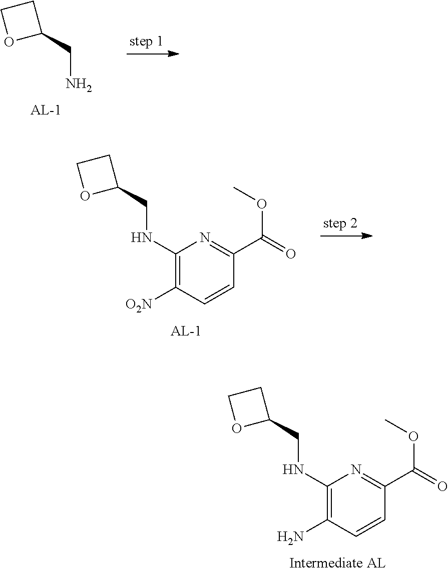 [SMILES: NC[C@@H]1CCO1 ; COC(=O)c1ccc([N+](=O)[O-])c(NC[C@@H]2CCO2)n1 ; COC(=O)c1ccc(N)c(NC[C@@H]2CCO2)n1]

### Step 1: AL-2

A mixture of methyl 6-chloro-5-nitro-pyridine-2-carboxylate (500 mg, 2.31 mmol), AL-1 (211.18 mg, 2.42 mmol) and DIPEA (596.74 mg, 4.62 mmol) in MeCN (20 mL) was stirred at 70° C. for 8 hrs. The resulting mixture was concentrated and re-dissolved in EA (30 mL). The organic phase was washed with water (10 mL) and brine, dried over Na₂SO₄, filtered and concentrated to give AL-2 (500 mg, 1.87 mmol, 81.0% yield). MS: m/z=268.1 (M+1).

### Step 2: Intermediate AL

A mixture of AL-2 (500 mg, 1.87 mmol) and Palladium 10% on Carbon (wetted with ca. 55% Water, 50 mg, 469.84 μmol) in Methanol (15 mL) was stirred at 25° C. for 2 hrs under H2 atmosphere. The resulting mixture was filtered and concentrated to give Intermediate AL (400 mg, 1.69 mmol, 90.1% yield). MS: m/z=238.2 (M+1).

The compound in Table F below was made according to the procedure of Intermediate AL.

TABLE F

| | | ¹H NMR |
|---|---|---|
| | | and/or |
| | | LC/MS |
| Name | Structure | data |
| Inter- medi- ate AM | 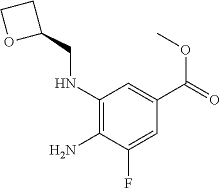 [SMILES: COC(=O)c1cc(F)c(N)c(NC[C@@H]2CCO2)c1] | MS: m/z = 255.2 (M + 1). |

### Intermediate AN:

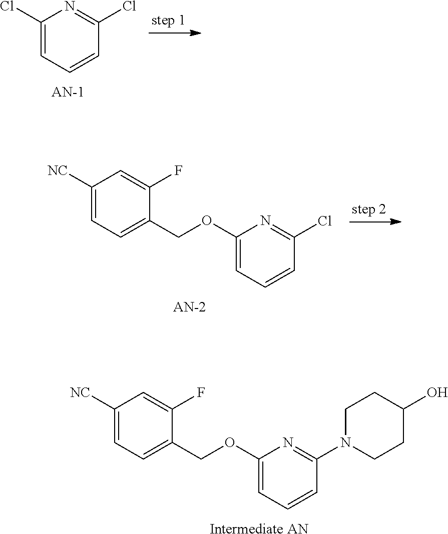 [SMILES: Clc1cccc(Cl)n1 ; N#Cc1ccc(COc2cccc(Cl)n2)c(F)c1 ; "AN-2" ; N#Cc1ccc(COc2cccc(N3CCC(O)CC3)n2)c(F)c1 ; "Intermediate AN"]

### Step 1: AN-2

To a solution of 3-fluoro-4-(hydroxymethyl)benzonitrile (2 g, 13.23 mmol) and AN-1 (2.35 g, 15.88 mmol) in THF (70 mL) was added t-BuOK (2.23 g, 19.85 mmol) at 0° C. The mixture was stirred at 0-15° C. overnight. The mixture was quenched with H₂O (20 mL), concentrated and filtered to give a crude, which was purified by flash eluting with petroleum ether:EA=9:1 to give AN-2 (1.75 g, 6.66 mmol, 50.3% yield). MS: m/z=263 (M+1).

### Step 2: Intermediate AN

To a solution of AN-2 (500 mg, 1.90 mmol) and piperidin-4-ol (211.79 mg, 2.09 mmol) in DMF (12 mL) was added N,N-diethylethanamine (577.86 mg, 5.71 mmol). The mixture was stirred at 100° C. overnight and concentrated to give a residue. The residue was diluted with EA (20 mL), and washed with brine (20 mL×3). The organic phase was dried over Na₂SO₄, filtered and concentrated to give a residue, which was purified by flash eluting with petroleum ether:EA=3:1 and further purified by prep-HPLC (0.1% HCOOH) to give Intermediate AN (51 mg, 155.80 μmol, 8.2% yield). MS: m/z=328 (M+1).

The compound in Table G below was made according to the procedure of Compound S-2.

TABLE G

| | | ¹H |
|---|---|---|
| | | NMR |
| | | and/or |
| | | LC/MS |
| Name | Structure | data |
| Inter- medi- ate AO | 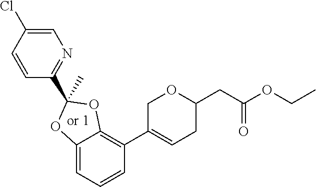 [SMILES: CCOC(=O)CC1CC=C(c2cccc3c2O[C@](C)(c2ccc(Cl)cn2)O3)CO1] | MS: m/z = 416 (M + 1). |

### Intermediate AP:

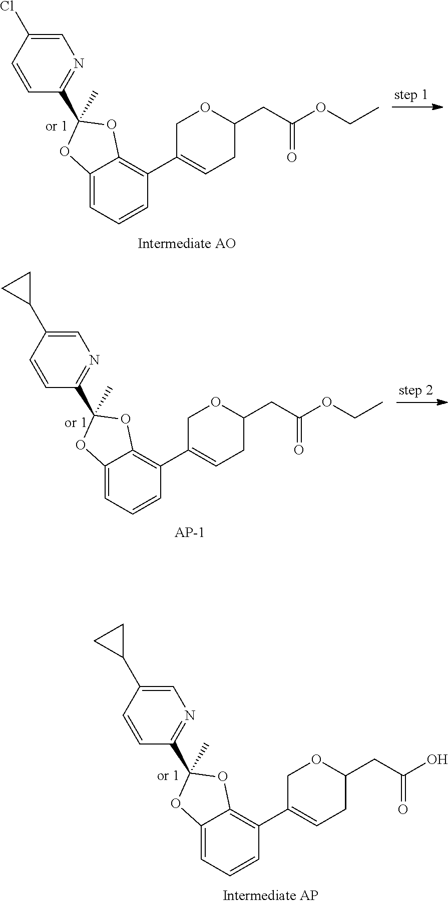 [SMILES: CCOC(=O)CC1CC=C(c2cccc3c2O[C@](C)(c2ccc(Cl)cn2)O3)CO1 ; CCOC(=O)CC1CC=C(c2cccc3c2O[C@](C)(c2ccc(C4CC4)cn2)O3)CO1 ; C[C@@]1(c2ccc(C3CC3)cn2)Oc2cccc(C3=CCC(CC(=O)O)OC3)c2O1]

### Step 1: AP-1

To a solution of cyclopropylboronic acid (37.18 mg, 432.83 μmol), Intermediate AO (120 mg, 288.55 μmol), tricyclohexylphosphane (16.18 mg, 57.71 μmol) and Palladium acetate (6.48 mg, 28.86 μmol) in Toluene (10 mL) was added K₃PO₄ (183.75 mg, 865.66 μmol). The mixture was stirred at 120° C. for 16 hrs. The resulting mixture was cooled to room temperature and concentrated to give a residue, which was purified by FCC (silica gel, EA in petroleum ether: 0˜10%) to give AP-1 (80 mg, 189.81 μmol, 65.8% yield). MS: m/z=422.3 (M+1).

### Step 2: Intermediate AP

To a solution of AP-1 (80 mg, 189.81 μmol) in THF (5 mL), MeOH (2 mL) and water (2 mL) was added Lithium hydroxide monohydrate (119.46 mg, 2.85 mmol). The reaction was stirred at 50° C. for 2 hrs. The mixture was extracted with EA (30 mL×3) and the aqueous layer was adjusted to pH˜5. The aqueous phase was extracted with EA (30 mL×3), and the combined organic layers were washed with brine (30 mL×3), dried over Na₂SO₄, filtered and concentrated to give a residue, which was purified by prep-HPLC (HCO₂H) to give Intermediate AP (35 mg, 88.96 μmol, 46.9% yield). MS: m/z=394.2 (M+1).

The compound in Table H below was made according to the procedure of Intermediate F.

TABLE H

| | | ¹H NMR |
|---|---|---|
| | | and/or |
| | | LC/MS |
| Name | Structure | data |
| Inter- medi- ate AQ | 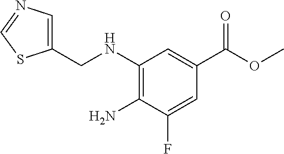 [SMILES: COC(=O)c1cc(F)c(N)c(NCc2cncs2)c1] | MS: m/z = 282 (M + 1). |
| Inter- medi- ate AR | 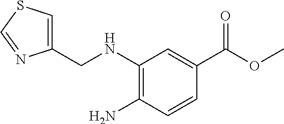 [SMILES: COC(=O)c1ccc(N)c(NCc2cscn2)c1] | MS: m/z = 264 (M + 1). |
| Inter- medi- ate AS | 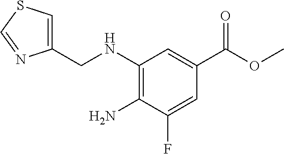 [SMILES: COC(=O)c1cc(F)c(N)c(NCc2cscn2)c1] | MS: m/z = 282 (M + 1). |

### Intermediate AT:

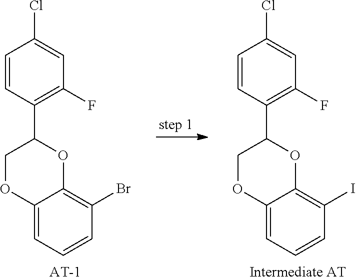

### Step 1: Intermediate AT

To a suspension of NaI (654.40 mg, 4.37 mmol), CuI (24.94 mg, 130.98 μmol), (1R,2R)-N1,N2-dimethylcyclohexane-1,2-diamine (37.26 mg, 261.95 μmol) and AT-1 (150 mg, 436.59 μmol, which is prepared according to the procedure of O-4) in dioxane (5 mL) was stirred at 110° C. for 4 hrs. The reaction mixture was concentrated and purified by column chromatography on silica gel (EA in PE, 0% to 2%) to give Intermediate AT (170.52 mg, 436.59 mol) as a yellow oil.

### Intermediate AU:

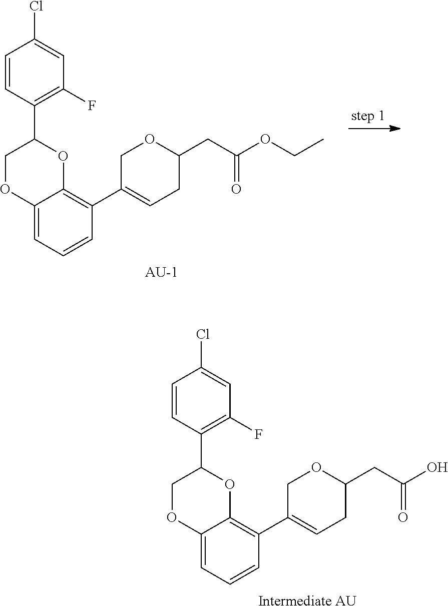

### Step 1: Intermediate AU

A solution of AU-1 (30 mg, 69.31 μmol, which is prepared according to the procedure of S-2) and lithium;hydroxide;hydrate (5.82 mg, 138.61 μmol) in THF (1 mL), MeOH (1 mL) and water (0.5 mL) was stirred for 18 hrs. The reaction mixture was quenched with water (50 mL), acidified to pH=3-4 with aqueous 1 M HCl and extracted with EA (30 mL×2). The combined organic layers were washed with brine (30 mL), dried over Na₂SO₄, filtered and concentrated to give Intermediate AU (15 mg, 37.05 μmol). MS: m/z=405 (M+1).

### Intermediate AV:

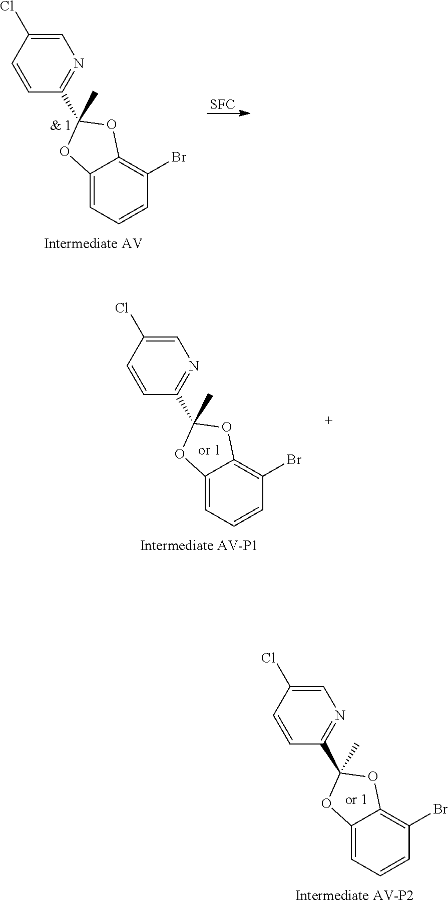 [SMILES: C[C@]1(c2ccc(Cl)cn2)Oc2cccc(Br)c2O1 ; "SFC" ; C[C@]1(c2ccc(Cl)cn2)Oc2cccc(Br)c2O1 ; C[C@@]1(c2ccc(Cl)cn2)Oc2cccc(Br)c2O1]

Intermediate AV (800 mg, which is prepared according to the procedure of B-2) was separated by SFC (Column: Daicel ChiralPak AY-H 250 mm×30 mm I.D., 5 μm, Mobile Phase: CO₂/EtOH=95:5, Flowrate: 50 g/min, Wavelength: 220 nm, Temperature: 40° C.) to give Intermediate AV-P1 (249 mg) and Intermediate AV-P2 (271 mg).

The compound in Table I below was made according to the procedure of Intermediate AU.

TABLE I

| Name | Structure | ¹H NMR and/or LC/MS data |
|---|---|---|
| Intermediate AW | 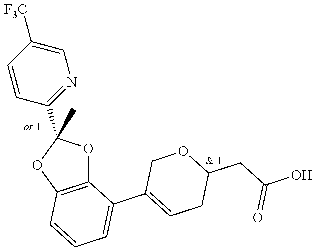 [SMILES: C[C@]1(c2ccc(C(F)(F)F)cn2)Oc2cccc(C3=CCC(CC(=O)O)OC3)c2O1] | MS: m/z = 422(M + H) |
| Intermediate AX | 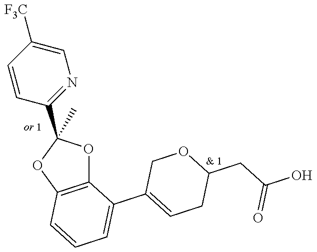 [SMILES: C[C@@]1(c2ccc(C(F)(F)F)cn2)Oc2cccc(C3=CCC(CC(=O)O)OC3)c2O1] | MS: m/z = 422(M + H) |

### Intermediate AY:

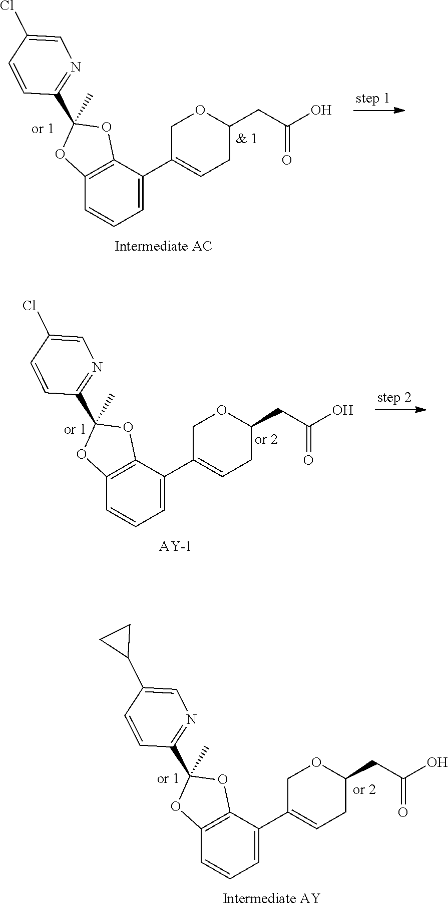

### Step 1: A Y-1

Intermediate AC (3.1 g) was separated by SFC (Column: Daicel ChiralPak AD-H 250 mm×30 mm I.D., 5 m; Mobile Phase: CO₂/EtOH=70:30; Flowrate: 50 g/min; Wavelength: 254 nm; Temperature: 40° C.) to give AY-1 (peak 1, 1.2 g) and another isomer (1.4 g).

### Step 2: Intermediate AY

To a solution of cyclopropylboronic acid (44.68 mg, 520.16 μmol), AY-1 (50 mg, 128.93 μmol), tricyclohexylphosphane (7.23 mg, 25.79 μmol) and Palladium acetate (2.89 mg, 12.89 μmol) in Toluene (10 mL) was added K₃PO₄ (82.10 mg, 386.78 μmol), and the mixture was stirred at 120° C. for 16 hrs. The mixture was cooled to room temperature and concentrated to give a residue, which was purified by FCC (silica gel, EA in petroleum ether: 0˜50%) to give Intermediate AY (30 mg, 76.25 μmol, 59.1% yield).

### Example 1: Compound 15

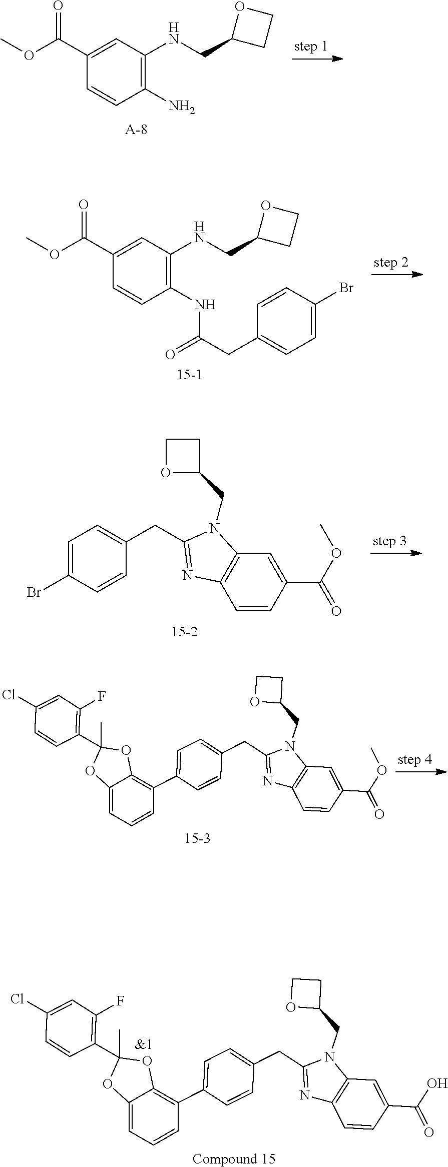

### Step 1: 15-1

To a solution of 2-(4-bromophenyl) acetic acid (136.53 mg, 634.88 μmol), A-8 (100 mg, 423.25 μmol) and HATU (257.49 mg, 677.20 μmol) in DMF (5 mL) was added DIPEA (164.10 mg, 1.27 mmol). The mixture was stirred at 25° C. overnight. The resulting mixture was concentrated and purified by FCC (silica gel, PE/EA=10/1 to 1/3) to give 15-1 (100 mg, 230.79 mol). MS: m/z=433 (M+1).

### Step 2: 15-2

A solution of 15-1 (100 mg, 230.79 μmol) in AcOH (10 mL) was stirred at 100° C. for 1 hr. Upon completion, the resulting mixture was cooled to room temperature and concentrated. The residue was purified by FCC (silica gel, PE/EA=1/1 to 0/1) to give 15-2 (80 mg, 192.64 mol) as yellow oil. MS: m/z=415 (M+1).

### Step 3: 15-3

To a mixture of 15-2 (70 mg, 168.56 μmol), Intermediate B (79.02 mg, 202.27 mol) in water (1 mL) and dioxane (5 mL) was added K₂CO₃ (69.89 mg, 505.68 μmol) and cyclopentyl(diphenyl)phosphane;dichloropalladium;iron (12.33 mg, 16.86 μmol) at 30° C. The mixture was stirred for 2 hrs at 100° C. The mixture was filtered and the filtrate was evaporated to give a residue, which was purified by column chromatography on silical gel (EA/PE=10:1) to give 15-3 (80 mg, 133.55 μmol). MS: m/z=599.1 (M+1).

### Step 4: Compound 15

A mixture of 15-3 (70 mg, 116.85 μmol) and NaOH (14.02 mg, 350.56 μmol) in THE (2 mL) and water (0.5 mL) was stirred at 20° C. for 3 hrs. The solution was acidified with 1 M HCl to PH=3, and the solids was collected by filtration and washed with water to give Compound 15 (45 mg, 76.92 μmol). ¹H NMR (400 MHz, DMSO-d6) δ 12.78 (s, 1H), 8.23 (s, 1H), 7.80 (d, J=8.4 Hz, 1H), 7.74-7.54 (m, 5H), 7.42 (d, J=7.6 Hz, 2H), 7.35 (d, J=8.4 Hz, 1H), 7.14 (s, 1H), 6.95 (d, J=4.8 Hz, 2H), 4.94 (s, 1H), 4.67 (t, J=11.2 Hz, 1H), 4.59-4.33 (m, 5H), 2.35 (s, 2H), 2.06 (s, 3H) ppm; MS: m/z=585.1 (M+1).

### Example 2: Compound 16

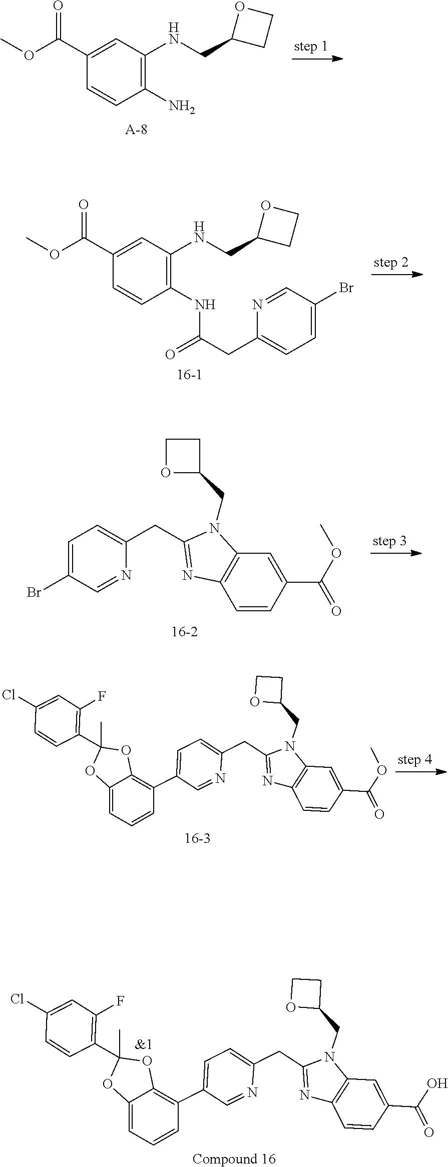

### Step 1: 16-1

To a mixture of A-8 (100 mg, 423.25 μmol), 2-(5-bromo-2-pyridyl)acetic acid (109.72 mg, 507.90 μmol) and HATU (177.03 mg, 465.58 μmol) in DMF (5 mL) was added DIPEA (164.11 mg, 1.27 mmol) at 25° C. The reaction mixture was stirred for 20 hrs at 25° C. The mixture was quenched with water (40 mL) and extracted by EA (30 mL×2). The combined organic layers were washed with brine (30 mL), dried over Na₂SO₄ and concentrated to give a residue, which was purified by silica gel chromatography (PE:EA=1:1) to give 16-1 (110 mg, 253.29 μmol, 59.8% yield). MS: m/z=434 (M+1).

### Step 2: 16-2

To a mixture of 16-1 (110 mg, 253.29 μmol) in AcOH (10 mL) was stirred at 100° C. for 1 hr. The reaction mixture was cooled and concentrated to give a residue, which was neutralized with saturated NaHCO₃ until pH˜8. The mixture was extracted by EA (30 mL×2), and the combined organic layers were washed with brine (30 mL), dried over Na₂SO₄ and concentrated to give a residue, which was purified by silica gel chromatography (PE:EA=1:2) to give 16-2 (70 mg, 168.16 μmol, 66.2% yield). MS: m/z=416 (M+1).

### Step 3: 16-3

To a mixture of 16-2 (70 mg, 168.16 μmol), Intermediate B (85 mg, 217.59 μmol) and K₂CO₃ (46.27 mg, 334.76 μmol) in dioxane (4 mL) and water (1 mL) was added 1,1′-Bis(diphenylphosphino)ferrocene-palladium(II)dichloride dichloromethane complex (6.83 mg, 8.37 μmol) at 25° C. The mixture was stirred at 120° C. for 0.75 hr in a Biotage microwave reactor. Then, the solution was concentrated, to which was added water (20 mL) and extracted by EA (30 mL×2). The combined organic layers were washed with brine (30 mL), dried over Na₂SO₄ and concentrated to give a residue, which was purified by silica gel chromatography (EA) to give 16-3 (62 mg, 103.33 μmol, 61.7% yield). MS: m/z=600 (M+1).

### Step 4: Compound 16

To a mixture of 16-3 (62 mg, 103.33 μmol) in THF (4 mL), methanol (4 mL) and water (1 mL) was added NaOH (20.66 mg, 516.64 μmol) at 25° C. The reaction solution was stirred at 30° C. for 18 hrs. Then, the solution was concentrated with a rotary evaporator. To the residue was added with water (20 mL) and neutralized with 6 M hydrochloric acid until the pH of the suspension reached 4˜5. The mixture was filtered and washed with water (10 mL) to give Compound 16 (44 mg, 75.08 μmol, 72.7% yield). ¹H NMR (400 MHz, DMSO-d6) δ 8.87 (d, J=2.4 Hz, 1H), 8.28 (s, 1H), 8.13 (d, J=8.34, 1H), 7.82 (d, J=8.4 Hz, 1H), 7.74-7.50 (m, 4H), 7.35 (d, J=8.4, 1H), 7.22 (d, J=7.4, 1H), 7.17-6.93 (m, 2H), 5.02 (d, J=7.6 Hz, 1H), 4.79 (d, J=15.6, 1H), 4.72-4.56 (m, 2H), 4.54-4.43 (m, 1H), 4.36-4.33 (m, 1H), 2.67-2.62 (m, 1H), 2.39-2.36 (m, 1H), 2.08 (s, 3H) ppm; MS: m/z=586 (M+1).

### Example 3: Compound 17

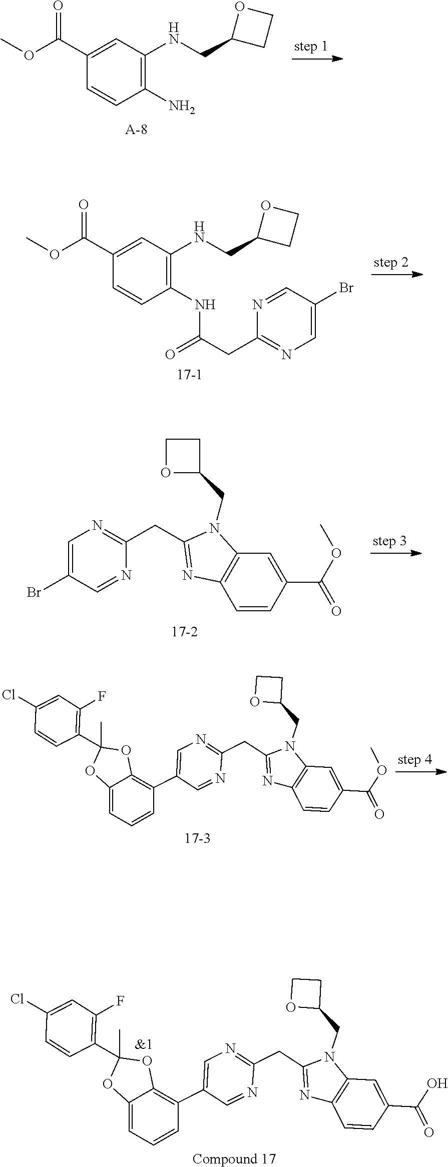

### Step 1: 17-1

To a solution of 2-(5-bromopyrimidin-2-yl) acetic acid (110.22 mg, 507.90 μmol), A-8 (100 mg, 423.25 μmol) and HATU (193.12 mg, 507.90 μmol) in DMF (3 mL) was added DIPEA (164.10 mg, 1.27 mmol). After addition, the solution was stirred at 25° C. overnight. The reaction mixture was quenched with water (50 mL) and extracted with EA (30 mL×3). The combined organic layers were washed with brine (50 mL), dried over Na₂SO₄, filtered and concentrated. The residue was purified by combi flash (MeOH in DCM from 0% to 20%) to give 17-1 (175 mg, 402.05 μmol, 95.0% yield). MS: m/z=435 (M+1).

### Step 2: 17-2

To a solution of 17-1 (175 mg, 402.05 μmol) in AcOH (5 mL) was stirred at 100° C. for 1 hr. The reaction mixture was quenched with aqueous NaHCO₃ (150 mL) to pH=8˜9 and extracted with EtOAc (50 mL×3). The combined organic layers were washed with brine (80 mL), dried over Na₂SO₄, filtered and concentrated to give 17-2 (140 mg, 335.53 μmol, 83.4% yield). MS: m/z=417 (M+1).

### Step 3: 17-3

To a solution of 17-2 (120 mg, 287.59 μmol) and Intermediate B (146.05 mg, 373.87 μmol) in Dioxane (3 mL) and water (1 mL) was added K₂CO₃ (119.24 mg, 862.78 μmol) and cyclopentyl(diphenyl)phosphane;dichloropalladium;iron (21.04 mg, 28.76 μmol), and the mixture was stirred at 100° C. for 4 hrs. The reaction was quenched with water (50 mL) and extracted with EtOAc (50 mL×2). The combined organic layers were washed with brine (50 mL), dried over Na₂SO₄, filtered and concentrated. The residue was purified by combi flash (EA in PE from 0% to 70%) to give 17-3 (80 mg, 133.11 μmol, 46.3% yield). MS: m/z=601 (M+1).

### Step 4: Compound 17

To a solution of 17-3 (80 mg, 133.11 μmol) in THF (3 mL) and water (1 mL) was added lithium;hydroxide;hydrate (22.34 mg, 532.43 μmol) and stirred for 18 hrs at 25° C. The mixture was added water (10 mL) and acidified to pH=3˜4 with 1 M HCl, and then extracted with EA (30 mL×3). The combined organic layers were washed with brine (50 mL), dried over Na₂SO₄, filtered and concentrated. The residue was purified by prep-HPLC (FA) to give Compound 17 (2.5 mg, 4.26 μmol, 3.2% yield). MS: m/z=587 (M+1).

### Example 4: Compound 21

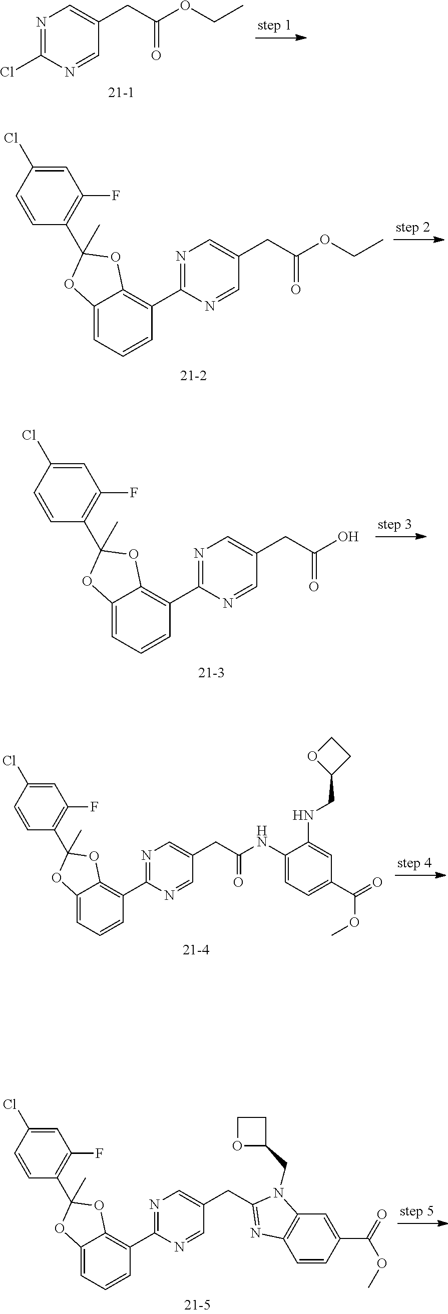

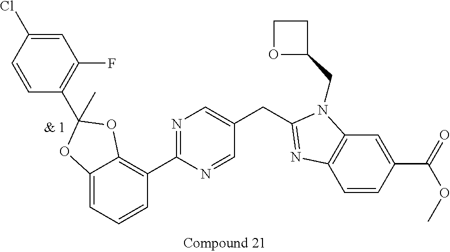 [SMILES: COC(=O)c1ccc2nc(Cc3cnc(-c4cccc5c4OC(C)(c4ccc(Cl)cc4F)O5)nc3)n(C[C@@H]3CCO3)c2c1]

### Step 1: 21-2

To a mixture of 21-1 (50 mg, 249.23 μmol), Intermediate B (126.56 mg, 323.99 mol) and K₂CO₃ (68.89 mg, 498.45 μmol) in dioxane (4 mL) and Water (1 mL) was added 1,1′-Bis(diphenylphosphino)ferrocene-palladium(II)dichloride dichloromethane complex (10.18 mg, 12.46 μmol) at 25° C. The reaction mixture was stirred at 120° C. for 0.75 hr in a Biotage microwave reactor. Then, the solution was concentrated to give a residue, which was charged with H₂O (30 mL) and extracted with EA (30 mL×2). The combined organic layers were washed with brine (30 mL), dried over Na₂SO₄, and concentrated to give the crude product, which was purified by silica gel chromatography (PE/EA=1/3) to give 21-2 (70 mg, 163.23 mol, 65.5% yield). MS: m/z=429.2 (M+1).

### Step 2: 21-3

To a mixture of 21-2 (70 mg, 163.23 μmol) in THF (2 mL), Water (0.5 mL) and Methanol (2 mL) was added NaOH (26.12 mg, 652.92 μmol) at 25° C. The reaction solution was stirred at 30° C. for 18 hrs. Then, the solution was concentrated, charged with H₂O (10 mL) and acidified with 6 M HCl until pH reached 4-5. The mixure was extracted with EA (20 mL×2), and the combined organic layers were washed with brine (30 mL), dried over Na₂SO₄, and concentrated to give 21-3 (58 mg, crude). MS: m/z=401.1 (M+1).

### Step 3: 21-4

To a mixture of A-8 (30 mg, 126.98 μmol), 21-3 (55.98 mg, 139.67 μmol) and HATU (53.11 mg, 139.67 μmol) in DMF (5 mL) was added DIPEA (49.23 mg, 380.93 μmol, 66.35 L) at 25° C. The reaction solution was stirred for 20 hrs at 25° C. Then, to the solution was added H₂O (40 mL) and extracted with EA (30 mL×2). The combined organic layers were washed with brine (30 mL), dried over Na₂SO₄, and concentrated to give a residue, which was purified by silica gel chromatography (PE/EA=1/3) to give 21-4 (50 mg, 80.77 μmol, 63.6% yield). MS: m/z=619.3 (M+1).

### Step 4: 21-5

A mixture of 21-4 (50 mg, 80.77 μmol) in CH₃COOH (10 mL) was stirred at 100° C. for 1 hr. Then, the mixture was concentrated, neutralized with saturated NaHCO₃ until the pH reached ˜8 and extracted with EA (30 mL×2). The combined organic layers were washed with brine (30 mL), dried over Na₂SO₄, and concentrated to give 21-5 (30 mg, crude). MS: m/z=601.3 (M+1).

### Step 5: Compound 21

To a mixture of 21-5 (30 mg, 49.91 μmol) in THF (4 mL), Methanol (4 mL) and Water (1 mL) was added NaOH (7.99 mg, 199.66 μmol) at 25° C. The reaction solution was stirred at 30° C. for 18 hrs. Then, the solution was concentrated, charged with H₂O (10 mL), and acidified with 6 M HCl until pH reached 4˜5. Then, the mixture was concentrated to give a residue, which was purified by prep-HPLC (column: XBridge@ Prep C18, 5 μm, 19×150 mm; A: 0.2% HCO₂H water, B: acetonitrile; gradient: 5-95% B; GT: 15 min; flow rate: 15 mL/min) to give Compound 21 (20 mg, 31.59 μmol, 63.3% yield, HCO₂H salt). ¹H NMR (400 MHz, DMSO-d₆) δ 8.88 (s, 2H), 8.20 (s, 1H), 7.78-7.61 (m, 4H), 7.59-7.43 (m, 3H), 7.28 (d, J=8.4 Hz, 1H), 7.00 (dd, J=7.8, 1.4 Hz, 1H), 6.92 (t, J=7.9 Hz, 2H), 5.03 (s, 1H), 4.73-4.68 (m, 1H), 4.65-4.59 (m, 1H), 4.49-4.45 (m, 3H), 4.39-4.34 (m, 1H), 2.64-2.60 (m, 1H), 2.26-2.22 (m, 1H), 2.05 (s, 3H); MS: m/z=587.2 (M+1).

The compound in Table J below was made according to the procedure of Compound 21.

TABLE J

| Name | Structure | ¹H NMR and/or LC/MS data |
|---|---|---|
| 25 | 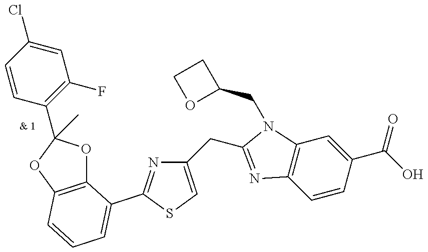 [SMILES: CC1(c2ccc(Cl)cc2F)Oc2cccc(-c3nc(Cc4nc5ccc(C(=O)O)cc5n4C[C@@H]4CCO4)cs3)c2O1] | 1H NMR (400 MHz, DMSO-d6) δ 8.25 (s, 1H), 7.79 (d, J = 8.5 Hz, 1H), 7.66 (s, 1H), 7.59 (d, J = 8.5 Hz, 1H), 7.56-7.44 (m, 3H), 7.24 (d, J = 8.5 Hz, 1H), 7.00 (d, J = 7.7 Hz, 1H), 6.91 (t, J = 7.9 Hz, 1H), 5.01-4.98 (m, 1H), 4.84-4.78 (m, 1H), 4.71-4.60 (m, 2H), 4.60- 4.55 (m, 1H), 4.42-4.38 (m, 1H), 4.32-4.28 (m, 1H), 2.65-2.50 (m, 1H), 2.32-2.28 (m, 1H), 2.07 (s, 3H); MS: m/z = 592.2 (M + 1). |

### Example 5: Compound 22

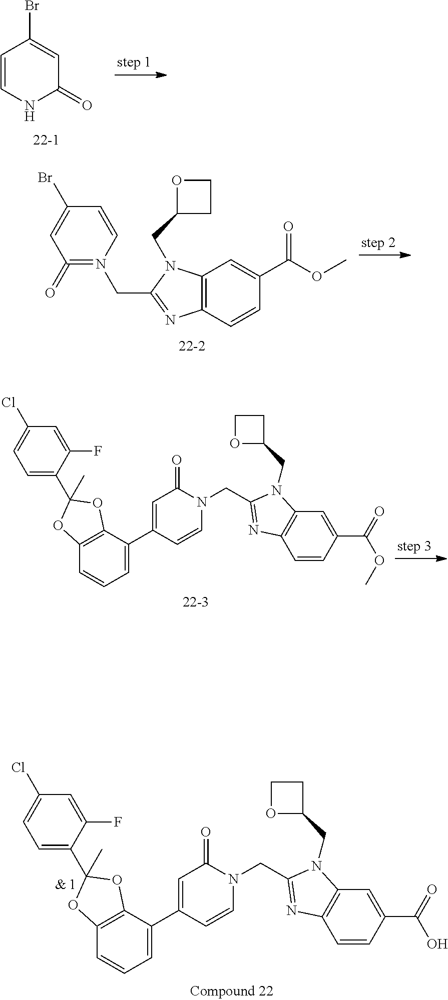

### Step 1: 22-2

A solution of Intermediate A (100 mg, 339.29 μmol), 22-1 (59.03 mg, 339.29 μmol) and Cs₂CO₃ (221.09 mg, 678.58 μmol) in DMF (5 mL) was stirred at 60° C. for 1 hr. The resulting solution was cooled to room temperature and concentrated to give a residue, which was purified by FCC (silica gel, PE/EA=5/1 to 1/2) to give 22-2 (140 mg, 323.87 μmol, 95.5% yield). MS: m/z=432.1 (M+1).

### Step 2: 22-3

A mixture of 22-2 (50 mg, 115.67 μmol), Intermediate B (67.78 mg, 173.50 μmol), cyclopentyl(diphenyl)phosphane;dichloromethane;dichloropalladium;iron (4.72 mg, 5.78 mol) and K₂CO₃ (31.97 mg, 231.34 μmol) in 1,4-dioxane (4 mL) and water (1 mL) was stirred at 120° C. for 40 minutes under microwave. The resulting mixture was cooled to room temperature and filtered. The filtrate was concentrated and purified by FCC (silica gel, PE/EA=3/1) to give 22-3 (30 mg, 48.70 μmol, 42.1% yield). MS: m/z=616.3 (M+1).

### Step 3: Compound 22

A mixture of 22-3 (30 mg, 48.70 μmol) and lithium hydroxide monohydrate (6.13 mg, 146.10 μmol) in THF (5 mL), methanol (5 mL) and water (5 mL) was stirred at 25° C. overnight. The resulting solution was acidified with 1 M HCl to pH=5-6 and extracted with EA (30 mL). The separated organic layer was washed with brine (30 mL), dried over Na₂SO₄, filtered and concentrated to give a residue, which was purified by prep-HPLC (column: XBridge@ Prep C18, 5 μm, 19×150 mm; gradient: 5-95% B, A: 0.2% HCOOH, B: MeCN, GT: 22 min, flow rate: 15 mL/min) to give Compound 22 (9.6 mg, 15.95 μmol, 32.7% yield). ¹H NMR (400 MHz, DMSO-d₆) δ 8.29 (s, 1H), 7.96 (d, J=7.2 Hz, 1H), 7.81 (dd, J=8.4, 1.5 Hz, 1H), 7.66-7.56 (m, 3H), 7.38 (dd, J=8.4, 2.1 Hz, 1H), 7.25 (dd, J=8.2, 1.2 Hz, 1H), 7.09 (dd, J=7.8, 1.1 Hz, 1H), 7.00 (t, J=7.9 Hz, 1H), 6.85 (d, J=2.0 Hz, 1H), 6.74 (dd, J=7.2, 2.1 Hz, 1H), 5.57-5.54 (m, 1H), 5.45-5.41 (m, 1H), 5.12-5.06 (m, 1H), 4.87-4.84 (m, 1H), 4.75-4.73 (m, 1H), 4.51-4.48 (m, 1H), 4.38-4.35 (m, 1H), 2.75-2.71 (m, 1H), 2.40-2.36 (m, 1H), 2.10 (s, 3H); MS: m/z=602.2 (M+1).

### Example 6: Compound 23

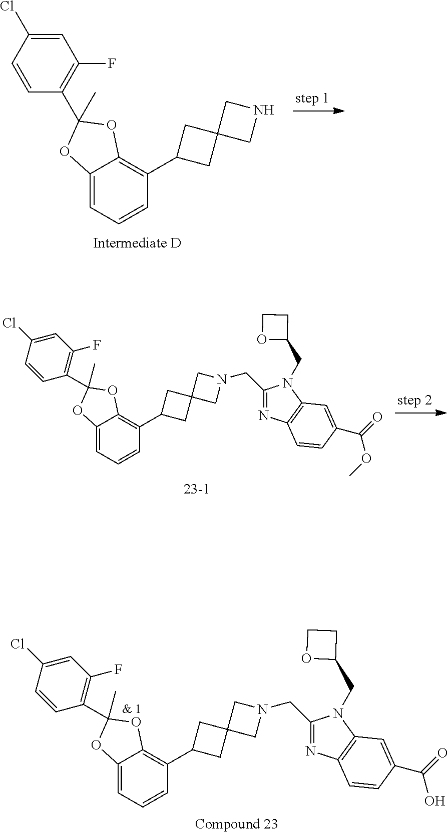 [SMILES: CC1(c2ccc(Cl)cc2F)Oc2cccc(C3CC4(CNC4)C3)c2O1 ; COC(=O)c1ccc2nc(CN3CC4(CC(c5cccc6c5OC(C)(c5ccc(Cl)cc5F)O6)C4)C3)n(C[C@@H]3CCO3)c2c1 ; CC1(c2ccc(Cl)cc2F)Oc2cccc(C3CC4(C3)CN(Cc3nc5ccc(C(=O)O)cc5n3C[C@@H]3CCO3)C4)c2O1]

### Step 1: 23-1

To a mixture of Intermediate A (87.37 mg, 296.44 μmol) and the crude Intermediate D (160 mg) in CH₃CN (5 mL) was added DIPEA (153.25 mg, 1.19 mmol, 206.54 L), the mixture was stirred for 18 hrs at 80° C. Then, the solution was concentrated with a rotary evaporator. The residue was charged with H₂O (40 mL) and extracted with EA (40 mL×2). The combined organic layers were washed with brine (50 mL), dried over Na₂SO₄, and concentrated to give a residue, which was purified by prep-HPLC (column: XBridge@ Prep C18, 5 μm, 19×150 mm; A: 0.2% HCO₂H water, B: acetonitrile; gradient: 5-95% B; GT: 18 min; flow rate: 15 mL/min) to give 23-1 (20 mg). MS: m/z=618.3 (M+1).

### Step 2: Compound 23

To a mixture of 23-1 (20 mg) in THF (4 mL), Methanol (4 mL) and Water (1 mL) was added NaOH (6.47 mg, 161.79 μmol) at 25° C. The reaction solution was stirred at 30° C. for 18 hrs. Then, the solution was concentrated with a rotary evaporator. The residue was charged with H₂O (10 mL) and acidified with 6 M HCl until pH reached 4-5. Then, the mixture was concentrated to give a residue, which was purified by prep-HPLC (column: XBridge@ Prep C18, 5 μm, 19×150 mm; A: 0.2% HCO₂H water, B: acetonitrile; gradient: 5-95% B; GT: 17 min; flow rate: 15 mL/min) to give Compound 23 (3.1 mg, 5.13 μmol). ¹H NMR (400 MHz, DMSO-d₆) δ 8.33 (s, 1H), 8.22 (s, 1H), 7.80 (d, J=8.6 Hz, 1H), 7.64-7.53 (m, 3H), 7.34 (d, J=9.1 Hz, 1H), 6.78 (d, J=4.5 Hz, 1H), 6.72 (d, J=4.4 Hz, 1H), 5.09-5.06 (m, 1H), 4.70-4.68 (m, 1H), 4.48-4.45 (m, 1H), 4.28-4.25 (m, 1H), 3.92-3.89 (m, 1H), 3.84-3.82 (m, 1H), 3.21-3.18 (m, 4H), 2.69-2.66 (m, 1H), 2.28 (s, 2H), 2.26-2.24 (m, 1H), 2.01 (s, 5H), 1.24 (s, 2H); MS: m/z=604.2 (M+1).

The compounds in Table K below were made according to the procedure of Compound 23.

TABLE K

| Name | Structure | ¹H NMR and/or LC/MS data |
|---|---|---|
| 27 | 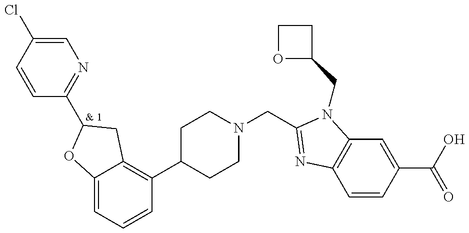 [SMILES: O=C(O)c1ccc2nc(CN3CCC(c4cccc5c4CC(c4ccc(Cl)cn4)O5)CC3)n(C[C@@H]3CCO3)c2c1] | ¹H NMR (400 MHz, MeOD) δ 8.49-8.43 (m, 1H), 8.30 (s, 1H), 7.96 (s, 1H), 7.80-7.76 (m, 1H), 7.65-7.64 (m, 1H), 7.22-7.10 (m, 2H), 6.76-6.69 (m, 2H), 5.23- 5.22 (m, 1H), 4.70-4.52 (m, 6H), 4.45-4.42 (m, 1H), 4.01-3.85 (m, 2H), 3.12-2.94 (m, 2H), 2.84- 2.74 (m, 2H), 2.50-2.45 (m, 1H), 2.37-2.34 (m, 1H), 2.27-2.19 (m, 1H), 1.97-1.90 (m, 1H), 1.88- 1.84 (m, 1H), 1.57-1.51 (m, 1H), 0.88-0.82 (m, 1H); MS: m/z = 559.1 (M + H). |

### Example 7: Compound 24

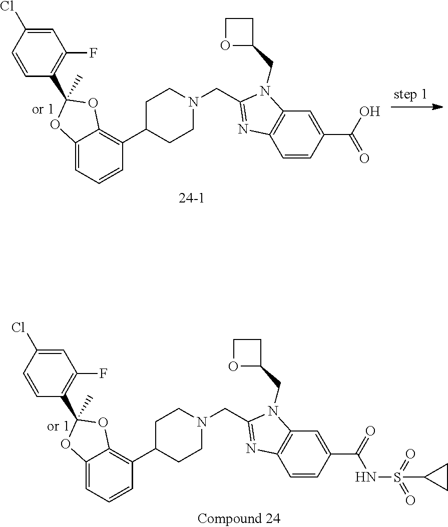 [SMILES: C[C@@]1(c2ccc(Cl)cc2F)Oc2cccc(C3CCN(Cc4nc5ccc(C(=O)O)cc5n4C[C@@H]4CCO4)CC3)c2O1 ; C[C@@]1(c2ccc(Cl)cc2F)Oc2cccc(C3CCN(Cc4nc5ccc(C(=O)NS(=O)(=O)C6CC6)cc5n4C[C@@H]4CCO4)CC3)c2O1]

### Step 1: Compound 24

To a mixture of 24-1 (20 mg, 33.78 μmol), cyclopropanesulfonamide (20.46 mg, 168.90 μmol) in DMF (1 mL) was added DIPEA (13.10 mg, 101.34 μmol) and EDC (9.71 mg, 50.67 μmol) at 30° C. The reaction solution was stirred for 16 hrs at 30° C. Then, EA (100 mL) was added, and the mixture was washed with H₂O (100 mL×3), dried over Na₂SO₄, filtered and concentrated to give a residue, which was purified by silica gel chromatography (PE/EA=5/1 to 1/2) to give Compound 24 (4 mg, 5.75 μmol, 17.0% yield). ¹H NMR (400 MHz, DMSO-d6) δ 7.52-7.46 (m, 1H), 7.26-7.21 (m, 6H), 6.97-6.93 (m, 2H), 6.78-6.73 (m, 1H), 5.57-5.54 (m, 1H), 5.45-5.41 (m, 1H), 5.12-5.06 (m, 1H), 4.87-4.84 (m, 1H), 4.75-4.73 (m, 1H), 4.51-4.48 (m, 1H), 4.38-4.35 (m, 1H), 2.49-2.44 (m, 2H), 2.33-2.26 (m, 1H), 2.17 (t, J=7.3 Hz, 1H), 2.06-1.99 (m, 2H), 1.99-1.94 (m, 1H), 1.69 (d, J=21.8 Hz, 2H), 1.48 (s, 3H), 1.19 (s, 2H), 1.14 (s, 1H), 0.86 (t, J=6.6 Hz, 4H); MS: m/z=650.2 (M+H).

### Example 8: Compound 26

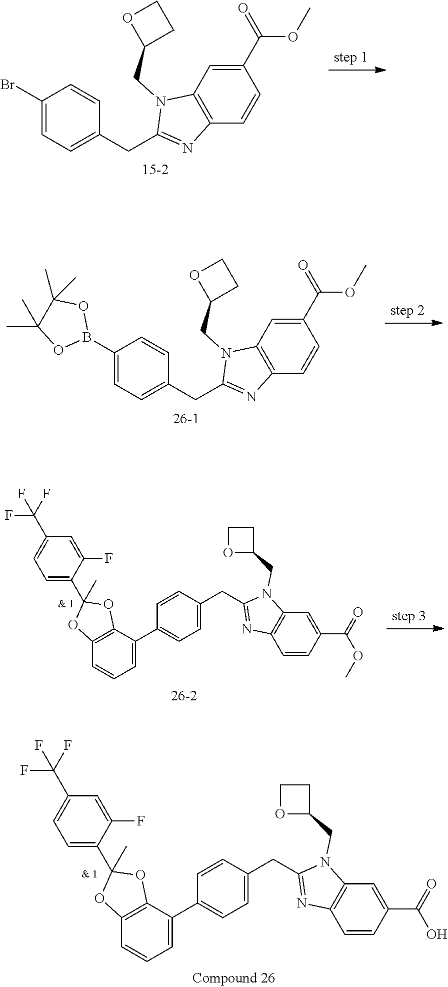

### Step 1: 26-1

A mixture of 15-2 (600 mg, 1.44 mmol), 4,4,5,5-tetramethyl-2-(4,4,5,5-tetramethyl-1,3,2-dioxaborolan-2-yl)-1,3,2-dioxaborolane (403.58 mg, 1.59 mmol), cyclopentyl(diphenyl)phosphane;dichloropalladium;iron (52.86 mg, 72.24 μmol) and KOAc (283.59 mg, 2.89 mmol) in dioxane (5 mL) was stirred at 100° C. for 40 minutes under microwave. The resulting mixture was cooled to room temperature and filtered. The filtrate was concentrated to give 26-1 (660 mg, crude). MS: m/z=463.3 (M+1).

### Step 2: 26-2

A mixture of 26-1 (147.12 mg, 318.19 μmol), Intermediate C (80 mg, 212.13 μmol), cyclopentyl(diphenyl)phosphane;dichloromethane;dichloropalladium;iron (8.66 mg, 10.61 mol) and K₂CO₃ (58.63 mg, 424.26 μmol) in 1,4-dioxane (4 mL) and water (1 mL) was stirred at 120° C. for 40 minutes under microwave. The resulting mixture was cooled to room temperature and filtered. The filtrate was concentrated and purified by FCC (silica gel, PE/EA=5/1) to give 26-2 (100 mg, 158.08 μmol, 74.5% yield). MS: m/z=633.3 (M+1).

### Step 3: Compound 26

A mixture of 26-2 (70 mg, 110.65 μmol) and lithium hydroxide monohydrate (13.93 mg, 331.96 μmol) in THF (5 mL), Methanol (5 mL) and Water (5 mL) was stirred at 25° C. for 6 hrs. The resulting solution was acidified with 1 M HCl to pH=5-6. The resulting mixture was filtered to give Compound 26 (30 mg, 48.50 μmol, 43.8% yield). ¹H NMR (400 MHz, DMSO-d₆) δ 8.22 (s, 1H), 7.83 (dd, J=12.0, 7.1 Hz, 3H), 7.73 (d, J=7.9 Hz, 2H), 7.67 (d, J=8.2 Hz, 1H), 7.60 (d, J=8.4 Hz, 1H), 7.43 (d, J=8.0 Hz, 2H), 7.15 (dd, J=6.9, 2.4 Hz, 1H), 6.98 (d, J=7.0 Hz, 2H), 4.98-4.94 (m, 1H), 4.68-4.63 (m, 1H), 4.57-4.42 (m, 4H), 4.39-4.33 (m, 1H), 2.70-2.62 (m, 1H), 2.39-2.31 (m, 1H), 2.11 (s, 3H); MS: m/z=619.3 (M+1).

The compounds in Table L below were made according to the procedure of Compound 26.

TABLE L

| Name | Structure | ¹H NMR and/or LC/MS data |
|---|---|---|
| Mixture of 40 and 41 | 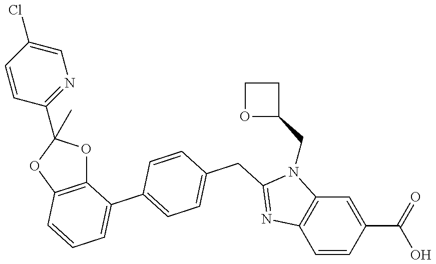 [SMILES: CC1(c2ccc(Cl)cn2)Oc2cccc(-c3ccc(Cc4nc5ccc(C(=O)O)cc5n4C[C@@H]4CCO4)cc3)c2O1] | ¹H NMR (400 MHz, DMSO-d₆) δ 8.73 (d, J = 2.5 Hz, 1H), 8.19 (d, J = 1.5 Hz, 1H), 8.01 (dd, J = 8.5, 2.5 Hz, 1H), 7.80 (dd, J = 8.4, 1.5 Hz, 1H), 7.68 (dd, J = 16.0, 8.4 Hz, 3H), 7.59 (d, J = 8.4 Hz, 1H), 7.44-7.37 (m, 2H), 7.13 (dd, J = 7.0, 2.3 Hz, 1H), 7.01-6.90 (m, 2H), 4.97-4.93 (m, 1H), 4.68-4.62 (m, 1H), 4.55-4.38 (m, 4H), 4.38-4.32 (m, 1H), 2.67-2.61 (m, 1H), 2.38-2.33 (m, 1H), 2.06 (s, 3H); MS: m/z = 568.2 (M + 1). |
| 42 | 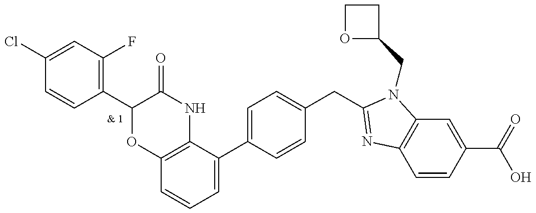 [SMILES: O=C(O)c1ccc2nc(Cc3ccc(-c4cccc5c4NC(=O)C(c4ccc(Cl)cc4F)O5)cc3)n(C[C@@H]3CCO3)c2c1] | ¹H NMR (400 MHz, DMSO-d₆) δ 10.08 (s, 1H), 8.22 (s, 1H), 7.82 (d, J = 8.4 Hz, 1H), 7.62 (d, J = 8.4 Hz, 1H), 7.53 (t, J = 8.2 Hz, 2H), 7.48-7.40 (m, 4H), 7.37 (dd, J = 8.3, 2.0 Hz, 1H), 7.07-6.95 (m, 3H), 5.90 (s, 1H), 5.08-4.93 (m, 1H), 4.67 (d, J = 15.6, 1H), 4.59-4.43 (m, 4H), 4.37 (q, J = 6.8, 6.3 Hz, 1H), 2.43-2.30 (m, 2H); MS: m/z = 598.1 (M + 1). |
| 43 | 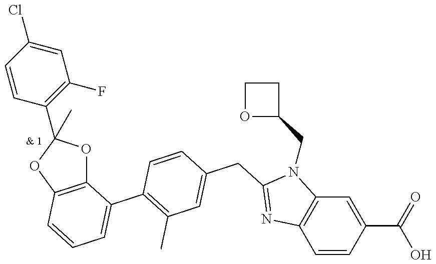 [SMILES: Cc1cc(Cc2nc3ccc(C(=O)O)cc3n2C[C@@H]2CCO2)ccc1-c1cccc2c1OC(C)(c1ccc(Cl)cc1F)O2] | ¹H NMR (400 MHz, DMSO-d₆) δ 8.15 (s, 1H), 7.82-7.68 (m, 1H), 7.56 (d, J = 8.5 Hz, 1H), 7.51-7.42 (m, 2H), 7.27 (d, J = 8.4, 1H), 7.18 (s, 1H), 7.12 (d, J = 2.8 Hz, 2H), 6.92 (d, J = 7.7 Hz, 1H), 6.86 (t, J = 7.8 Hz, 1H), 6.68 (d, J = 7.6 Hz, 1H), 4.88- 4.84 (m, 1H), 4.63-4.59 (m, 1H), 4.49- 4.44 (m, 1H), 4.42-4.36 (m, 1H), 4.33- 4.27 (m, 3H), 2.62-2.51 (m, 1H), 2.32- 2.23 (m, 1H), 2.02 (s, 3H), 1.93 (s, 3H); MS: m/z = 599.1 (M + H). |
| 7 | 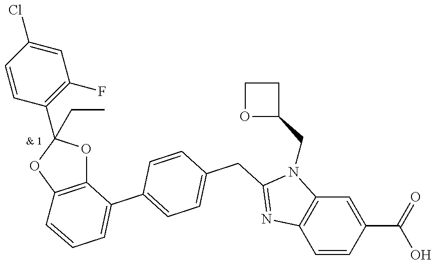 [SMILES: CCC1(c2ccc(Cl)cc2F)Oc2cccc(-c3ccc(Cc4nc5ccc(C(=O)O)cc5n4C[C@@H]4CCO4)cc3)c2O1] | ¹H NMR (400 MHz, DMSO-d₆) δ 8.19 (s, 1H), 7.82 (d, J = 8.3 Hz, 1H), 7.73 (d, J = 8.0 Hz, 2H), 7.59-7.52 (m, 3H), 7.43 (d, J = 8.1 Hz, 2H), 7.34 (dd, J = 8.4, 2.1 Hz, 1H), 7.13 (dd, J = 7.4, 1.9 Hz, 1H), 6.99-6.91 (m, 2H), 4.95 (d, J = 7.6 Hz, 1H), 4.65 (dd, J = 15.5, 7.1 Hz, 1H), 4.55- 4.42 (m, 4H), 4.35 (dd, J = 10.4, 4.9 Hz, 1H), 2.69-2.61 (m, 1H), 2.40-2.33 (m, 3H), 0.95 (t, J = 7.3 Hz, 3H); MS: m/z = 599.3 (M + 1). |

### Example 9: Compound 28 and Compound 29

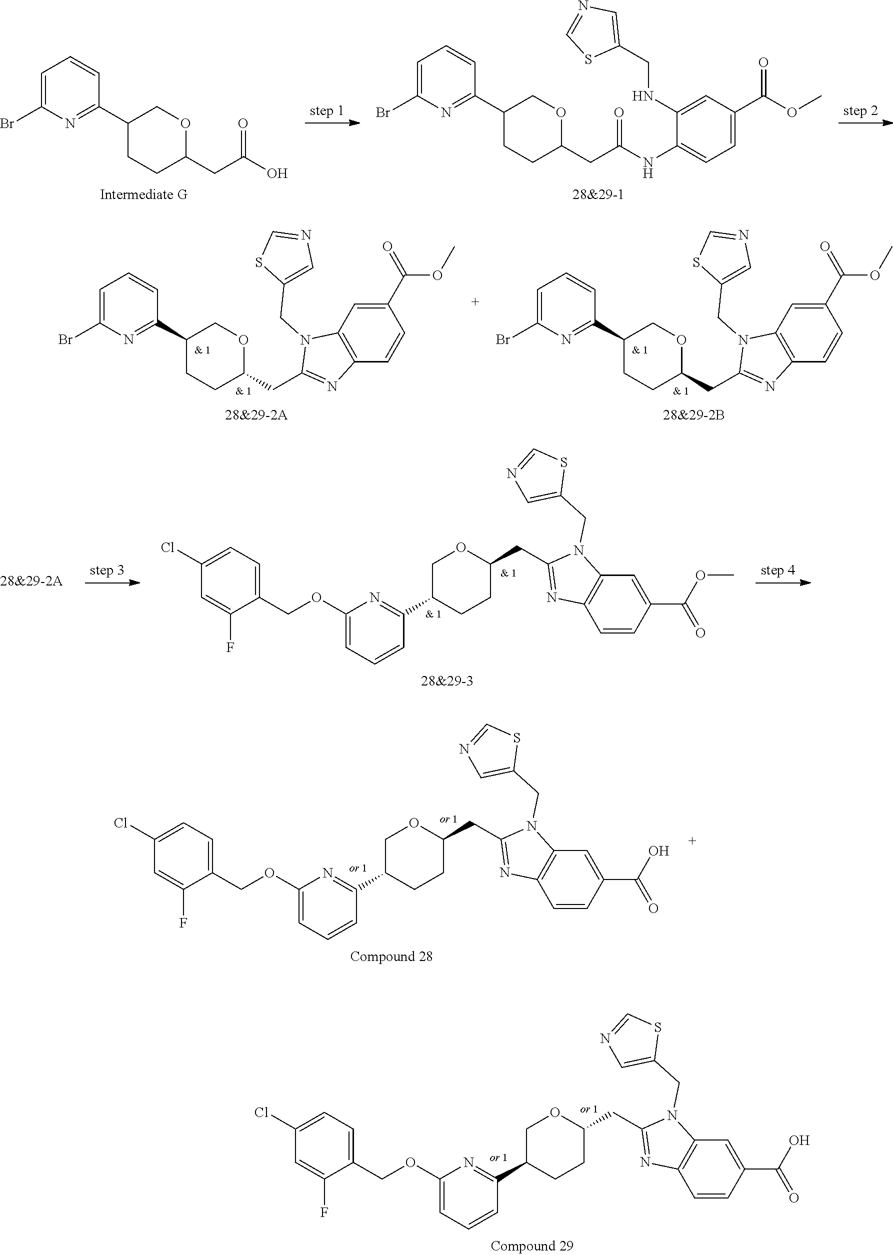

### Step 1: 28&29-1

To a solution of Intermediate G (1.4 g, 4.66 mmol), Intermediate F (1.35 g, 5.13 mmol) and HATU (2.13 g, 5.60 mmol) in DCM (20 mL) was added DIPEA (1.81 g, 13.99 mmol, 2.44 mL). The mixture was stirred at 25° C. overnight, concentrated and purified by FCC (silica gel, PE/EA=10/1 to 1/3) to give 28&29-1 (2.5 g, 4.58 mmol, 98.3% yield). MS: m/z=545.2 (M+1).

### Step 2: 28&29-2A

A solution of 28&29-1 (1.0 g, 1.83 mmol) in AcOH (10 mL) was stirred at 100° C. for hr. The resulting mixture was cooled to room temperature and concentrated. The residue was purified by prep-HPLC (column: XBridge@ Prep C18, 5 μm, 19×150 mm; A: 0.2% HCO₂H water, B: acetonitrile; gradient: 5-50% B) to give 28&29-2A (330 mg, 625.67 μmol, 34.1% yield) and 28&29-2B. MS: m/z=527.2 (M+1).

### Step 3: 28&29-3

A mixture of 28&29-2A (330 mg, 625.67 μmol), (4-chloro-2-fluoro-phenyl)methanol (120.56 mg, 750.81 μmol), CuI (25.05 mg, 131.52 μmol), 3,4,7,8-tetramethyl-1,10-phenanthroline (62.62 mg, 264.99 μmol) and Cs₂CO₃ (413.30 mg, 1.27 mmol) in 1,4-dioxane (3 mL) was stirred at 120° C. in a microwave reactor for 9 hrs. The resulting mixture was cooled to room temperature and filtered. The filtrate was concentrated and purified by FCC (PE/EA=3/1 to 1/2) to give 28&29-3 (140 mg, 230.61 μmol, 36.9% yield). MS: m/z=607.3 (M+1).

### Step 4: Compound 28 & Compound 29

A mixture of 28&29-3 (140 mg, 230.61 μmol) and lithium;hydroxide;hydrate (29.03 mg, 691.82 μmol) in THF (5 mL) and water (5 mL) was stirred at 25° C. overnight. The resulting mixture was acidified with aqueous citric acid to pH=5-6 and extracted with EA (30 mL). The separated organic layer was washed with brine (30 mL), dried over Na₂SO₄, filtered and concentrated to give a residue, which was purified by prep-HPLC (column: XBridge@ Prep C18, 5 μm, 19×150 mm; A: 0.2% HCO₂H water, B: acetonitrile; gradient: 40-95% B; GT: 22 min, flow rate: 15 mL/min) to give a mixture (80 mg, 134.89 μmol, 58.5% yield). ¹H NMR (400 MHz, Methanol-d₄) δ 8.84 (s, 1H), 8.07 (d, J=1.5 Hz, 1H), 7.91-7.82 (m, 2H), 7.57 (d, J=8.5 Hz, 1H), 7.45 (dd, J=8.3, 7.3 Hz, 1H), 7.36 (t, J=8.0 Hz, 1H), 7.08 (ddd, J=12.9, 9.0, 2.1 Hz, 2H), 6.69 (d, J=7.3 Hz, 1H), 6.53 (d, J=8.2 Hz, 1H), 5.82 (s, 2H), 5.27 (s, 2H), 3.85-3.79 (m, 2H), 3.37 (t, J=11.2 Hz, 1H), 3.15 (d, J=6.0 Hz, 2H), 2.78-2.69 (m, 1H), 1.89-1.83 (m, 3H), 1.53-1.48 m, 1H); MS: m/z=593.2 (M+1).

The mixture was further separated by SFC (column: IC, mobile phase: Hex:EtOH:(NH₃/MeOH)=50%/50%/0.2%, flow rate: 1 mL/min, temperature: 30° C., time: 20 min) to give Compound 29 (14 mg, 25.01 μmol, 100% ee) and Compound 28 (12 mg, 20.44 μmol, 96% ee).

The compounds in Table M below were made according to the procedure of Compound 28 and Compound 29.

TABLE M

| Name | Structure | ¹H NMR and/or LC/MS data |
|---|---|---|
| 30 | 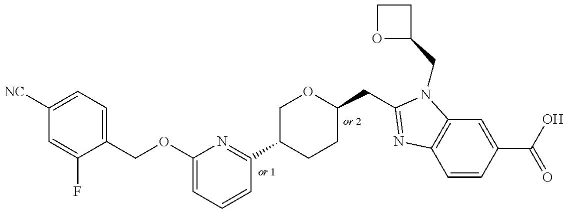 [SMILES: N#Cc1ccc(COc2cccc([C@H]3CC[C@H](Cc4nc5ccc(C(=O)O)cc5n4C[C@@H]4CCO4)OC3)n2)c(F)c1] | ¹H NMR (400 MHz, Methanol-d₄) δ 8.17 (d, J = 1.5 Hz, 1H), 7.86 (dd, J = 8.5, 1.5 Hz, 1H), 7.55 (t, J = 7.7 Hz, 2H), 7.52- 7.40 (m, 3H), 6.73 (d, J = 7.1 Hz, 1H), 6.58 (dd, J = 8.3, 0.8 Hz, 1H), 5.44-5.33 (m, 2H), 5.10-5.05 (m, 1H), 4.72-4.68 (m, 1H), 4.55-4.51 (m, 1H), 4.49-4.35 (m, 2H), 3.90-3.78 (m, 2H), 3.36 (t, J = 11.2 Hz, 1H), 3.10-3.05 (m, 2H), 2.80- 2.65 (m, 2H), 2.47-2.35 (m, 1H), 1.94- 1.75 (m, 3H), 1.57-1.48 (m, 1H); MS: m/z = 557.3 (M + 1). SFC condition for 30 and 31: Column: |
| | | Daicel ChiralPak AD-H, Column size |
| | | 250 mm × 30 mm I.D., 5 μm; Mobile |
| | | phase: CO₂:MeOH (0.1% DEA) = 60:40, |
| | | Flow rate: 50 g/min; Wavelength: 254 |
| | | nm, Temperature: 40° C. |
| 31 | 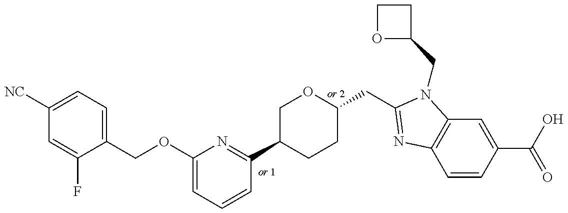 [SMILES: N#Cc1ccc(COc2cccc([C@@H]3CC[C@@H](Cc4nc5ccc(C(=O)O)cc5n4C[C@@H]4CCO4)OC3)n2)c(F)c1] | ¹H NMR (400 MHz, Methanol-d₄) δ 8.17 (d, J = 1.5 Hz, 1H), 7.86 (dd, J = 8.5, 1.5 Hz, 1H), 7.55 (t, J = 7.7 Hz, 2H), 7.52- 7.40 (m, 3H), 6.73 (d, J = 7.1 Hz, 1H), 6.58 (dd, J = 8.3, 0.8 Hz, 1H), 5.44-5.33 (m, 2H), 5.10-5.05 (m, 1H), 4.72-4.68 (m, 1H), 4.55-4.51 (m, 1H), 4.49-4.35 (m, 2H), 3.90-3.78 (m, 2H), 3.36 (t, J = 11.2 Hz, 1H), 3.10-3.05 (m, 2H), 2.80- 2.65 (m, 2H), 2.47-2.35 (m, 1H), 1.94- 1.75 (m, 3H), 1.57-1.48 (m, 1H); MS: m/z = 557.3 (M + 1). |
| 32 | 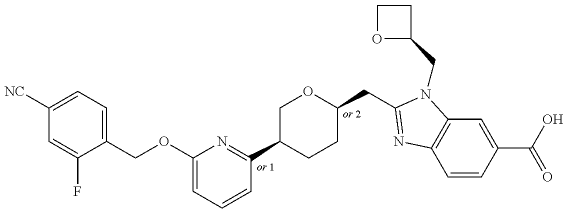 [SMILES: N#Cc1ccc(COc2cccc([C@@H]3CC[C@H](Cc4nc5ccc(C(=O)O)cc5n4C[C@@H]4CCO4)OC3)n2)c(F)c1] | ¹H NMR (400 MHz, Methanol-d₄) δ 8.16 (d, J = 1.5 Hz, 1H), 7.86 (dd, J = 8.5, 1.5 Hz, 1H), 7.59-7.39 (m, 5H), 6.73 (d, J = 7.2 Hz, 1H), 6.58 (dd, J = 8.3, 0.7 Hz, 1H), 5.38 (d, J = 1.1 Hz, 2H), 5.10-5.06 (m, 1H), 4.71-4.66 (m, 1H), 4.55-4.34 (m, 3H), 3.90-3.78 (m, 2H), 3.36 (t, J = 11.2 Hz, 1H), 3.19 (d, J = 8.1 Hz, 1H), 3.10-3.06 (m, 1H), 2.82-2.66 (m, 2H), 2.47-2.38 (m, 1H), 1.94-1.74 (m, 3H), 1.58-1.46 (m, 1H); MS: m/z = 557.3 (M + 1). SFC condition for 32 and 33: Column: |
| | | Daicel ChiralPak AD-H, Column size |
| | | 250 mm × 30 mm I.D., 5 μm; Mobile |
| | | phase: CO₂:MeOH (0.1% DEA) = 60:40, |
| | | Flow rate: 50 g/min; Wavelength: 254 |
| | | nm, Temperature: 40° C. |
| 33 | 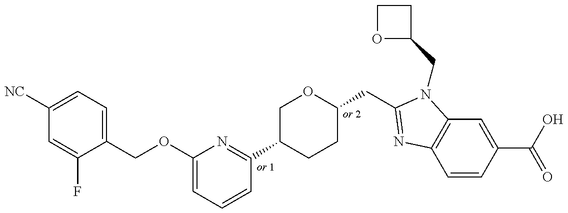 [SMILES: N#Cc1ccc(COc2cccc([C@H]3CC[C@@H](Cc4nc5ccc(C(=O)O)cc5n4C[C@@H]4CCO4)OC3)n2)c(F)c1] | ¹H NMR (400 MHz, Methanol-d₄) δ 8.16 (d, J = 1.5 Hz, 1H), 7.86 (dd, J = 8.5, 1.5 Hz, 1H), 7.59-7.39 (m, 5H), 6.73 (d, J = 7.2 Hz, 1H), 6.58 (dd, J = 8.3, 0.7 Hz, 1H), 5.38 (d, J = 1.1 Hz, 2H), 5.10-5.06 (m, 1H), 4.71-4.66 (m, 1H), 4.55-4.34 (m, 3H), 3.90-3.78 (m, 2H), 3.36 (t, J = 11.2 Hz, 1H), 3.19 (d, J = 8.1 Hz, 1H), 3.10-3.06 (m, 1H), 2.82-2.66 (m, 2H), 2.47-2.38 (m, 1H), 1.94-1.74 (m, 3H), 1.58-1.46 (m, 1H); MS: m/z = 557.3 (M + H). |

### Example 10: Compound 34

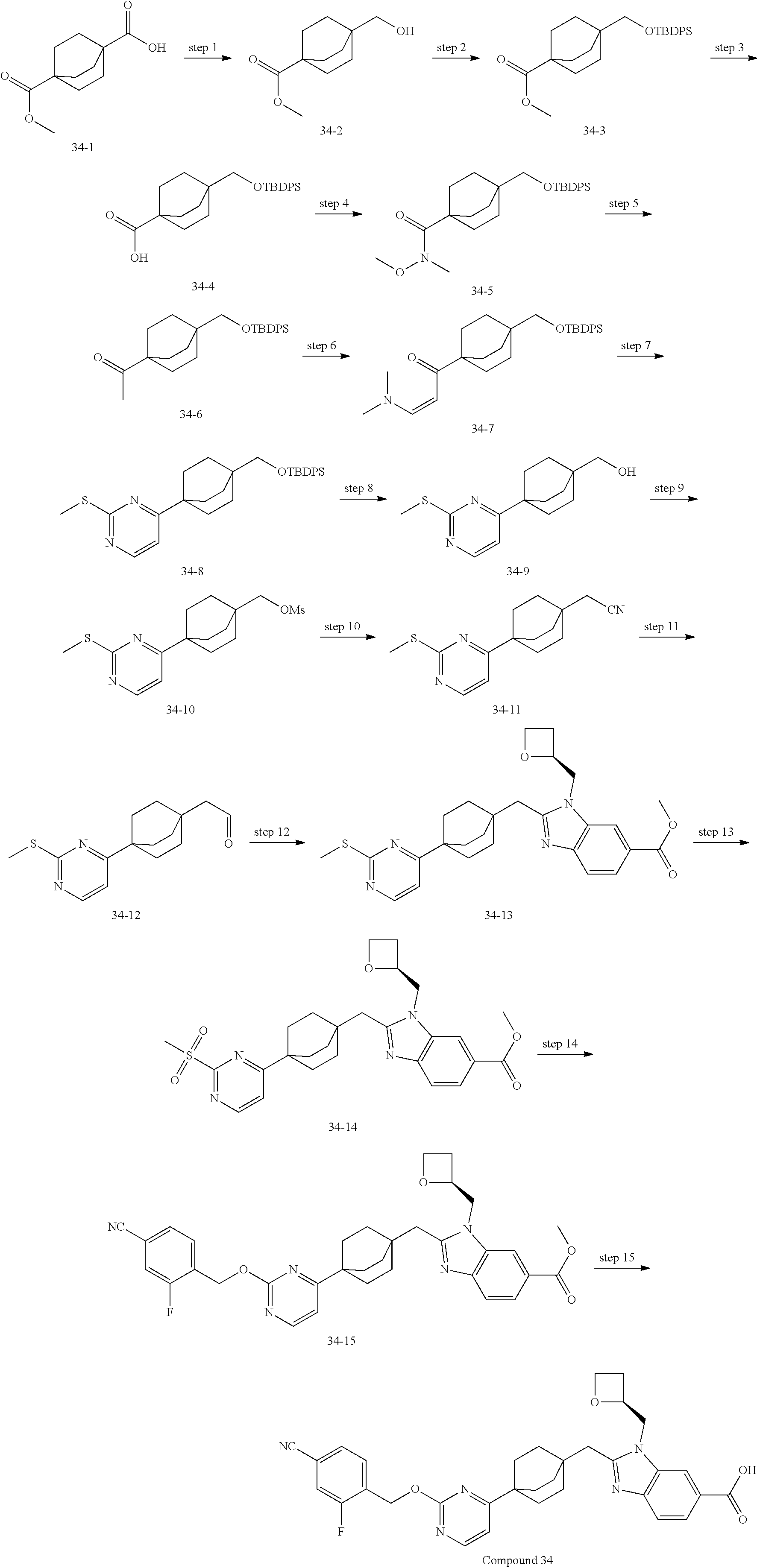

### Step 1: 34-2

To a solution of 34-1 (10 g, 47.12 mmol) in THF (300 mL) was added Borane-tetrahydrofuran complex (1 M, 70.67 mL) at 0° C. dropwise. After addition, the mixture was stirred at 25° C. for 2 hrs. The resulting mixture was quenched with MeOH (100 mL) and concentrated to give 34-2 (9.3 g, crude). MS: m/z=199.1 (M+1).

### Step 2: 34-3

A mixture of 34-2 (9 g, 45.40 mmol), tert-butyl-chloro-diphenyl-silane (18.72 g, 68.09 mmol, 17.49 mL), and Imidazole (9.27 g, 136.19 mmol) in DCM (200 mL) was stirred for 2 hrs. The resulting mixture was washed with H₂O (200 mL). The separated organic layer was concentrated to give 34-3 (19 g, crude). MS: m/z=437.3 (M+1).

### Step 3: 34-4

A mixture of 34-3 (19 g, 43.51 mmol) and lithium;hydroxide;hydrate (5.48 g, 130.54 mmol) in THF (50 mL), Water (50 mL) and Methanol (50 mL) was stirred at 50° C. for 3 hrs. The resulting mixture was concentrated and re-dissolved in EA (100 mL), which was then acidified with 1 M HCl to pH=4-5 and extracted with EA (200 mL). The separated organic layer was washed with brine (100 mL), dried over Na₂SO₄, filtered, and concentrated to give 34-4 (14.0 g, 33.13 mmol, 76.1% yield). MS: m/z=445.2 (M+23).

### Step 4: 34-5

To a solution of 34-4 (14.0 g, 33.13 mmol), N-methoxymethanamine (4.85 g, 49.69 mmol, HCl salt) and HATU (13.85 g, 36.44 mmol) in DMF (150 mL) was added DIPEA (12.84 g, 99.38 mmol, 17.31 mL), and the mixture was stirred for 2 hrs. The resulting mixture was diluted with water (300 mL) and extracted with EA (300 mL). The separated organic layer was washed with water (300 mL×3) and brine (50 mL×3), dried over Na₂SO₄, filtered, and concentrated to give 34-5 (15 g, crude). MS: m/z=466.3 (M+1).

### Step 5: 34-6

To a solution of 34-5 (15 g, 32.21 mmol) in THF (200 mL) was added methylmagnesium bromide (3 M, 53.68 mL) at 0° C. After addition, the mixture was stirred at 25° C. for 2 hrs. The resulting mixture was quenched with saturated aqueous NH₄Cl, washed with 1 M HCl and extracted with EA (300 mL). The separated organic layer was concentrated to give 34-6 (13 g, crude). MS: m/z=421.3 (M+1).

### Step 6: 34-7

A mixture of 34-6 (13 g, 30.90 mmol) and 1-tert-butoxy-N,N,N′,N′-tetramethyl-methanediamine (10.77 g, 61.81 mmol, 12.76 mL) was stirred at 60° C. overnight. The resulting mixture was concentrated and purified by FCC (silica gel, PE/EA=10/1 to 1/1) to give 34-7 (12.9 g, 27.12 mmol, 87.7% yield). MS: m/z=476.3 (M+1).

### Step 7: 34-8

A mixture of 34-7 (12.9 g, 27.12 mmol), 2-methylisothiourea (10.21 g, 54.23 mmol, H₂SO₄ salt) and sodium ethoxide (9.23 g, 135.58 mmol) in Ethanol (100 mL) was stirred at 75° C. overnight. The resulting mixture was concentrated, and the residue was dissolved in water (200 mL) and extracted with EA (200 mL×2). The separated organic layers were concentrated and purified by FCC (silica gel, PE/EA=100/1 to 15/1) to give 34-8 (6.8 g, 13.52 mmol, 49.9% yield). MS: m/z=503.3 (M+1).

### Step 8: 34-9

To a solution of 34-8 (3.5 g, 6.96 mmol) in THF (10 mL) was added TBAF (3.64 g, 13.92 mmol, 4.03 mL), the reaction was stirred at 20° C. for 16 hrs. Solvent was removed to give a residue, which was purified by column chromatography (PE/EA=100/0 to 0/100) to give 34-9 (1.2 g, 4.54 mmol, 65.2% yield). MS: m/z=265.1 (M+1).

### Step 9: 34-10

To a solution of 34-9 (850 mg, 3.22 mmol) in DCM (8 mL) was added DIPEA (1.66 g, 12.86 mmol) and methane sulfonyl chloride (739 mg, 6.43 mmol) dropwise with ice cooling, the reaction was stirred at 0-20° C. for 3 hrs. After most solvent was removed, the residue was diluted with EA (20 mL), the organic layer was washed with 1 M HCl (30 mL×2), dried over Na₂SO₄, and concentrated to give 34-10 (1 g, crude). MS: m/z=343.1 (M+1).

### Step 10: 34-11

To a solution of 34-10 (850 mg, 2.48 mmol) in DMSO (5 mL) was added KCN (1.5 g, 21.69 mmol), the reaction was stirred at 100° C. for 48 hrs. The mixture was poured into 20 mL of water and extracted with EA (20 mL×3), the combined organic layers were dried over Na₂SO₄ and concentrated to give a residue, which was purified by column (PE/EA=100/0 to 100/60) to give 34-11 (600 mg, 2.19 mmol, 88.4% yield). MS: m/z=274.1 (M+1).

### Step 11: 34-12

To a solution of 34-11 (200 mg, 731.54 μmol) in THF (5 mL) was added diisobutylaluminum hydride (2.9 mL, 1 M in hexane) at 20° C., the reaction was stirred for 30 minutes. The reaction was quenched by NH₄Cl solution, then extracted with EA (20 mL×3). The combined organic layers were dried over Na₂SO₄ and concentrated to give 34-12 (150 mg, crude). MS: m/z=274.2 (M+1).

### Step 12: 34-13

To a solution of 34-12 (150 mg, 542.70 μmol) in MeOH (3 mL) was added A-8 (128 mg, 542.70 μmol) and AcOH (3 mL) at 20° C., the reaction was stirred at 20° C. for 1 hr. Solvent was removed to give a residue, which was purified by column chromatography (PE/EA=100/0 to 100/40) to give 34-13 (70 mg, 142.09 μmol, 26.2% yield). MS: m/z=493.1 (M+1).

### Step 13: 34-14

To a solution of 34-13 (70 mg, 142.09 μmol) in DCM (3 mL) was added 3-chlorobenzenecarboperoxoic acid (74 mg, 426.28 μmol) in portions at 20° C., the reaction was stirred for 1 hr at 20° C. The mixture was concentrated and diluted with 20 mL of EA, the organic layer was washed with 1 M NaOH (10 mL×3) and brine (10 mL×3), dried over Na₂SO₄ and concentrated to give 34-14 (50 mg crude). MS: m/z=525.2 (M+1).

### Step 14: 34-15

To a solution of 3-fluoro-4-(hydroxymethyl)benzonitrile (14 mg, 95.31 μmol) in THE (5 mL) was treated with sodium hydride (11 mg, 60% purity), to which, a solution of 34-14 (50 mg, 95.31 μmol) in THF (5 mL) was added at 20° C., the reaction was stirred for 1 hr. The reaction was quenched with saturated NH₄Cl solution and extracted with EA (10 mL×3), the combined organic layers were dried over Na₂SO₄ and concentrated to give 34-15 (50 mg, 83.94 mol, 88.1% yield). MS: m/z=596.2 (M+1).

### Step 15: Compound 34

To a solution of 34-15 (50 mg, 83.94 μmol) in MeOH (3 mL) was added 2 M NaOH (1 mL), the reaction was stirred at 20° C. for 5 hrs. The reaction was treated with 1 M HCl to pH=2 and extracted with EA (10 mL×3), the combined organic layers were dried over Na₂SO₄ and concentrated to give a residue, which was purified by pre-HPLC (column: XBridge@ Prep C18, 5 μm, 19×150 mm; A: 0.1% HCO₂H water, B: acetonitrile; gradient: 35-100% B; GT: 13 min; flow rate: 20 mL/min) to give Compound 34 (9 mg, 15.47 μmol, 18.4% yield). ¹H NMR (400 MHz, CD₃Cl) δ 8.40 (d, J=8.0 Hz, 1H), 8.19 (s, 1H), 8.06 (d, J=8.0 Hz, 1H), 7.14 (d, J=8.0 Hz, 1H), 7.67 (t, J=8.0 Hz, 1H), 7.44 (d, J=8.0 Hz, 1H), 7.37 (d, J=8.0 Hz, 1H), 6.87 (d, J=8.0 Hz, 1H), 5.52 (s, 2H), 5.19-5.17 (m, 1H), 4.65-4.64 (m, 1H), 4.57-4.45 (m, 2H), 4.39-4.34 (m, 1H), 3.00-2.95 (m, 2H), 2.76-2.71 (m, 1H), 2.43-2.39 (m, 1H), 1.88-1.74 (m, 12H); MS: m/z=582.1 (M+1).

### Example 11: Compound 35

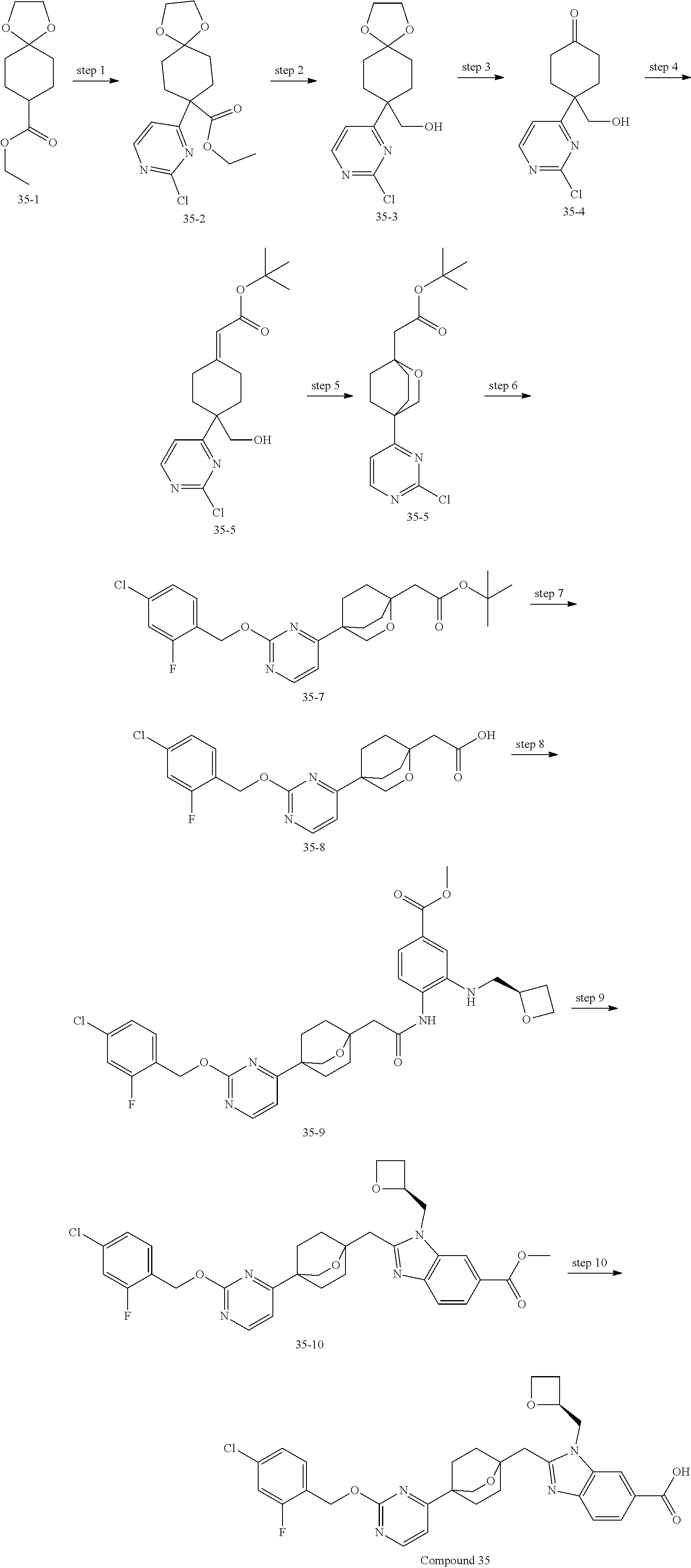

### Step 1: 35-2

To a solution of 35-1 (10.79 g, 50.34 mmol) in THF (150 mL) was added LDA (100 mL, 1 M in hexane) at −30° C. The mixture was stirred at −30° C. for 1 hr, then 2,4-dichloropyrimidine (5 g, 33.56 mmol) in THF (20 mL) was added. The mixture was slowly heated to 60° C. for 2 hrs. The reaction mixture was quenched by saturated NH₄Cl solution and extracted with EA (100 mL×2), dried over Na₂SO₄, and filtered. The filtrate was concentrated to give a residue, which was purified by silica gel chromatography (PE/EA=20/1 to 2/1) to give 35-2 (4.68 g, 43% yield). MS: m/z=327.1 (M+1).

### Step 2: 35-3

To a solution of 35-2 (3.86 g, 11.81 mmol) in THF (30 mL) was added DIBAL-H (35 mL, 1 M in hexane) at 0° C. The mixture was stirred at 25° C. for 16 hrs. The mixture was quenched by H₂O (10 mL), diluted with EA (20 mL). The reaction mixture was extracted with EA (20 mL×2). The combined organic layers were washed with brine (20 mL×2), dried over Na₂SO₄, and filtered. The filtrate was concentrated to give a residue, which was purified by silica gel chromatography (PE/EA=1/1) to give 35-3 (674 mg, 20% yield). MS: m/z=285.1 (M+1).

### Step 3: 35-4

To a solution of 35-3 (674 mg, 2.37 mmol) in THF (10 mL) was added HCl (1 M, 23.67 mL), the mixture was stirred at 25° C. for 2 hrs. The mixture was adjusted to pH=13 with saturated NaHCO₃ solution, then the mixture was diluted with EA (10 mL). The organic layer was washed with brine (10 mL×2), dried over anhydrous Na₂SO₄, filtered and concentrated to give 35-4 (500 mg, 87.8% yield). MS: m/z=241.1 (M+1).

### Step 4: 35-5

To a round bottom flask containing THF (20 mL) was added NaH (100 mg, 60% purity) portion-wise under stirring, tert-butyl 2-dimethoxyphosphorylacetate (558 mg, 2.49 mmol) was added and the resulting mixture was stirred at 25° C. for 30 minutes. To this mixture was added 35-4 (500 mg, 2.08 mmol) in several portions and stirred at 25° C. for 3 hrs. The mixture was then quenched with a saturated solution of ammonium chloride. The mixture was extracted with EA (20 mL×2). The combined organic layers were dried over anhydrous Na₂SO₄ and concentrated to give a residue, which was purified by silica gel chromatography (PE/EA=5/1 to 1/1) to give 35-5 (505 mg, 72% yield). MS: m/z=361.1 (M+23).

### Step 5: 35-6

To a solution of 35-5 (500 mg, 1.48 mmol) in THF (50 mL) was added NaH (59 mg, 60% purity) and stirred for 4 hrs. The reaction mixture was quenched by a saturated solution of ammonium chloride. The mixture was extracted with EA (20 mL×2), and then combined organic layers were dried over anhydrous Na₂SO₄ and concentrated to give a residue, which was purified by silica gel chromatography (PE/EA=5/1 to 1/1) to give 35-6 (184 mg, 37% yield). MS: m/z=283.1 (M−56+1).

### Step 6: 35-7

To a solution of 35-6 (180 mg, 531.24 μmol), (4-chloro-2-fluoro-phenyl)methanol (85 mg, 531.24 μmol) in dioxane (30 mL) was added Pd(dba)₂ (49 mg, 53.12 μmol), Johnphos (32 mg, 106.25 μmol), and Cs₂CO₃ (520 mg, 1.59 mmol). The mixture was stirred at 95° C. for 16 hrs. The reaction mixture was extracted with EA (50 mL×2), and the combined organic layers were washed with brine (50 mL×2), dried over Na₂SO₄, filtered, and concentrated to give a residue, which was purified by silica gel column (PE/EA=5/1) to give 35-7 (140 mg, 57% yield). MS: m/z=463.2 (M+1).

### Step 7: 35-8

To a solution of 35-7 (140 mg, 302.41 μmol) in DCM (10 mL) was added TFA (2.33 mL) and stirred at 25° C. for 3 hrs. The mixture was concentrated to give 35-8 (100 mg, 81% yield). MS: m/z=407.1 (M+1).

### Step 8: 35-9

To a solution of 35-8 (100 mg, 245.80 μmol), A-8 (58 mg, 245.80 μmol) and HATU (140 mg, 368.70 μmol) in NMP (10 mL) was added DIPEA (95 mg, 737.40 μmol). The reaction was stirred at 25° C. for 16 hrs. The reaction mixture was extracted with EA (50 mL×2), and the combined organic layers were washed with brine (20 mL), dried over Na₂SO₄, and concentrated to give 35-9 (80 mg, 52% yield). MS: m/z=626.1 (M+1).

### Step 9: 35-10

A solution of 35-9 (80 mg, 127.98 μmol) in AcOH (50 mL) was stirred at 60° C. for 16 hrs. The mixture was concentrated and extracted with EA (50 mL×2). The combined organic layers were washed with brine (20 mL), dried over Na₂SO₄, and concentrated to give 35-10 (50 mg, 64% yield). MS: m/z=607.1 (M+1).

### Step 10: Compound 35

To a solution of 35-10 (50 mg, 82.36 μmol) in MeOH (5 mL) was added NaOH (2 M, 4.12 mL). The reaction mixture was stirred at 50° C. for 4 hrs. The mixture was adjusted to pH=7 with 1 M HCl and extracted with EA (50 mL×2). The combined organic layers were washed with brine (20 mL), dried over Na₂SO₄, concentrated to give a residue, which was purified by prep-HPLC (column: XBridge@ Prep C18, 5 μm, 19×150 mm; A: 0.1% HCO₂H water, B: acetonitrile; gradient: 5-95% B) to give Compound 35 (4.8 mg, 10% yield). ¹H NMR (400 MHz, MeOD) δ 8.45 (d, J=5.2 Hz, 1H), 8.31 (s, 1H), 7.97 (d, J=8.0 Hz, 1H), 7.65 (d, J=8.4 Hz, 1H), 7.52-7.48 (m, 1H), 7.24-7.20 (m, 2H), 7.03 (d, J=5.6 Hz, 1H), 5.44 (s, 2H), 5.18 (d, J=5.2 Hz, 1H), 4.73-4.69 (m, 2H), 4.63-4.59 (m, 1H), 4.46-4.40 (m, 1H), 4.07-4.02 (m, 2H), 2.77 (s, 2H), 2.48 (s, 2H), 2.13-1.90 (m, 8H); MS: m/z=593.2 (M+1).

### Example 12: Compound 36

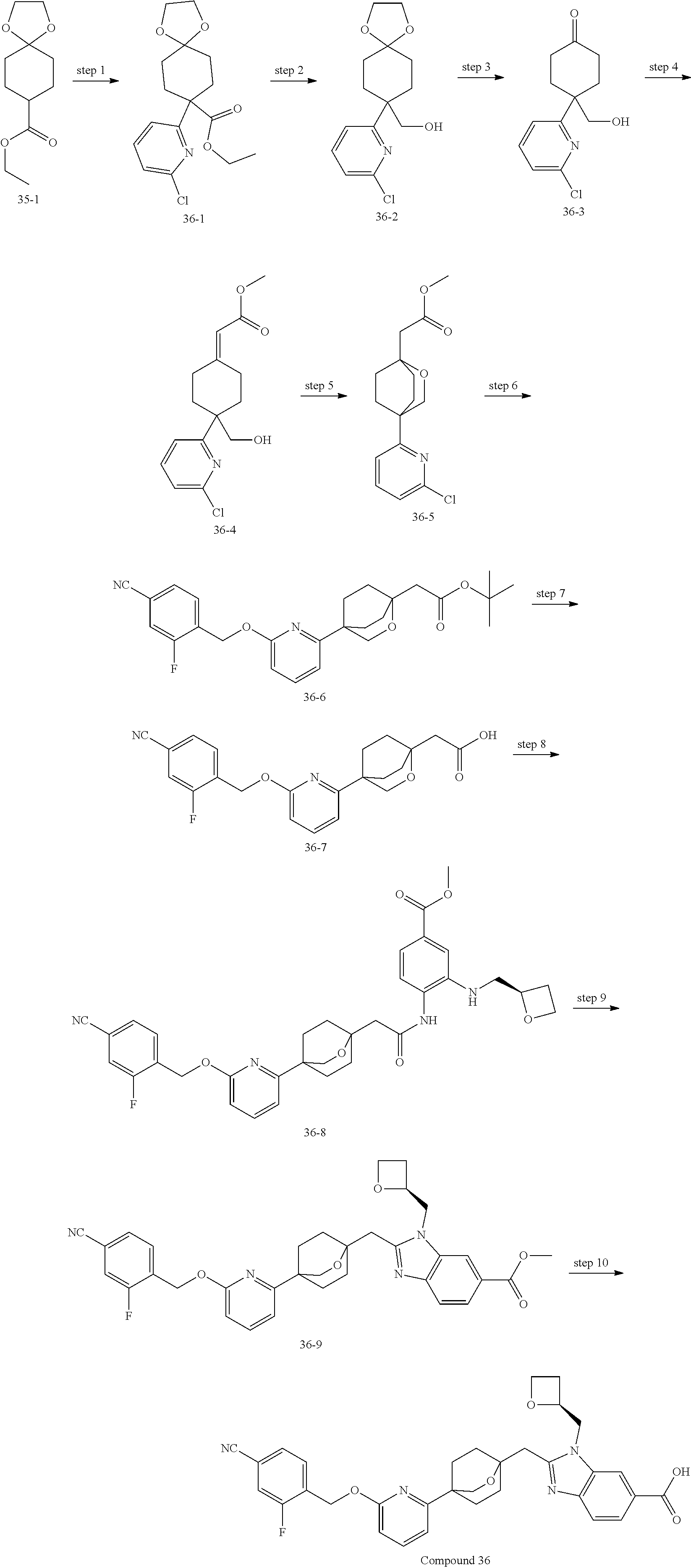

### Step 1: 36-1

To a solution of 35-1 (5 g, 23.34 mmol) in 20 mL of THF was added LiHMDS (35 mL, 1 M) at −78° C., the mixture was stirred at −78° C. for 1 hr, then 2,6-dichloropyridine (3.45 g, 23.34 mmol) in THE was added, and the mixture was stirred at −78° C. to −20° C. for 16 hrs. The reaction mixture was diluted with EA (10 mL), washed with brine (10 mL×2), dried over Na₂SO₄, and filtered. The filtrate was concentrated to give a residue, which was purified by silica gel chromatography (PE/EA=10/1 to 5/1) to give 36-1 (4.5 g, 13.81 mmol, 59.2% yield). MS: m/z=326.1 (M+1).

### Step 2: 36-2

To a solution of 36-1 (3.5 g, 10.74 mmol) in THF (50 mL) was added LAH (1 M, 10.74 mL) at 0° C. slowly, the mixture was stirred at 0° C. for 5 minutes. The reaction was quenched by Na₂SO₄-10H₂O (10 g) at 0° C. and diluted with EA (20 mL). The mixture was filtered, the solid was washed with EA (20 mL), the filtrate was concentrated to give 36-2 (2.6 g, 9.16 mmol, 85.3% yield). MS: m/z=284.1 (M+1).

### Step 3: 36-3

To a solution of 36-2 (2.6 g, 9.16 mmol) in THF (20 mL) was added HCl (2 M, 20 mL), the mixture was stirred at 20° C. for 2 hrs. The mixture was adjusted to pH=13 with saturated aqueous NaHCO₃, diluted with EA (10 mL), washed with brine (10 mL×2), dried over anhydrous Na₂SO₄, and filtered. The filtrate was concentrated to give 36-3 (2.0 g, 8.34 mmol, 91.1% yield). MS: m/z=240.1 (M+1).

### Step 4: 36-4

To MeOH (30 mL) was added NaH (345 mg, 60% purity) portion-wise with stirring, then methyl 2-dimethoxyphosphorylacetate (1.64 g, 9.01 mmol) was added and the resulting mixture was stirred for 30 minutes. To this mixture was added 36-3 (1.8 g, 7.51 mmol) in portions and stirred overnight. The reaction mixture was quenched with a saturated solution of ammonium chloride and concentrated. The residue was extracted with EA (20 mL×2). The combined organic layers were dried over sodium sulfate, filtered, and concentrated to give a residue, which was purified by silica gel chromatography (PE/EA=5/1 to 2/1) to give 36-4 (1.5 g, 5.07 mmol, 67.5% yield). MS: m/z=296.1 (M+1).

### Step 5: 36-5

To a solution of 36-4 (900 mg, 3.04 mmol) in THF (20 mL) was added sodium hydride (128 mg, 60% purity) at 0° C., then the mixture was stirred at 20° C. for 16 hrs. To the mixture was added saturated aqueous NH₄Cl (1 mL) and diluted with EA (50 mL). The organic phase was washed with brine (10 mL×2), dried over anhydrous Na₂SO₄, filtered, and concentrated to give 36-5 (800 mg, 2.70 mmol, 88.9% yield). MS: m/z=296.1 (M+1).

### Step 6: 36-6

To a solution of 36-5 (200 mg, 676.22 μmol), 3-fluoro-4-(hydroxymethyl)benzonitrile (102 mg, 676.22 μmol) in dioxane (5 mL) was added Cs₂CO₃ (659 mg, 2.03 mmol), Pd₂(dba)₃ (62 mg, 67.62 μmol) and Johnphos (40 mg, 135.24 μmol). The mixture was stirred at 100° C. for 16 hrs. The reaction mixture was extracted with EA (50 mL×2), and the combined organic layers were washed with brine (50 mL×2), dried over Na₂SO₄, filtered and concentrated to give a residue, which was purified by silica gel column (PE/EA=5/1) to give 36-6 (120 mg, 292.37 mol, 43.2% yield). MS: m/z=411.2 (M+1).

### Step 7: 36-7

To a solution of 36-6 (120 mg, 292.37 μmol) in MeOH (3 mL) was added LiGH (2 M, 1 mL), the reaction mixture was stirred at 25° C. for 16 hrs. Solvent was removed to give a residue, which was adjusted to pH=7 with 1 M HCl. The mixture was extracted with EA (50 mL×2), the combined organic layers were washed with brine (20 mL), dried over Na₂SO₄, filtered and concentrated to give 36-7 (100 mg, 252.26 μmol, 86.3% yield). MS: m/z=397.1 (M+1).

### Step 8: 36-8

To a solution of 36-7 (100 mg, 252.26 μmol), A-8 (60 mg, 252.26 μmol) and HATU (144 mg, 378.40 μmol) in DMF (3 mL) was added DIPEA (325 mg, 2.52 mmol). The reaction was stirred at 25° C. for 16 hrs. The mixture was extracted with EtOAc (50 mL×2), and the combined organic layers were washed with brine (20 mL), dried over Na₂SO₄, filtered and concentrated to give 36-8 (100 mg, 162.69 μmol, 64.5% yield). MS: m/z=615.3 (M+1).

### Step 9: 36-9

A solution of 36-8 (100 mg, 162.69 μmol) in AcOH (3 mL) was stirred at 60° C. for 16 hrs. Solvent was removed, and the mixture was extracted with EA (50 mL×2). The combined organic layers were washed with brine (20 mL), dried over Na₂SO₄, filtered, and concentrated to give 36-9 (90 mg, 150.84 μmol, 92.7% yield). MS: m/z=597.2 (M+1).

### Step 10: Compound 36

To a solution of 36-9 (90 mg, 150.84 μmol) in THF (4 mL) was added LiOH (2 M, 4 mL), the reaction mixture was stirred at 25° C. for 4 hrs. The mixture was adjusted to pH=7 with 1 M HCl and extracted with EA (50 mL×2). The combined organic layers were washed with brine (20 mL), dried over Na₂SO₄, filtered, and concentrated to give a residue, which was purified by prep-HPLC (column: XBridge@ Prep C18, 5 μm, 19×150 mm; A: 0.1% HCO₂H water, B: acetonitrile; gradient: 5-95% B) to give Compound 36 (8.3 mg, 14.25 μmol, 9.4% yield). ¹H NMR (400 MHz, CD₃OD) δ 8.29 (s, 1H), 7.97-7.94 (m, 1H), 7.65-7.50 (m, 5H), 6.82 (d, J=7.2 Hz, 1H), 6.67 (d, J=8.0 Hz, 1H), 5.47 (s, 2H), 5.16-5.16 (m, 1H), 4.88-4.85 (m, 1H), 4.78-4.74 (m, 1H), 4.63-4.59 (m, 1H), 4.45-4.40 (m, 1H), 3.97-3.92 (m, 2H), 3.25-3.14 (m, 2H), 2.78-2.74 (m, 1H), 2.50-2.45 (m, 1H), 2.03-1.92 (m, 8H); MS: m/z=583.2 (M+1).

### Example 13: Compound 37

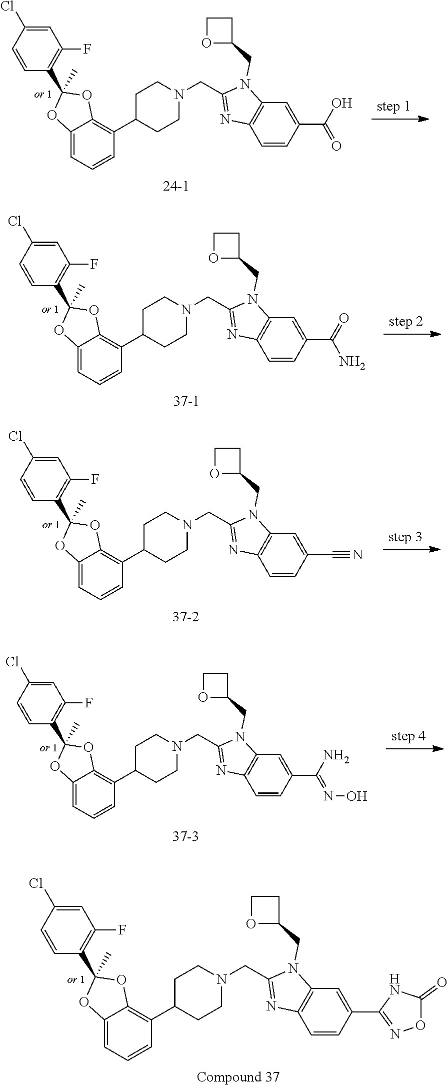 [SMILES: C[C@@]1(c2ccc(Cl)cc2F)Oc2cccc(C3CCN(Cc4nc5ccc(C(=O)O)cc5n4C[C@@H]4CCO4)CC3)c2O1 ; C[C@@]1(c2ccc(Cl)cc2F)Oc2cccc(C3CCN(Cc4nc5ccc(C(N)=O)cc5n4C[C@@H]4CCO4)CC3)c2O1 ; C[C@@]1(c2ccc(Cl)cc2F)Oc2cccc(C3CCN(Cc4nc5ccc(C#N)cc5n4C[C@@H]4CCO4)CC3)c2O1 ; C[C@@]1(c2ccc(Cl)cc2F)Oc2cccc(C3CCN(Cc4nc5ccc(/C(N)=N/O)cc5n4C[C@@H]4CCO4)CC3)c2O1 ; C[C@@]1(c2ccc(Cl)cc2F)Oc2cccc(C3CCN(Cc4nc5ccc(-c6noc(=O)[nH]6)cc5n4C[C@@H]4CCO4)CC3)c2O1]

### Step 1: 37-1

To a mixture of 24-1 (30 mg, 50.67 μmol), NH₄Cl (27.10 mg, 506.71 μmol) in DMF (2 mL) was added DIPEA (19.65 mg, 152.01 μmol) and HATU (28.90 mg, 76.01 μmol) at 30° C. The mixture was stirred for 3 hrs at 30° C. Then, EA (100 mL) was added, and the mixture was washed with H₂O (100 mL×3), dried over Na₂SO₄, filtered, and concentrated to give a residue, which was purified by silica gel chromatography (PE/EA=5/1 to 1/5) to give 37-1 (29 mg, 49.06 μmol, 96.8% yield). MS: m/z=591.2 (M+1, ESI).

### Step 2: 37-2

To a solution of 37-1 (25 mg, 42.30 μmol) in DCM (2 mL) was added Burgess Reagent (30.24 mg, 126.89 μmol) and the mixture was stirred at 25° C. for 2 hrs. The reaction mixture was concentrated and purified by column chromatography on silica gel (EA/PE=1/10 to 1/2) to give 37-2 (20 mg, 34.90 μmol, 82.5% yield). MS: m/z=573.2 (M+1).

### Step 3: 37-3

To a mixture of 37-2 (20 mg, 34.90 μmol), KOAc (34.60 mg, 349.01 μmol) in EtOH (2 mL) was added hydroxylamine;hydrochloride (12.13 mg, 174.50 μmol) at 20° C. The reaction was stirred for 4 hrs at 60° C. The mixture was concentrated to give a residue, which was purified by column chromatography on silical gel (EA/PE=1/5 to 1/2) to give 37-3 (19 mg, 31.35 μmol, 89.8% yield). MS: m/z=606.2 (M+1).

### Step 4: Compound 37

To a mixture of 37-3 (10 mg, 16.50 μmol) and DIEA (6.40 mg, 49.50 μmol) in THF (3 mL) was added di(imidazol-1-yl)methanone (4.01 mg, 24.75 μmol) at 20° C. The reaction was stirred for 1 hr at 70° C. The mixture was concentrated to give a residue, which was purified by silica gel chromatography (DCM/EA=10/1 to 1/1) to give Compound 37 (2 mg, 3.16 μmol, 19.2% yield). ¹H NMR (400 MHz, DMSO-d₆) δ 8.01 (s, 1H), 7.66 (d, J=8.4, 1.4 Hz, 1H), 7.59-7.53 (m, 4H), 7.37-7.33 (m, 1H), 6.81-6.76 (m, 3H), 5.12 (d, J=7.6 Hz, 1H), 4.73 (d, J=15.4 Hz, 1H), 4.61 (d, J=14.8 Hz, 1H), 4.47 (t, J=7.2 Hz, 1H), 4.40 (s, 1H), 3.93 (d, J=13.5 Hz, 1H), 3.76 (d, J=13.5 Hz, 1H), 2.90 (d, J=7.2 Hz, 3H), 2.62-2.58 (m, 1H), 2.42-2.38 (m, 2H), 2.03 (s, 3H), 1.98 (s, 1H), 1.71 (s, 2H), 1.46 (s, 2H); MS. m/z=632.2 (M+1).

### Example 14: Compound 56

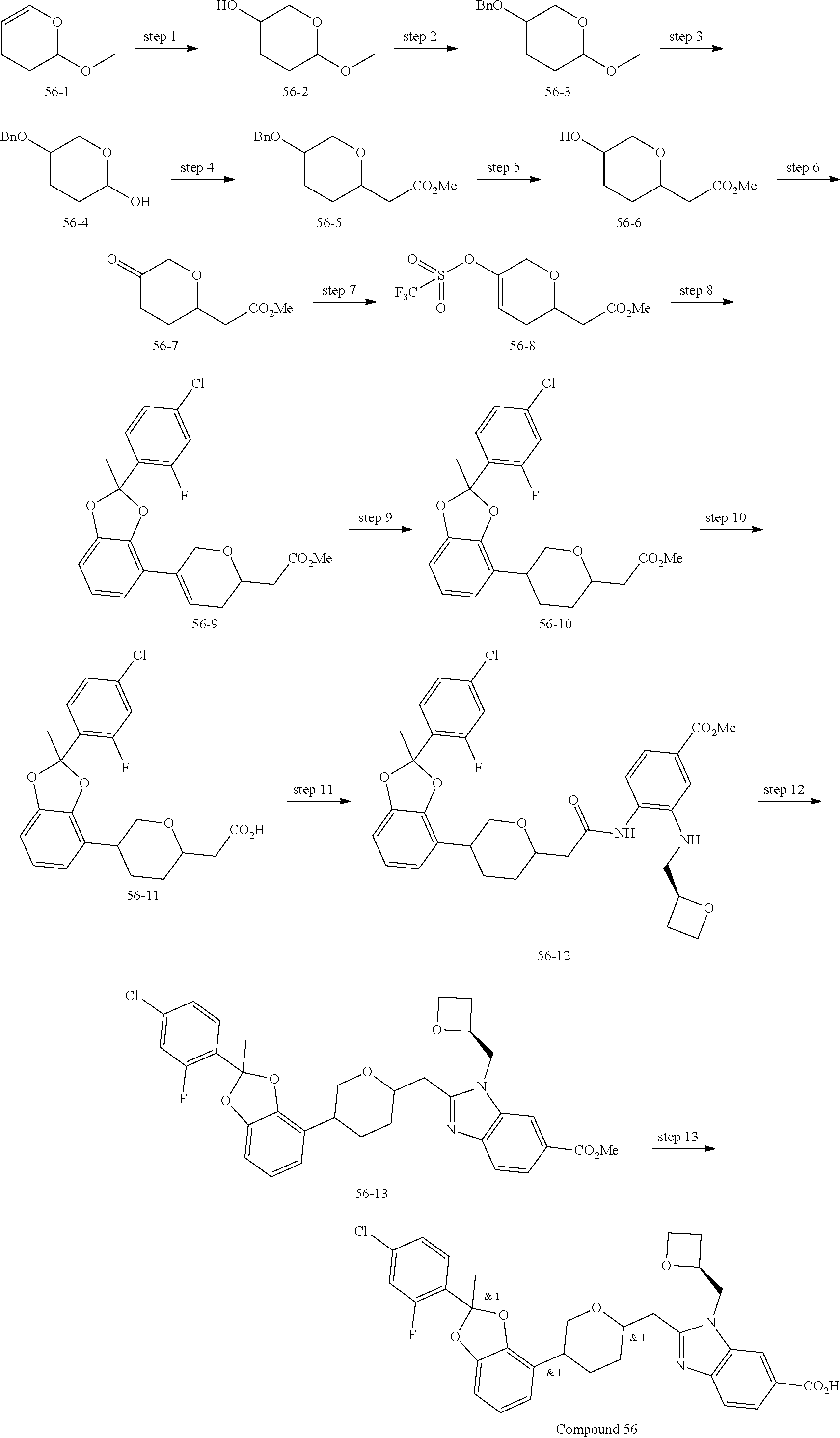

### Step 1: 56-2

To a solution of 56-1 (20 g, 175.22 mmol) in THF (100 mL) was added borane (1 M in THF, 70 mL) at 0° C. The reaction mixture was stirred at 20° C. for 24 hrs. The solution was cooled to 0° C. and hydrogen peroxide (36.12 mL, 30% purity) was introduced into the reaction, followed by NaOH (2.5 M, 77.10 mL). The reaction mixture was stirred at 20° C. for another 24 hrs. K₂CO₃ (100 g) was added, and the mixture was stirred at for 2 hrs. The mixture was extracted with EA (50 mL×3) and the combined organic layers were washed with brine (50 mL), dried and concentrated to give a residue, which was purified by column chromatography (PE/EA=1/1) to give 56-2 (12 g, 52% yield).

### Step 2: 56-3

To a suspension of NaH (4.35 g, 60% purity) in DMF (50 mL) was added 56-2 (10 g, 75.67 mmol) at 0° C. The reaction mixture was stirred for 5 minutes before bromomethyl benzene (15.53 g, 90.80 mmol) was added. The resulting mixture was stirred at 20° C. for 2 hrs. The mixture was partitioned between 30 mL of water and 30 mL of EA. The aqueous was extracted with EA (10 mL×3). The combined organic layers were washed with 1 M HCl (20 mL) and brine (20 mL), dried and concentrated to give a residue, which was purified by flash chromatography (PE/EA=4/1) to give 56-3 (9.65 g, 57% yield). MS: m/z=245.1 (M+23).

### Step 3: 56-4

A solution of 56-3 (9.65 g, 43.41 mmol) in AcOH (45 mL) and Water (22 mL) was heated at 55° C. for 16 hrs. The mixture was concentrated to give a residue, to which saturated aqueous NaHCO₃ was added until bubbling ceased. The mixture was extracted with EA (20 mL×3), and the organic layers was washed with brine (30 mL), dried and concentrated to give 56-4 (9.2 g, crude). MS: m/z=231.1 (M+23).

### Step 4: 56-5

To a suspension of NaH (919 mg, 60% purity) in dry THF (20 mL) was added a solution of methyl 2-dimethoxyphosphorylacetate (3.50 g, 19.21 mmol) in THF (2 mL) dropwise at 0° C. After stirring for 8 hrs at 0-20° C., a solution of 56-4 (2 g, 9.60 mmol) in THF (6 mL) was slowly added. After stirred for 20 minutes at 20° C., the mixture was refluxed for 6 hrs. The mixture was treated with HCl (0.01 M) to PH=2 and extracted with EA (20 mL×3). The combined extracts were washed with brine (30 mL), dried and concentrated to give a residue, which was purified by flash chromatography (PE/EA=5/1) to give 56-5 (2.54 g, 67% yield). MS: m/z=265.2 (M+1).

### Step 5: 56-6

A solution of 56-5 (2.5 g, 9.46 mmol) and Pd/C (1.15 g, 50% wet) in methanol (30 mL) was degasses under vacuum and then purged with hydrogen, this evacuation-purge cycle was carried out for a total of three times. The reaction mixture was then stirred at 40° C. under hydrogen balloon for 20 hrs. The mixture was filtered, and concentrated to give a residue, which was purified by flash chromatography (PE/EA=1/1) to give 56-6 (1.26 g, 76% yield). MS: m/z=175.1 (M+1).

### Step 6: 56-7

To a solution of 56-6 (1.26 g, 7.23 mmol) in DCM (10 mL) was added Dess-Martin Periodinane (4.60 g, 10.85 mmol). The reaction mixture was stirred at 20° C. for 4 hrs. Water (10 mL) was added to this solution, and the mixture was extracted with DCM (20 mL×2). The combined organic phases were washed with NaHCO₃ solution (20 mL), Na₂S203 solution (20 mL) and brine (20 mL), dried and concentrated to give a residue, which was purified by flash chromatography (PE/EA=1/1) to give 56-7 (800 mg, 64% yield). ¹H NMR (400 MHz, CDCl₃) δ 4.20-4.14 (m, 2H), 4.03-3.99 (d, J=16.4 Hz, 1H), 3.72 (s, 3H), 2.70-2.46 (m, 4H), 2.20-2.13 (m, 1H), 1.97-1.88 (m, 1H).

### Step 7: 56-8

To the solution of 56-7 (710 mg, 4.12 mmol) in THF (50 mL) was added 1,1,1-trifluoro-N-phenyl-N-(trifluoromethylsulfonyl)methanesulfonamide (1.92 g, 5.36 mmol) and lithium;bis(trimethylsilyl)azanide (1 M, 8.25 mL) at −78° C. After addition, the mixture was stirred at 25° C. for 18 hrs. The resulting mixture was diluted with water and extracted with EA (50 mL). The organic layer was washed with water (100 mL×2) and brine, dried over Na₂SO₄, filtered, and concentrated to give 56-8 (600 mg, 1.97 mmol, 47.8% yield).

### Step 8: 56-9

To a mixture of Intermediate B (300 mg, 767.97 μmol), 56-8 (280.38 mg, 921.56 mol) in Water (4 mL) and dioxane (20 mL) was added K₂CO₃ (212.28 mg, 1.54 mmol) and cyclopentyl(diphenyl)phosphane;dichloropalladium;iron (56.19 mg, 76.80 μmol) at 30° C. The reaction solution was stirred for 2 hrs at 100° C. The mixture was concentrated and purified by column chromatography on silica gel (PE/EA=10/1 to 3/1) to give 56-9 (200 mg, 477.51 μmol, 62.18% yield).

### Step 9: 56-10

A mixture of 56-9 (200 mg, 477.51 μmol) and dioxoplatinum (108.43 mg, 477.51 mol) in methanol (30 mL) was stirred at 25° C. under H2 atmosphere for 1 hr. The resulting mixture was filtered and concentrated to give 56-10 (190 mg, 451.46 μmol, 94.5% yield). MS: m/z=421.1 (M+1).

### Step 10: 56-11

To a solution of 56-10 (190 mg, 451.46 μmol) in water (1 mL), THF (4 mL) and methanol (4 mL) was added NaOH (36.1 mg, 902.92 μmol) and stirred at 25° C. for 3 hrs. The reaction mixture was extracted with EA (50 mL×3). The aqueous layer was adjusted to pH=4-5 with 2 M HCl, and to the mixture was added EA (200 mL) and stirred for 0.2 hr. The mixture was filtered and extracted with EA (100 mL×6). The organic layers were washed with brine (50 mL×3), dried over Na₂SO₄, filtered, and concentrated to give a residue, which was slurried in (PE/EA=1/1) for 0.5 hr and filtered to give 56-11 (140 mg, 375.95 μmol, 83.2% yield).

### Step 11: 56-12

To a mixture of 56-11 (130 mg, 319.54 μmol), A-8 (75.50 mg, 319.54 μmol) in DMF (5 mL) was added DIPEA (82.60 mg, 639.09 μmol, 111.32 μL) and HATU (121.50 mg, 319.54 mol) at 30° C., and the mixture was stirred for 3 hrs at 30° C. Then, EA (100 mL) was added, and the mixture was washed with H₂O (100 mL×3), dried over Na₂SO₄, filtered, and concentrated to give a residue, which was purified by silica gel chromatography (PE/EA=5/1 to 1/1) to give 56-12 (120 mg, 191.97 μmol, 60.1% yield). MS: m/z=625.2 (M+1).

### Step 12: 56-13

A solution of 56-12 (80 mg, 127.98 μmol) in AcOH (5 mL) was stirred at 100° C. for 1 hr. The resulting mixture was cooled to room temperature and concentrated to give a residue, which was purified by FCC (PE/EA=10/1 to 1/1) to give 56-13 (35 mg, 57.65 μmol, 45.0% yield). MS: m/z=607.1 (M+1).

### Step 13: Compound 56

A mixture of 56-13 (35 mg, 57.65 μmol) and NaOH (4.61 mg, 115.31 μmol) in THF (2 mL), methanol (2 mL) and water (1 mL) was stirred at 20° C. for 3 hrs. The mixture was acidified with 1 M HCl to pH=3, filtered and washed with water to give Compound 56 (20 mg, 33.72 mol, 58.5% yield). ¹H NMR (400 MHz, DMSO-d₆) δ 8.23 (s, 1H), 7.82 (d, J=8.6 Hz, 1H), 7.62 (d, J=10.4 Hz, 3H), 7.37 (d, J=9.8 Hz, 1H), 6.89-6.67 (m, 3H), 5.40-5.30 (m, 1H), 5.04 (s, 1H), 4.67 (s, 1H), 4.53-4.48 (m, 3H), 4.36 (s, 1H), 3.79 (s, 1H), 3.03-2.96 (m, 2H), 2.86-2.78 (m, 1H), 2.74-2.67 (m, 1H), 2.36-2.33 (m, 3H), 2.04 (d, J=6.4 Hz, 3H), 1.93 (s, 2H); MS: m/z=593.1 (M+1).

### Example 15: Compound 4 and Compound 18

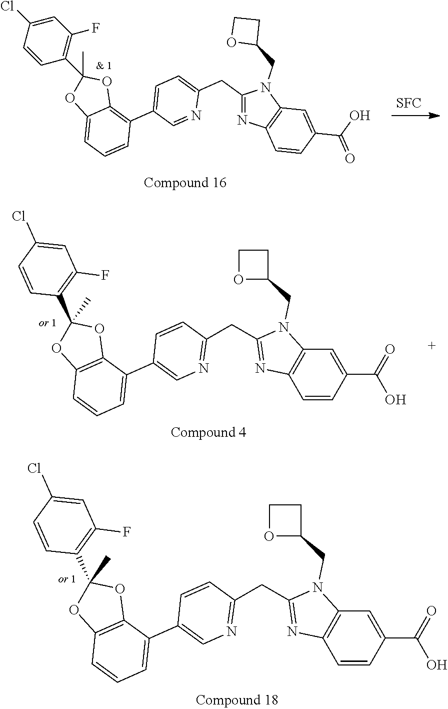

Compound 16 (44 mg) was further separated by SFC (Column: IG 30×250 mm, 10 um (Daicel), Column temperature: 35° C., Mobile phase: CO₂:MeOH (0.2% Methanol Ammonia) 60/40, Flow rate: 80 g/min, Detection wavelength: 214 nm, Cycle time: 6 min) to give Compound 4 (21 mg, 35.84 μmol, 100% ee) Compound 18 (19 mg, 32.42 μmol, 98% ee). MS: m/z=586.2 (M+1).

The compounds of in Table N below were made according to the procedure of Compound 4 and Compound 18.

TABLE N

| Name | Structure | SFC condition |
|---|---|---|
| 3 | 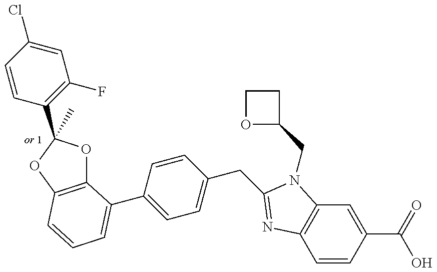 [SMILES: C[C@@]1(c2ccc(Cl)cc2F)Oc2cccc(-c3ccc(Cc4nc5ccc(C(=O)O)cc5n4C[C@@H]4CCO4)cc3)c2O1] | Column: Daicel CHIRALCEL AZ, Column size: 250 mm × 30 mm I.D., 10 μm; Mobile phase: (CO₂:MeOH [0.2% NH₃ (7 M solution of MeOH)] = 75:25, Flow rate: 80 g/min, Wavelength: 214 nm, Temperature: 35° C. |
| 19 | 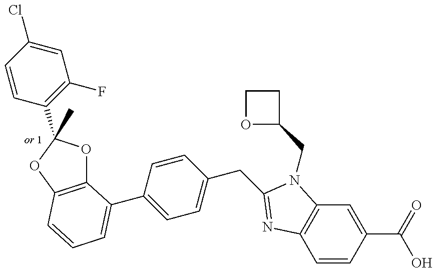 [SMILES: C[C@]1(c2ccc(Cl)cc2F)Oc2cccc(-c3ccc(Cc4nc5ccc(C(=O)O)cc5n4C[C@@H]4CCO4)cc3)c2O1] | |
| 40 | 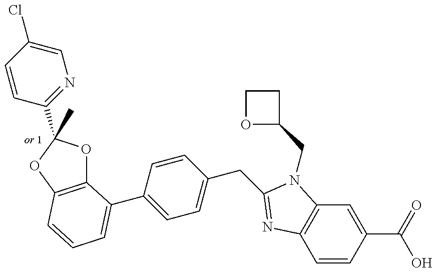 [SMILES: C[C@]1(c2ccc(Cl)cn2)Oc2cccc(-c3ccc(Cc4nc5ccc(C(=O)O)cc5n4C[C@@H]4CCO4)cc3)c2O1] | Column: Daicel CHIRALCEL AD, Column size: 250 mm × 30 mm I.D., 10 μm; Mobile phase: CO₂:MeOH [0.2% NH₃ (7 M solution of MeOH)] = 65:35, Flow rate: 70 g/min, Wavelength: 214 nm, Temperature: 35° C. |
| 41 | 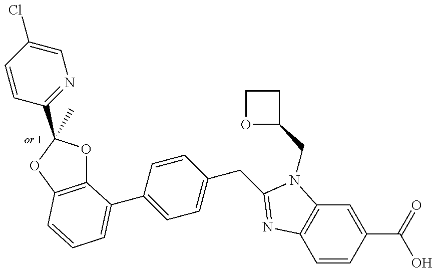 [SMILES: C[C@@]1(c2ccc(Cl)cn2)Oc2cccc(-c3ccc(Cc4nc5ccc(C(=O)O)cc5n4C[C@@H]4CCO4)cc3)c2O1] | |

### Example 16: Compound 58

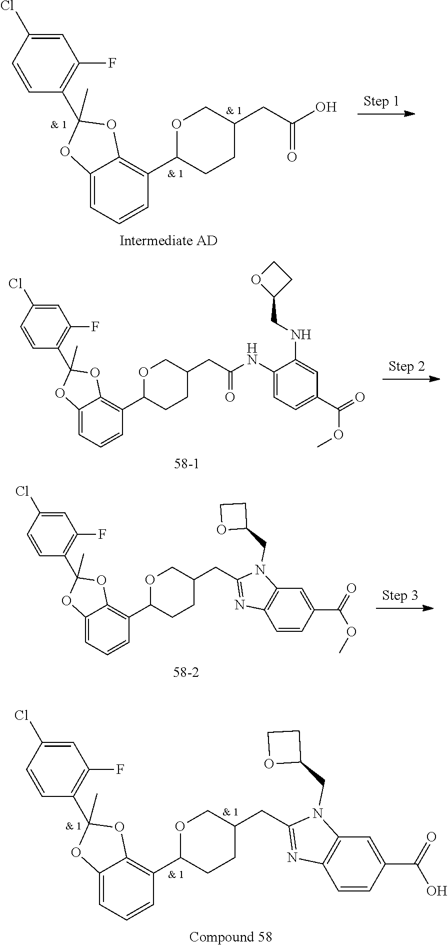 [SMILES: CC1(c2ccc(Cl)cc2F)Oc2cccc(C3CCC(CC(=O)O)CO3)c2O1 ; COC(=O)c1ccc(NC(=O)CC2CCC(c3cccc4c3OC(C)(c3ccc(Cl)cc3F)O4)OC2)c(NC[C@@H]2CCO2)c1 ; COC(=O)c1ccc2nc(CC3CCC(c4cccc5c4OC(C)(c4ccc(Cl)cc4F)O5)OC3)n(C[C@@H]3CCO3)c2c1 ; CC1(c2ccc(Cl)cc2F)Oc2cccc(C3CCC(Cc4nc5ccc(C(=O)O)cc5n4C[C@@H]4CCO4)CO3)c2O1]

### Step 1: 58-1

To a mixture of Intermediate AD (218 mg, 535.85 μmol), methyl 4-amino-3-[[(2S)-oxetan-2-yl]methylamino]benzoate (126.60 mg, 535.85 μmol) in DMF (6 mL) was added HATU (305.62 mg, 803.77 μmol) and DIPEA (207.76 mg, 1.61 mmol). The reaction was stirred for 18 hrs at 25° C. before quenched with water (50 mL) and extracted with EA (30 mL×3). The combined organic layers were washed with brine (50 mL), dried over Na₂SO₄, filtered and concentrated to give a residue, which was purified by silica gel chromatography (EA in petroleum ether from 0% to 70%) to give 58-1 (288 mg, 460.74 μmol, 86.0% yield). MS: m/z=625 (M+1).

### Step 2: 58-2

To a mixture of 58-1 (288 mg, 460.74 μmol) in AcOH (6 mL) was stirred for 1 hr at 100° C. The reaction mixture was concentrated to give crude 58-2 (279 mg, 459.59 μmol, 99.7% yield).

### Step 3: Compound 58

To a mixture of 58-2 (279 mg, 459.59 μmol) in THF (2 mL), MeOH (2 mL) and Water (2 mL) was added NaOH (55.15 mg, 1.38 mmol) and stirred for 3 hrs at 25° C. The reaction mixture was quenched with water (50 mL), acidified with 1 M HCl to pH=3-4 and extracted with EA (30 mL×3). The combined organic layers were wash by brine (50 mL), dried over Na₂SO₄, filtered and concentrated to give a residue, which was purified by prep-HPLC (HCOOH) to give Compound 58 (114 mg, 192.23 μmol, 41.8% yield). MS: m/z=593 (M+1). ¹H NMR (400 MHz, DMSO-d6) δ 8.20 (s, 1H), 7.80 (d, J=8.4 Hz, 1H), 7.62 (d, J=8.4 Hz, 1H), 7.53 (q, J=7.6 Hz, 1H), 7.35 (dd, J=8.6, 5.9 Hz, 1H), 6.97 (dd, J=5.7, 3.3 Hz, 2H), 6.90-6.73 (m, 2H), 5.55-5.19 (m, 1H), 5.12-4.93 (m, 1H), 4.62 (dd, J=15.4, 7.2 Hz, 1H), 4.53-4.38 (m, 2H), 4.36-4.23 (m, 1H), 4.12-4.00 (m, 1H), 3.15-3.00 (m, 2H), 2.72-2.53 (m, 1H), 2.43-2.06 (m, 6H), 2.03 (d, J=6.2 Hz, 3H), 1.92-1.70 (m, 1H).

The compounds in Table O below were made according to the procedure of Compound 58.

TABLE O

| Name | structure | ¹H NMR and/or LC/MS data |
|---|---|---|
| 73 | 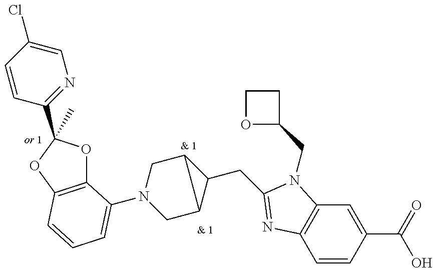 [SMILES: C[C@@]1(c2ccc(Cl)cn2)Oc2cccc(N3CC4C(Cc5nc6ccc(C(=O)O)cc6n5C[C@@H]5CCO5)C4C3)c2O1] | MS: m/z = 573.2 (M + 1). ¹H NMR (400 MHz, DMSO- d6) δ 8.75-8.68 (m, 1H), 8.20- 8.15 (s, 1H), 8.02-7.96 (m, 1H), 7.82-7.76 (d, J = 8.3 Hz, 1H), 7.64-7.56 (d, J = 8.3 Hz, 2H), 6.70-6.64 (t, J = 8.0 Hz, 1H), 6.35-6.29 (d, J = 7.8 Hz, 1H), 6.21-6.15 (d, J = 8.3 Hz, 1H), 5.06-4.98 (m, 1H), 4.66- 4.56 (dd, J = 15.6, 7.3 Hz, 1H), 4.52-4.46 (m, 1H), 4.45- 4.40 (m, 1H), 4.32-4.26 (m, 1H), 3.80-3.75 (d, J = 9.6 Hz, 1H), 3.66-3.62 (d, J = 9.4 Hz, 1H), 3.22-3.18 (m, 1H), 3.00- 2.94 (m, 1H), 2.70-2.64 (m, |
| | | 1H), 2.38-2.32 (m, 1H), 1.97 |
| | | (s, 3H), 1.66-1.60 (m, 2H), |
| | | 1.36-1.32 (m, 1H), 1.26- |
| | | 1.22 (m, 2H). |
| 75 | 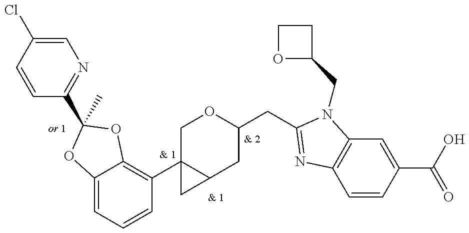 [SMILES: C[C@@]1(c2ccc(Cl)cn2)Oc2cccc(C34COC(Cc5nc6ccc(C(=O)O)cc6n5C[C@@H]5CCO5)CC3C4)c2O1] | MS: m/z = 588.2 (M + 1). |
| 77 | 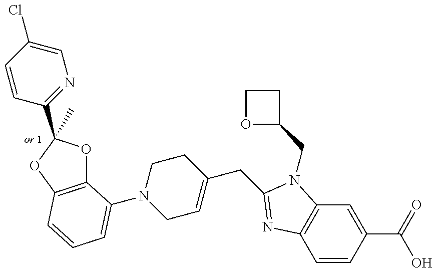 [SMILES: C[C@@]1(c2ccc(Cl)cn2)Oc2cccc(N3CC=C(Cc4nc5ccc(C(=O)O)cc5n4C[C@@H]4CCO4)CC3)c2O1] | MS: m/z = 573.3 (M + 1). |
| 78 & 79 | 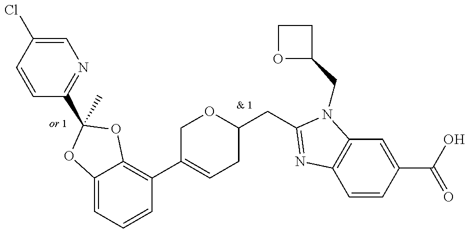 [SMILES: C[C@@]1(c2ccc(Cl)cn2)Oc2cccc(C3=CCC(Cc4nc5ccc(C(=O)O)cc5n4C[C@@H]4CCO4)OC3)c2O1] | MS: m/z = 574.3 (M + 1). |
| 82 | 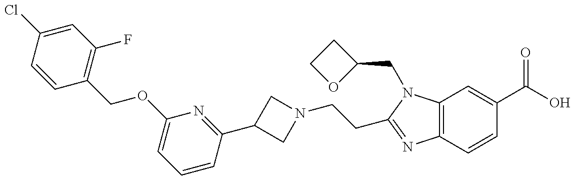 [SMILES: O=C(O)c1ccc2nc(CCN3CC(c4cccc(OCc5ccc(Cl)cc5F)n4)C3)n(C[C@@H]3CCO3)c2c1] | MS: m/z = 551.3 (M + 1). ¹H NMR (400 MHz, DMSO- d6) δ 8.19 (s, 1H), 7.80-7.76 (d, J = 8.4 Hz, 1H), 7.66-7.56 (m, 3H), 7.48-7.43 (m, 1H), 7.32-7.28 (m, 1H), 6.94- 6.88 (d, J = 7.2 Hz, 1H), 6.73- 6.68 (d, J = 8.1 Hz, 1H), 5.41 (s, 2H), 5.06-4.98 (d, J = 5.2 Hz, 1H), 4.66-4.60 (m, 1H), 4.54-4.48 (m, 1H), 4.46- |
| | | 4.40 (m, 1H), 4.32-4.26 (m, |
| | | 1H), 3.70-3.57 (m, 2H), 3.27- |
| | | 3.22 (m, 2H), 2.98-2.94 (m, |
| | | 2H), 2.72-2.62 (m, 1H), 2.40- |
| | | 2.30 (m, 1H), 2.00-1.94 (m, |
| | | 1H), 1.26-1.20 (m, 2H). |
| 100 | 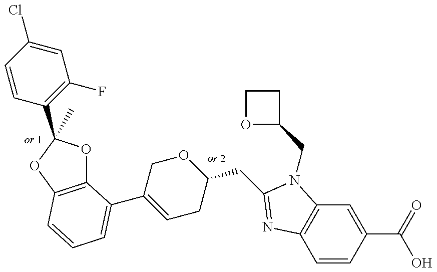 [SMILES: C[C@@]1(c2ccc(Cl)cc2F)Oc2cccc(C3=CC[C@@H](Cc4nc5ccc(C(=O)O)cc5n4C[C@@H]4CCO4)OC3)c2O1] | MS: m/z = 591.3 (M + 1). 1H NMR (400 MHz, DMSO- d6) δ 8.19 (s, 1H), 7.79 (d, J = 8.5 Hz, 1H), 7.62-7.49 (m, 3H), 7.33 (d, J = 7.1 Hz, 1H), 6.90-6.75 (m, 3H), 6.43 (s, 1H), 5.05-4.96 (m, 1H), 4.73- 4.63 (m, 1H), 4.59-4.50 (m, 1H), 4.44-4.41 (m, 3H), 4.37- 4.30 (m, 1H), 4.22-4.12 (m, 1H), 3.22-3.17 (m, 1H), 2.73- 2.62 (m, 1H), 2.39-2.31 (m, 2H), 2.01 (s, 3H), 1.25-1.18 (m, 2H). |
| 101 | 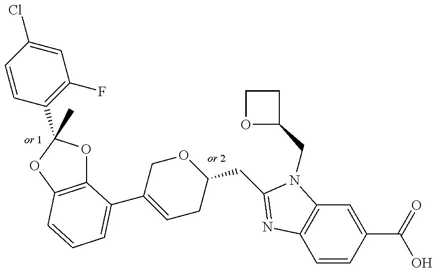 [SMILES: C[C@]1(c2ccc(Cl)cc2F)Oc2cccc(C3=CC[C@@H](Cc4nc5ccc(C(=O)O)cc5n4C[C@@H]4CCO4)OC3)c2O1] | MS: m/z = 591.3 (M + 1). |
| 106 | 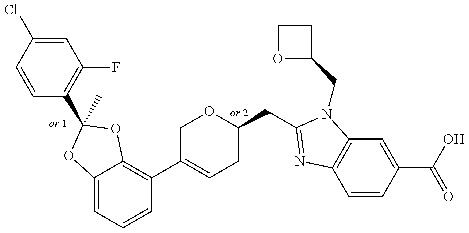 [SMILES: C[C@@]1(c2ccc(Cl)cc2F)Oc2cccc(C3=CC[C@H](Cc4nc5ccc(C(=O)O)cc5n4C[C@@H]4CCO4)OC3)c2O1] | MS: m/z = 591.3 (M + 1). |
| 107 | 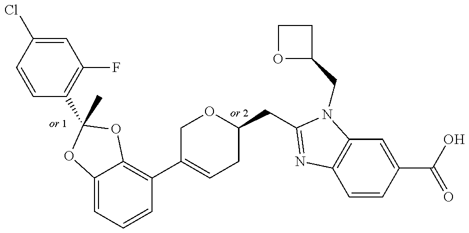 [SMILES: C[C@]1(c2ccc(Cl)cc2F)Oc2cccc(C3=CC[C@H](Cc4nc5ccc(C(=O)O)cc5n4C[C@@H]4CCO4)OC3)c2O1] | MS: m/z = 591.3 (M + 1). |
| 109 | 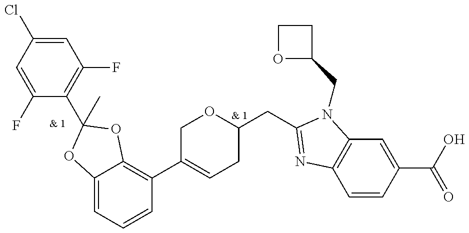 [SMILES: CC1(c2c(F)cc(Cl)cc2F)Oc2cccc(C3=CCC(Cc4nc5ccc(C(=O)O)cc5n4C[C@@H]4CCO4)OC3)c2O1] | MS: m/z = 609.3 (M + 1). |
| 111 | 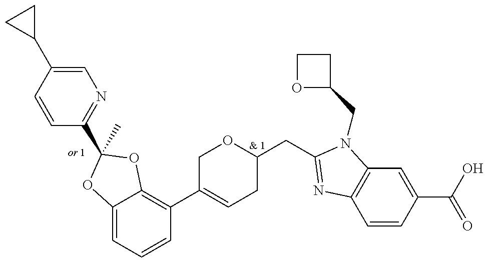 [SMILES: C[C@@]1(c2ccc(C3CC3)cn2)Oc2cccc(C3=CCC(Cc4nc5ccc(C(=O)O)cc5n4C[C@@H]4CCO4)OC3)c2O1] | MS: m/z = 580.3 (M + 1). ¹H NMR (400 MHz, DMSO- d₆) δ 8.47 (s, 1H), 8.22 (s, 1H), 7.79 (d, J = 8.4 Hz, 1H), 7.62 (dd, J = 8.3, 2.8 Hz, 1H), 7.51- 7.39 (m, 2H), 6.85-6.74 (m, 3H), 6.42 (s, 1H), 5.03 (s, 1H), 4.75-4.65 (m, 1H), 4.57 (d, J = 13.0 Hz, 1H), 4.52-4.40 (m, 3H), 4.39-4.26 (m, 1H), 4.19- 4.13 (m, 1H), 3.28-3.17 (m, 2H), 2.75-2.65 (m, 1H), 2.45- 2.28 (m, 3H), 2.03-1.94 (m, 4H), 1.05-0.96 (m, 2H), 0.78- 0.72 (m, 2H). |
| 112 | 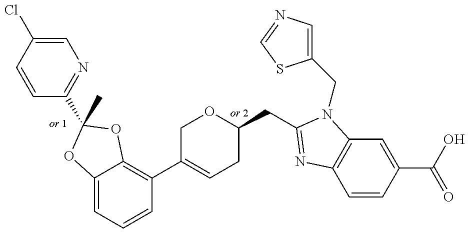 [SMILES: C[C@]1(c2ccc(Cl)cn2)Oc2cccc(C3=CC[C@H](Cc4nc5ccc(C(=O)O)cc5n4Cc4cncs4)OC3)c2O1] | MS: m/z = 601 (M + 1). 1H NMR (400 MHz, DMSO- d6) δ 8.99 (s, 1H), 8.71 (s, 1H), 8.20 (s, 1H), 8.02-7.94 (m, 2H), 7.80 (d, J = 8.1 Hz, 1H), 7.61 (dd, J = 14.2, 8.5 Hz, 2H), 6.87-6.79 (m, 2H), 6.76 (d, J = 7.1 Hz, 1H), 6.40 (s, 1H), 5.92 (s, 2H), 4.40 (d, J = 18.8 Hz, 2H), 4.17-4.05 (m, 1H), 3.25 (d, J = 5.9 Hz, 2H), 2.41-2.27 (m, 2H), 2.00 (s, 3H). |
| 113 | 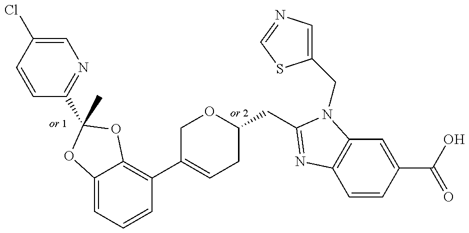 [SMILES: C[C@]1(c2ccc(Cl)cn2)Oc2cccc(C3=CC[C@@H](Cc4nc5ccc(C(=O)O)cc5n4Cc4cncs4)OC3)c2O1] | MS: m/z = 601 (M + 1). 1H NMR (400 MHz, DMSO- d6) δ 8.99 (s, 1H), 8.71 (s, 1H), 8.19 (s, 1H), 7.97 (s, 2H), 7.79 (s, 1H), 7.60 (dd, J = 17.3, 8.3 Hz, 2H), 6.83 (s, 1H), 6.83-6.75 (m, 2H), 6.40 (s, 1H), 5.91 (s, 2H), 4.57-4.34 (m, 2H), 4.18-4.05 (m, 1H), 3.29-3.21 (m, 2H), 2.41- 2.28 (m, 2H), 2.00 (s, 3H). |
| 114 | 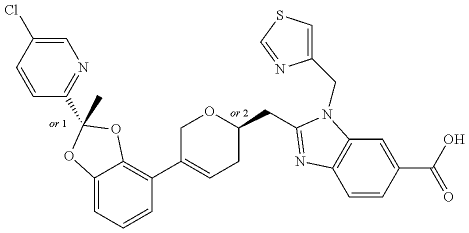 [SMILES: C[C@]1(c2ccc(Cl)cn2)Oc2cccc(C3=CC[C@H](Cc4nc5ccc(C(=O)O)cc5n4Cc4cscn4)OC3)c2O1] | MS: m/z = 601.3 (M + 1). 1H NMR (400 MHz, DMSO- d₆) δ 9.03 (s, 1H), 8.71 (s, 1H), 8.16 (s, 1H), 8.00 (dd, J = 8.5, 2.4 Hz, 1H), 7.78 (d, J = 8.3 Hz, 1H), 7.72-7.52 (m, 3H), 6.85-6.75 (m, 3H), 6.45- 6.35 (m, 1H), 5.70 (s, 2H), 4.42 (s, 2H), 4.18-4.08 (m, 1H), 2.58-2.45 (m, 2H), 2.43- 2.31 (m, 2H), 2.00 (s, 3H). |
| 115 | 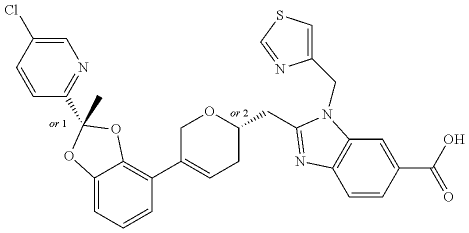 [SMILES: C[C@]1(c2ccc(Cl)cn2)Oc2cccc(C3=CC[C@@H](Cc4nc5ccc(C(=O)O)cc5n4Cc4cscn4)OC3)c2O1] | MS: m/z = 601.2 (M + 1). 1H NMR (400 MHz, DMSO- d₆) δ 9.03 (s, 1H), 8.71 (s, 1H), 8.16 (s, 1H), 7.99 (d, J = 8.5 Hz, 1H), 7.77 (d, J = 9.4 Hz, 1H), 7.67 (s, 1H), 7.58 (d, J = 8.4 Hz, 2H), 6.90-6.71 (m, 3H), 6.45-6.35 (m, 1H), 5.70 (s, 2H), 4.51-4.25 (m, 2H), 4.20-4.18 (m, 1H), 2.60- 2.48 (m, 2H), 2.38-2.23 (m, 2H), 2.01 (s, 3H). |
| 116 | 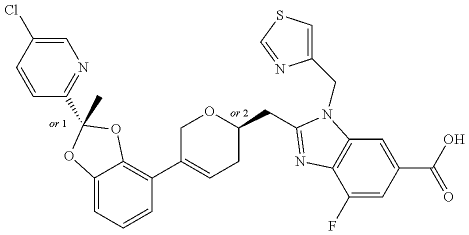 [SMILES: C[C@]1(c2ccc(Cl)cn2)Oc2cccc(C3=CC[C@H](Cc4nc5c(F)cc(C(=O)O)cc5n4Cc4cscn4)OC3)c2O1] | MS: m/z = 619.2 (M + 1). ¹H NMR (400 MHz, DMSO- d₆) δ 9.05 (d, J = 1.8 Hz, 1H), 8.72 (d, J = 1.8 Hz, 1H), 8.03- 7.98 (m, 2H), 7.67 (s, 1H), 7.61 (d, J = 8.5 Hz, 1H), 7.51 (d, J = 11.9 Hz, 1H), 6.88- 6.76 (m, 3H), 6.40 (s, 1H), 5.70 (s, 2H), 4.43 (s, 2H), 4.18- 4.14 (m, 1H), 2.46-2.38 (m, 4H), 2.01 (s, 3H). |
| 117 | 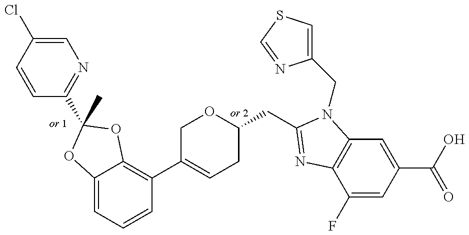 [SMILES: C[C@]1(c2ccc(Cl)cn2)Oc2cccc(C3=CC[C@@H](Cc4nc5c(F)cc(C(=O)O)cc5n4Cc4cscn4)OC3)c2O1] | MS: m/z = 619.2 (M + 1). ¹H NMR (400 MHz, DMSO- d₆) δ 9.05 (d, J = 1.8 Hz, 1H), 8.73 (d, J = 2.2 Hz, 1H), 8.05- 7.98 (m, 2H), 7.70 (s, 1H), 7.60 (d, J = 8.5 Hz, 1H), 7.51 (d, J = 11.5 Hz, 1H), 6.88- 6.78 (m, 3H), 6.40 (s, 1H), 5.73 (s, 2H), 4.49 (s, 1H), 4.38 (d, J = 15.9 Hz, 1H), 4.13 (s, 1H), 2.48-2.34 (m, 4H), 2.02 (s, 3H). |
| 118 | 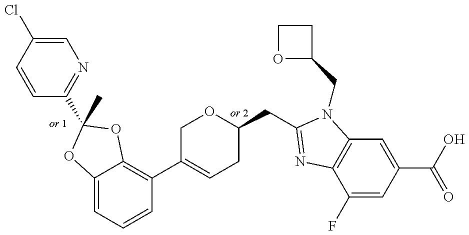 [SMILES: C[C@]1(c2ccc(Cl)cn2)Oc2cccc(C3=CC[C@H](Cc4nc5c(F)cc(C(=O)O)cc5n4C[C@@H]4CCO4)OC3)c2O1] | MS: m/z = 592.3 (M + 1). 1H NMR (400 MHz, DMSO- d6) δ 8.71 (s, 1H), 8.08 (s, 1H), 8.03-7.96 (m, 1H), 7.60 (d, J = 8.4 Hz, 1H), 7.48 (d, J = 11.6 Hz, 1H), 6.85-6.74 (m, 3H), 6.43-6.38 (m, 1H), 5.06- 4.96 (m, 1H), 4.76-4.67 (m, 1H), 4.66-4.45 (m, 2H), 4.44 (s, 2H), 4.36-4.26 (m, 1H), 4.20-4.06 (m, 1H), 2.68 (d, J = 16.8 Hz, 1H), 2.41-2.30 (m, 4H), 2.00 (s, 3H). |
| 119 | 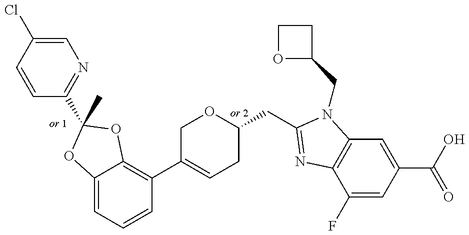 [SMILES: C[C@]1(c2ccc(Cl)cn2)Oc2cccc(C3=CC[C@@H](Cc4nc5c(F)cc(C(=O)O)cc5n4C[C@@H]4CCO4)OC3)c2O1] | MS: m/z = 592.3 (M + 1). 1H NMR (400 MHz, DMSO- d6) δ 8.71 (s, 1H), 8.07 (s, 1H), 7.99 (d, J = 8.5 Hz, 1H), 7.59 (d, J = 7.9 Hz, 1H), 7.48 (d, J = 11.4 Hz, 1H), 6.87- 6.75 (m, 3H), 6.40 (d, J = 10.2 Hz, 1H), 5.09-4.96 (m, 1H), 4.65 (d, J = 4.7 Hz, 1H), 4.62- 4.47 (m, 2H), 4.47-4.35 (m, 2H), 4.31-4.22 (m, 1H), 4.22- 4.10 (m, 1H), 2.65 (s, 1H), 2.43-2.29 (m, 4H), 2.00 (s, 3H). |
| 120 | 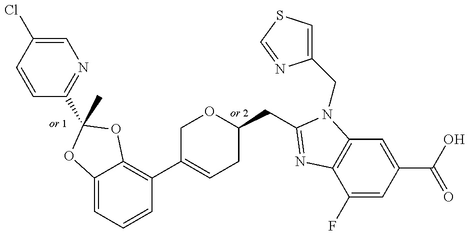 | MS: m/z = 580.3 (M + 1). ¹H NMR (400 MHz, DMSO- d₆) δ 8.99 (s, 1H), 8.71 (s, 1H), 8.01-7.94 (m, 3H), 7.60 (d, J = 8.3 Hz, 1H), 7.51 (d, J = 10.4 Hz, 1H), 6.84- 6.76 (m, 3H), 6.40 (s, 1H), 5.90 (s, 2H), 4.42 (s, 2H), 4.15- 4.09 (m, 1H), 3.28-3.20 (m, 2H), 2.40-2.34 (m, 4H), 2.00 (s, 3H). |
| 70 | 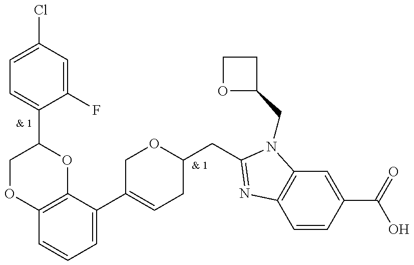 [SMILES: O=C(O)c1ccc2nc(CC3CC=C(c4cccc5c4OC(c4ccc(Cl)cc4F)CO5)CO3)n(C[C@@H]3CCO3)c2c1] | MS: m/z = 591 (M + 1). |
| 121 | 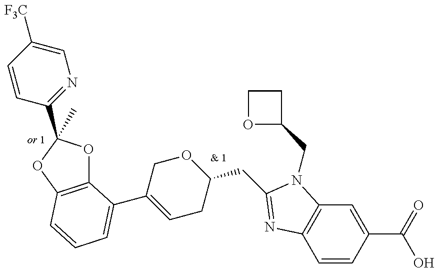 [SMILES: C[C@@]1(c2ccc(C(F)(F)F)cn2)Oc2cccc(C3=CC[C@@H](Cc4nc5ccc(C(=O)O)cc5n4C[C@@H]4CCO4)OC3)c2O1] | MS: m/z = 608 (M + 1). ¹H NMR (400 MHz, DMSO- d₆) δ 9.07 (s, 1H), 8.35-8.26 (m, 2H), 8.18 (s, 1H), 7.83- 7.75 (m, 2H), 7.60-7.52 (m, 1H), 6.90-6.77 (m, 3H), 6.45- 6.39 (m, 1H), 5.08-4.96 (m, 1H), 4.72-4.61 (m, 1H), 4.56- 4.40 (m, 4H), 4.36-4.23 (m, 1H), 4.16 (s, 1H), 3.25-3.19 (m, 1H), 2.65 (s, 2H), 2.39- 2.29 (m, 3H), 2.04 (s, 3H). |
| 122 | 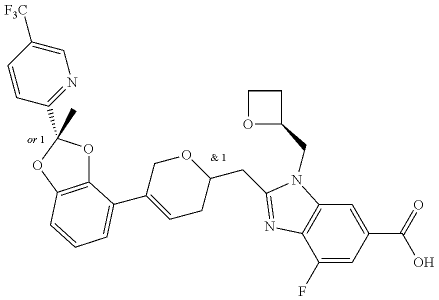 [SMILES: C[C@]1(c2ccc(C(F)(F)F)cn2)Oc2cccc(C3=CCC(Cc4nc5c(F)cc(C(=O)O)cc5n4C[C@@H]4CCO4)OC3)c2O1] | MS: m/z = 626 (M + 1). |
| 123 | 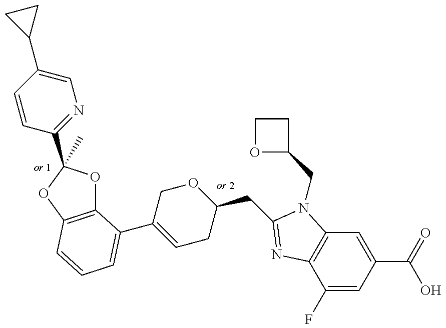 [SMILES: C[C@@]1(c2ccc(C3CC3)cn2)Oc2cccc(C3=CC[C@H](Cc4nc5c(F)cc(C(=O)O)cc5n4C[C@@H]4CCO4)OC3)c2O1] | MS: m/z = 598 (M + 1). |

### Example 17: Compound 78 and Compound 79

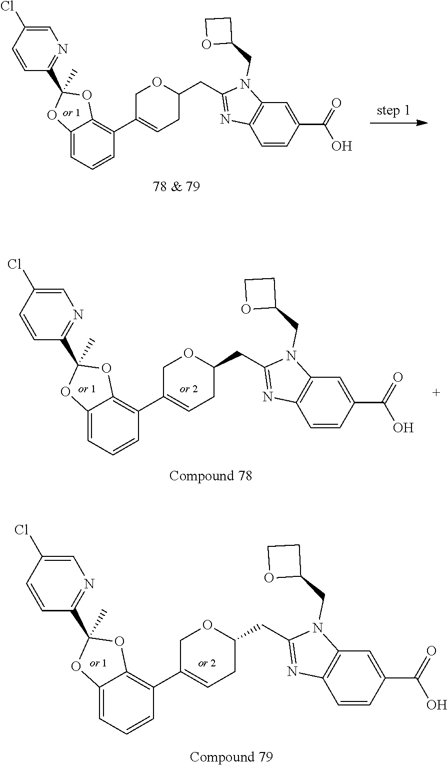 [SMILES: C[C@@]1(c2ccc(Cl)cn2)Oc2cccc(C3=CCC(Cc4nc5ccc(C(=O)O)cc5n4C[C@@H]4CCO4)OC3)c2O1 ; C[C@@]1(c2ccc(Cl)cn2)Oc2cccc(C3=CC[C@H](Cc4nc5ccc(C(=O)O)cc5n4C[C@@H]4CCO4)OC3)c2O1 ; C[C@@]1(c2ccc(Cl)cn2)Oc2cccc(C3=CC[C@@H](Cc4nc5ccc(C(=O)O)cc5n4C[C@@H]4CCO4)OC3)c2O1]

### Step 1: Compound 78 and Compound 79

Compounds 78 & 79 (25 mg) was purified by SFC (column: Daicel ChiralPak AD-H 250 mm×30 mm I.D., 5 m; Mobile Phase: CO₂/MeOH (0.1% DEA)=60/40; Flowrate: 50 g/min; Wavelength: 254 nm; Temperature: 40° C.) to give Compound 78 (7 mg, 12.19 mol) and Compound 79 (4 mg, 6.97 μmol) as white solid.

Compound 78 (peak 1): ¹H NMR (400 MHz, DMSO-d6) δ 8.71 (s, 1H), 8.13 (s, 1H), 7.99 (d, J=8.6 Hz, 1H), 7.82 (s, 1H), 7.59 (d, J=8.6 Hz, 1H), 7.51 (d, J=8.0 Hz, 1H), 6.88-6.73 (m, 3H), 6.40 (s, 1H), 5.12-4.98 (m, 1H), 4.69-4.58 (m, 1H), 4.58-4.50 (m, 1H), 4.48-4.39 (m, 2H), 4.33-4.25 (m, 1H), 4.21-4.12 (m, 1H), 3.28-3.05 (m, 1H), 3.15 (d, J=4.8 Hz, 1H), 2.71-2.60 (m, 2H), 2.38-2.25 (m, 2H), 2.00 (s, 3H), 1.25-1.18 (m, 2H).

Compound 79 (peak 2): ¹H NMR (400 MHz, DMSO-d6) δ 8.71 (s, 1H), 8.13 (s, 1H), 7.99 (d, J=8.6 Hz, 1H), 7.80 (s, 1H), 7.58 (d, J=8.6 Hz, 1H), 7.50 (d, J=8.0 Hz, 1H), 6.93-6.64 (m, 3H), 6.40 (s, 1H), 5.12-4.98 (m, 1H), 4.69-4.58 (m, 1H), 4.58-4.50 (m, 1H), 4.48-4.39 (m, 2H), 4.33-4.25 (m, 1H), 4.21-4.12 (m, 1H), 3.28-3.05 (m, 1H), 3.15 (d, J=4.8 Hz, 1H), 2.71-2.60 (m, 2H), 2.38-2.25 (m, 2H), 2.00 (s, 3H), 1.25-1.18 (m, 2H).

The compounds in Table P below were made according to the procedure of Compound 23.

TABLE P

| Name | Structure | ¹H NMR and/or LC/MS data |
|---|---|---|
| 68 | 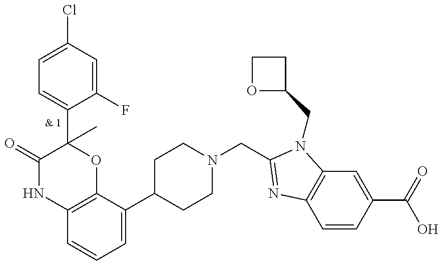 [SMILES: CC1(c2ccc(Cl)cc2F)Oc2c(cccc2C2CCN(Cc3nc4ccc(C(=O)O)cc4n3C[C@@H]3CCO3)CC2)NC1=O] | MS: m/z = 619.3 (M + 1). ¹H NMR (400 MHz, DMSO-d₆) δ 10.95 (s, 1H), 8.22 (s, 1H), 7.85- 7.78 (m, 1H), 7.63-7.55 (m, 1H), 7.50-7.43 (d, J = 10.6 Hz, 1H), 7.42-7.37 (m, 1H), 7.31-7.25 (m, 1H), 6.90-6.80 (m, 2H), 6.75- 6.69 (m, 1H), 5.10-5.02 (m, 1H), 4.79-4.69 (m, 1H), 4.66-4.56 (m, 1H), 4.52-4.43 (m, 1H), 4.39- 4.32 (m, 1H), 3.95-3.86 (m, 1H), 3.80-3.73 (m, 1H), 2.98-2.82 (m, 4H), 2.70-2.64 (m, 1H), 2.22- 2.12 (m, 2H), 1.82 (s, 3H), 1.67- 1.63 (m, 1H), 1.58-1.53 (m, 1H), 1.50-1.43 (m, 1H). |
| 72 | 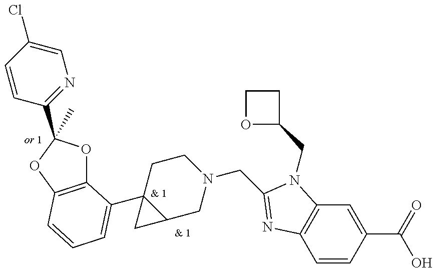 [SMILES: C[C@@]1(c2ccc(Cl)cn2)Oc2cccc(C34CCN(Cc5nc6ccc(C(=O)O)cc6n5C[C@@H]5CCO5)CC3C4)c2O1] | MS: m/z = 587.1 (M + 1). ¹H NMR (400 MHz, DMSO-d₆) δ 8.70 (s, 1H), 8.24 (s, 1H), 7.99 (d, J = 8.4 Hz, 1H), 7.78 (d, J = 8.8 Hz, 1H), 7.64-7.57 (m, 2H), 6.78- 6.71 (m, 3H), 5.10-4.97 (m, 1H), 4.84-4.75 (m, 1H), 4.70-4.59 (m, 1H), 4.44 (d, J = 6.4 Hz, 1H), 4.42- 4.30 (m, 1H), 3.97-3.67 (m, 2H), 2.85-2.74 (m, 2H), 2.69-2.58 (m, 1H), 2.41-3.31 (m, 1H), 2.29- 2.18 (m, 1H), 2.12 (s, 3H), 1.94- 1.82 (m, 1H), 1.39-1.30 (m, 2H), 1.28-1.19 (m, 1H), 1.09-0.95 (m, 1H), 0.91-0.85 (m, 1H). |
| 74 | 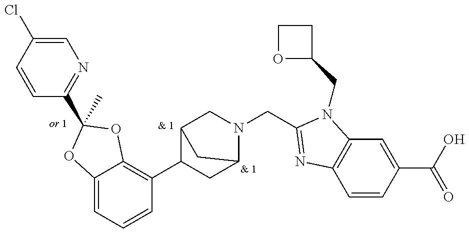 [SMILES: C[C@@]1(c2ccc(Cl)cn2)Oc2cccc(C3CC4CC3CN4Cc3nc4ccc(C(=O)O)cc4n3C[C@@H]3CCO3)c2O1] | MS: m/z = 587.3 (M + 1). |
| 76 | 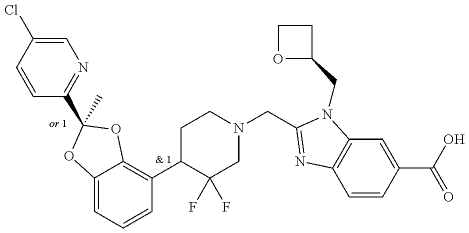 [SMILES: C[C@@]1(c2ccc(Cl)cn2)Oc2cccc(C3CCN(Cc4nc5ccc(C(=O)O)cc5n4C[C@@H]4CCO4)CC3(F)F)c2O1] | MS: m/z = 611.3 (M + 1). |
| 81 | 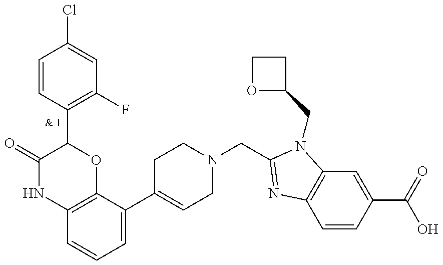 [SMILES: O=C(O)c1ccc2nc(CN3CC=C(c4cccc5c4OC(c4ccc(Cl)cc4F)C(=O)N5)CC3)n(C[C@@H]3CCO3)c2c1] | MS: m/z = 603 (M + 1). 1H NMR (400 MHz, DMSO-d6) δ 11.00 (s, 1H), 8.22 (s, 1H), 7.78 (d, J = 8.3 Hz, 1H), 7.56 (dd, J = 16.5, 9.2 Hz, 2H), 7.41 (t, J = 8.0 Hz, 1H), 7.33 (d, J = 8.0 Hz, 1H), 6.92 (t, J = 7.7 Hz, 1H), 6.82 (dd, J = 16.8, 7.1 Hz, 2H), 5.85 (s, 1H), 5.08- 4.99 (m, 1H), 4.77-4.68 (m, 1H), 4.58 (d, J = 15.4 Hz, 1H), 4.49- 4.42 (m, 1H), 4.36-4.28 (m, 1H), 3.86 (d, J = 13.0 Hz, 1H), 3.72 (d, J = 13.2 Hz, 2H), 2.97-2.85 (m, 1H), 2.74-2.62 (m, 2H), 2.42-2.29 (m, 1H), 2.11-2.04 (m, 1H), 1.70- 1.57 (m, 2H), 1.54-1.39 (m, 2H). |
| 83 | 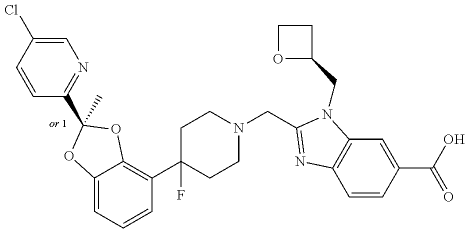 [SMILES: C[C@@]1(c2ccc(Cl)cn2)Oc2cccc(C3(F)CCN(Cc4nc5ccc(C(=O)O)cc5n4C[C@@H]4CCO4)CC3)c2O1] | MS: m/z = 593.2 (M + 1). ¹H NMR (400 MHz, DMSO-d₆) δ 8.72 (d, J = 2.4 Hz, 1H), 8.26 (s, 1H), 8.03 (d, J = 7.2 Hz, 1H), 7.80 (d, J = 8.4 Hz, 1H), 7.66-7.59 (m, 2H), 6.97-6.91 (m, 3H), 5.33 (s, 1H), 5.11 (s, 2H), 4.72 (d, J = 6.4 Hz, 2H), 4.60 (d, J = 14.8 Hz, 2H), 4.44-4.32 (m, 3H), 4.01-3.92 (m, 1H), 3.90-3.84 (m, 1H), 2.04 (s, 3H), 2.01-1.90 (m, 4H), 1.49- 1.35 (m, 2H). |
| 85 | 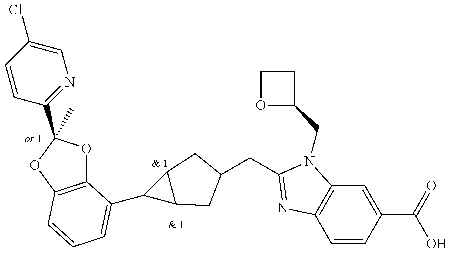 [SMILES: C[C@@]1(c2ccc(Cl)cn2)Oc2cccc(C3C4CC(Cc5nc6ccc(C(=O)O)cc6n5C[C@@H]5CCO5)CC43)c2O1] | MS: m/z = 573.2 (M + 1). ¹H NMR (400 MHz, DMSO-d₆) δ 8.70 (s, 1H), 8.26 (s, 1H), 8.06- 7.96 (m, 1H), 7.82-7.76 (m, 1H), 7.68-7.54 (m, 2H), 6.96-6.80 (m, 1H), 6.75-6.68 (m, 1H), 6.48- 6.42 (m, 1H), 5.08-5.00 (m, 1H), 4.73-4.65 (m, 1H), 4.59-4.51 (m, 1H), 4.48-4.42 (m, 1H), 4.40- 4.33 (m, 1H), 4.11-4.04 (m, 1H), 3.93-3.84 (m, 1H), 3.13-3.07 (m, 1H), 2.98-2.92 (m, 1H), 2.73- 2.60 (m, 2H), 2.45-2.37 (m, 1H), 2.00 (s, 3H), 1.96-1.91 (m, 2H), 1.87-1.76 (m, 2H). |
| 86 | 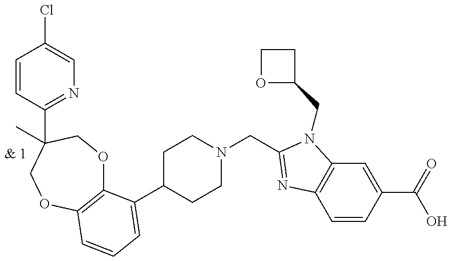 [SMILES: CC1(c2ccc(Cl)cn2)COc2cccc(C3CCN(Cc4nc5ccc(C(=O)O)cc5n4C[C@@H]4CCO4)CC3)c2OC1] | MS: m/z = 603.1 (M + 1). ¹H NMR (400 MHz, DMSO-d₆) δ 8.61 (s, 1H), 8.23 (s, 1H), 7.98- 7.89 (m, 1H), 7.78 (d, J = 8.5 Hz, 1H), 7.62 (dd, J = 16.6, 8.5 Hz, 2H), 6.87 (d, J = 3.7 Hz, 2H), 6.82- 6.77 (m, 1H), 5.08 (d, J = 7.4 Hz, 1H), 4.87-4.74 (m, 1H), 4.68- 4.54 (m, 3H), 4.48 (d, J = 6.4 Hz, 1H), 4.36 (d, J = 8.6 Hz, 1H), 4.06 (dd, J = 11.6, 7.6 Hz, 2H), 3.92 (d, J = 13.6 Hz, 1H), 3.76 (d, J = 13.8 Hz, 1H), 2.99-2.91 (s, 1H), 2.90- 2.79 (s, 2H), 2.75-2.58 (m, 1H), 2.57-2.34 (m, 1H), 2.30-2.12 (m, 2H), 1.70-1.53 (m, 5H), 1.27 (s, |
| | | 3H). |
| 104 | 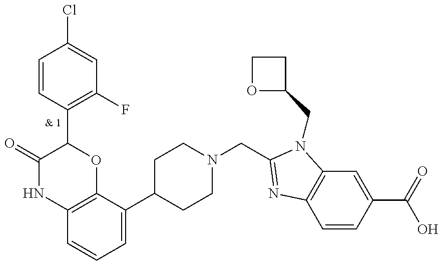 [SMILES: O=C(O)c1ccc2nc(CN3CCC(c4cccc5c4OC(c4ccc(Cl)cc4F)C(=O)N5)CC3)n(C[C@@H]3CCO3)c2c1] | MS: m/z = 605 (M + 1). ¹H NMR (400 MHz, DMSO-d6) δ 11.02 (s, 1H), 8.22 (s, 1H), 7.78 (d, J = 8.8 Hz, 1H), 7.60 (d, J = 8.3 Hz, 1H), 7.52 (d, J = 10.2 Hz, 1H), 7.46- 7.40 (m, 1H), 7.32 (d, J = 8.0 Hz, 1H), 6.96-6.91 (m, 1H), 6.83 (dd, J = 14.9, 7.6 Hz, 2H), 5.85 (s, 1H), 5.76 (s, 1H), 5.03-4.92 (m, 1H), 4.75-4.63 (m, 1H), 4.55 (d, J = 14.7 Hz, 1H), 4.39-4.34 (m, 1H), 4.33-4.26 (m, 1H), 3.97 (d, J = 13.6 Hz, 1H), 3.80 (d, J = 14.6 Hz, 1H), 3.13-2.96 (m, 3H), 2.67- 2.57 (m, 2H), 2.39-2.18 (m, 4H). |
| 105 | 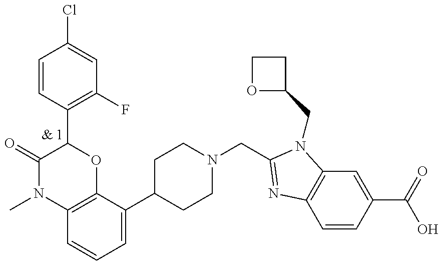 [SMILES: CN1C(=O)C(c2ccc(Cl)cc2F)Oc2c(C3CCN(Cc4nc5ccc(C(=O)O)cc5n4C[C@@H]4CCO4)CC3)cccc21] | MS: m/z = 619 (M + 1). ¹H NMR (400 MHz, DMSO-d6) δ 8.11 (s, 1H), 7.79 (d, J = 7.8 Hz, 1H), 7.54 (d, J = 10.4 Hz, 1H), 7.43 (d, J = 8.0 Hz, 1H), 7.37 (t, J = 7.5 Hz, 1H), 7.30 (d, J = 8.4 Hz, 1H), 7.08-7.00 (m, 2H), 6.94 (d, J = 5.8 Hz, 1H), 5.92 (s, 1H), 5.09-4.96 (m, 1H), 4.72-4.60 (m, 1H), 4.53 (d, J = 14.4 Hz, 1H), 4.47-4.41 (m, 1H), 4.38-4.28 (m, 1H), 3.83 (d, J = 13.0 Hz, 1H), 3.70 (d, J = 12.9 Hz, 1H), 3.48-3.37 (m, 3H), 2.96- 2.85 (m, 1H), 2.84-2.73 (m, 2H), 2.68-2.57 (m, 1H), 2.42-2.31 (m, 1H), 2.17-1.94 (m, 3H), 1.76- 1.60 (m, 3H). |

The compound in Table Q below was made according to the procedure of Compounds 28&29.

TABLE Q

| | | ¹H NMR and/or LC/MS |
|---|---|---|
| Name | Structure | data |
| 71 | 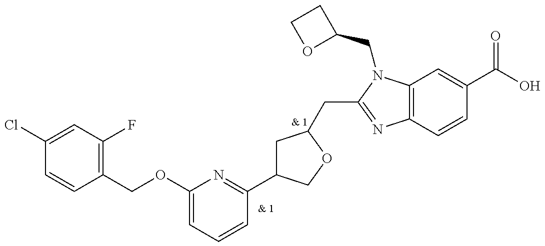 [SMILES: O=C(O)c1ccc2nc(CC3CC(c4cccc(OCc5ccc(Cl)cc5F)n4)CO3)n(C[C@@H]3CCO3)c2c1] | MS: m/z = 552.2 (M + 1). |

### Example 18: Compound 80

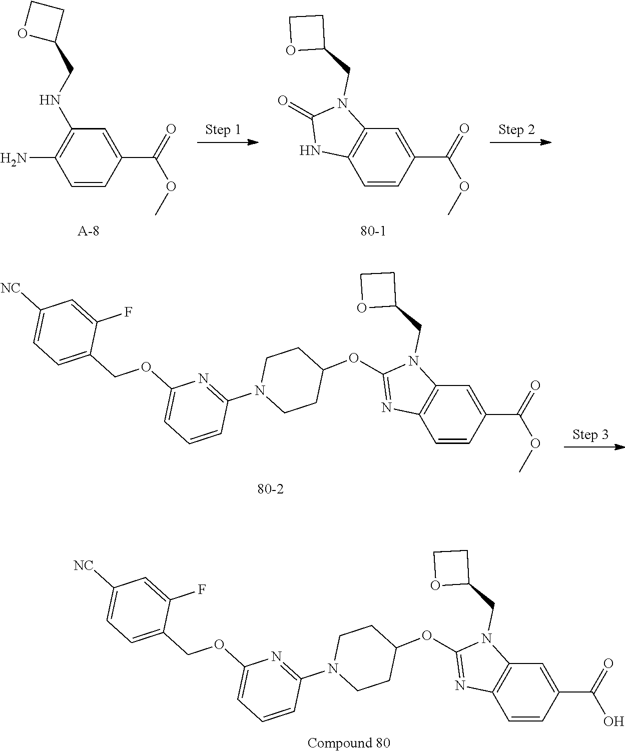 [SMILES: COC(=O)c1ccc(N)c(NC[C@@H]2CCO2)c1 ; COC(=O)c1ccc2[nH]c(=O)n(C[C@@H]3CCO3)c2c1 ; "A-8" ; COC(=O)c1ccc2nc(OC3CCN(c4cccc(OCc5ccc(C#N)cc5F)n4)CC3)n(C[C@@H]3CCO3)c2c1 ; N#Cc1ccc(COc2cccc(N3CCC(Oc4nc5ccc(C(=O)O)cc5n4C[C@@H]4CCO4)CC3)n2)c(F)c1]

### Step 1: 80-1

To a mixture of A-8 (200 mg, 846.50 μmol) in THF (10 mL) was added CDI (182.78 mg, 1.27 mmol), and the mixture was stirred for 18 hrs at 60° C. The mixture was concentrated and purified by FCC (Gradient: 0-50% EA in petroleum ether) to give 80-1 (200 mg, 762.60 mol, 90.1% yield). MS: m/z=263.1 (M+1).

### Step 2: 80-2

To a mixture of Intermediate AN (20 mg, 61.10 μmol) and 80-1 (16.02 mg, 61.10 mol) in THF (3 mL) was added triphenylphosphane (19.23 mg, 73.32 μmol) and DIAD (14.83 mg, 73.32 μmol). The mixture was stirred for 2 hrs at 25° C. The mixture was concentrated and purified by FCC (Gradient: 0-50% EA in petroleum ether) to give 80-2 (10 mg, 17.49 μmol, 28.6% yield). MS: m/z=572.3 (M+1).

### Step 3: Compound 80

A solution of 80-2 (10 mg, 17.49 μmol) and NaOH (80 mg, 2.0 mmol) in Methanol (2 mL), THF (1 mL) and Water (1 mL) was stirred at 50° C. for 2 hrs. The reaction mixture was concentrated and adjusted to pH˜5, filtered, and washed by water (10 mL×3). The crude product was purified by prep-HPLC to give Compound 80 (5 mg, 9.0 μmol, 51.3% yield). MS: m/z=558.4 (M+1).

### Example 19: Compound 102 and Compound 103

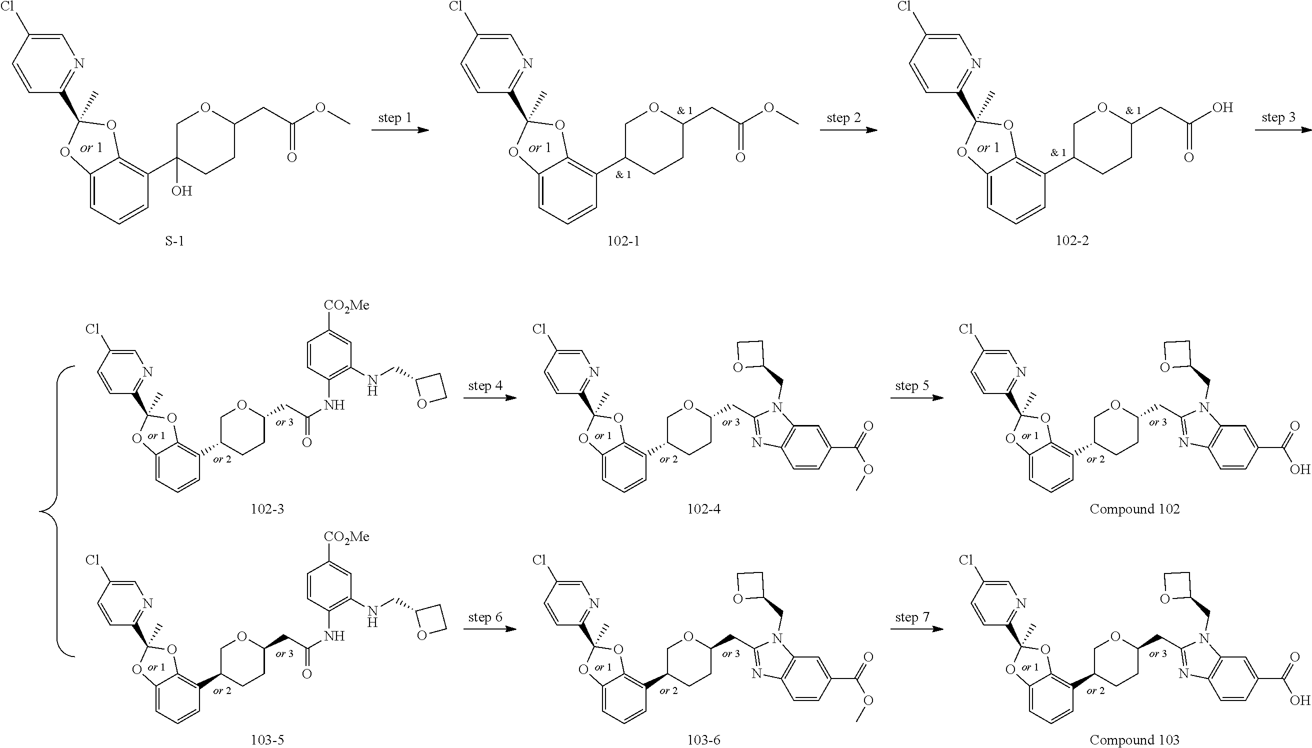

### Step 1: 102-1

To a mixture of S-1 (0.4 g, 952.71 μmol) in TFA (10 mL) was added triethylsilane (1.11 g, 9.53 mmol, 1.52 mL) at 0° C. The reaction was stirred for 6 hrs at 60° C. The mixture was concentrated and purified by silica gel chromatography (petroleum ether/ethyl acetate=4/1, v/v) to give 102-1 (300 mg, 742.84 μmol, 78.0% yield).

### Step 2: 102-2

To a solution of 102-1 (300 mg, 742.84 μmol) in THF (5 mL), Methanol (5 mL) and Water (3 mL) was added NaOH (89.14 mg, 2.23 mmol). The reaction was stirred at 40° C. for 1 hr and extracted with EA (30 mL×3). The aqueous layer was adjusted to pH˜5 with 1 M HCl. The mixture was extracted with EA (30 mL×3), and the combined organic layers were with brine (30 mL×3), dried over Na₂SO₄, filtered and concentrated to a residue, which was purified by SFC (Column: Daicel ChiralPak AD-H 250 mm×30 mm I.D., 5 m; Mobile Phase: CO₂/EtOH (0.1% DEA)=70:30; Flowrate: 50 g/min; Wavelength: 254 nm; Temperature: 40° C.) to give 102-2 (125 mg, 320.65 μmol, 43.2% yield). MS: m/z=390.1 (M+1).

### Step 3: 102-3 & 103-5

To a mixture of 102-2 (70 mg, 179.57 μmol), methyl 4-amino-3-[[(2S)-oxetan-2-yl]methylamino]benzoate (42.43 mg, 179.57 μmol) in DMF (6 mL) was added DIPEA (69.62 mg, 538.70 μmol) and HATU (102.41 mg, 269.35 μmol) at 30° C. The reaction solution was stirred for 4 hrs at 30° C. before EA (100 mL) was added. The mixture was washed with H₂O (100 mL×3), and the organic layer was dried over Na₂SO₄, filtered and concentrated to give a residue, which was purified by silica gel chromatography (petroleum ether/ethyl acetate=5:1-1:1, v/v) to give a mixture, which was further separated by SFC (Column: Daicel ChiralPak AD-H 250 mm×30 mm I.D., 5 m; Mobile Phase: CO₂/EtOH (0.1% DEA)=55:45; Flowrate: 50 g/min; Wavelength: 254 nm; Temperature: 40° C.) to give 102-3 (peak 1, 36 mg, 59.20 μmol, 33.0% yield) and 103-5 (peak 2, 37 mg, 60.85 μmol, 33.9% yield). MS: m/z=608.2 (M+1).

### Step 4: 102-4

A solution of 102-3 (36 mg, 59.20 μmol) in AcOH (8 mL) was stirred at 100° C. for 1 hr. The resulting mixture was cooled to room temperature and concentrated to give a residue, which was purified by FCC (silica gel, petroleum ether/EA=10/1 to 1/1) to give 102-4 (30 mg, 50.84 μmol, 85.9% yield). MS: m/z=590.1 (M+1).

### Step 5: Compound 102

A mixture of 102-4 (25 mg, 42.37 μmol) and NaOH (5.08 mg, 127.10 μmol) in THF (5 mL), Water (2 mL) and Methanol (3 mL) was stirred at 25° C. for 2 hrs. The resulting mixture was acidified with 1 M HCl to pH=5-6 and filtered to give Compound 102 (23 mg, 39.93 mol, 94.2% yield). MS: m/z=576.2 (M+1).

¹H NMR (400 MHz, DMSO-d6) δ 8.70 (d, J=1.7 Hz, 1H), 8.17 (s, 1H), 7.99 (dd, J=8.4, 2.4 Hz, 1H), 7.77 (d, J=7.8 Hz, 1H), 7.62-7.53 (m, 2H), 6.79-6.74 (m, 3H), 5.08-4.98 (m, 1H), 4.67-4.57 (m, 1H), 4.55-4.47 (m, 1H), 4.45-4.39 (m, 1H), 4.27-4.20 (m, 1H), 4.02-3.91 (m, 1H), 3.84-3.75 (m, 1H), 3.20-3.12 (m, 2H), 2.91-2.80 (m, 1H), 2.70-2.58 (m, 1H), 2.37-2.25 (m, 1H), 1.99 (s, 3H), 1.96-1.82 (m, 3H), 1.57-1.39 (m, 1H), 1.23-1.19 (m, 1H).

### Step 6: 103-6

A solution of 103-5 (35 mg, 57.56 μmol) in AcOH (5 mL) was stirred at 100° C. for 1 hr. The resulting mixture was cooled to room temperature and concentrated to give a residue, which was purified by FCC (silica gel, petroleum ether/EA=10/1 to 1/1) to give 103-6 (30 mg, 50.84 μmol, 88.3% yield). MS: m/z=590.1 (M+1).

### Step 7: Compound 103

A mixture of 103-6 (30 mg, 50.84 μmol) and NaOH (6.10 mg, 152.53 μmol) in THF (5 mL), Water (2 mL) and Methanol (2 mL) was stirred at 25° C. for 2 hrs. The resulting mixture was acidified with 1 M HCl to pH=5-6 and filtered to give Compound 103 (20 mg, 34.72 mol, 68.3% yield). MS: m/z=576.2 (M+1).

¹H NMR (400 MHz, DMSO-d6) δ 8.70 (d, J=1.7 Hz, 1H), 8.17 (s, 1H), 7.99 (dd, J=8.4, 2.4 Hz, 1H), 7.78 (d, J=7.8 Hz, 1H), 7.62-7.52 (m, 2H), 6.79-6.73 (m, 3H), 5.08-4.97 (m, 1H), 4.66-4.57 (m, 1H), 4.55-4.47 (m, 1H), 4.45-4.40 (m, 1H), 4.27-4.21 (m, 1H), 4.02-3.91 (m, 1H), 3.84-3.73 (m, 1H), 3.20-3.10 (m, 2H), 2.91-2.79 (m, 1H), 2.70-2.59 (m, 1H), 2.37-2.26 (m, 1H), 1.99 (s, 3H), 1.96-1.82 (m, 3H), 1.57-1.39 (m, 1H), 1.23-1.20 (m, 1H).

### Example 20: Compound 110

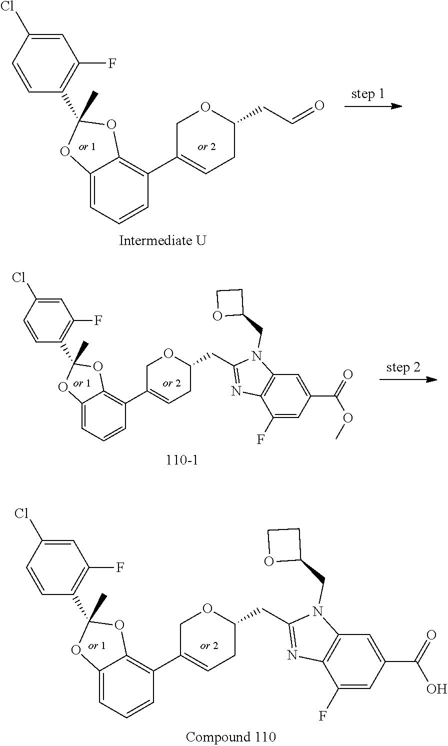 [SMILES: C[C@]1(c2ccc(Cl)cc2F)Oc2cccc(C3=CC[C@@H](CC=O)OC3)c2O1 ; COC(=O)c1cc(F)c2nc(C[C@@H]3CC=C(c4cccc5c4O[C@@](C)(c4ccc(Cl)cc4F)O5)CO3)n(C[C@@H]3CCO3)c2c1 ; C[C@]1(c2ccc(Cl)cc2F)Oc2cccc(C3=CC[C@@H](Cc4nc5c(F)cc(C(=O)O)cc5n4C[C@@H]4CCO4)OC3)c2O1]

### Step 1: 110-1

A solution of Intermediate U (35 mg, 90.02 μmol) and Intermediate AM (27.46 mg, 108.02 μmol) in Tol. (50 mL) was stirred at 100° C. for 24 hrs. The mixture was concentrated to give a residue, which was purified by FCC (silica gel, petroleum ether/EA=10/1 to 1/1) to give 110-1 (20 mg, 32.10 μmol, 35.7% yield). MS: m/z=623.3 (M+1).

### Step 2: Compound 110

A mixture of 110-1 (20 mg, 32.10 μmol) and NaOH (6.42 mg, 160.50 μmol) in Water (2 mL) Methanol (5 mL) and THF (10 mL) was stirred at 25° C. for 4 hrs. The resulting mixture was acidified with 1 M HCl to pH=5-6 and extracted with EA (30 mL). The separated organic layer was washed with brine, dried over Na₂SO₄, filtered and concentrated to give a residue, which was purified by prep-HPLC (gradient: 5-95% B, A: 0.2% HCOOH, B: MeCN, GT: 22 min, flow rate: 15 mL/min) to give Compound 110 (10 mg, 16.42 μmol, 51.1% yield). MS: m/z=609.2 (M+1).

¹H NMR (400 MHz, Methanol-d₄) δ 8.01 (s, 1H), 7.63 (d, J=12.0 Hz, 1H), 7.55 (s, 1H), 7.28 (d, J=8.8 Hz, 1H), 7.22 (s, 1H), 6.82-6.66 (m, 3H), 6.44 (s, 1H), 5.19-5.12 (m, 2H), 4.51-4.45 (m, 3H), 4.19-4.09 (m, 1H), 2.88-2.75 (m, 2H), 2.58-2.46 (m, 1H), 2.45-2.39 (m, 2H), 2.20-2.14 (m, 1H), 2.02 (s, 3H), 1.31-1.21 (m, 2H).

The compound in Table R below was made according to the procedure of Compound 110.

TABLE R

| Name | Structure | ¹H NMR and/or LC/MS data |
|---|---|---|
| 108 | 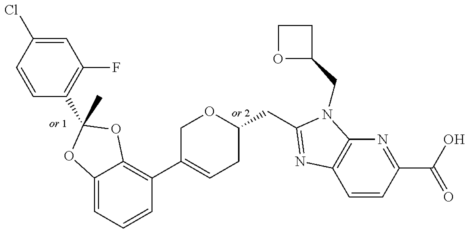 [SMILES: C[C@]1(c2ccc(Cl)cc2F)Oc2cccc(C3=CC[C@@H](Cc4nc5ccc(C(=O)O)nc5n4C[C@@H]4CCO4)OC3)c2O1] | MS: m/z = 592.3 (M + 1) |

### Biological Assays

The biological activities of the compounds of the present application can be assessed with methods and assays known in the art. Exemplary methods are described in the Examples, such as GLP1R cAMP assay and human GLP-1 activity assay.

The compounds of the present application also possess favorable pharmacokinetic properties compared to known small molecule GLP-1 receptor agonists. These properties can be evaluated with methods and assays available in the art, such as those described and/or exemplified herein.

### Example 21: h-GLP-1 Activity Assay

Human GLP-1 agonizing activity was detected in HEK293 cells with stable expression of human GLP-1 by Cisbio cAMP Gs dynamic kit (Catalog #62AM4PEC) according to manufacturer's protocol. Briefly, cells were collected and resuspended in assay buffer containing 0.1% BSA and 0.5 mM of IBMX at concentration to 2.5×10⁵ cells/mL. Two μL 5× compound solution and 8 μL cell suspension were added to each well of low-volume 384 white assay plate. After 30 min incubation at 37° C., 5 μL cAMP-d2 working solution and 5 μL anti-cAMP antibody-cryptate were added to each well, and incubate at room temperature for 1 hr. Series dilution of cAMP was used as standard. Human GLP-1 (7-37) was used as positive control and 10 nM human GLP-1 was set as 100% response. HTRF signals were read at 665 and 615 nm with EnVision plate reader and calculated cAMP concentration by intrapolation to the standard curve. The EC50 value of the tested compounds was calculated by fitting the dose response curve using a 4-parameter non-linear regression routine and relative EC50 was presented in Table S.

As shown in Table S, the compounds exhibit potent h-GLP-1 agonism activity (“A” means >0 nM and <20 nM; “B” means >20 nM and 100 nM; “C” means >100 nM).

TABLE S
h-GLP-1 activity of compounds of the application

| Compound No. | h-GLP-1 Activity (EC₅₀, nM) |
|---|---|
| 3 | C |
| 4 | C |
| 7 | C |
| 15 | A |
| 16 | A |
| 17 | A |
| 18 | A |
| 19 | A |
| 21 | B |
| 22 | B |
| 23 | C |
| 24 | A |
| 25 | C |
| 26 | A |
| 27 | C |
| 28 | A |
| 29 | C |
| 30 | A |
| 31 | A |
| 32 | A |
| 33 | A |
| 34 | C |
| 35 | C |
| 36 | C |
| 37 | A |
| 40 | A |
| 41 | C |
| 42 | C |
| 43 | A |
| 56 | A |
| 58 | C |
| 60 | A |
| 61 | A |
| 68 | B |
| 70 | B |
| 71 | C |
| 72 | A |
| 73 | C |
| 74 | C |
| 75 | C |
| 76 | A |
| 77 | A |
| 78 | A |
| 79 | C |
| 80 | C |
| 81 | C |
| 82 | B |
| 83 | B |
| 85 | B |
| 86 | C |
| 100 | A |
| 101 | B |
| 102 | C |
| 103 | B |
| 104 | B |
| 105 | C |
| 106 | C |
| 107 | C |
| 108 | A |
| 109 | A |
| 110 | A |
| 111 | A |
| 112 | A |
| 113 | B |
| 114 | B |
| 115 | C |
| 116 | A |
| 117 | C |
| 118 | A |
| 119 | B |
| 120 | A |
| 121 | A |
| 122 | C |
| 123 | A |

### Example 22: Pharmacokinetics of the Compounds of the Application

Compounds of the present application were formulated in 0.50% MC, and administered via oral gavage (PO) at the dosages of 5 or 10 mg per kilogram body weight, or formulated in 5% DMSO:15% Solutol 80% (SBECD), and administered via intravenous injection (IV) at the dosages of 1 mg per kilogram body weight, in fasted SD rats. Plasma samples were collected at 0.25, 0.5, 1, 2, 4, 6, 8, and 24 hours post dosing (PO); and were collected at 0.083, 0.25, 0.5, 1, 2, 4, 6, 8, and 24 hours post dosing (IV). Compound concentration was determined by LC-MS and pharmacokinetics parameters were calculated by WinNonlin 8.2 using Non-Compartmental Analysis model. PK parameters of representative compounds were listed in Table T.

TABLE T

| | PO parameters | | | IV parameters | |
|---|---|---|---|---|---|
| | Dose | AUC_{0-inf} | | CL | AUC_{0-inf} |
| Cmpd No. | (mg/kg) | (ng · hr/mL) | F (%) | (mL/min/kg) | (ng · hr/mL) |
| Ref. Cmpd. | 10 | 143 | 7.3 | 86.7 | 195 |
| 19 | 5 | 4414 | 26.1 | 5.5 | 3376 |
| 28 | 5 | 526 | 21.7 | 28.8 | 588 |
| 100 | 5 | 3186 | 36.3 | 9.7 | 1755 |

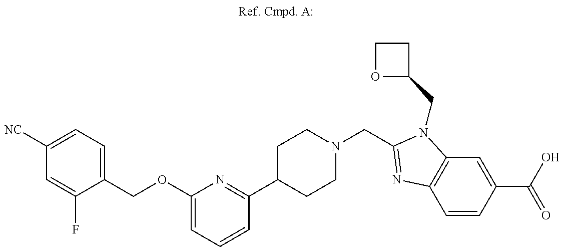 [SMILES: N#Cc1ccc(COc2cccc(C3CCN(Cc4nc5ccc(C(=O)O)cc5n4C[C@@H]4CCO4)CC3)n2)c(F)c1]

### Example 23: Liver Microsome Stability of the Compounds of the Application

Compounds of the present application was incubated with human or rat liver microsomes in the presence of NADPH at 37° C. The concentration of compound at pre-incubation and after 30 min incubation was quantified by LC-MS. The percentage of remaining compound was then calculated and indicative of microsome stability of each compound. Results of representative compounds were listed in Table U.

TABLE U

| Cmpd No. | Human (% remaining @ 30 min) | Rat (% remaining @ 30 min) |
|---|---|---|
| Ref. Cmpd. B | <75% | >75% |
| 78 | >75% | >75% |
| 100 | >75% | >75% |
| 108 | >75% | >75% |
| 110 | >75% | >75% |
| 118 | >75% | >75% |

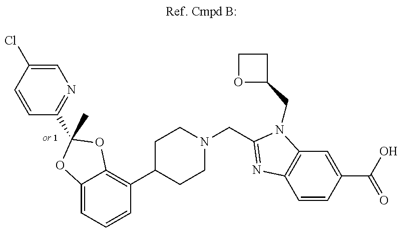 [SMILES: C[C@]1(c2ccc(Cl)cn2)Oc2cccc(C3CCN(Cc4nc5ccc(C(=O)O)cc5n4C[C@@H]4CCO4)CC3)c2O1]

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A compound of Formula (Y): 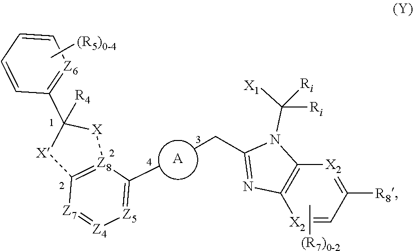
or a pharmaceutically acceptable salt, solvate, prodrug, stereoisomer, or tautomer thereof, wherein:
X₁ is H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S, wherein the carbocyclyl, heterocyclyl, aryl, or heteroaryl is optionally substituted with one or more substituents independently selected from C₁-C₄ alkyl, halo-C₁-C₄ alkyl, C₁-C₄ alkoxy, halo-C₁-C₄ alkoxy, halogen, OH, CN, and NH₂;
each Rᵢ is independently H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S; or two Rᵢ, together with the carbon atom to which they are attached, form a C₃-C₆ carbocyclyl or 4- to 6-membered heterocyclyl comprising 1 or 2 heteroatoms selected from N, O, and S; or one Rᵢ and X₁, together with the carbon atom to which they are attached, form a C₃-C₆ carbocyclyl or 4- to 6-membered heterocyclyl comprising one or two 5- or 6-membered rings and 1 to 4 heteroatoms selected from N, O, and S;
each X₂ is independently CR₇ or N;
Ring A is 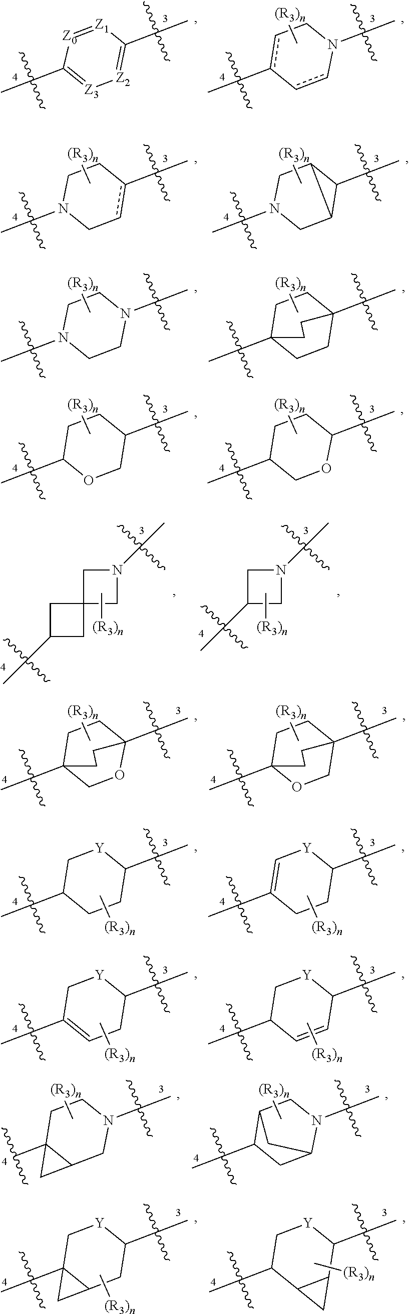 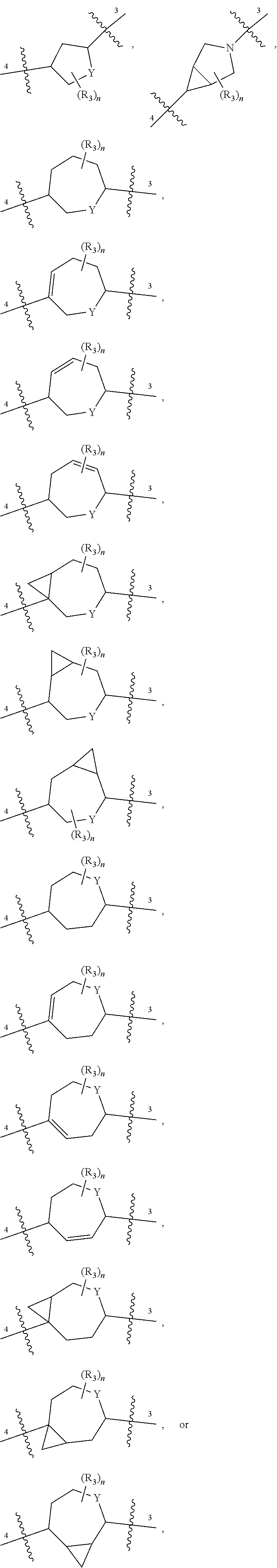
wherein
Z₀, Z₁, Z₂, and Z₃ are each independently CR₃₁, S, or N;
each R₃₁ is independently H or R₃;
Y is O, NR_{b}, NR_{b}C(O), NR_{b}S(O)₂, S, SO₂, or C(R)₂, wherein
R_{b} is H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S;
each R_{c} is independently H, C₁-C₄ alkyl, halo-C₁-C₄ alkyl, C₁-C₄ alkoxy, halo-C₁-C₄ alkoxy, halogen, OH, CN, NH₂, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S; or
two R_{c} are taken together to form an oxo; or
two R_{c}, together with the carbon atom to which they are attached, form a C₃-C₆ carbocylyl or 4- to 6-membered heterocylyl comprising 1 or 2 heteroatoms selected from N, O, and S;
each R₃ is independently C₁-C₄ alkyl, halo-C₁-C₄ alkyl, C₁-C₄ alkoxy, halo-C₁-C₄ alkoxy, halogen, OH, CN, or NH₂; or
two geminal R₃ are taken together to form an oxo; and
n is an integer selected from 0 to 8; 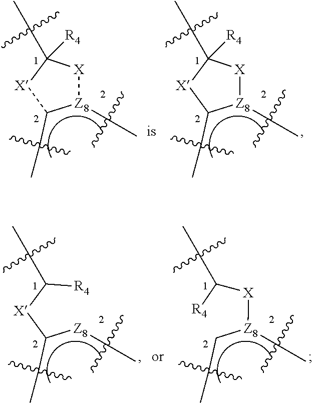
X and X′ are each independently O, NR₁₀, CR₁R₂, C(R₁₁)₂O, or C(R₁₁)₂NR₁₀;
R₁ and R₂ are each independently H, C₁-C₄ alkyl, halo-C₁-C₄ alkyl, C₁-C₄ alkoxy, halo-C₁-C₄ alkoxy, halogen, or OH, or R₁ and R₂, together with the carbon atom to which they are attached, form a C₃-C₆ carbocyclic ring or 4- to 6-membered heterocyclic ring comprising 1 or 2 heteroatoms selected from N, O, and S;
R₁₀ is H, C₁-C₄ alkyl, halo-C₁-C₄ alkyl, or C(O)Rₐ;
Rₐ is H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S;
each R₁₁ is independently H, C₁-C₄ alkyl, halo-C₁-C₄ alkyl, C₁-C₄ alkoxy, halo-C₁-C₄ alkoxy, halogen, or OH; or two R₁₁, together with the carbon atom to which they are attached, form a C₃-C₆ carbocylyl or 4- to 6-membered heterocylyl comprising 1 or 2 heteroatoms selected from N, O, and S; or two R₁₁ are taken together to form an oxo;
Z₄, Z₅, Z₆, and Z₇ are independently CR₆ or N;
Z₈ is C or N, wherein when Z₈ is N, then X is absent;
each R₆ is independently H, C₁-C₄ alkyl, halo-C₁-C₄ alkyl, C₁-C₄ alkoxy, halo-C₁-C₄ alkoxy, halogen, OH, CN, or NH₂;
R₄ is H, C₁-C₄ alkyl, halo-C₁-C₄ alkyl, or C₃-C₆ carbocyclyl;
each R₅ is independently C₁-C₄ alkyl, halo-C₁-C₄ alkyl, C₁-C₄ alkoxy, halo-C₁-C₄ alkoxy, C₃-C₆ carbocyclyl, halogen, OH, CN, or NH₂;
each R₇ is independently C₁-C₄ alkyl, halo-C₁-C₄ alkyl, C₁-C₄ alkoxy, halo-C₁-C₄ alkoxy, halogen, OH, CN, or NH₂; and
R₈′ is C(O)R₈, NHC(O)Rᵢᵢ, or 5- to 10-membered heteroaryl comprising one or two 5- or 6-membered rings and 1 to 4 heteroatoms selected from N, O, and S;
R₈ is ORᵢᵢ, N(Rᵢᵢ)₂, or NRᵢᵢSO₂Rᵢᵢᵢ; wherein
each Rᵢᵢ is independently H, C₁-C₄ alkyl, halo-C₁-C₄ alkyl, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S;
Rᵢᵢᵢ is C₁-C₄ alkyl, halo-C₁-C₄ alkyl, C₁-C₄ alkoxy, C₁-C₄ alkylamino, C₁-C₄ dialkylamino, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S; and
provided that:
1) when Ring A is 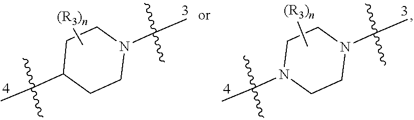
X and X′ are each O, and Z₄ and Z₅ are each CH, then R₄ is not H or methyl;
2) when 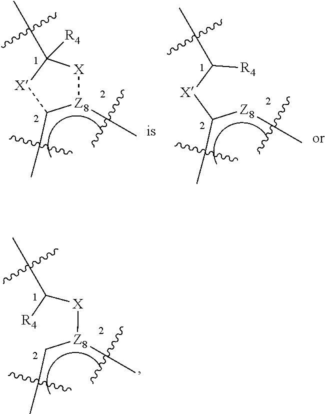
then Ring A is not 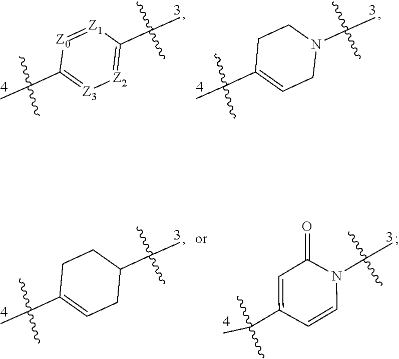
3) when 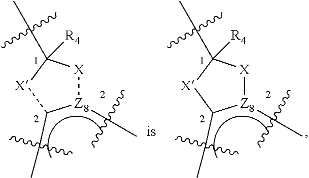
Ring A is 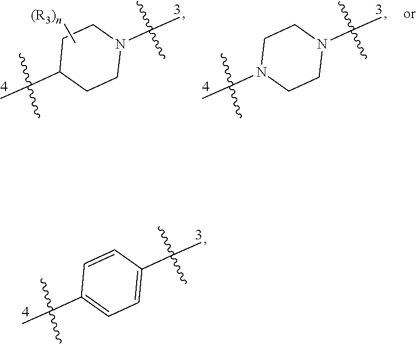
each of Z₄, Z₅, Z₆, Z₇, and Z₈ is CR₆, and X is C(R₁₁)₂O, then X′ is not O, NR₁₀, or CR₁R₂;
4) when 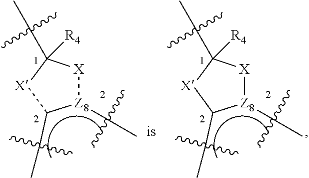
Ring A is 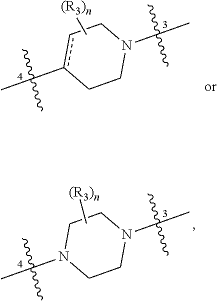
each of Z₄, Z₅, Z₇, and Z₈ is CR₆, Z₆ is CR₆ or N, and X is O, then X′ is not C(R₁₁)₂O;
5) when 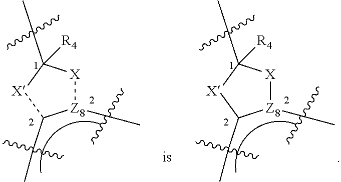
Ring A is 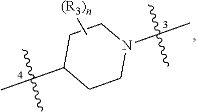
each of Z₄, Z₅, Z₆, Z₇, and Z₈ is CR₆, and X is C(R₁₁)₂O, then X′ is not O;
6) when 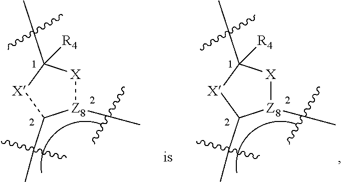
Ring A is 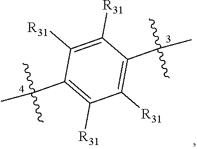
at least one R₃₁ is F, and X is O, NR₁₀, or CR₁R₂, then X′ is not O, NR₁₀, or CR₁R₂; and
7) the compound is not
2-((4-(2-(hydroxymethyl)-2-phenylbenzo[d][1,3]dioxol-4-yl)piperidin-1-yl)methyl)-1-(2-methoxyethyl)-1H-benzo[d]imidazole-6-carboxylic acid;
2-((4-(2-(4-cyano-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid;
2-((4-(2-(4-chloro-2-fluorophenyl)-2-methylbenzo[d][1,3]dioxol-4-yl)-3,6-dihydropyridin-1(2H)-yl)methyl)-1-(((S)-oxetan-2-yl)methyl)-1H-benzo[d]imidazole-6-carboxylic acid,
2-((5-(2-(5-chloropyridin-2-yl)-2-methylbenzo[d][1,3]dioxol-4-yl)-3-fluoropyridin-2-yl)methyl)-1-(((S)-oxetan-2-yl)methyl)-1H-benzo[d]imidazole-6-carboxylic acid,
2-((4-(2-(5-chloropyridin-2-yl)-2-methylbenzo[d][1,3]dioxol-4-yl)-2-oxopyridin-1(2H)-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)pyridin-3-yl)methyl)-1-(((S)-oxetan-2-yl)methyl)-1H-benzo[d]imidazole-6-carboxylic acid,
2-((6-(2-(4-chloro-2-fluorophenyl)-2-methylbenzo[d][1,3]dioxol-4-yl)-2-methoxypyridin-3-yl)methyl)-1-(2-methoxyethyl)-1H-benzo[d]imidazole-6-carboxylic acid, or
2-((6-(2-(4-chloro-2-fluorophenyl)-2-methylbenzo[d][1,3]dioxol-4-yl)-2-oxo-1,2-dihydropyridin-3-yl)methyl)-1-(2-methoxyethyl)-1H-benzo[d]imidazole-6-carboxylic acid.

2.-4. (canceled)

5. The compound of claim 1, wherein the compound is of Formula (I), (Ia), or (Ia′): 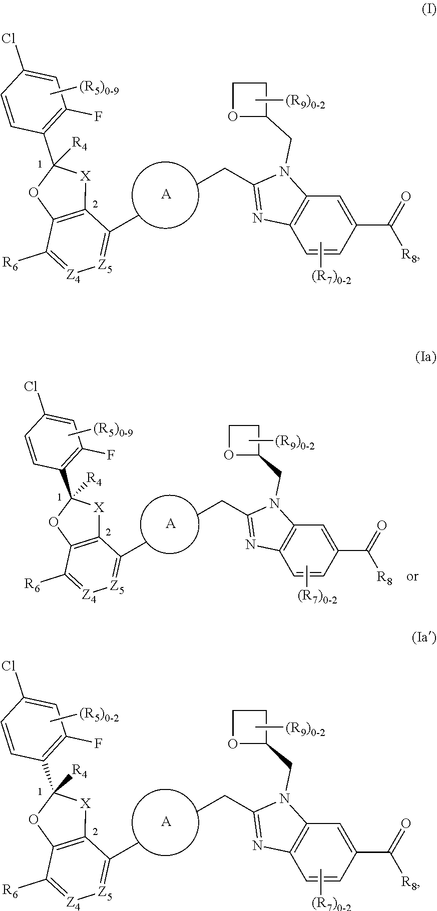
or a pharmaceutically acceptable salt, solvate, prodrug, stereoisomer, or tautomer thereof, wherein each R₉ is independently C₁-C₄ alkyl, halo-C₁-C₄ alkyl, C₁-C₄ alkoxy, halo-C₁-C₄ alkoxy, halogen, OH, CN, or NH₂.

6. (canceled)

7. The compound of claim 1, wherein the compound is of Formula (I1), (I1a), (I1a′) (II1), (II2), (II3), (II4), (II5), (II6), (II7), (II8), (II9), (II10), (II11), (II12), (II13), (II14), (II15), (II16), (II17), or (II18): 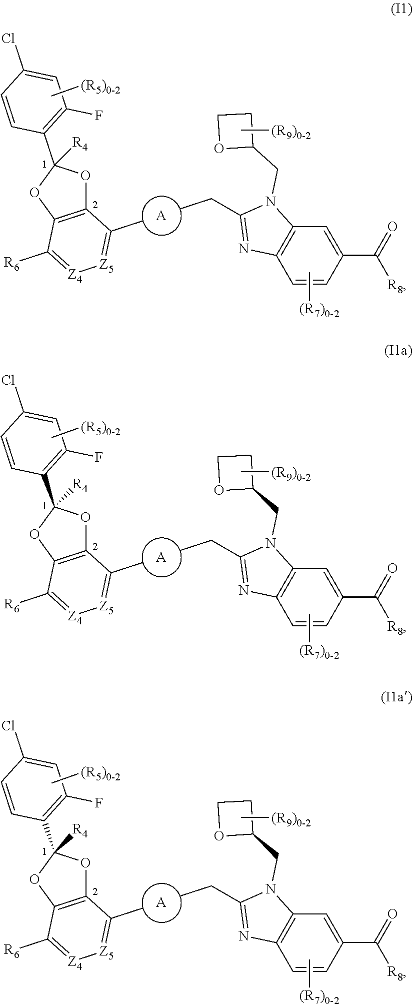 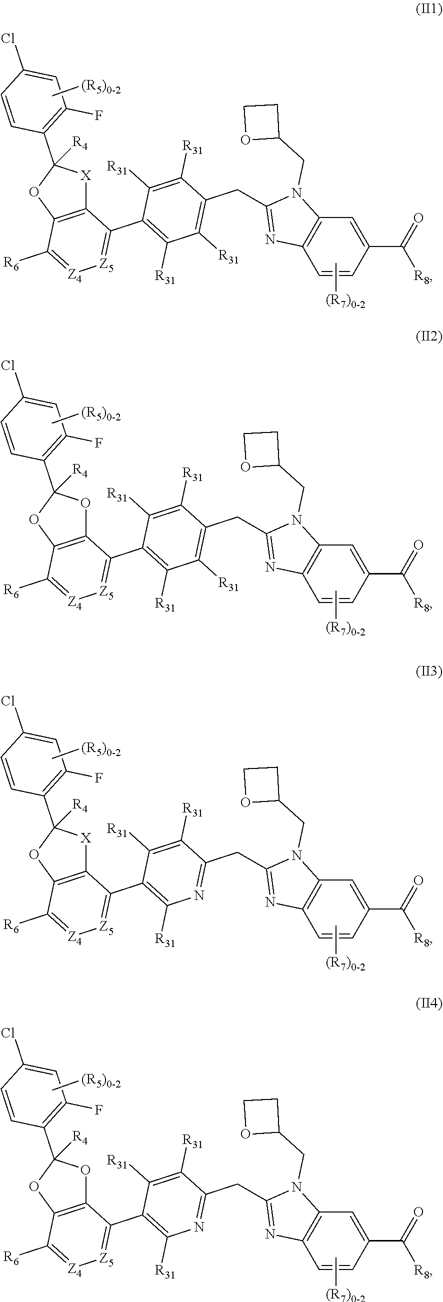 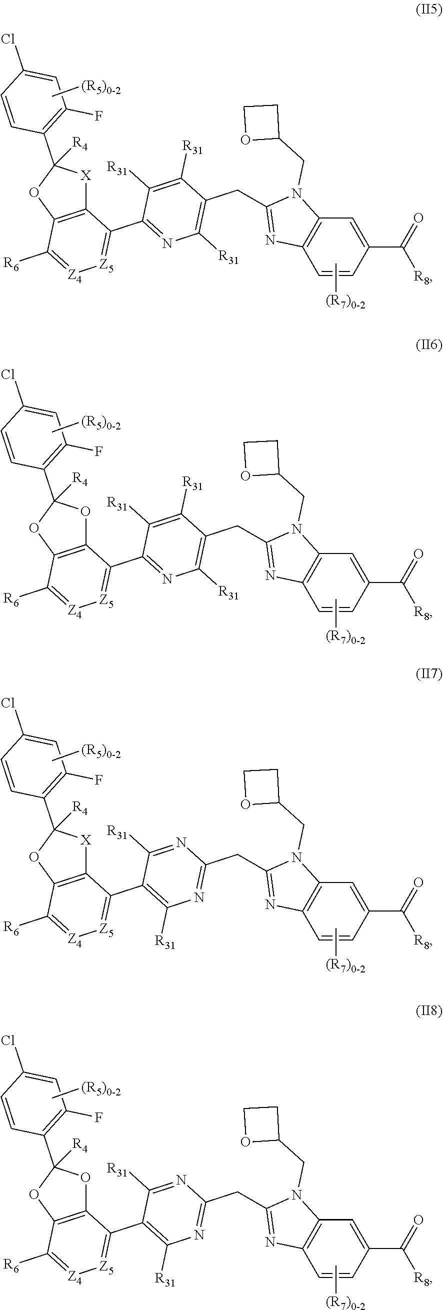 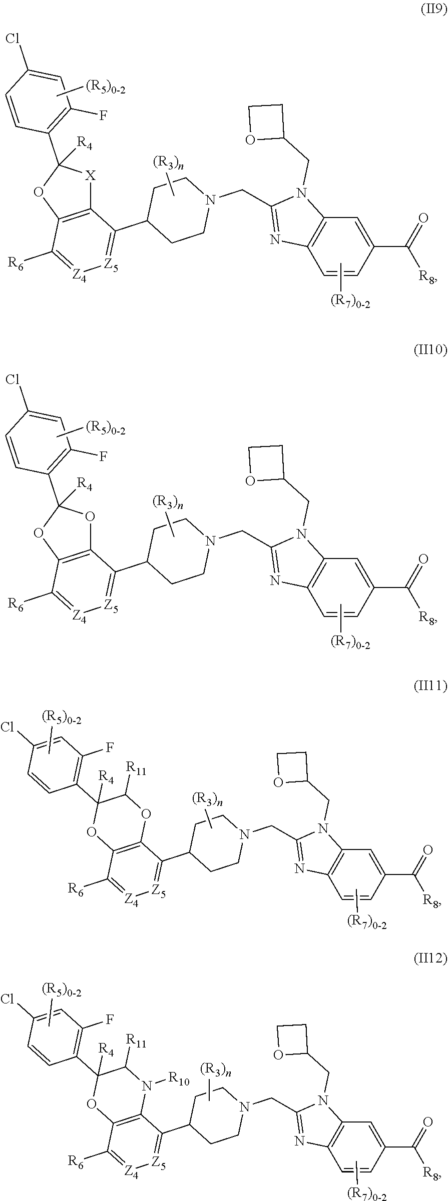 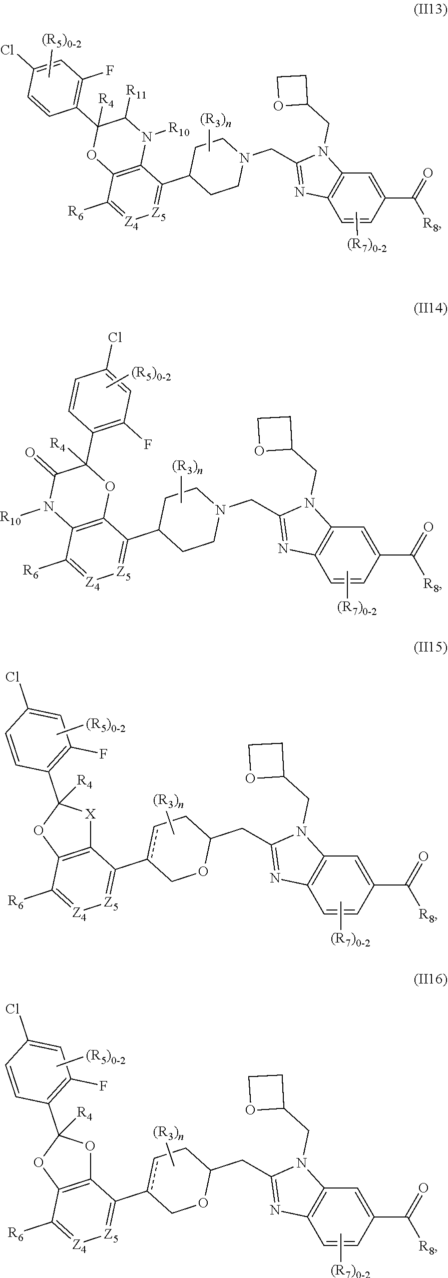 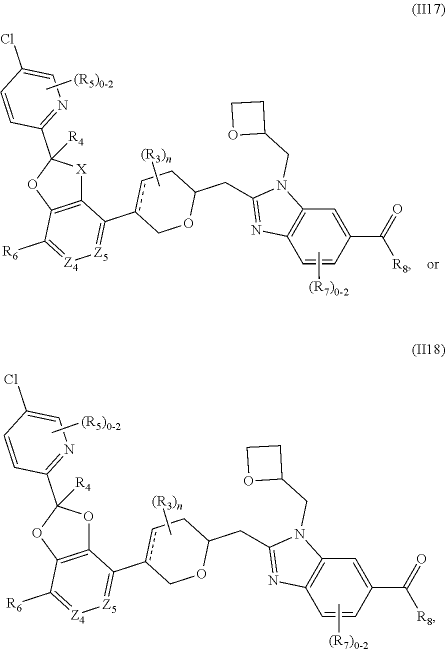
or a pharmaceutically acceptable salt, solvate, prodrug, stereoisomer, or tautomer thereof, wherein each R₉ is independently C₁-C₄ alkyl, halo-C₁-C₄ alkyl, C₁-C₄ alkoxy, halo-C₁-C₄ alkoxy, halogen, OH, CN, or NH₂.

8.-9. (canceled)

10. The compound of claim 1, wherein the compound is of Formula (III1), (III2), (III3), (III4), (III5), (III6), (III7), (III8), (III9), (III10), (III11), (III12), (III13), (III14), III15), (IV1), (IV2), (IV3), (IV4), (IV5), (IV6), (IV7), (IV8), (IV9), (IV10), (IV11), (IV12), (IV13), (IV14), (IV15), (IV16), (IV17), (IV18), (V1), (V2), (V3), (V4), (V5), (V6), (V7), (V8), (V9), (V10), (V11), (V12), (V13), (VI1), (VI2), (VI3), (VI4), (VI5), (VI6), (VI7), (VI8), (VI9), (VI10), (VI11), (VI12), or (VI13): 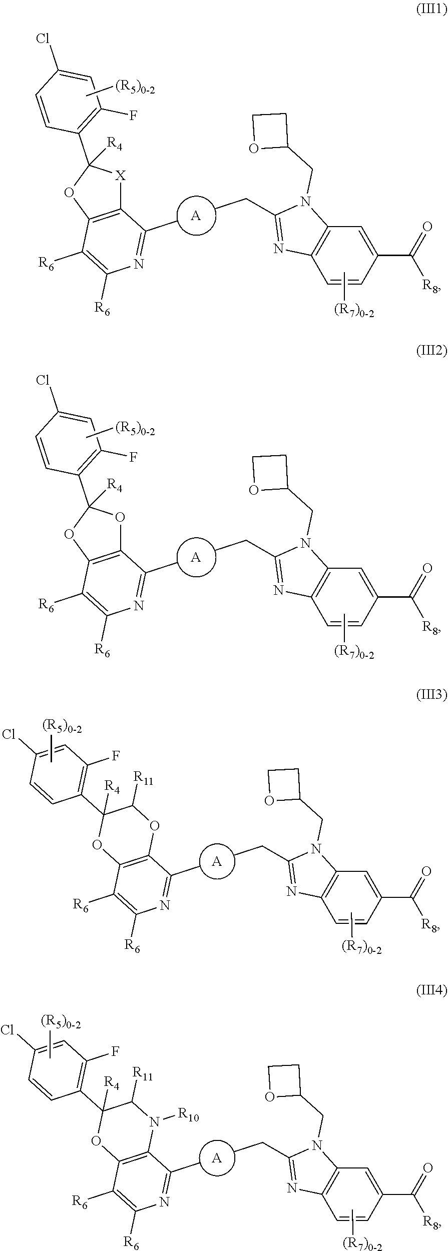 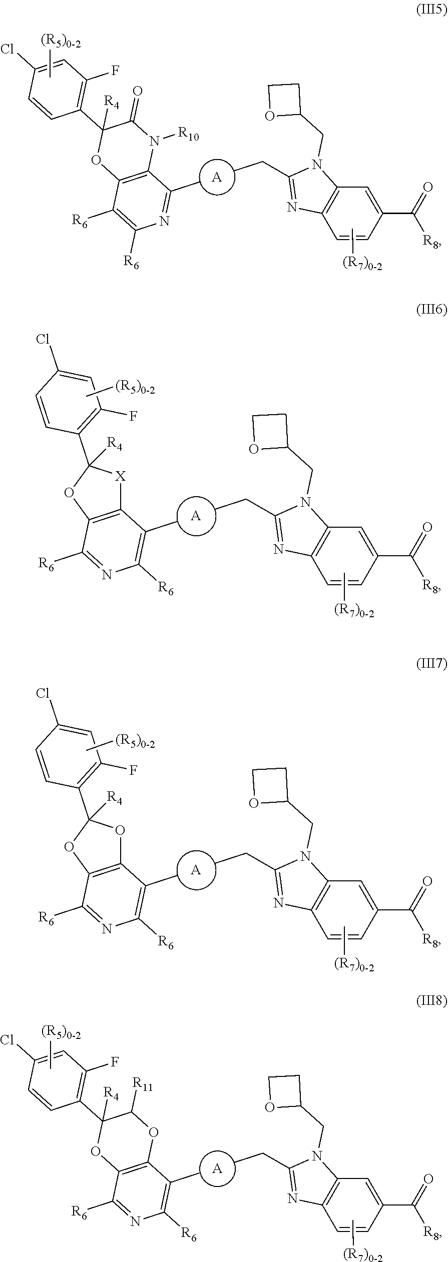 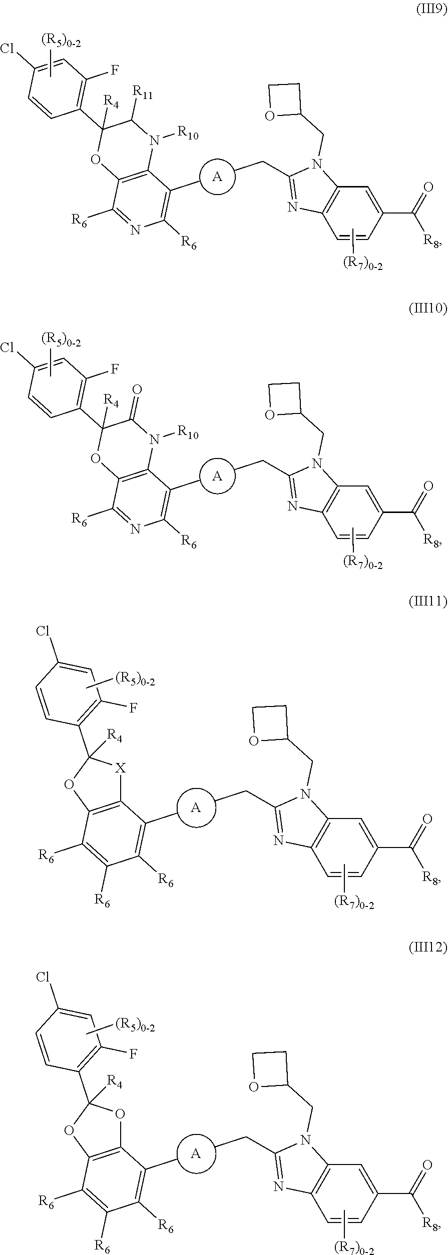 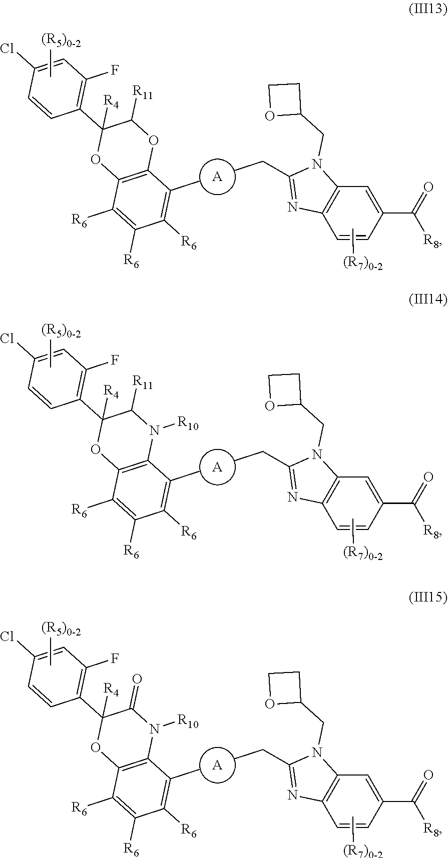 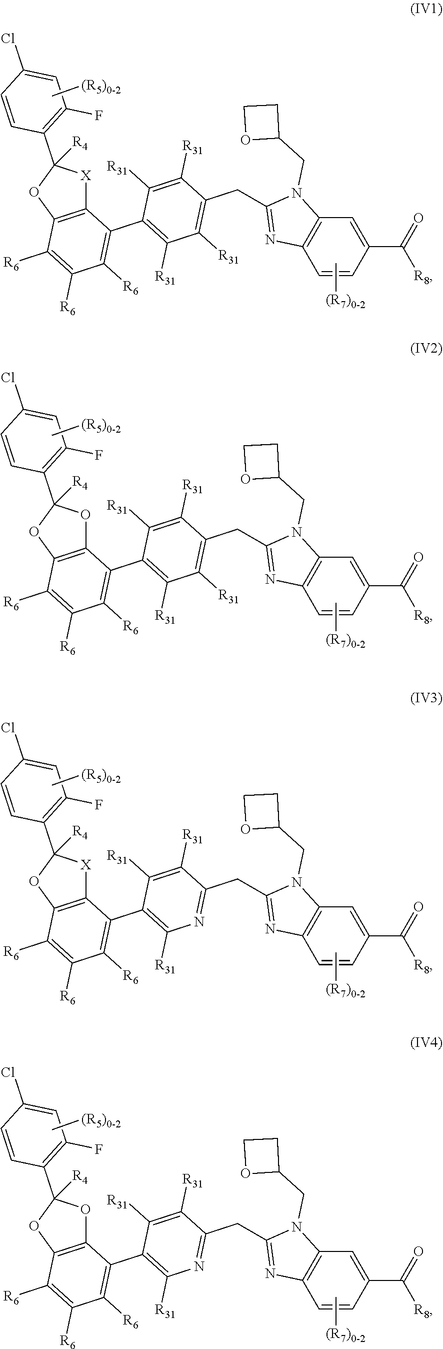 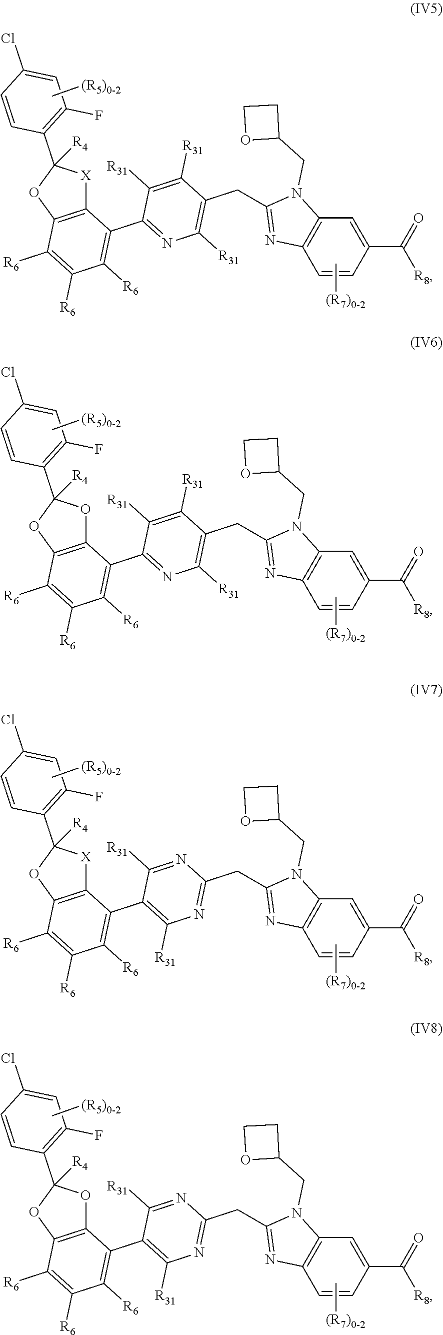 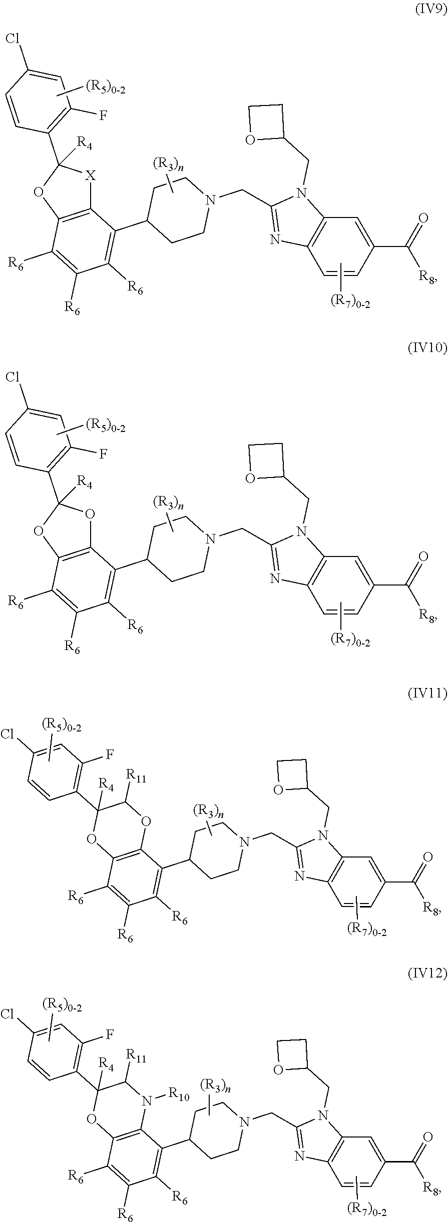 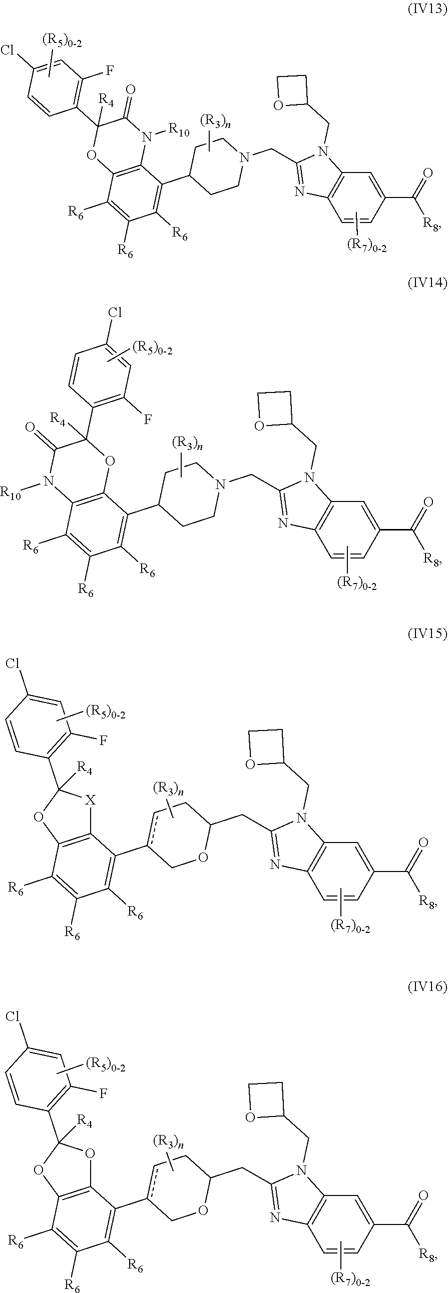 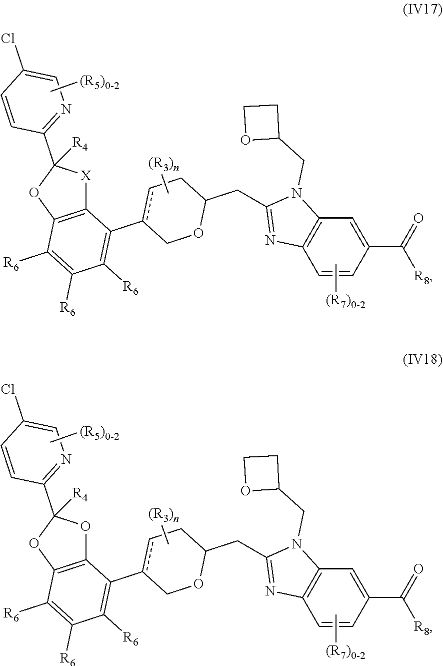 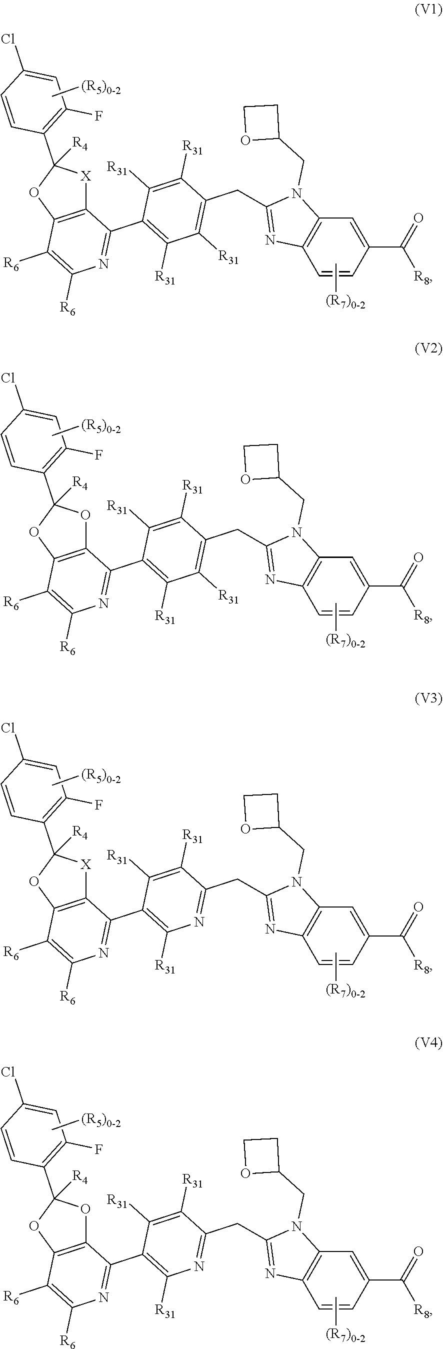 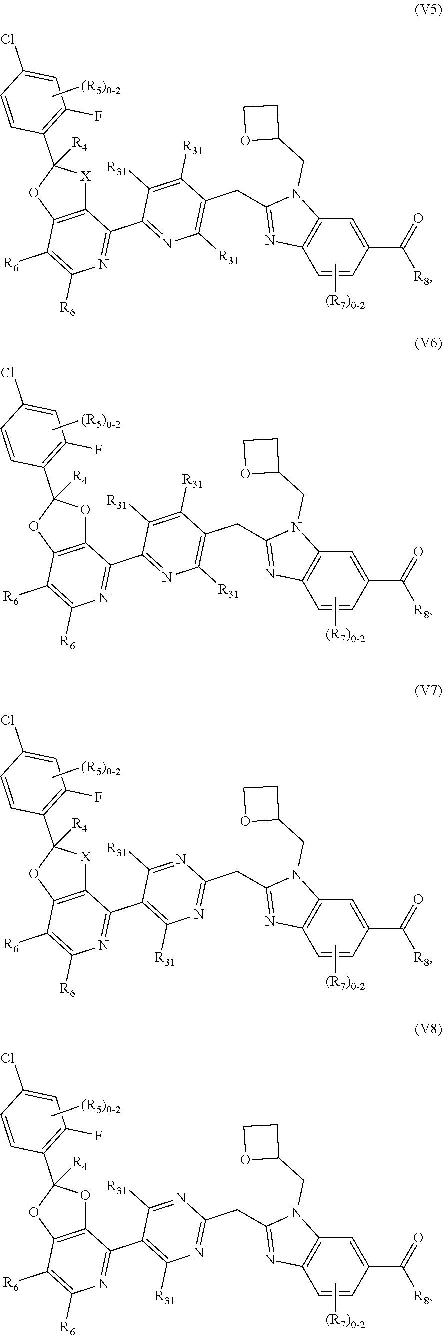 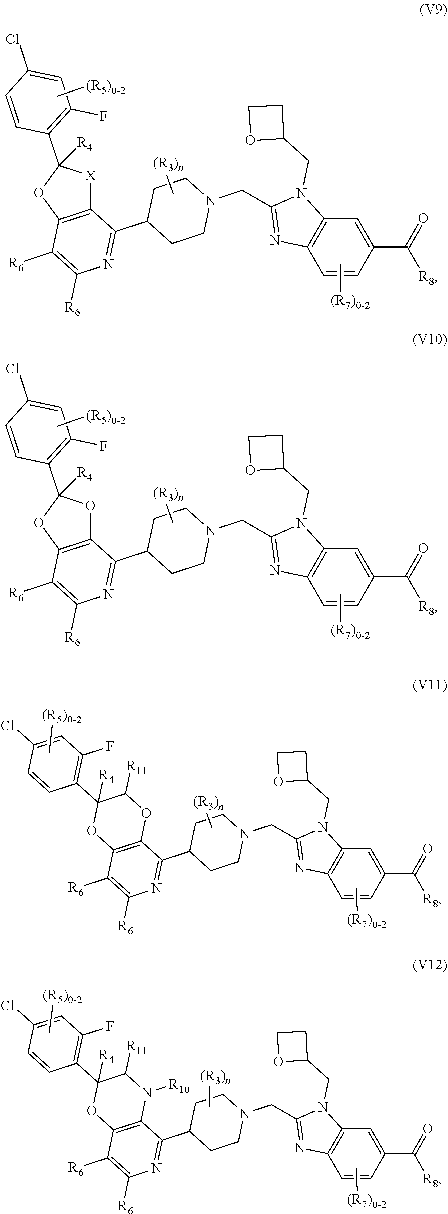 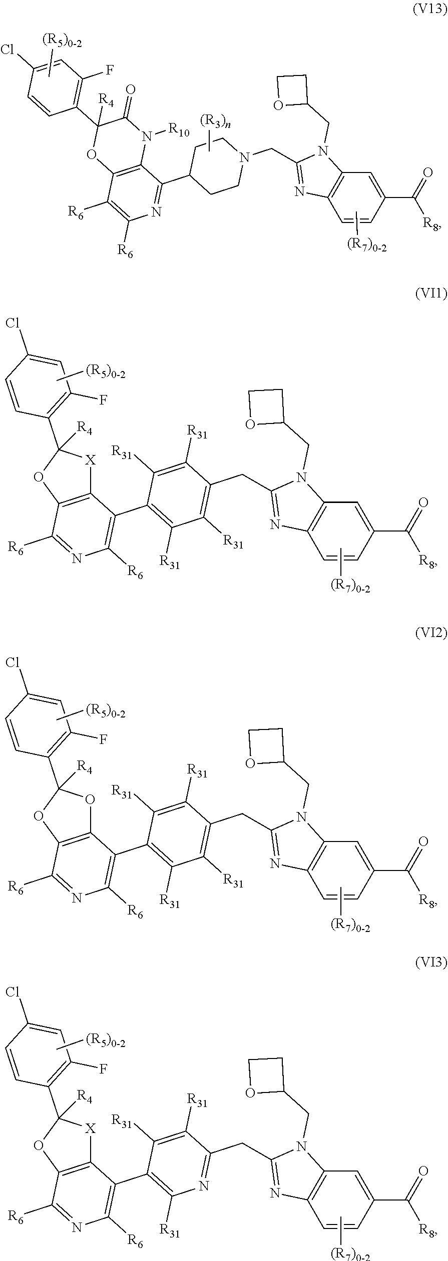 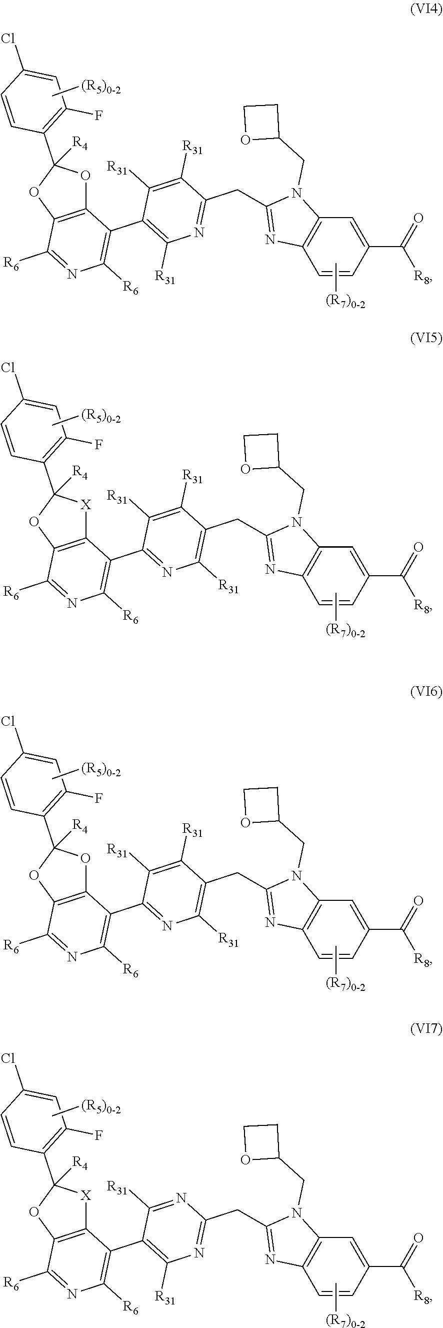 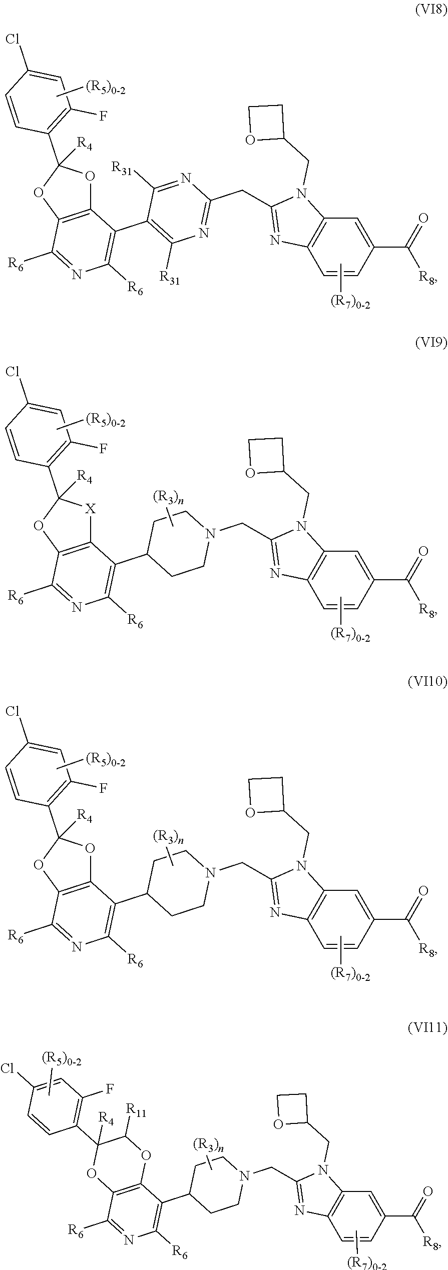 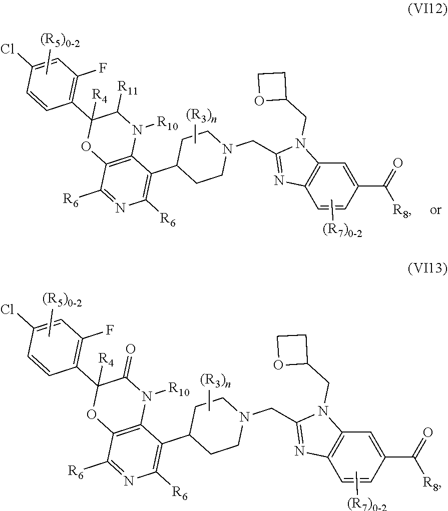
or a pharmaceutically acceptable salt, solvate, prodrug, stereoisomer, or tautomer thereof.

11.-13. (canceled)

14. The compound of claim 1, wherein the compound is of Formula (A′) or (I′): 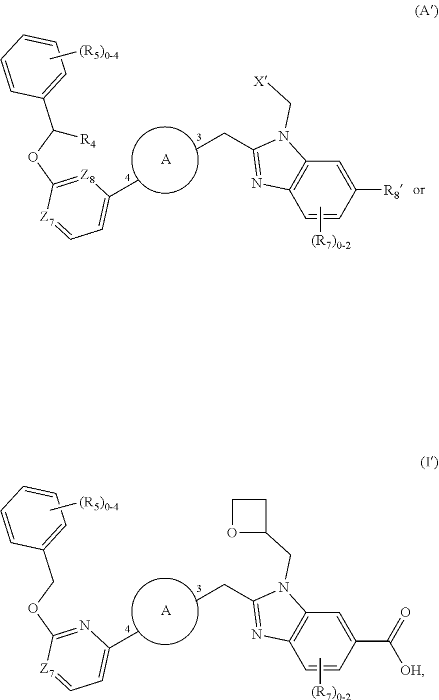
or a pharmaceutically acceptable salt, solvate, prodrug, stereoisomer, or tautomer thereof.

15.-18. (canceled)

19. The compound of claim 1, wherein X₁ is C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S, wherein the carbocyclyl, heterocyclyl, aryl, or heteroaryl is optionally substituted with one or more substituents independently selected from C₁-C₄ alkyl, halo-C₁-C₄ alkyl, C₁-C₄ alkoxy, halo-C₁-C₄ alkoxy, halogen, OH, CN, and NH₂.

20. (canceled)

21. The compound of claim 19, wherein X₁ is oxetanyl.

22. The compound of claim 1, wherein each Rᵢ is independently H, C₁-C₄ alkyl, halo-C₁-C₄ alkyl, C₁-C₄ alkoxy, halo-C₁-C₄ alkoxy, halogen, OH, C₃-C₆ carbocyclyl, 4- to 6-membered heterocyclyl comprising 1 or 2 heteroatoms selected from N, O, and S, C₆-C₁₀ aryl, or 5- to 10-membered heteroaryl comprising one or two 5- or 6-membered rings and 1 to 4 heteroatoms selected from N, O, and S.

23.-26. (canceled)

27. The compound of claim 1, wherein Ring A is

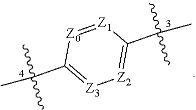

28. The compound of claim 1, wherein Ring A is

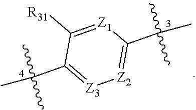

29. The compound of claim 1, wherein Ring A is

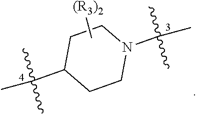

30. The compound of claim 1, wherein Ring A is

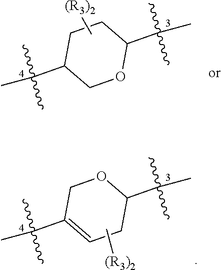

31. The compound of claim 1, wherein Ring A is

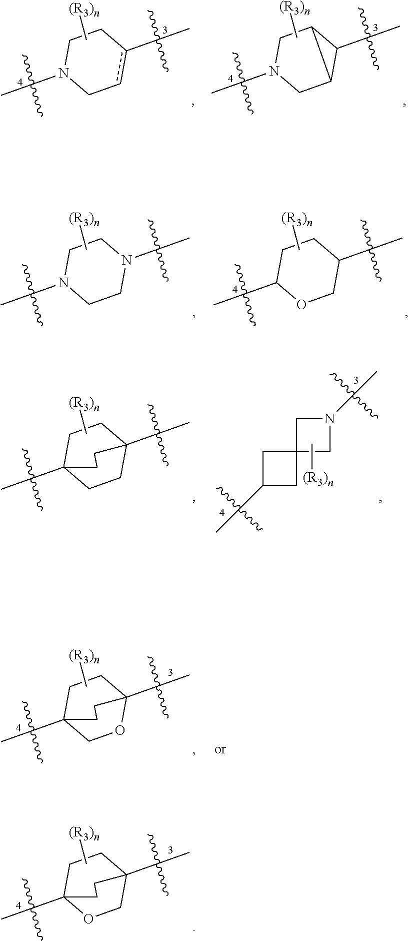

32. The compound of claim 1, wherein Ring A is

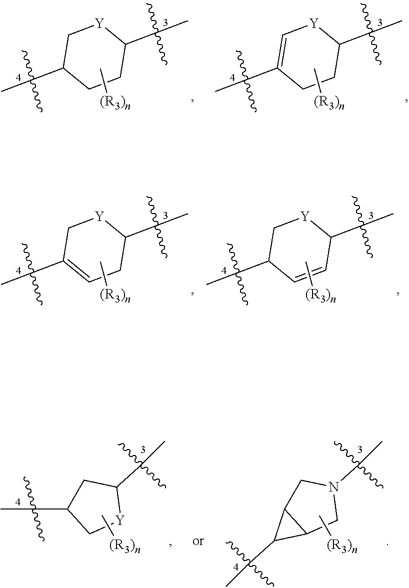

33. The compound of claim 1, wherein Ring A is

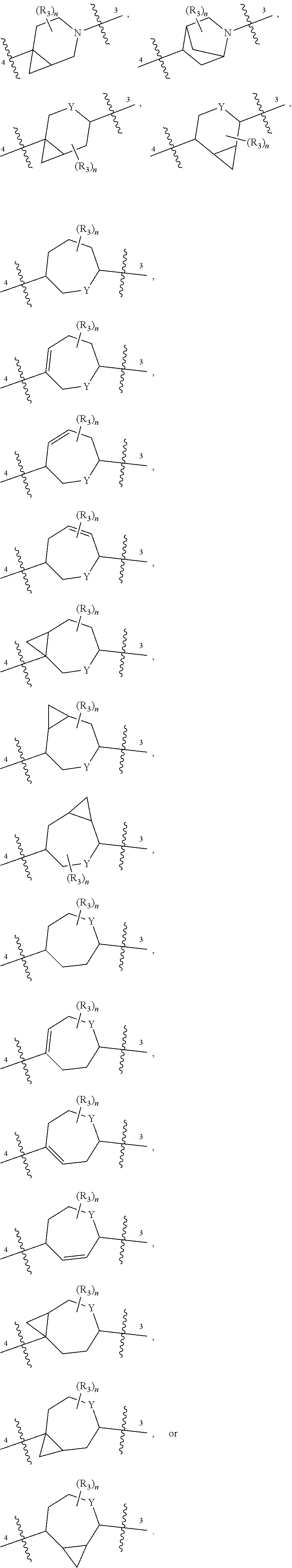

34. The compound of claim 1, wherein Y is O, NR_{b}, or C(R_{c})₂.

35. (canceled)

36. The compound of claim 1, wherein Z₀, Z₁, Z₂, and Z₃ are each independently CR₃₁; only one of Z₀, Z₁, Z₂, and Z₃ is CR₃₁, and three of Z₀, Z₁, Z₂, and Z₃ are N; two of Z₀, Z₁, Z₂, and Z₃ is CR₃₁, and two of Z₀, Z₁, Z₂, and Z₃ are N; or only one of Z₀, Z₁, Z₂, and Z₃ is N, and three of Z₀, Z₁, Z₂, and Z₃ are CR₃₁.

37.-45. (canceled)

46. The compound of claim 1, wherein

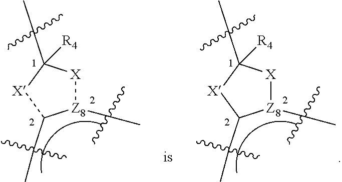

47. The compound of claim 46, wherein X and X′ are each O; X is CR₁R₂; and X′ is O; X is CHR₁₁O, CHR₁₁NR₁₀, or C(O)NR₁₀, and X′ is O; X is O, and X′ is CHR₁₁O, CHR₁₁NR₁₀, or C(O)NR₁₀; X is O and X′ is CHR₁₁O; X is CHR₁₁O and X′ is O; X is C(R₁₁)₂NR₁₀ and X′ is O; or X is O and X′ is C(R₁₁)₂NR₁₀.

48.-54. (canceled)

55. The compound of claim 1, wherein

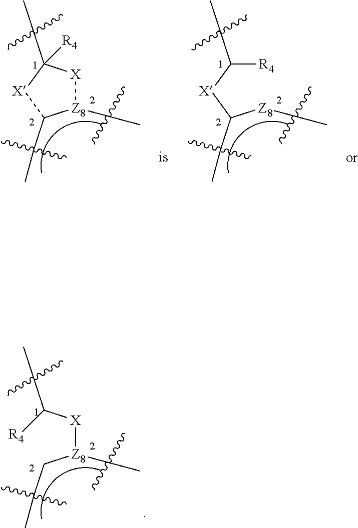

56. The compound of claim 55, wherein X and X′ are each independently O, NR₁₀, CR₁R₂, or C(R₁₁)₂O or C(R₁₁)₂NR₁₀, wherein the carbon atom is bonded with the atom marked “1”; or X and X′ are each independently O.

57.-68. (canceled)

69. The compound of claim 1, wherein Z₄, Z₅, and Z₇ are each CR₆; Z₄ and Z₅ are each N, and Z₇ is CR₆; at least one of Z₄, Z₅, and Z₇ is N; or only one of Z₄, Z₅, and Z₇ is N.

70.-100. (canceled)

101. A compound selected from Table A:

| Cmpd | | |
|---|---|---|
| No. | Structure | Chemical name |
| 2 | 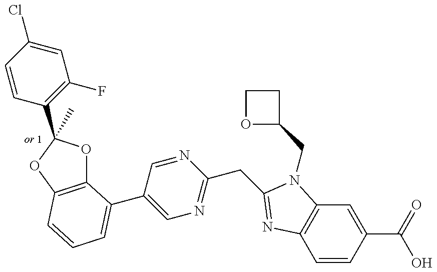 [SMILES: C[C@@]1(c2ccc(Cl)cc2F)Oc2cccc(-c3cnc(Cc4nc5ccc(C(=O)O)cc5n4C[C@@H]4CCO4)nc3)c2O1] | 2-((5-((R)-2-(4-chloro-2- fluorophenyl)-2- methylbenzo[d][1,3]dioxol-5- yl)pyrimidin-2-yl)methyl)-1- (((S)-oxetan-2-yl)methyl)-1H- benzo[d]imidazole-6-carboxylic acid |
| 3 | 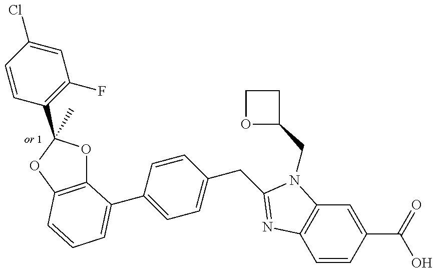 [SMILES: C[C@@]1(c2ccc(Cl)cc2F)Oc2cccc(-c3ccc(Cc4nc5ccc(C(=O)O)cc5n4C[C@@H]4CCO4)cc3)c2O1] | 2-(4-((R)-2-(4-chloro-2- fluorophenyl)-2- methylbenzo[d][1,3]dioxol-4- yl)benzyl)-1-(((S)-oxetan-2- yl)methyl)-1H- benzo[d]imidazole-6-carboxylic acid |
| 4 | 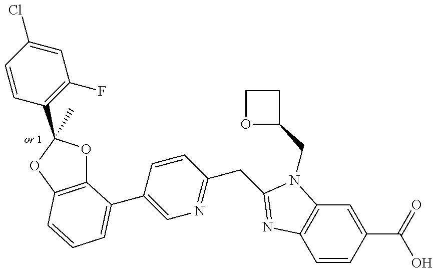 [SMILES: C[C@@]1(c2ccc(Cl)cc2F)Oc2cccc(-c3ccc(Cc4nc5ccc(C(=O)O)cc5n4C[C@@H]4CCO4)nc3)c2O1] | 2-((5-((R)-2-(4-chloro-2- fluorophenyl)-2- methylbenzo[d][1,3]dioxol-4- yl)pyridin-2-yl)methyl)-1-(((S)- oxetan-2-yl)methyl)-1H- benzo[d]imidazole-6-carboxylic acid |
| 5 | 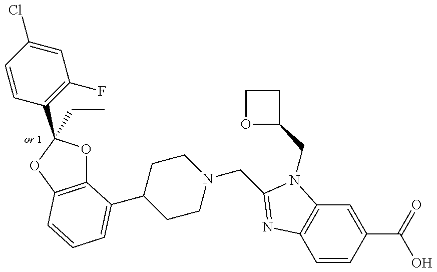 [SMILES: CC[C@@]1(c2ccc(Cl)cc2F)Oc2cccc(C3CCN(Cc4nc5ccc(C(=O)O)cc5n4C[C@@H]4CCO4)CC3)c2O1] | 2-((4-((R)-2-(4-chloro-2- fluorophenyl)-2- ethylbenzo[d][1,3]dioxol-4- yl)piperidin-1-yl)methyl)-1-(((S)- oxetan-2-yl)methyl)-1H- benzo[d]imidazole-6-carboxylic acid |
| 6 | 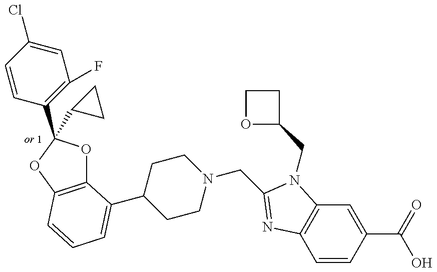 [SMILES: O=C(O)c1ccc2nc(CN3CCC(c4cccc5c4O[C@@](c4ccc(Cl)cc4F)(C4CC4)O5)CC3)n(C[C@@H]3CCO3)c2c1] | 2-((4-((R)-2-(4-chloro-2- fluorophenyl)-2- cyclopropylbenzo[d][1,3]dioxol- 4-yl)piperidin-1-yl)methyl)-1- (((S)-oxetan-2-yl)methyl)-1H- benzo[d]imidazole-6-carboxylic acid |
| 7 | 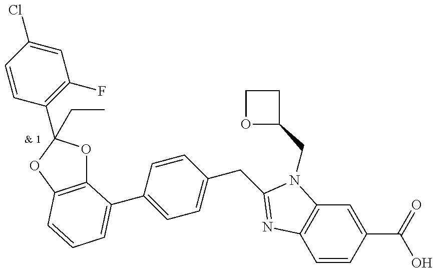 [SMILES: CCC1(c2ccc(Cl)cc2F)Oc2cccc(-c3ccc(Cc4nc5ccc(C(=O)O)cc5n4C[C@@H]4CCO4)cc3)c2O1] | 2-(4-(2-(4-chloro-2- fluorophenyl)-2- ethylbenzo[d][1,3]dioxol-4- yl)benzyl)-1-(((S)-oxetan-2- yl)methyl)-1H- benzo[d]imidazole-6-carboxylic acid |
| 8 | 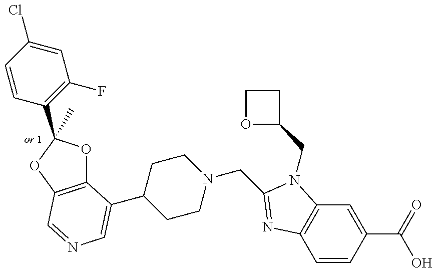 [SMILES: C[C@@]1(c2ccc(Cl)cc2F)Oc2cncc(C3CCN(Cc4nc5ccc(C(=O)O)cc5n4C[C@@H]4CCO4)CC3)c2O1] | 2-((4-((S)-2-(4-chloro-2- fluorophenyl)-2-methyl- [1,3]dioxolo[4,5-c]pyridin-7- yl)piperidin-1-yl)methyl)-1-(((S)- oxetan-2-yl)methyl)-1H- benzo[d]imidazole-6-carboxylic acid |
| 9 | 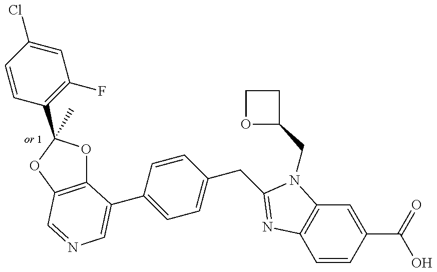 [SMILES: C[C@@]1(c2ccc(Cl)cc2F)Oc2cncc(-c3ccc(Cc4nc5ccc(C(=O)O)cc5n4C[C@@H]4CCO4)cc3)c2O1] | 2-(4-((S)-2-(4-chloro-2- fluorophenyl)-2-methyl- [1,3]dioxolo[4,5-c]pyridin-7- yl)benzyl)-1-(((S)-oxetan-2- yl)methyl)-1H- benzo[d]imidazole-6-carboxylic acid |
| 11 | 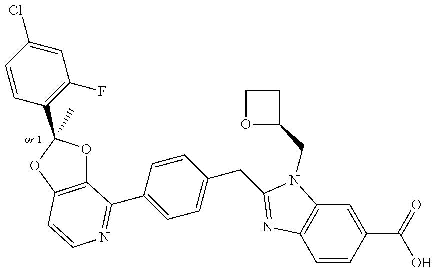 [SMILES: C[C@@]1(c2ccc(Cl)cc2F)Oc2ccnc(-c3ccc(Cc4nc5ccc(C(=O)O)cc5n4C[C@@H]4CCO4)cc3)c2O1] | 2-(4-((R)-2-(4-chloro-2- fluorophenyl)-2-methyl- [1,3]dioxolo[4,5-c]pyridin-4- yl)benzyl)-1-(((S)-oxetan-2- yl)methyl)-1H- benzo[d]imidazole-6-carboxylic acid |
| 12 | 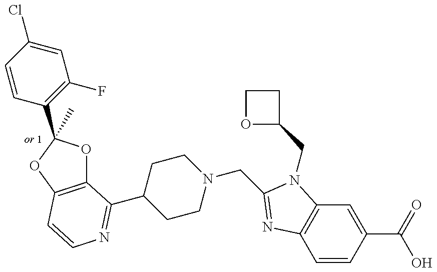 [SMILES: C[C@@]1(c2ccc(Cl)cc2F)Oc2ccnc(C3CCN(Cc4nc5ccc(C(=O)O)cc5n4C[C@@H]4CCO4)CC3)c2O1] | 2-((4-((R)-2-(4-chloro-2- fluorophenyl)-2-methyl- [1,3]dioxolo[4,5-c]pyridin-4- yl)piperidin-1-yl)methyl)-1-(((S)- oxetan-2-yl)methyl)-1H- benzo[d]imidazole-6-carboxylic acid |
| 13 | 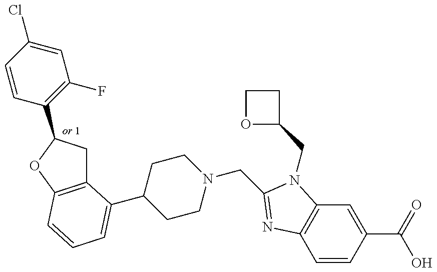 [SMILES: O=C(O)c1ccc2nc(CN3CCC(c4cccc5c4C[C@H](c4ccc(Cl)cc4F)O5)CC3)n(C[C@@H]3CCO3)c2c1] | 2-((4-((R)-2-(4-chloro-2- fluorophenyl)-2,3- dihydrobenzofuran-4- yl)piperidin-1-yl)methyl)-1-(((S)- oxetan-2-yl)methyl)-1H- benzo[d]imidazole-6-carboxylic acid |
| 14 | 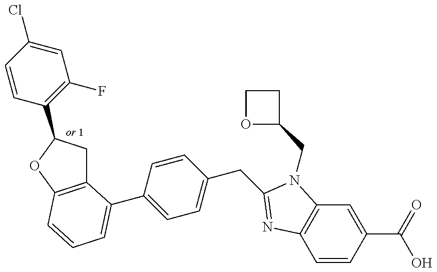 [SMILES: O=C(O)c1ccc2nc(Cc3ccc(-c4cccc5c4C[C@H](c4ccc(Cl)cc4F)O5)cc3)n(C[C@@H]3CCO3)c2c1] | 2-(4-((R)-2-(4-chloro-2- fluorophenyl)-2,3- dihydrobenzofuran-2-yl)benzyl)- 1-(((S)-oxetan-2-yl)methyl)-1H- benzo[d]imidazole-6-carboxylic acid |
| 15 | 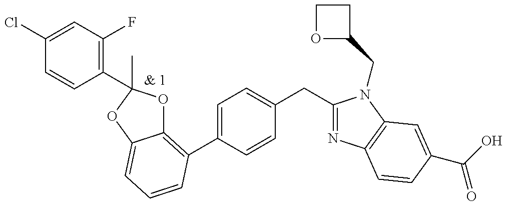 | 2-(4-(2-(4-chloro-2- fluorophenyl)-2- methylbenzo[d][1,3]dioxol-4- yl)benzyl)-1-(((S)-oxetan-2- yl)methyl)-1H- benzo[d]imidazole-6-carboxylic acid |
| 16 | 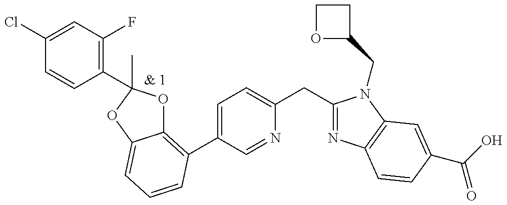 | 2-((5-(2-(4-chloro-2- fluorophenyl)-2- methylbenzo[d][1,3]dioxol-4- yl)pyridin-2-yl)methyl)-1-(((S)- oxetan-2-yl)methyl)-1H- benzo[d]imidazole-6-carboxylic acid |
| 17 | 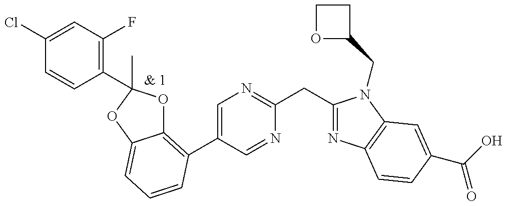 | 2-((5-(2-(4-chloro-2- fluorophenyl)-2- methylbenzo[d][1,3]dioxol-4- yl)pyrimidin-2-yl)methyl)-1- (((S)-oxetan-2-yl)methyl)-1H- benzo[d]imidazole-6-carboxylic acid |
| 18 | 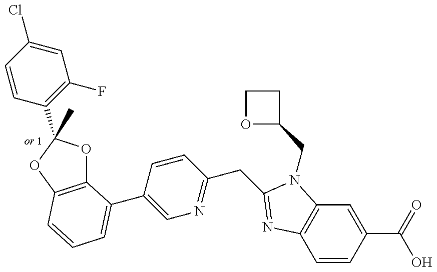 [SMILES: C[C@]1(c2ccc(Cl)cc2F)Oc2cccc(-c3ccc(Cc4nc5ccc(C(=O)O)cc5n4C[C@@H]4CCO4)nc3)c2O1] | 2-((5-((S)-2-(4-chloro-2- fluorophenyl)-2- methylbenzo[d][1,3]dioxol-4- yl)pyridin-2-yl)methyl)-1-(((S)- oxetan-2-yl)methyl)-1H- benzo[d]imidazole-6-carboxylic acid |
| 19 | 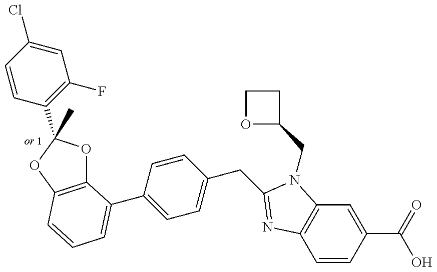 [SMILES: C[C@]1(c2ccc(Cl)cc2F)Oc2cccc(-c3ccc(Cc4nc5ccc(C(=O)O)cc5n4C[C@@H]4CCO4)cc3)c2O1] | 2-(4-((S)-2-(4-chloro-2- fluorophenyl)-2- methylbenzo[d][1,3]dioxol-4- yl)benzyl)-1-(((S)-oxetan-2- yl)methyl)-1H- benzo[d]imidazole-6-carboxylic acid |
| 21 | 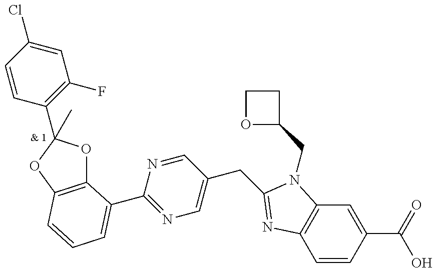 [SMILES: CC1(c2ccc(Cl)cc2F)Oc2cccc(-c3ncc(Cc4nc5ccc(C(=O)O)cc5n4C[C@@H]4CCO4)cn3)c2O1] | 2-((2-(2-(4-chloro-2- fluorophenyl)-2- methylbenzo[d][1,3]dioxol-4- yl)pyrimidin-5-yl)methyl)-1- (((S)-oxetan-2-yl)methyl)-1H- benzo[d]imidazole-6-carboxylic acid |
| 22 | 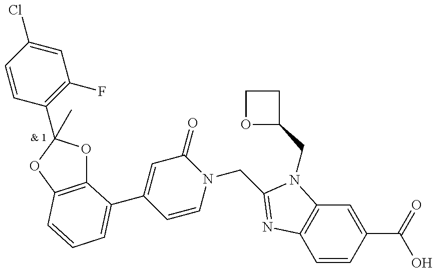 [SMILES: CC1(c2ccc(Cl)cc2F)Oc2cccc(-c3ccn(Cc4nc5ccc(C(=O)O)cc5n4C[C@@H]4CCO4)c(=O)c3)c2O1] | 2-((4-(2-(4-chloro-2- fluorophenyl)-2- methylbenzo[d][1,3]dioxol-4-yl)- 2-oxopyridin-1(2H)-yl)methyl)-1- (((S)-oxetan-2-yl)methyl)-1H- benzo[d]imidazole-6-carboxylic acid |
| 23 | 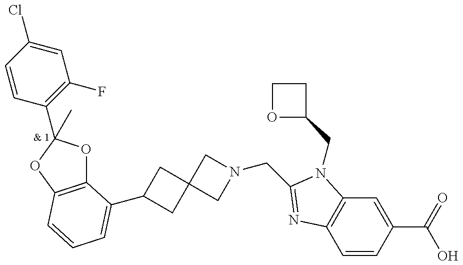 [SMILES: CC1(c2ccc(Cl)cc2F)Oc2cccc(C3CC4(C3)CN(Cc3nc5ccc(C(=O)O)cc5n3C[C@@H]3CCO3)C4)c2O1] | 2-((6-(2-(4-chloro-2- fluorophenyl)-2- methylbenzo[d][1,3]dioxol-4-yl)- 2-azaspiro[3.3]heptan-2- yl)methyl)-1-(((S)-oxetan-2- yl)methyl)-1H- benzo[d]imidazole-6-carboxylic acid |
| 24 | 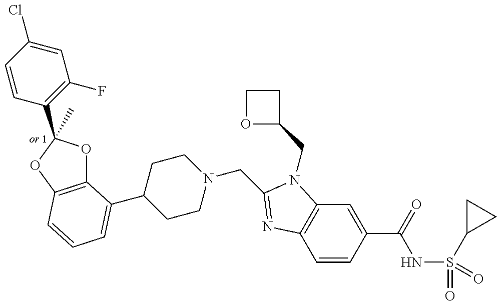 [SMILES: C[C@@]1(c2ccc(Cl)cc2F)Oc2cccc(C3CCN(Cc4nc5ccc(C(=O)NS(=O)(=O)C6CC6)cc5n4C[C@@H]4CCO4)CC3)c2O1] | 2-((4-((R)-2-(4-chloro-2- fluorophenyl)-2- methylbenzo[d][1,3]dioxol-4- yl)piperidin-1-yl)methyl)-N- (cyclopropylsulfonyl)-1-(((S)- oxetan-2-yl)methyl)-1H- benzo[d]imidazole-6- carboxamide |
| 25 | 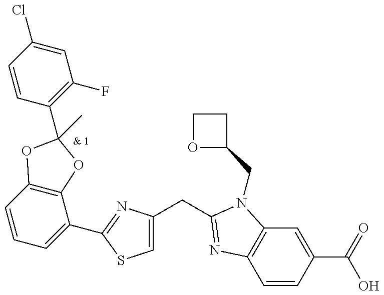 [SMILES: CC1(c2ccc(Cl)cc2F)Oc2cccc(-c3nc(Cc4nc5ccc(C(=O)O)cc5n4C[C@@H]4CCO4)cs3)c2O1] | 2-((2-(2-(4-chloro-2- fluorophenyl)-2- methylbenzo[d][1,3]dioxol-4- yl)thiazol-4-yl)methyl)-1-(((S)- oxetan-2-yl)methyl)-1H- benzo[d]imidazole-6-carboxylic acid |
| 26 | 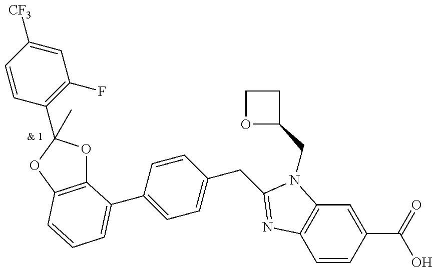 [SMILES: CC1(c2ccc(C(F)(F)F)cc2F)Oc2cccc(-c3ccc(Cc4nc5ccc(C(=O)O)cc5n4C[C@@H]4CCO4)cc3)c2O1] | 2-(4-(2-(2-fluoro-4- (trifluoromethyl)phenyl)-2- methylbenzo[d][1,3]dioxol-4- yl)benzyl)-1-(((S)-oxetan-2- yl)methyl)-1H- benzo[d]imidazole-6-carboxylic acid |
| 27 | 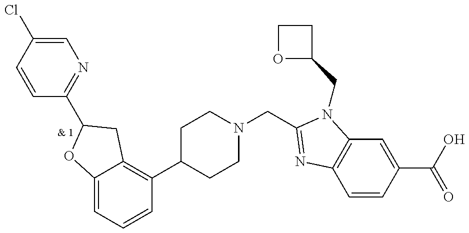 [SMILES: O=C(O)c1ccc2nc(CN3CCC(c4cccc5c4CC(c4ccc(Cl)cn4)O5)CC3)n(C[C@@H]3CCO3)c2c1] | 2-((4-(2-(5-chloropyridin-2-yl)- 2,3-dihydrobenzofuran-4- yl)piperidin-1-yl)methyl)-1-(((S)- oxetan-2-yl)methyl)-1H- benzo[d]imidazole-6-carboxylic acid |
| 28 | 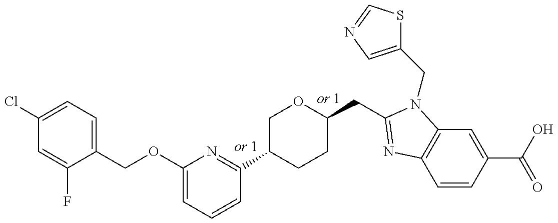 [SMILES: O=C(O)c1ccc2nc(C[C@H]3CC[C@H](c4cccc(OCc5ccc(Cl)cc5F)n4)CO3)n(Cc3cncs3)c2c1] | 2-(((2R,5R)-5-(6-((4-chloro-2- fluorobenzyl)oxy)pyridin-2- yl)tetrahydro-2H-pyran-2- yl)methyl)-1-(thiazol-5- ylmethyl)-1H-benzo[d]imidazol-3- 6-carboxylic acid |
| 29 | 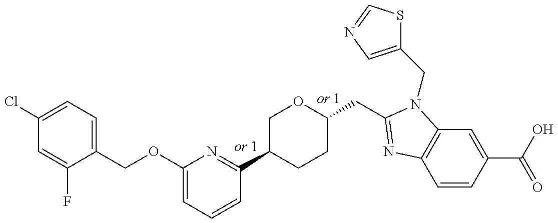 [SMILES: O=C(O)c1ccc2nc(C[C@@H]3CC[C@@H](c4cccc(OCc5ccc(Cl)cc5F)n4)CO3)n(Cc3cncs3)c2c1] | 2-(((2S,5S)-5-(6-((4-chloro-2- fluorobenzyl)oxy)pyridin-2- yl)tetrahydro-2H-pyran-2- yl)methyl)-1-(thiazol-5- ylmethyl)-1H-benzo[d]imidazole- 6-carboxylic acid |
| 30 | 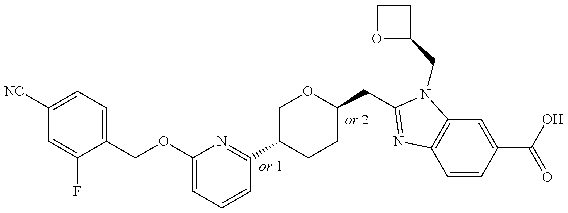 [SMILES: N#Cc1ccc(COc2cccc([C@H]3CC[C@H](Cc4nc5ccc(C(=O)O)cc5n4C[C@@H]4CCO4)OC3)n2)c(F)c1] | 2-(((2R,5R)-5-(6-((4-cyano-2- fluroobenzyl)oxy)pyridin-2- yl)tetrahydro-2H-pyran-2- yl)methyl)-1-(((S)-oxetan-2- yl)methyl)-1H- benzo[d]imidazole-6-carboxylic acid |
| 31 | 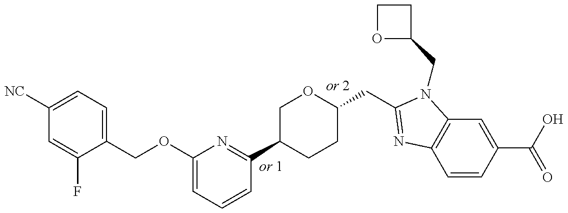 [SMILES: N#Cc1ccc(COc2cccc([C@@H]3CC[C@@H](Cc4nc5ccc(C(=O)O)cc5n4C[C@@H]4CCO4)OC3)n2)c(F)c1] | 2-(((2S,5S)-5-(6-((4-cyano-2- fluorobenzyl)oxy)pyridin-2- yl)tetrahydro-2H-pyran-2- yl)methyl)-1-(((S)-oxetan-2- yl)methyl)-1H- benzo[d]imidazole-6-carboxylic acid |
| 32 | 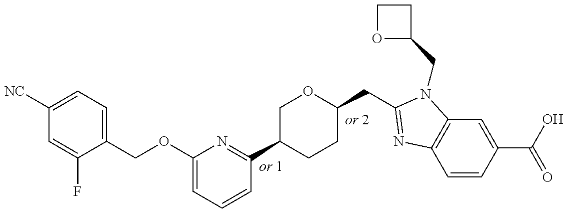 [SMILES: N#Cc1ccc(COc2cccc([C@@H]3CC[C@H](Cc4nc5ccc(C(=O)O)cc5n4C[C@@H]4CCO4)OC3)n2)c(F)c1] | 2-(((2R,5S)-5-(6-((4-cyano-2- fluorobenzyl)oxy)pyridin-2- yl)tetrahydro-2H-pyran-2- yl)methyl)-1-(((S)-oxetan-2- yl)methyl)-1H- benzo[d]imidazole-6-carboxylic acid |
| 33 | 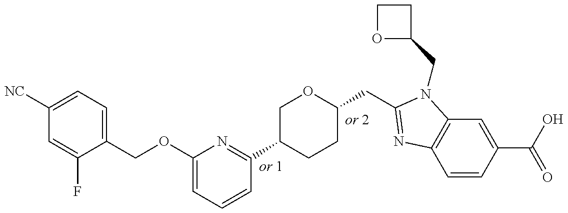 [SMILES: N#Cc1ccc(COc2cccc([C@H]3CC[C@@H](Cc4nc5ccc(C(=O)O)cc5n4C[C@@H]4CCO4)OC3)n2)c(F)c1] | 2-(((2S,5R)-5-(6-((4-cyano-2- fluorobenzyl)oxy)pyridin-2- yl)tetrahydro-2H-pyran-2- yl)methyl)-1-(((S)-oxetan-2- yl)methyl)-1H- benzo[d]imidazole-6-carboxylic acid |
| 34 | 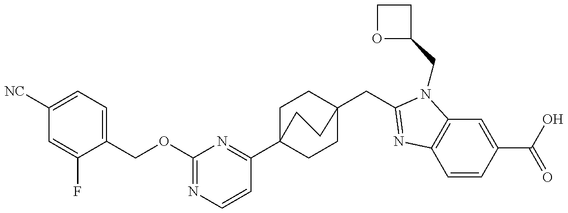 [SMILES: N#Cc1ccc(COc2nccc(C34CCC(Cc5nc6ccc(C(=O)O)cc6n5C[C@@H]5CCO5)(CC3)CC4)n2)c(F)c1] | (S)-2-((4-(2-((4-cyano-2- fluorobenzyl)oxy)pyrimidin-4- yl)bicyclo[2.2.2]octan-2- yl)methyl)-1-(oxetan-2- ylmethyl)-1H-benzo[d]imidazole- 6-carboxylic acid |
| 35 | 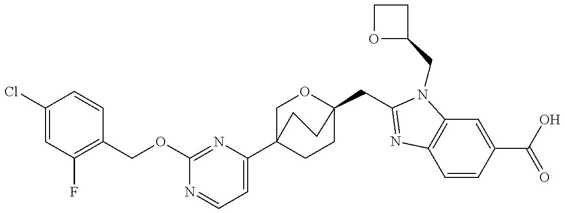 | (S)-2-((4-(2-((4-chloro-2- fluorobenzyl)oxy)pyrimidin-4- yl)-2-oxabicyclo[2.2.2]octan-1- yl)methyl)-1-(oxetan-2- ylmethyl)-1H-benzo[d]imidazole- 6-carboxylic acid |
| 36 | 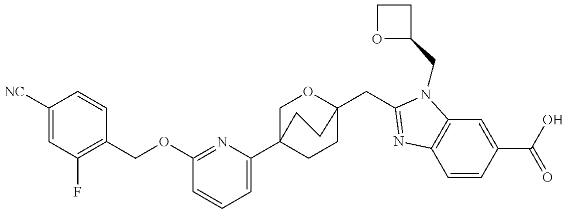 [SMILES: N#Cc1ccc(COc2cccc(C34CCC(Cc5nc6ccc(C(=O)O)cc6n5C[C@@H]5CCO5)(CC3)OC4)n2)c(F)c1] | (S)-2-((4-(6-((4-cyano-2- fluorobenzyl)oxy)pyridin-2-yl)-2- oxabicyclo[2.2.2]octan-1- yl)methyl)-1-(oxetan-2- ylmethyl)-1H-benzo[d]imidazole- 6-carboxylic acid |
| 37 | 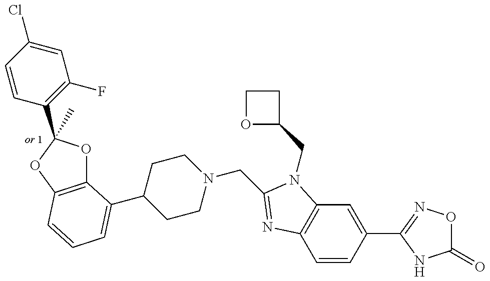 [SMILES: C[C@@]1(c2ccc(Cl)cc2F)Oc2cccc(C3CCN(Cc4nc5ccc(-c6noc(=O)[nH]6)cc5n4C[C@@H]4CCO4)CC3)c2O1] | 3-(2-((4-((R)-2-(4-chloro-2- fluorophenyl)-2- methylbenzo[d][1,3]dioxol-4- yl)piperidin-1-yl)methyl)-1-(((S)- oxetan-2-yl)methyl)-1H- benzo[d]imidazol-6-yl)-1,2,4- oxadiazol-5(4H)-one |
| 40 | 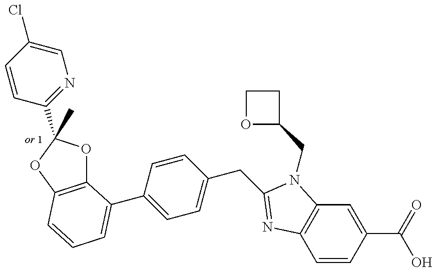 [SMILES: C[C@]1(c2ccc(Cl)cn2)Oc2cccc(-c3ccc(Cc4nc5ccc(C(=O)O)cc5n4C[C@@H]4CCO4)cc3)c2O1] | 2-(4-((S)-2-(5-chloropyridin-2- yl)-2-methylbenzo[d][1,3]dioxol- 4-yl)benzyl)-1-(((S)-oxetan-2- yl)methyl)-1H- benzo[d]imidazole-6-carboxylic acid |
| 41 | 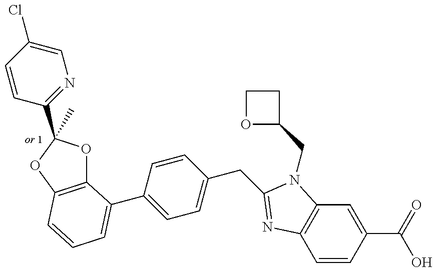 [SMILES: C[C@@]1(c2ccc(Cl)cn2)Oc2cccc(-c3ccc(Cc4nc5ccc(C(=O)O)cc5n4C[C@@H]4CCO4)cc3)c2O1] | 2-(4-((R)-2-(5-chloropyridin-2- yl)-2-methylbenzo[d][1,3]dioxol- 4-yl)benzyl)-1-(((S)-oxetan-2- yl)methyl)-1H- benzo[d]imidazole-6-carboxylic acid |
| 42 | 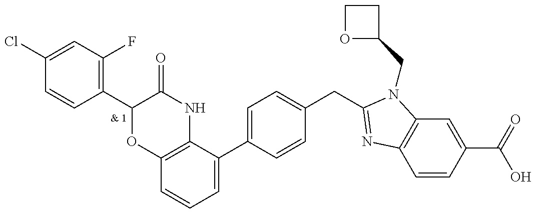 [SMILES: O=C(O)c1ccc2nc(Cc3ccc(-c4cccc5c4NC(=O)C(c4ccc(Cl)cc4F)O5)cc3)n(C[C@@H]3CCO3)c2c1] | 2-(4-(2-(4-chloro-2- fluorophenyl)-3-oxo-3,4-dihydro- 2H-benzo[b][1,4]oxazin-5- yl)benzyl)-1-(((S)-oxetan-2- yl)methyl)-1H- benzo[d]imidazole-6-carboxylic acid |
| 43 | 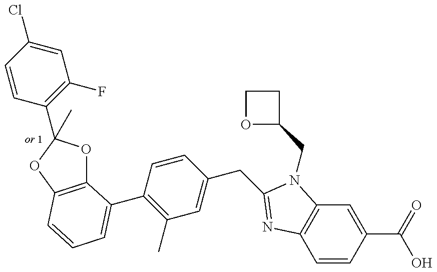 [SMILES: Cc1cc(Cc2nc3ccc(C(=O)O)cc3n2C[C@@H]2CCO2)ccc1-c1cccc2c1OC(C)(c1ccc(Cl)cc1F)O2] | 2-(4-(2-(4-chloro-2- fluorophenyl)-2- methylbenzo[d][1,3]dioxol-4-yl)- 3-methylbenzyl)-1-(((S)-oxetan- 2-yl)methyl)-1H- benzo[d]imidazole-6-carboxylic acid |
| 56 | 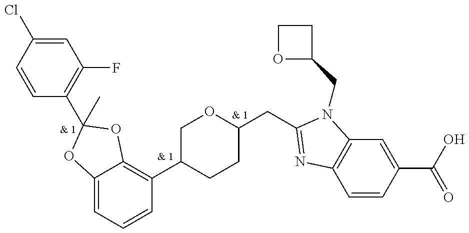 [SMILES: CC1(c2ccc(Cl)cc2F)Oc2cccc(C3CCC(Cc4nc5ccc(C(=O)O)cc5n4C[C@@H]4CCO4)OC3)c2O1] | 2-((5-(2-(4-chloro-2- fluorophenyl)-2- methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 57 | 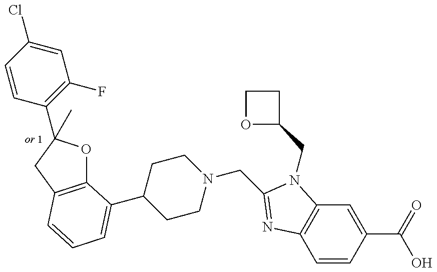 [SMILES: CC1(c2ccc(Cl)cc2F)Cc2cccc(C3CCN(Cc4nc5ccc(C(=O)O)cc5n4C[C@@H]4CCO4)CC3)c2O1] | 2-((4-(2-(4-chloro-2- fluorophenyl)-2-methyl-2,3- dihydrobenzofuran-7- yl)piperidin-1-yl)methyl)-1-(((S)- oxetan-2-yl)methyl)-1H- benzo[d]imidazole-6-carboxylic acid |
| 58 | 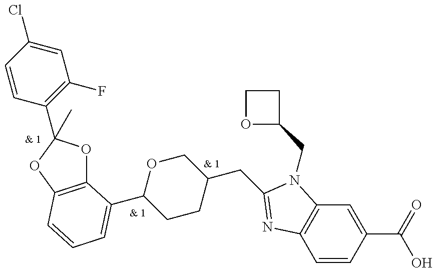 [SMILES: CC1(c2ccc(Cl)cc2F)Oc2cccc(C3CCC(Cc4nc5ccc(C(=O)O)cc5n4C[C@@H]4CCO4)CO3)c2O1] | 2-((6-(2-(4-chloro-2- fluorophenyl)-2- methylbenzo[d][1,3]dioxol-4- yl)tetrahydro-2H-pyran-3- yl)methyl)-1-(((S)-oxetan-2- yl)methyl)-1H- benzo[d]imidazole-6-carboxylic acid |
| 59 | 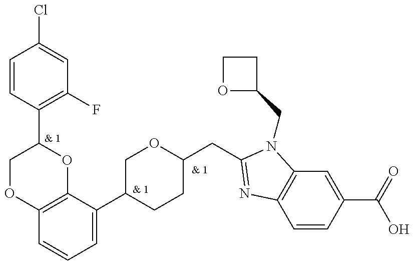 [SMILES: O=C(O)c1ccc2nc(CC3CCC(c4cccc5c4OC(c4ccc(Cl)cc4F)CO5)CO3)n(C[C@@H]3CCO3)c2c1] | 2-((5-(3-(4-chloro-2- fluorophenyl)-2,3- dihydrobenzo[b][1,4]dioxin-5- yl)tetrahydro-2H-pyran-2- yl)methyl)-1-(((S)-oxetan-2- yl)methyl)-1H- benzo[d]imidazole-6-carboxylic acid |
| 60 | 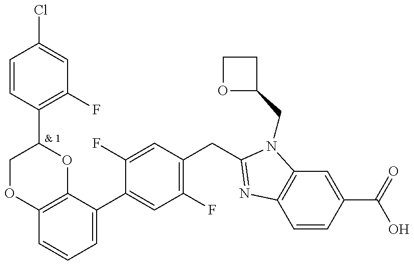 [SMILES: O=C(O)c1ccc2nc(Cc3cc(F)c(-c4cccc5c4OC(c4ccc(Cl)cc4F)CO5)cc3F)n(C[C@@H]3CCO3)c2c1] | 2-(4-(3-(4-chloro-2- fluorophenyl)-2,3- dihydrobenzo[b][1,4]dioxin-5- yl)-2,5-difluorobenzyl)-1-(((S)- oxetan-2-yl)methyl)-1H- benzo[d]imidazole-6-carboxylic acid |
| 61 | 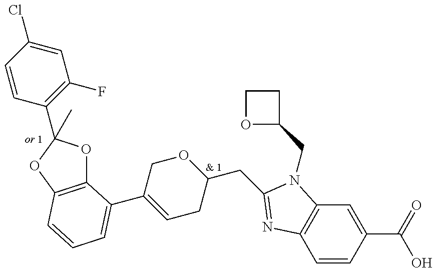 [SMILES: CC1(c2ccc(Cl)cc2F)Oc2cccc(C3=CCC(Cc4nc5ccc(C(=O)O)cc5n4C[C@@H]4CCO4)OC3)c2O1] | 2-((5-(2-(4-chloro-2- fluorophenyl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 62 | 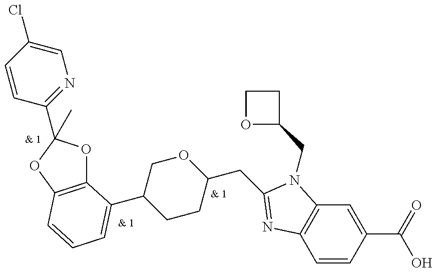 [SMILES: CC1(c2ccc(Cl)cn2)Oc2cccc(C3CCC(Cc4nc5ccc(C(=O)O)cc5n4C[C@@H]4CCO4)OC3)c2O1] | 2-((5-(2-(5-chloropyridin-2-yl)-2- methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 63 | 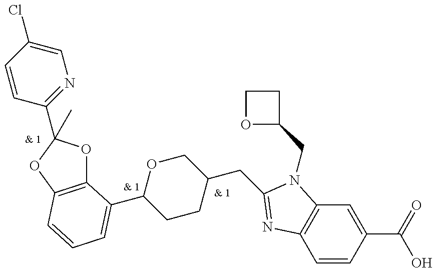 [SMILES: CC1(c2ccc(Cl)cn2)Oc2cccc(C3CCC(Cc4nc5ccc(C(=O)O)cc5n4C[C@@H]4CCO4)CO3)c2O1] | 2-((6-(2-(5-chloropyridin-2-yl)-2- methylbenzo[d][1,3]dioxol-4- yl)tetrahydro-2H-pyran-3- yl)methyl)-1-(((S)-oxetan-2- yl)methyl)-1H- benzo[d]imidazole-6-carboxylic acid |
| 64 | 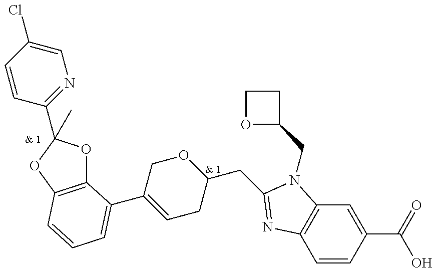 [SMILES: CC1(c2ccc(Cl)cn2)Oc2cccc(C3=CCC(Cc4nc5ccc(C(=O)O)cc5n4C[C@@H]4CCO4)OC3)c2O1] | 2-((5-(2-(5-chloropyridin-2-yl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 65 | 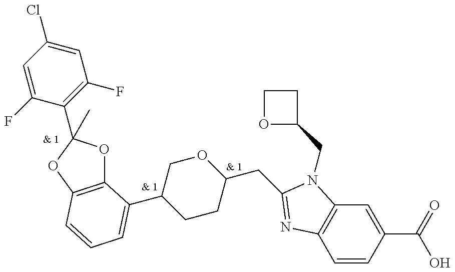 [SMILES: CC1(c2c(F)cc(Cl)cc2F)Oc2cccc(C3CCC(Cc4nc5ccc(C(=O)O)cc5n4C[C@@H]4CCO4)OC3)c2O1] | 2-((5-(2-(4-chloro-2,6- difluorophenyl)-2- methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 66 | 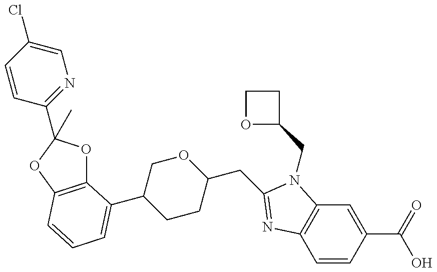 [SMILES: CC1(c2ccc(Cl)cn2)Oc2cccc(C3CCC(Cc4nc5ccc(C(=O)O)cc5n4C[C@@H]4CCO4)OC3)c2O1] | 2-((5-(2-(5-chloropyridin-2-yl)-2- methylbenzo[d][1,3]dioxol-4- yl)tetrahydro-2H-pyran-2- yl)methyl)-3-(((S)-oxetan-2- yl)methyl)-3H-imidazo[4,5- b]pyridine-5-carboxylic acid |
| 67 | 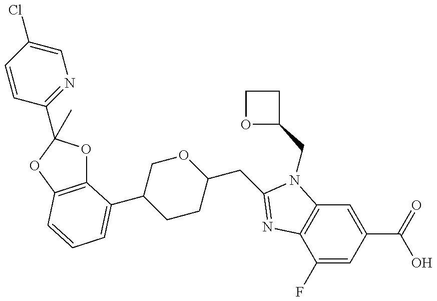 [SMILES: CC1(c2ccc(Cl)cn2)Oc2cccc(C3CCC(Cc4nc5c(F)cc(C(=O)O)cc5n4C[C@@H]4CCO4)OC3)c2O1] | 2-((5-(2-(5-chloropyridin-2-yl)-2- methylbenzo[d][1,3]dioxol-4- yl)tetrahydro-2H-pyran-2- yl)methyl)-4-fluoro-1-(((S)- oxetan-2-yl)methyl)-1H- benzo[d]imidazole-6-carboxylic acid |
| 68 | 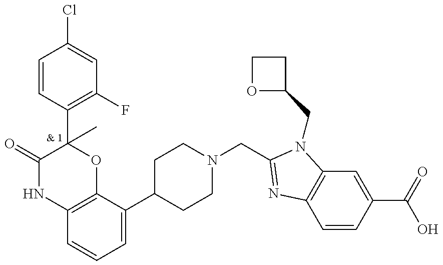 [SMILES: CC1(c2ccc(Cl)cc2F)Oc2c(cccc2C2CCN(Cc3nc4ccc(C(=O)O)cc4n3C[C@@H]3CCO3)CC2)NC1=O] | 2-((4-(2-(4-chloro-2- fluorophenyl)-2-methyl-3-oxo- 3,4-dihydro-2H- benzo[b][1,4]oxazin-8- yl)piperidin-1-yl)methyl)-1-(((S)- oxetan-2-yl)methyl)-1H- benzo[d]imidazole-6-carboxylic acid |
| 69 | 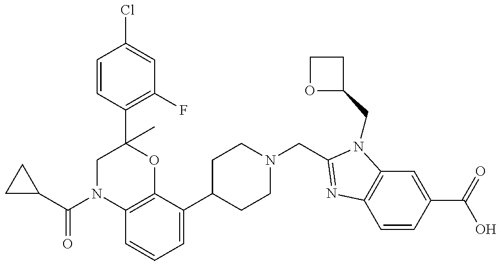 [SMILES: CC1(c2ccc(Cl)cc2F)CN(C(=O)C2CC2)c2cccc(C3CCN(Cc4nc5ccc(C(=O)O)cc5n4C[C@@H]4CCO4)CC3)c2O1] | 2-((4-(2-(4-chloro-2- fluorophenyl)-4- (cyclopropanecarbonyl)-2- methyl-3,4-dihydro-2H- benzo[b][1,4]oxazin-8- yl)piperidin-1-yl)methyl)-1-(((S)- oxetan-2-yl)methyl)-1H- benzo[d]imidazole-6-carboxylic acid |
| 70 | 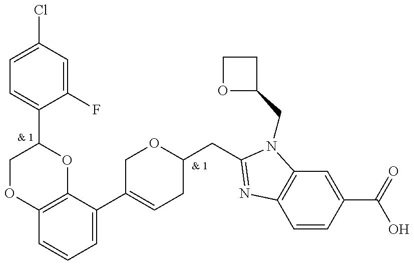 [SMILES: O=C(O)c1ccc2nc(CC3CC=C(c4cccc5c4OC(c4ccc(Cl)cc4F)CO5)CO3)n(C[C@@H]3CCO3)c2c1] | 2-((5-(3-(4-chloro-2- fluorophenyl)-2,3- dihydrobenzo[b][1,4]dioxin-5- yl)-3,6-dihydro-2H-pyran-2- yl)methyl)-1-(((S)-oxetan-2- yl)methyl)-1H- benzo[d]imidazole-6-carboxylic acid |
| 71 | 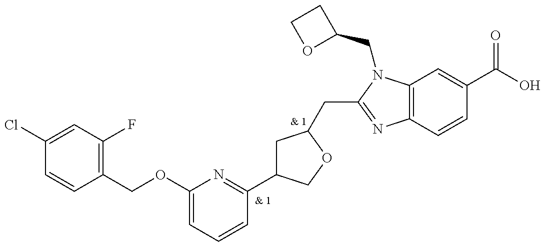 [SMILES: O=C(O)c1ccc2nc(CC3CC(c4cccc(OCc5ccc(Cl)cc5F)n4)CO3)n(C[C@@H]3CCO3)c2c1] | 2-((4-(6-((4-chloro-2- fluorobenzyl)oxy)pyridin-2- yl)tetrahydrofuran-2-yl)methyl)- 1-(((S)-oxetan-2-yl)methyl)-1H- benzo[d]imidazole-6-carboxylic acid |
| 72 | 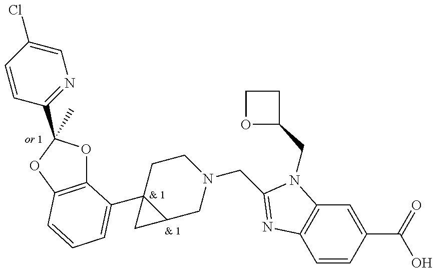 [SMILES: C[C@@]1(c2ccc(Cl)cn2)Oc2cccc(C34CCN(Cc5nc6ccc(C(=O)O)cc6n5C[C@@H]5CCO5)CC3C4)c2O1] | 2-((6-((S)-2-(5-chloropyridin-2- yl)-2-methylbenzo[d][1,3]dioxol- 4-yl)-3-azabicyclo[4.1.0]heptan- 3-yl)methyl)-1-(((S)-oxetan-2- yl)methyl)-1H- benzo[d]imidazole-6-carboxylic acid |
| 73 | 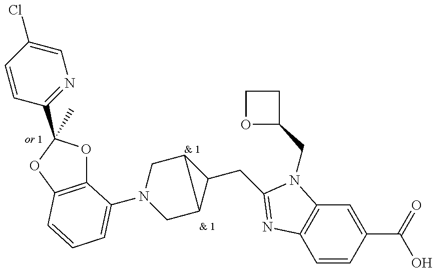 [SMILES: C[C@@]1(c2ccc(Cl)cn2)Oc2cccc(N3CC4C(Cc5nc6ccc(C(=O)O)cc6n5C[C@@H]5CCO5)C4C3)c2O1] | 2-((3-((R)-2-(5-chloropyridin-2- yl)-2-methylbenzo[d][1,3]dioxol- 4-yl)-3-azabicyclo[3.1.0]hexan-6- yl)methyl)-1-(((S)-oxetan-2- yl)methyl)-1H- benzo[d]imidazole-6-carboxylic acid |
| 74 | 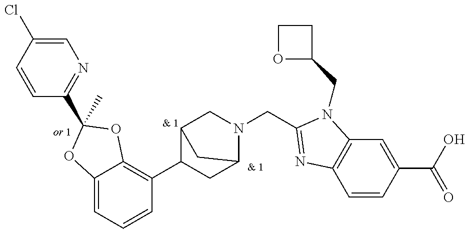 [SMILES: C[C@@]1(c2ccc(Cl)cn2)Oc2cccc(C3CC4CC3CN4Cc3nc4ccc(C(=O)O)cc4n3C[C@@H]3CCO3)c2O1] | 2-((5-((R)-2-(5-chloropyridin-2- yl)-2-methylbenzo[d][1,3]dioxol- 4-yl)-2-azabicyclo[2.2.1]heptan- 2-yl)methyl)-1-(((S)-oxetan-2- yl)methyl)-1H- benzo[d]imidazole-6-carboxylic acid |
| 75 | 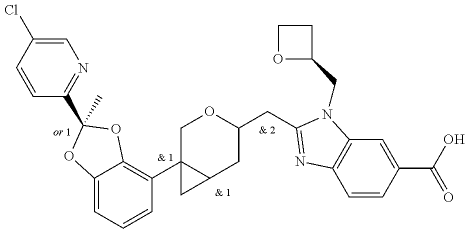 [SMILES: C[C@@]1(c2ccc(Cl)cn2)Oc2cccc(C34COC(Cc5nc6ccc(C(=O)O)cc6n5C[C@@H]5CCO5)CC3C4)c2O1] | 2-((1-((R)-2-(5-chloropyridin-2- yl)-2-methylbenzo[d][1,3]dioxol- 4-yl)-3-oxabicyclo[4.1.0]heptan- 4-yl)methyl)-1-(((S)-oxetan-2- yl)methyl)-1H- benzo[d]imidazole-6-carboxylic acid |
| 76 | 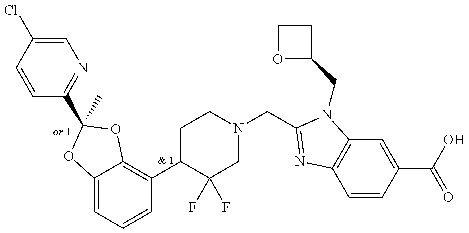 [SMILES: C[C@@]1(c2ccc(Cl)cn2)Oc2cccc(C3CCN(Cc4nc5ccc(C(=O)O)cc5n4C[C@@H]4CCO4)CC3(F)F)c2O1] | 2-((4-((R)-2-(5-chloropyridin-2- yl)-2-methylbenzo[d][1,3]dioxol- 4-yl)-3,3-difluoropiperidin-1- yl)methyl)-1-(((S)-oxetan-2- yl)methyl)-1H- benzo[d]imidazole-6-carboxylic acid |
| 77 | 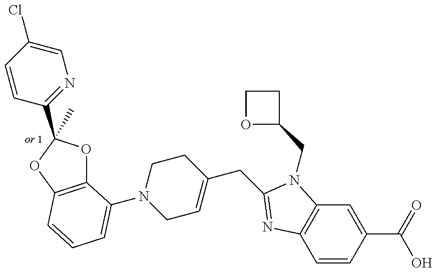 [SMILES: C[C@@]1(c2ccc(Cl)cn2)Oc2cccc(N3CC=C(Cc4nc5ccc(C(=O)O)cc5n4C[C@@H]4CCO4)CC3)c2O1] | 2-((1-((R)-2-(5-chloropyridin-2- yl)-2-methylbenzo[d][1,3]dioxol- 4-yl)-1,2,3,6-tetrahydropyridin-4- yl)methyl)-1-(((S)-oxetan-2- yl)methyl)-1H- benzo[d]imidazole-6-carboxylic acid |
| 78 | 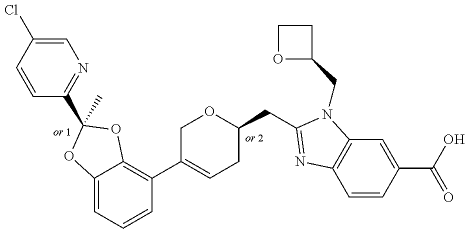 [SMILES: C[C@@]1(c2ccc(Cl)cn2)Oc2cccc(C3=CC[C@H](Cc4nc5ccc(C(=O)O)cc5n4C[C@@H]4CCO4)OC3)c2O1] | 2-(((R)-5-((R)-2-(5- chloropyridin-2-yl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 79 | 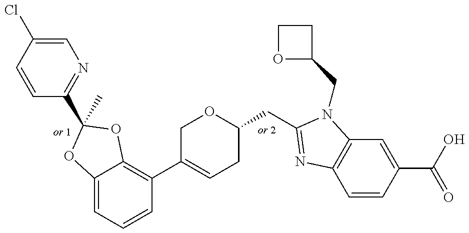 [SMILES: C[C@@]1(c2ccc(Cl)cn2)Oc2cccc(C3=CC[C@@H](Cc4nc5ccc(C(=O)O)cc5n4C[C@@H]4CCO4)OC3)c2O1] | 2-(((S)-5-((R)-2-(5-chloropyridin- 2-yl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 80 | 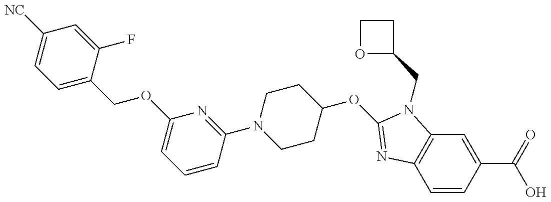 [SMILES: N#Cc1ccc(COc2cccc(N3CCC(Oc4nc5ccc(C(=O)O)cc5n4C[C@@H]4CCO4)CC3)n2)c(F)c1] | (S)-2-((1-(6-((4-cyano-2- fluorobenzyl)oxy)pyridin-2- yl)piperidin-4-yl)oxy)-1-(oxetan- 2-ylmethyl)-1H- benzo[d]imidazole-6-carboxylic acid |
| 81 | 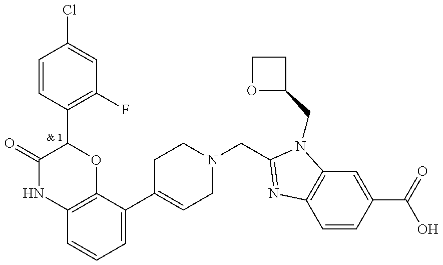 [SMILES: O=C(O)c1ccc2nc(CN3CC=C(c4cccc5c4OC(c4ccc(Cl)cc4F)C(=O)N5)CC3)n(C[C@@H]3CCO3)c2c1] | 2-((4-(2-(4-chloro-2- fluorophenyl)-3-oxo-3,4-dihydro- 2H-benzo[b][1,4]oxazin-8-yl)- 3,6-dihydropyridin-1(2H)- yl)methyl)-1-(((S)-oxetan-2- yl)methyl)-1H- benzo[d]imidazole-6-carboxylic acid |
| 82 | 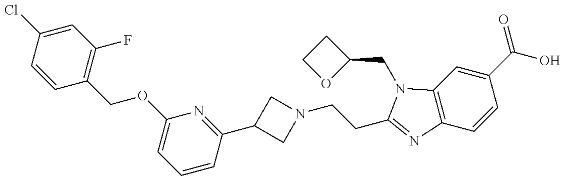 [SMILES: O=C(O)c1ccc2nc(CCN3CC(c4cccc(OCc5ccc(Cl)cc5F)n4)C3)n(C[C@@H]3CCO3)c2c1] | (S)-2-(2-(3-(6-((4-chloro-2- fluorobenzyl)oxy)pyridin-2- yl)azetidin-1-yl)ethyl)-1-(oxetan- 2-ylmethyl)-1H- benzo[d]imidazole-6-carboxylic acid |
| 83 | 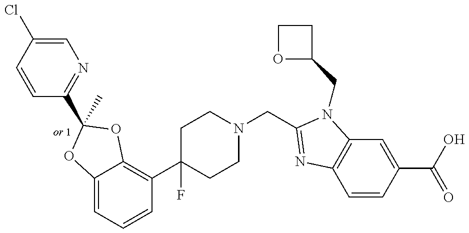 [SMILES: C[C@@]1(c2ccc(Cl)cn2)Oc2cccc(C3(F)CCN(Cc4nc5ccc(C(=O)O)cc5n4C[C@@H]4CCO4)CC3)c2O1] | 2-((4-((R)-2-(5-chloropyridin-2- yl)-2-methylbenzo[d][1,3]dioxol- 4-yl)-4-fluoropiperidin-1- yl)methyl)-1-(((S)-oxetan-2- yl)methyl)-1H- benzo[d]imidazole-6-carboxylic acid |
| 84 | 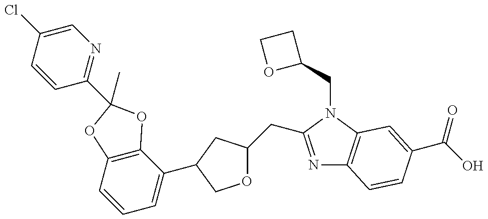 [SMILES: CC1(c2ccc(Cl)cn2)Oc2cccc(C3COC(Cc4nc5ccc(C(=O)O)cc5n4C[C@@H]4CCO4)C3)c2O1] | 2-((4-(2-(5-chloropyridin-2-yl)-2- methylbenzo[d][1,3]dioxol-4- yl)tetrahydrofuran-2-yl)methyl)- 1-(((S)-oxetan-2-yl)methyl)-1H- benzo[d]imidazole-6-carboxylic acid |
| 85 | 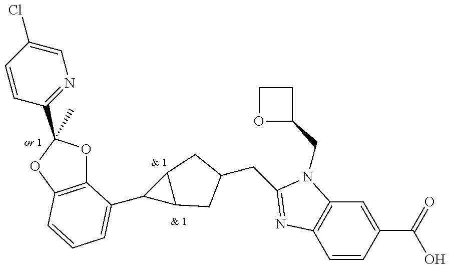 [SMILES: C[C@@]1(c2ccc(Cl)cn2)Oc2cccc(C3C4CC(Cc5nc6ccc(C(=O)O)cc6n5C[C@@H]5CCO5)CC43)c2O1] | 2-((6-((R)-2-(5-chloropyridin-2- yl)-2-methylbenzo[d][1,3]dioxol- 4-yl)-3-azabicyclo[3.1.0]hexan-3- yl)methyl)-1-(((S)-oxetan-2- yl)methyl)-1H- benzo[d]imidazole-6-carboxylic acid |
| 86 | 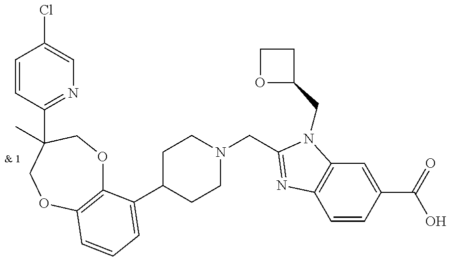 [SMILES: CC1(c2ccc(Cl)cn2)COc2cccc(C3CCN(Cc4nc5ccc(C(=O)O)cc5n4C[C@@H]4CCO4)CC3)c2OC1] | 2-((4-(3-(5-chloropyridin-2-yl)-3- methyl-3,4-dihydro-2H- benzo[b][1,4]dioxepin-6- yl)piperidin-1-yl)methyl)-1-(((S)- oxetan-2-yl)methyl)-1H- benzo[d]imidazole-6-carboxylic acid |
| 87 | 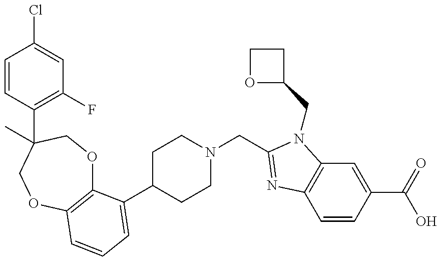 [SMILES: CC1(c2ccc(Cl)cc2F)COc2cccc(C3CCN(Cc4nc5ccc(C(=O)O)cc5n4C[C@@H]4CCO4)CC3)c2OC1] | 2-((4-(3-(4-chloro-2- fluorophenyl)-3-methyl-3,4- dihydro-2H- benzo[b][1,4]dioxepin-6- yl)piperidin-1-yl)methyl)-1-(((S)- oxetan-2-yl)methyl)-1H- benzo[d]imidazole-6-carboxylic acid |
| 88 | 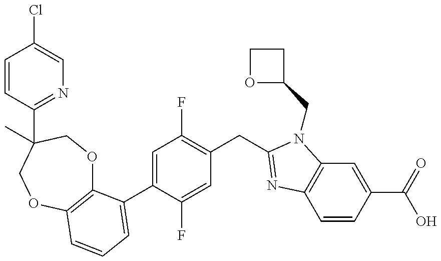 [SMILES: CC1(c2ccc(Cl)cn2)COc2cccc(-c3cc(F)c(Cc4nc5ccc(C(=O)O)cc5n4C[C@@H]4CCO4)cc3F)c2OC1] | 2-(4-(3-(5-chloropyridin-2-yl)-3- methyl-3,4-dihydro-2H- benzo[b][1,4]dioxepin-6-yl)-2,5- difluorobenzyl)-1-(((S)-oxetan-2- yl)methyl)-1H- benzo[d]imidazole-6-carboxylic acid |
| 89 | 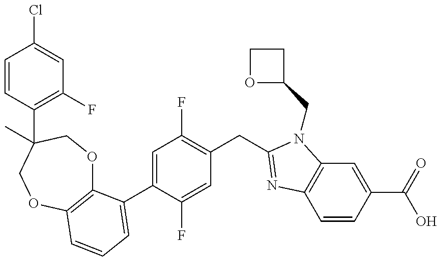 [SMILES: CC1(c2ccc(Cl)cc2F)COc2cccc(-c3cc(F)c(Cc4nc5ccc(C(=O)O)cc5n4C[C@@H]4CCO4)cc3F)c2OC1] | 2-(4-(3-(4-chloro-2- fluorophenyl)-3-methyl-3,4- dihydro-2H- benzo[b][1,4]dioxepin-6-yl)-2,5- difluorobenzyl)-1-(((S)-oxetan-2- yl)methyl)-1H- benzo[d]imidazole-6-carboxylic acid |
| 90 | 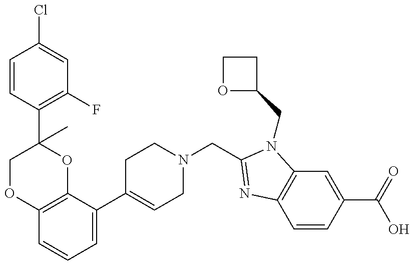 [SMILES: CC1(c2ccc(Cl)cc2F)COc2cccc(C3=CCN(Cc4nc5ccc(C(=O)O)cc5n4C[C@@H]4CCO4)CC3)c2O1] | 2-((4-(3-(4-chloro-2- fluorophenyl)-3-methyl-2,3- dihydrobenzo[b][1,4]dioxin-5- yl)-3,6-dihydropyridin-1(2H)- yl)methyl)-1-(((S)-oxetan-2- yl)methyl)-1H- benzo[d]imidazole-6-carboxylic acid |
| 91 | 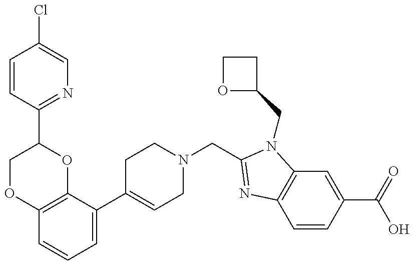 [SMILES: O=C(O)c1ccc2nc(CN3CC=C(c4cccc5c4OC(c4ccc(Cl)cn4)CO5)CC3)n(C[C@@H]3CCO3)c2c1] | 2-((4-(3-(5-chloropyridin-2-yl)- 2,3-dihydrobenzo[b][1,4]dioxin- 5-yl)-3,6-dihydropyridin-1(2H)- yl)methyl)-1-(((S)-oxetan-2- yl)methyl)-1H- benzo[d]imidazole-6-carboxylic acid |
| 92 | 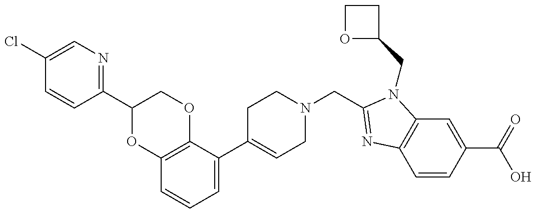 [SMILES: O=C(O)c1ccc2nc(CN3CC=C(c4cccc5c4OCC(c4ccc(Cl)cn4)O5)CC3)n(C[C@@H]3CCO3)c2c1] | 2-((4-(2-(5-chloropyridin-2-yl)- 2,3-dihydrobenzo[b][1,4]dioxin- 5-yl)-3,6-dihydroypridin-1(2H)- yl)methyl)-1-(((S)-oxetan-2- yl)methyl)-1H- benzo[d]imidazole-6-carboxylic acid |
| 93 | 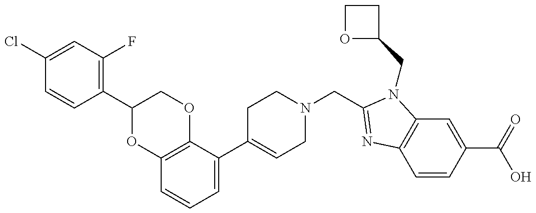 [SMILES: O=C(O)c1ccc2nc(CN3CC=C(c4cccc5c4OCC(c4ccc(Cl)cc4F)O5)CC3)n(C[C@@H]3CCO3)c2c1] | 2-((4-(2-(4-chloro-2- fluorophenyl)-2,3- dihydrobenzo[b][1,4]dioxin-5- yl)-3,6-dihydropyridin-1(2H)- yl)methyl)-1-(((S)-oxetan-2- yl)methyl)-1H- benzo[d]imidazole-6-carboxylic acid |
| 94 | 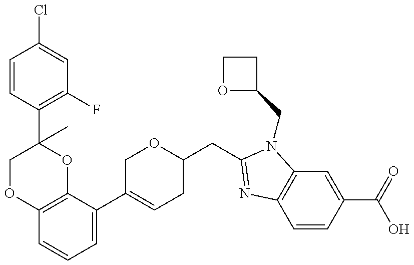 [SMILES: CC1(c2ccc(Cl)cc2F)COc2cccc(C3=CCC(Cc4nc5ccc(C(=O)O)cc5n4C[C@@H]4CCO4)OC3)c2O1] | 2-((5-(3-(4-chloro-2- fluorophenyl)-3-methyl-2,3- dihydrobenzo[b][1,4]dioxin-5- yl)-3,6-dihydro-2H-pyran-2- yl)methyl)-1-(((S)-oxetan-2- yl)methyl)-1H- benzo[d]imidazole-6-carboxylic acid |
| 95 | 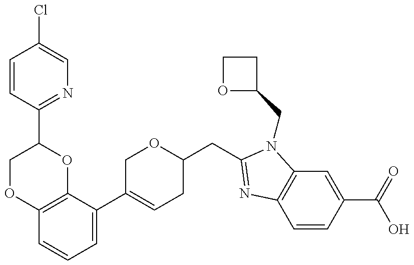 [SMILES: O=C(O)c1ccc2nc(CC3CC=C(c4cccc5c4OC(c4ccc(Cl)cn4)CO5)CO3)n(C[C@@H]3CCO3)c2c1] | 2-((5-(3-(5-chloropyridin-2-yl)- 2,3-dihydrobenzo[b][1,4]dioxin- 5-yl)-3,6-dihydro-2H-pyran-2- yl)methyl)-1-(((S)-oxetan-2- yl)methyl)-1H- benzo[d]imidazole-6-carboxylic acid |
| 96 | 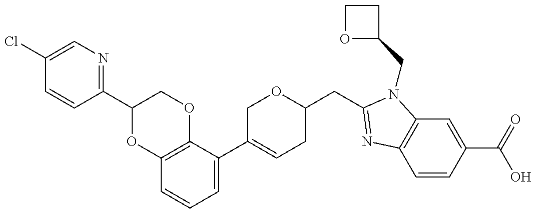 [SMILES: O=C(O)c1ccc2nc(CC3CC=C(c4cccc5c4OCC(c4ccc(Cl)cn4)O5)CO3)n(C[C@@H]3CCO3)c2c1] | 2-((5-(2-(5-chloropyridin-2-yl)- 2,3-dihydrobenzo[b][1,4]dioxin- 5-yl)-3,6-dihydro-2H-pyran-2- yl)methyl)-1-(((S)-oxetan-2- yl)methyl)-1H- benzo[d]imidazole-6-carboxylic acid |
| 97 | 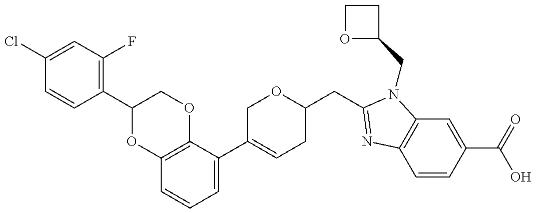 [SMILES: O=C(O)c1ccc2nc(CC3CC=C(c4cccc5c4OCC(c4ccc(Cl)cc4F)O5)CO3)n(C[C@@H]3CCO3)c2c1] | 2-((5-(2-(4-chloro-2- fluorophenyl)-2,3- dihydrobenzo[b][1,4]dioxin-5- yl)-3,6-dihydro-2H-pyran-2- yl)methyl)-1-((S)-oxetan-2- yl)methyl)-1H- benzo[d]imidazole-6-carboxylic acid |
| 98 | 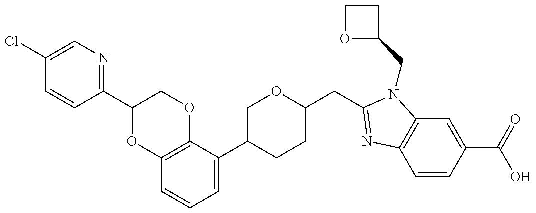 [SMILES: O=C(O)c1ccc2nc(CC3CCC(c4cccc5c4OCC(c4ccc(Cl)cn4)O5)CO3)n(C[C@@H]3CCO3)c2c1] | 2-((5-(2-(5-chloropyridin-2-yl)- 2,3-dihydrobenzo[b][1,4]dioxin- 5-yl)tetrahydro-2H-pyran-2- yl)methyl)-1-(((S)-oxetan-2- yl)methyl)-1H- benzo[d]imidazole-6-carboxylic acid |
| 99 | 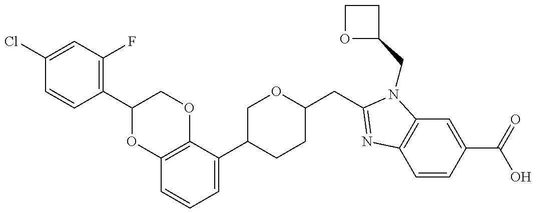 [SMILES: O=C(O)c1ccc2nc(CC3CCC(c4cccc5c4OCC(c4ccc(Cl)cc4F)O5)CO3)n(C[C@@H]3CCO3)c2c1] | 2-((5-(2-(4-chloro-2- fluorophenyl)-2,3- dihydrobenzo[b][1,4]dioxin-5- yl)tetrahydro-2H-pyran-2- yl)methyl)-1-(((S)-oxetan-2- yl)methyl)-1H- benzo[d]imidazole-6-carboxylic acid |
| 100 | 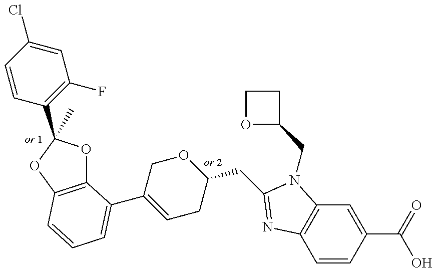 [SMILES: C[C@@]1(c2ccc(Cl)cc2F)Oc2cccc(C3=CC[C@@H](Cc4nc5ccc(C(=O)O)cc5n4C[C@@H]4CCO4)OC3)c2O1] | 2-(((S)-5-((R)-2-(4-chloro-2- fluorophenyl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 101 | 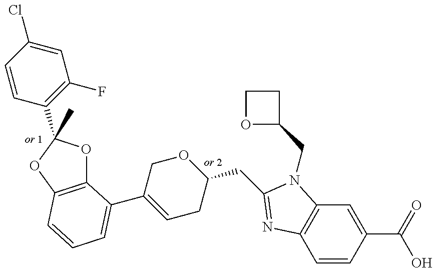 [SMILES: C[C@]1(c2ccc(Cl)cc2F)Oc2cccc(C3=CC[C@@H](Cc4nc5ccc(C(=O)O)cc5n4C[C@@H]4CCO4)OC3)c2O1] | 2-(((S)-5-((S)-2-(4-chloro-2- fluorophenyl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 102 | 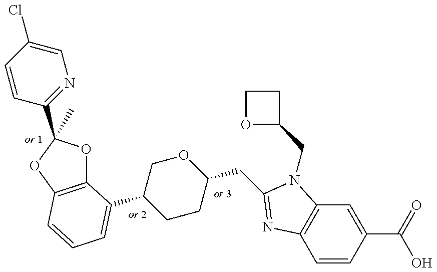 [SMILES: C[C@@]1(c2ccc(Cl)cn2)Oc2cccc([C@H]3CC[C@@H](Cc4nc5ccc(C(=O)O)cc5n4C[C@@H]4CCO4)OC3)c2O1] | 2-(((2S,5R)-5-((R)-2-(5- chloropyridin-2-yl)-2- methylbenzo[d][1,3]dioxol-4- yl)tetrahydro-2H-pyran-2- yl)methyl)-1-(((S)-oxetna-2- yl)methyl)-1H- benzo[d]imidazole-6-carboxylic acid |
| 103 | 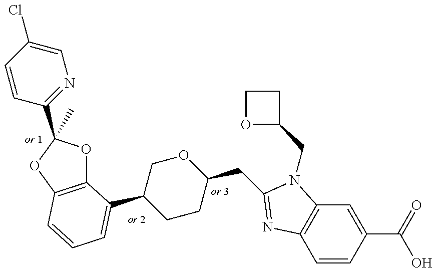 [SMILES: C[C@@]1(c2ccc(Cl)cn2)Oc2cccc([C@@H]3CC[C@H](Cc4nc5ccc(C(=O)O)cc5n4C[C@@H]4CCO4)OC3)c2O1] | 2-(((2R,5S)-5-((R)-2-(5- chloropyridin-2-yl)-2- methylbenzo[d][1,3]dioxol-4- yl)tetrahydro-2H-pyran-2- yl)methyl)-1-(((S)-oxetan-2- yl)methyl)-1H- benzo[d]imidazole-6-carboxylic acid |
| 104 | 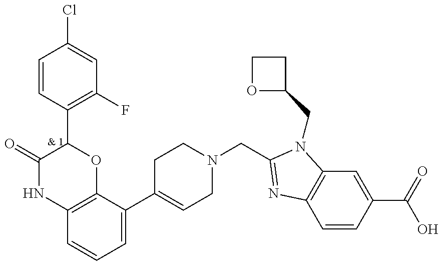 [SMILES: O=C(O)c1ccc2nc(CN3CC=C(c4cccc5c4OC(c4ccc(Cl)cc4F)C(=O)N5)CC3)n(C[C@@H]3CCO3)c2c1] | 2-((4-(2-(4-chloro-2- fluorophenyl)-3-oxo-3,4-dihydro- 2H-benzo[b][1,4]oxazin-8- yl)piperidin-1-yl)methyl)-1-(((S)- oxetan-2-yl)methyl)-1H- benzo[d]imidazole-6-carboxylic acid |
| 105 | 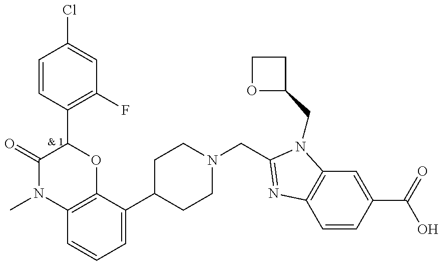 [SMILES: CN1C(=O)C(c2ccc(Cl)cc2F)Oc2c(C3CCN(Cc4nc5ccc(C(=O)O)cc5n4C[C@@H]4CCO4)CC3)cccc21] | 2-((4-(2-(4-chloro-2- fluorophenyl)-4-methyl-3-oxo- 3,4-dihydro-2H- benzo[b][1,4]oxazin-8- yl)piperidin-1-yl)methyl)-1-(((S)- oxetan-2-yl)methyl)-1H- benzo[d]imidazole-6-carboxylic acid |
| 106 | 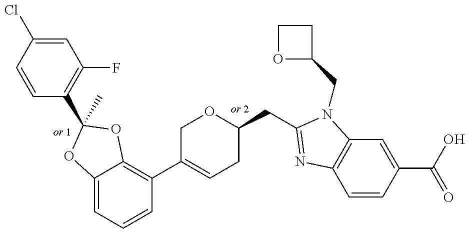 [SMILES: C[C@@]1(c2ccc(Cl)cc2F)Oc2cccc(C3=CC[C@H](Cc4nc5ccc(C(=O)O)cc5n4C[C@@H]4CCO4)OC3)c2O1] | 2-(((R)-5-((R)-2-(4-chloro-2- fluorophenyl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 107 | 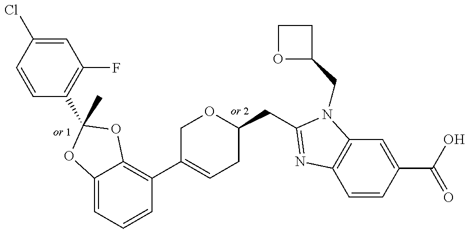 [SMILES: C[C@]1(c2ccc(Cl)cc2F)Oc2cccc(C3=CC[C@H](Cc4nc5ccc(C(=O)O)cc5n4C[C@@H]4CCO4)OC3)c2O1] | 2-(((R)-5-((S)-2-(4-chloro-2- fluorophenyl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 108 | 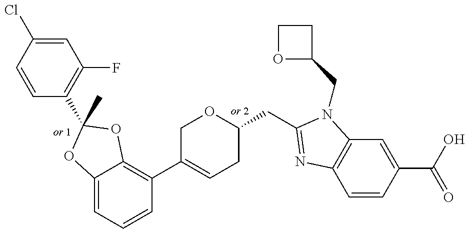 [SMILES: C[C@]1(c2ccc(Cl)cc2F)Oc2cccc(C3=CC[C@@H](Cc4nc5ccc(C(=O)O)cc5n4C[C@@H]4CCO4)OC3)c2O1] | 2-(((S)-5-((S)-2-(4-chloro-2- fluorophenyl)-2- methylbenzo[d][1,3]dioxol-4-yl)- 3,6-dihydro-2H-pyran-2- yl)methyl)-3-(((S)-oxetan-2- yl)methyl)-3H-imidazo[4,5- b]pyridine-5-carboxylic acid |
| 109 | 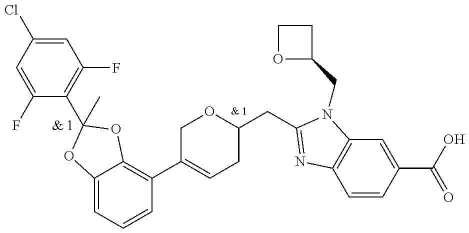 [SMILES: CC1(c2c(F)cc(Cl)cc2F)Oc2cccc(C3=CCC(Cc4nc5ccc(C(=O)O)cc5n4C[C@@H]4CCO4)OC3)c2O1] | 2-((5-(2-(4-chloro-2,6- difluorophenyl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 110 | 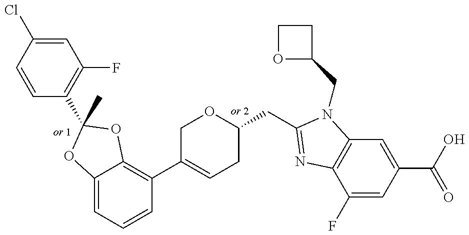 [SMILES: C[C@]1(c2ccc(Cl)cc2F)Oc2cccc(C3=CC[C@@H](Cc4nc5c(F)cc(C(=O)O)cc5n4C[C@@H]4CCO4)OC3)c2O1] | 2-(((S)-5-((S)-2-(4-chloro-2- fluorophenyl)-2- methylbenzo[d][1,3]dioxol-4-yl)- 3,6-dihydro-2H-pyran-2- yl)methyl)-4-fluoro-1-(((S)- oxetan-2-yl)methyl)-1H- benzo[d]imidazole-6-carboxylic acid |
| 111 | 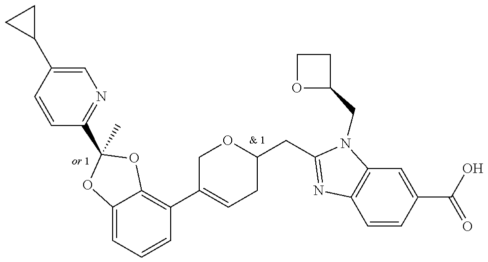 [SMILES: C[C@@]1(c2ccc(C3CC3)cn2)Oc2cccc(C3=CCC(Cc4nc5ccc(C(=O)O)cc5n4C[C@@H]4CCO4)OC3)c2O1] | 2-((5-((R)-2-(5- cyclopropylpyridin-2-yl)-2- methylbenzo[d][1,3]dioxol-4-yl)- 3,6-dihydro-2H-pyran-2- yl)methyl)-1-(((S)-oxetan-2- yl)methyl)-1H- benzo[d]imidazole-6-carboxylic acid |
| 112 | 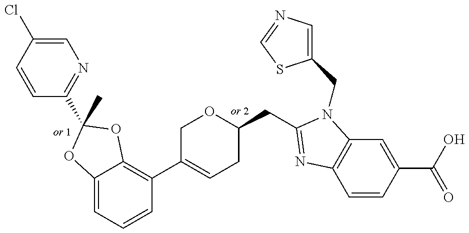 | 2-(((R)-5-((S)-2-(5-chloropyridin- 2-yl)-2- methylbenzo[d][1,3]dioxol-4-yl)- 3,6-dihydro-2H-pyran-2- yl)methyl)-1-(thiazol-5- ylmethyl)-1H-benzo[d]imidazole- 6-carboxylic acid |
| 113 | 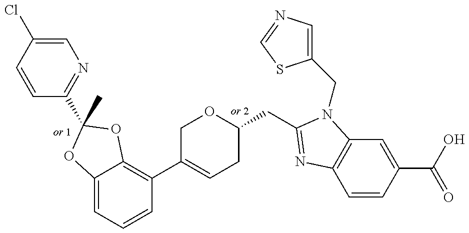 [SMILES: C[C@]1(c2ccc(Cl)cn2)Oc2cccc(C3=CC[C@@H](Cc4nc5ccc(C(=O)O)cc5n4Cc4cncs4)OC3)c2O1] | 2-(((S)-5-((S)-2-(5-chloropyridin- 2-yl)-2- methylbenzo[d][1,3]dioxol-4-yl)- 3,6-dihydro-2H-pyran-2- yl)methyl)-1-(thiazol-5- ylmethyl)-1H-benzo[d]imidazole- 6-carboxylic acid |
| 114 | 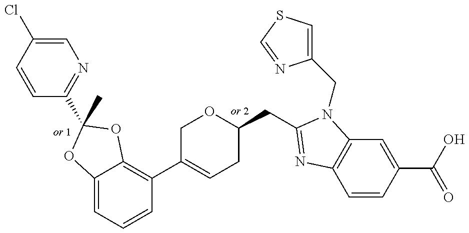 [SMILES: C[C@]1(c2ccc(Cl)cn2)Oc2cccc(C3=CC[C@H](Cc4nc5ccc(C(=O)O)cc5n4Cc4cscn4)OC3)c2O1] | 2-(((R)-5-((S)-2-(5-chloropyridin- 2-yl)-2- methylbenzo[d][1,3]dioxol-4-yl)- 3,6-dihydro-2H-pyran-2- yl)methyl)-1-(thiazol-4- ylmethyl)-1H-benzo[d]imidazole- 6-carboxylic acid |
| 115 | 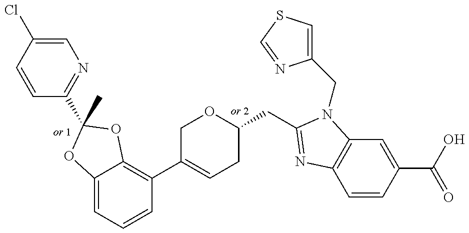 [SMILES: C[C@]1(c2ccc(Cl)cn2)Oc2cccc(C3=CC[C@@H](Cc4nc5ccc(C(=O)O)cc5n4Cc4cscn4)OC3)c2O1] | 2-(((S)-5-((S)-2-(5-chloropyridin- 2-yl)-2- methylbenzo[d][1,3]dioxol-4-yl)- 3,6-dihydro-2H-pyran-2- yl)methyl)-1-(thiazol-4- ylmethyl)-1H-benzo[d]imidazole- 6-carboxylc acid |
| 116 | 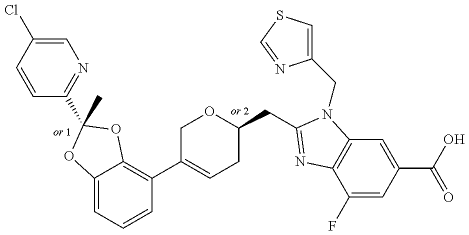 [SMILES: C[C@]1(c2ccc(Cl)cn2)Oc2cccc(C3=CC[C@H](Cc4nc5c(F)cc(C(=O)O)cc5n4Cc4cscn4)OC3)c2O1] | 2-(((R)-5-((S)-2-(5-chloropyridin- 2-yl)-2- methylbenzo[d][1,3]dioxol-4-yl)- 3,6-dihydro-2H-pyran-2- yl)methyl)-4-fluoro-1-(thiazol-4- ylmethyl)-1H-benzo[d]imidazole- 6-carboxylic acid |
| 117 | 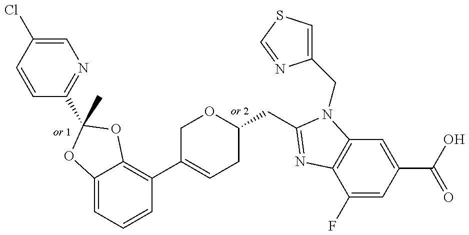 [SMILES: C[C@]1(c2ccc(Cl)cn2)Oc2cccc(C3=CC[C@@H](Cc4nc5c(F)cc(C(=O)O)cc5n4Cc4cscn4)OC3)c2O1] | 2-(((S)-5-((S)-2-(5-chloropyridin- 2-yl)-2- methylbenzo[d][1,3]dioxol-4-yl)- 3,6-dihydro-2H-pyran-2- y)methyl)-4-fluoro-1-(thiazol-4- ylmethyl)-1H-benzo[d]imidazole- 6-carboxylic acid |
| 118 | 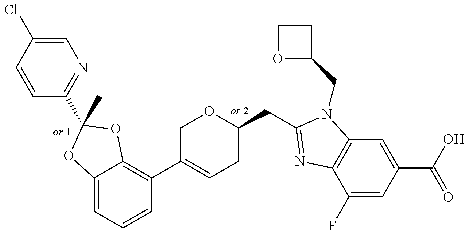 [SMILES: C[C@]1(c2ccc(Cl)cn2)Oc2cccc(C3=CC[C@H](Cc4nc5c(F)cc(C(=O)O)cc5n4C[C@@H]4CCO4)OC3)c2O1] | 2-(((R)-5-((S)-2-(5-chloropyridin- 2-yl)-2- methylbenzo[d][1,3]dioxol-4-yl)- 3,6-dihydro-2H-pyran-2- yl)methyl)-4-fluoro-1-(((S)- oxetan-2-yl)methyl)-1H- benzo[d]imdiazole-6-carboxylic acid |
| 119 | 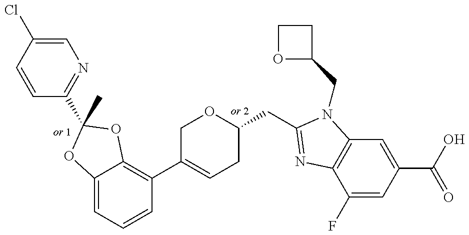 [SMILES: C[C@]1(c2ccc(Cl)cn2)Oc2cccc(C3=CC[C@@H](Cc4nc5c(F)cc(C(=O)O)cc5n4C[C@@H]4CCO4)OC3)c2O1] | 2-(((S)-5-((S)-2-(5-chloropyridin- 2-yl)-2- methylbenzo[d][1,3]dioxol-4-yl)- 3,6-dihydro-2H-pyran-2- yl)methyl)-4-fluoro-1-(((S)- oxetan-2-yl)methyl)-1H- benzo[d]imidazole-6-carboxylic acid |
| 120 | 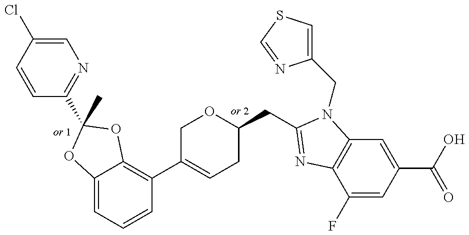 [SMILES: C[C@]1(c2ccc(Cl)cn2)Oc2cccc(C3=CC[C@H](Cc4nc5c(F)cc(C(=O)O)cc5n4Cc4cscn4)OC3)c2O1] | 2-(((R)-5-((S)-2-(5-chloropyridin- 2-yl)-2- methylbenzo[d][1,3]dioxol-4-yl)- 3,6-dihydro-2H-pyran-2- yl)methyl)-4-fluoro-1-(thiazol-5- ylmethyl)-1H-benzo[d]imidazole- 6-carboxylic acid |
| 121 | 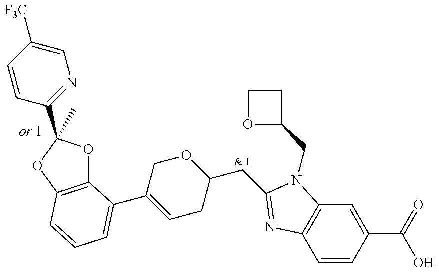 [SMILES: C[C@@]1(c2ccc(C(F)(F)F)cn2)Oc2cccc(C3=CCC(Cc4nc5ccc(C(=O)O)cc5n4C[C@@H]4CCO4)OC3)c2O1] | 2-((5-((R)-2-methyl-2-(5- (trifluoromethyl)pyridin-2- yl)benzo[d][1,3]dioxol-4-yl)-3,6- dihydro-2H-pyran-2-yl)methyl)- 1-(((S)-oxetan-2-yl)methyl)-1H- benzo[d]imidazole-6-carboxylic acid |
| 122 | 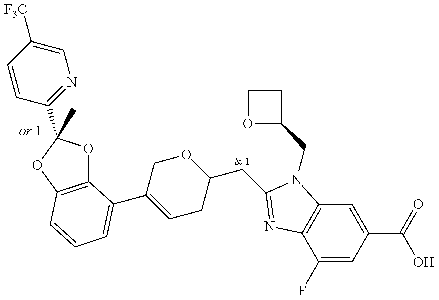 [SMILES: C[C@]1(c2ccc(C(F)(F)F)cn2)Oc2cccc(C3=CCC(Cc4nc5c(F)cc(C(=O)O)cc5n4C[C@@H]4CCO4)OC3)c2O1] | 4-fluoro-2-((5-((S)-2-methyl-2- (5-(trifluoromethyl)pyridin-2- yl)benzo[d][1,3]dioxol-4-yl)-3,6- dihydro-2H-pyran-2-yl)methyl)- 1-(((S)-oxetan-2-yl)methyl)-1H- benzo[d]imidazole-6-carboxylic acid |
| 123 | 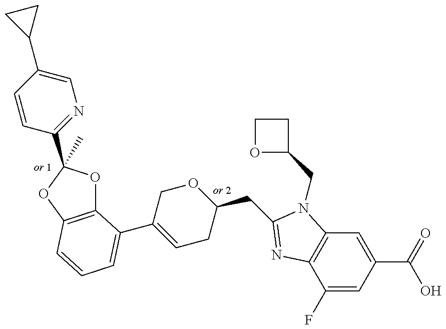 [SMILES: C[C@@]1(c2ccc(C3CC3)cn2)Oc2cccc(C3=CC[C@H](Cc4nc5c(F)cc(C(=O)O)cc5n4C[C@@H]4CCO4)OC3)c2O1] | 2-(((R)-5-((R)-2-(5- cyclopropylpyridin-2-yl)-2- methylbenzo[d][1,3]dioxol-4-yl)- 3,6-dihydro-2H-pyran-2- yl)methyl)-4-fluoro-1-(((S)- oxetan-2-yl)methyl)-1H- benzo[d]imidazole-6-carboxylic acid. |

or a pharmaceutically acceptable salt thereof.

102. A pharmaceutical composition comprising the compound of claim 1 or a pharmaceutically acceptable salt, solvate, prodrug, stereoisomer, or tautomer thereof, and a pharmaceutically acceptable diluent, carrier, or excipient.

103. A method of treating or preventing a GLP-1 receptor-mediated disease or disorder or of modulating GLP-1 receptor, comprising administering to a subject in need thereof a therapeutically effective amount of the compound of claim 1 or a pharmaceutically acceptable salt, solvate, prodrug, stereoisomer, or tautomer thereof.

104.-106. (canceled)
